(19) United States
(12) Patent Application Publication
(10) Pub. No.: US 2025/0319139 A1
(43) Pub. Date: Oct. 16, 2025

(54) METHODS FOR TREATING NEUROINFLAMMATION

(71) Applicant: University of Virginia Patent Foundation, Charlottesville, VA (US)

(72) Inventor: Zhiyi Zuo, Charlottesville, VA (US)

(73) Assignee: University of Virginia Patent Foundation, Charlottesville, VA (US)

(21) Appl. No.: 18/692,937

(22) PCT Filed: Sep. 19, 2022
(86) PCT No.: PCT/US2022/076664

§ 371 Date: Mar. 18, 2024

Related U.S. Application Data
(60) Provisional application No. 63/245,672, filed on Sep. 17, 2021.

(51) Int. Cl. A61K 35/74; A61K 31/397; A61P 25/28; A61P 29/00; C07K 16/24; C07K 16/28; C12N 15/113

(52) U.S. Cl. CPC A61K 35/74; A61K 31/397; A61P 25/28; A61P 29/00; C07K 16/244; C07K 16/28; C12N 15/1136; C12N 2310/14

(57) ABSTRACT

A method of treating neuroinflammation in a subject, the method including administering to a subject a therapy for modulating a valeric acid-interleukin (IL)-17 pathway in the subject so that neuroinflammation in the subject is treated. The therapy administered to the subject can include exercise, fecal transplantation, an agent to decrease bacteria producing valeric acid, an agent for reducing IL-17, and/or an agent for reducing FFAR2, C3ar1, C3, Iba-1, IL-1β, and/or IL-6. Such agents can include RNA interference constructs and/or antibodies. Also provided are methods for improving neurological outcome and/or mediating inflammatory response in a subject suffering from ischemic stroke and/or surgery-induced neuroinflammation.

### CROSS-REFERENCE TO RELATED APPLICATIONS

This application claims priority to and the benefit of U.S. Provisional Patent Application Ser. No. 63/245,672, filed Sep. 17, 2021, herein incorporated by reference in its entirety.

### GRANT STATEMENT

This invention was made with government support under Grant Nos. HD089999, AG061047, and NS099118 awarded by The National Institutes of Health. The government has certain rights in the invention

### REFERENCE TO SEQUENCE LISTING XML

The Sequence Listing XML associated with the instant disclosure has been electronically submitted to the United States Patent and Trademark Office via the Patent Center as a 126,848 byte UTF-8-encoded XML file created on Sep. 18, 2022 and entitled “Sequence_Listing_3062-169_PCT.xml”. The content of the Sequence Listing submitted via Patent Center is herein incorporated by reference in its entirety.

### TECHNICAL FIELD

Provided are methods of treating neurologic inflammation, neuroinflammation, and related conditions. More particularly, provided are methods and systems to attenuate gut dysbiosis and alter the valeric acid-IL-17 pathway to improved neurologic outcome and cognitive function related to neurologic inflammation in subjects.

### BACKGROUND

Postoperative cognitive dysfunction (POCD) affects the outcome of millions of patients each year. Aging is a risk factor for POCD. Currently the mechanisms for POCD are not fully understood and effective interventions to reduce POCD have not been identified.

Moreover, stroke is a leading cause of death and morbidity in the world and often occurs in elderly patients. Ironically, the outcome of ischemic stroke in elderly patients is worse than that in young patients. A similar situation has been shown in animal studies. Multiple factors may contribute to this phenomenon. For example, brain immune cells in old rodents may be in primed status and can have an exaggerated response to stimulation to produce proinflammatory cytokines that are known to worsen neurological outcome after brain ischemia.

Neuroinflammation has been implied in the development of POCD and associated with poor outcome after stroke. There is currently some belief that gut microbiota can impact the severity, susceptibility and/or outcome of neuroinflammation and/or neurological inflammatory conditions and events, including for example POCD and stroke. However, little is known about this purported relationship between gut microbiota and such conditions, the mechanics of the same, and/or the mechanism of action.

What is needed, therefore, are methods of treating, preventing and/or reducing neuroinflammation, including post-surgery neuroinflammation, and/or impairment of cognition and/or neuroplasticity, particularly by modulating the gut microbiota and related pathways.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this summary or not. To avoid excessive repetition, this Summary does not list or suggest all possible combinations of such features.

In some embodiments, provided herein are methods of treating neuroinflammation in a subject, the methods comprising administering to a subject a therapy for modulating a valeric acid-interleukin (IL)-17 pathway in the subject, whereby neuroinflammation in the subject is treated. In some embodiments, the neuroinflammation in the subject comprises post-surgery neuroinflammation, and/or impairment of cognition, and/or postoperative cognitive dysfunction (POCD), and/or neuroplasticity, optionally wherein the subject is a human subject. In some embodiments, the therapy administered to the subject comprises exercise, optionally wherein the exercise decreases gut microbiota changes in the subject and/or reduces valeric acid concentrations in the subject. In some embodiments, the therapy administered to the subject comprises fecal transplantation, optionally wherein the fecal transplantation decreases bacteria producing valeric acid, optionally wherein the fecal transplantation decreases Megasphaera massiliensis. In some embodiments, the therapy administered to the subject comprises an agent for reducing IL-17 in the subject, optionally wherein the agent comprises an antibody targeting IL-17, optionally wherein the antibody targets a polypeptide of IL-17 comprising an amino acid sequence of SEQ ID NO. 2.

In some embodiments, the therapy administered to the subject comprises a RNA interference (RNAi) construct targeting an IL-17 gene in the subject, optionally wherein the RNAi construct targets an IL-17 gene comprising a nucleic acid sequence of SEQ ID NO. 1. In some embodiments, the therapy administered to the subject interrupts the gut microbiota-valeric acid-free fatty acid receptor (FFAR) 2-IL-17 pathway, whereby the concentration of valeric acid and/or IL-17 in the subject is reduced. In some embodiments, the therapy administered to the subject comprises an agent to decrease and/or block FFAR2 in mediating an increase of IL-17 caused by valeric acid, optionally wherein the agent comprises a FFAR2 antagonist, optionally wherein the FFAR2 antagonist is GLPG-0974. In some embodiments, the therapy comprises an agent for reducing IL-17 downstream immune and neuroinflammatory targets selected from the group consisting of FFAR2, C3ar1, C3, Iba-1, IL-1β, IL-6, and combinations thereof, optionally wherein the agent comprises an antagonist and/or antibody against FFAR2, C3ar1, C3, Iba-1, IL-1β, and/or IL-6, optionally wherein the antibody targets a polypeptide of FFAR2 comprising an amino acid sequence of SEQ ID NO. 4, C3ar1 comprising an amino acid sequence of SEQ ID NO. 6, C3 comprising an amino acid sequence of SEQ ID NO. 8, Iba-1 comprising an amino acid sequence of SEQ ID NO. 10, IL-1B comprising an amino acid sequence of SEQ ID NO. 12, or IL-6 comprising an amino acid sequence of SEQ ID NO. 14. In some embodiments, the therapy administered to the subject comprises a RNA interference (RNAi) construct targeting an FFAR2, C3ar1, C3, Iba-1, IL-1β, and/or IL-6 gene in the subject, optionally wherein the RNAi construct targets a gene encoding FFAR2 comprising an nucleic acid sequence of SEQ ID NO. 3, C3ar1 comprising a nucleic acid sequence of SEQ ID NO. 5, C3 comprising a nucleic acid sequence of SEQ ID NO. 7, Iba-1 comprising a nucleic acid sequence of SEQ ID NO. 9, IL-1B comprising a nucleic acid sequence of SEQ ID NO. 11, or IL-6 comprising a nucleic acid sequence of SEQ ID NO. 13. In some embodiments, such methods can further comprise measuring blood valeric acid concentration in the subject prior to, during and/or after administration of the therapy.

Provided herein in some embodiments is a method for improving neurological outcome and/or mediating inflammatory response in a subject suffering from ischemic stroke and/or surgery-induced neuroinflammation, the method comprising: providing a subject suffering from ischemic stroke and/or surgery-induced neuroinflammation; and administering to the subject a therapy for reducing valeric acid in a subject, the therapy comprising: a) an agent for modulating, including antagonizing and/or disrupting, the valeric acid interleukin (IL)-17 pathway in the subject, including a) the gut microbiota-valeric acid-free fatty acid receptor (FFAR) 2-IL-17 pathway, b) exercise, c) fecal transplantation and/or d) an agent to decrease bacteria producing valeric acid, whereby the neurological outcome in the subject is improved and/or the inflammatory response in the subject is reduced. In some embodiments, the exercise decreases gut microbiota changes in the subject and/or reduces valeric acid concentrations in the subject. In some embodiments, the fecal transplantation decreases bacteria producing valeric acid, optionally wherein the fecal transplantation decreases Megasphaera massiliensis to reduce valeric acid. In some embodiments, such a method can further comprise measuring blood valeric acid concentration in the subject prior to, during and/or after administration of the therapy. In some embodiments, such a method can further comprise modulating a valeric acid interleukin (IL)-17 pathway in the subject.

Also provided herein are uses of a therapy for modulating a valeric acid IL-17 pathway for treating neuroinflammation in a subject, whereby neuroinflammation in the subject is treated. In some embodiments, the neuroinflammation in the subject comprises post-surgery neuroinflammation, and/or impairment of cognition, and/or postoperative cognitive dysfunction (POCD), and/or neuroplasticity, optionally wherein the subject is a human subject. In some embodiments, the therapy administered to the subject comprises exercise, optionally wherein the exercise decreases gut microbiota changes in the subject and/or reduces valeric acid concentrations in the subject. In some embodiments, the therapy administered to the subject comprises fecal transplantation, optionally wherein the fecal transplantation decreases bacteria producing valeric acid, optionally wherein the fecal transplantation decreases Megasphaera massiliensis to reduce valeric acid. In some embodiments, the therapy administered to the subject comprises an agent for reducing IL-17 in the subject, optionally wherein the agent comprises an antibody targeting IL-17, optionally wherein the antibody targets a polypeptide of IL-17 comprising an amino acid sequence of SEQ ID NO. 2. In some embodiments, the therapy administered to the subject comprises a RNA interference (RNAi) construct targeting an IL-17 gene in the subject, optionally wherein the RNAi construct targets an IL-17 gene encoding a nucleic acid sequence of SEQ ID NO. 1. In some embodiments, the therapy administered to the subject modulates the gut microbiota-valeric acid-free fatty acid receptor (FFAR) 2-IL-17 pathway, whereby the concentration of valeric acid and/or IL-17 in the subject is reduced. In some embodiments, the therapy administered to the subject comprises an agent to decrease and/or block FFAR2 in mediating an increase of IL-17 caused by valeric acid, optionally wherein the agent comprises a FFAR2 antagonist, optionally wherein the FFAR2 antagonist is GLPG-0974. In some embodiments, the therapy comprises an agent for reducing IL-17 downstream immune and neuroinflammatory targets selected from C3ar1, C3, Iba-1, IL-1β, IL-6, and combinations thereof, optionally wherein the agent comprises an antagonist and/or antibody targets FFAR2, C3ar1, C3, Iba-1, IL-1β, and/or IL-6, optionally wherein the antibody targets a polypeptide of FFAR2 comprising an amino acid sequence of SEQ ID NO. 4, C3ar1 comprising an amino acid sequence of SEQ ID NO. 6, C3 comprising an amino acid sequence of SEQ ID NO. 8, Iba-1 comprising an amino acid sequence of SEQ ID NO. 10, IL-1B comprising an amino acid sequence of SEQ ID NO. 12, or IL-6 comprising an amino acid sequence of SEQ ID NO. 14. In some embodiments, the therapy administered to the subject comprises a RNA interference (RNAi) construct targeting an FFAR2, C3ar1, C3, Iba-1, IL-1β, and/or IL-6 gene in the subject, optionally wherein the RNAi construct targets a gene encoding FFAR2 comprising a nucleic sequence of SEQ ID NO. 3, C3ar1 comprising a nucleic acid sequence of SEQ ID NO. 5, C3 comprising a nucleic acid sequence of SEQ ID NO. 7, Iba-1 comprising a nucleic acid sequence of SEQ ID NO. 9, IL-1β comprising a nucleic acid sequence of SEQ ID NO. 11, or IL-6 comprising a nucleic acid sequence of SEQ ID NO. 13. In some embodiments, such uses can further comprise measuring blood valeric acid concentration in the subject prior to, during and/or after administration of the therapy.

Accordingly, these and other objects are achieved in whole or in part by the presently disclosed subject matter. Further, objects of the presently disclosed subject matter having been stated above, other objects and advantages of the presently disclosed subject matter will become apparent to those skilled in the art after a study of the following description, Drawings and Examples.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter (often schematically). In the figures, like reference numerals designate corresponding parts throughout the different views. A further understanding of the presently disclosed subject matter can be obtained by reference to an embodiment set forth in the illustrations of the accompanying drawings. Although the illustrated embodiment is merely exemplary of systems for carrying out the presently disclosed subject matter, both the organization and method of operation of the presently disclosed subject matter, in general, together with further objectives and advantages thereof, may be more easily understood by reference to the drawings and the following description. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the following drawings in which:

FIGS. 1A-IF. Exercise improved learning and memory. Nine-week old male mice with or without exercise for 4 weeks were subjected to left carotid artery exposure (surgery) under isoflurane anesthesia (FIGS. 1A to 1C). In another experiment, 9-week old mice were treated with antibiotics to eliminate their native gut microbiota and then transplanted with feces from exercise mice (Trans-Exe) or control mice (Trans-Control) 2 weeks before the surgery (FIGS. 1D to 1F). FIGS. 1A, 1D training sessions of Barnes maze test. FIGS. 1B, 1E memory assessment of Barnes maze test. FIGS. 1C, 1F novel object recognition test. Results are mean±S.D. with (FIGS. 1E and 1F) or without (FIGS. 1A and 1D) presentation of value of individual mouse or median±interquartile range with presentation of value of individual mouse (FIGS. 1B and 1C) (n=17-20 for FIGS. 1A to 1C, =13-15 for FIGS. 1D to 1F). Exe-I/Exercise-I: low intensity exercise, Exe-m: middle intensity exercise, Exe-h: high intensity exercise, Sur: surgery. * P<0.05 compared the two curves.

FIGS. 2A-2H. Exercise stabilized gut microbiota of mice with surgery. Nine-week old male mice with or without exercise for 4 weeks were subjected to left carotid artery exposure (surgery) under isoflurane anesthesia. In another experiment, 9-week old mice were treated with antibiotics to eliminate their native gut microbiota and then transplanted with feces from exercise mice (ET) or control mice (CT). Presentation of a diversity is in FIGS. 2A, 2C, 2E and 2G. Presentation of β diversity is in FIGS. 2B, 2D, 2F and 2H. n=16 for FIGS. 2A and 2B, and =8 for FIGS. 2C-2H. C-S: control mouse samples harvested before surgery, E-ES: exercise mouse samples harvested before surgery, S: surgery mouse samples harvested just before surgery, SP3: surgery mouse samples harvested on post-surgery day 3, SP7: surgery mouse samples harvested on post-surgery day 7, ES: exercise mouse samples harvested just before surgery, ESP3: exercise mouse samples harvested on post-surgery day 3, ESP7: exercise mouse samples harvested on post-surgery day 7, CT: samples harvested from mice transplanted with feces from control mice, ET: samples harvested from mice transplanted with feces from exercise mice.

FIGS. 3A-3F. mRNA expression profile of mice. Hippocampal samples were subjected to RNA-seq analysis. FIG. 2A, 3B volcano plot. FIG. 3C Heatmap of mRNA abundance of genes whose expression was different among the three groups of animals. FIGS. 3D-3F quantitative data of real-time PCR analysis. Results in FIGS. 3D to 3F are mean±S.D. with presentation of value of individual mouse (n=5 for FIGS. 3A to 3C, and =3 for FIGS. 3D to 3F). C: control, S or Sur: surgery, ES or Exe+Sur: exercise plus surgery, Trans-Control: mice transplanted with feces from control mice, Trans-Exe: mice transplanted with feces from exercise mice, NS: normal saline, Val: valeric acid.

FIGS. 4A-4F. Exercise attenuated surgery-induced immune and inflammatory responses. Hippocampus was harvested at various times after surgery for immunostaining or ELISA. FIG. 4A representative Iba-1 and C3ar immunostaining images of hippocampus harvested 48 h after surgery. FIG. 4B quantification of Iba-1 and C3ar immunostaining of hippocampus harvested 48 h after surgery. FIG. 4C representative C3 immunostaining images of hippocampus harvested 48 h after surgery. FIG. 4D quantification of C3 by ELISA. FIG. 4E IL-1β quantified by ELISA. FIG. 4F IL-6 quantified by ELISA. Results in FIGS. 4B, E and F are mean±S.D. with presentation of value of individual mouse and result in FIG. 4D is median±interquartile range with presentation of value of individual mouse (n=6 for FIG. 4B, =14 for FIG. 4D, =9 for FIGS. 4E and F). Exe: exercise, Sur: surgery, Post: post-surgery.

FIGS. 5A-5D. Exercise attenuated surgery-induced decrease of brain cell genesis and dendritic arborization in young adult mice. Brain was harvested 19 days after surgery for immunostaining or Golgi staining. FIG. 5A representative GFAP and BrdU immunostaining images of hippocampus. FIG. 5B quantification of GFAP and BrdU positively stained cells in the hippocampus. For each set of bars, from left to right, Control, Exercise, Surgery, Exercise plus Surgery (Exe+Sur) (see also legend). FIG. 5C representative Golgi staining images of hippocampus. FIG. 5D quantification of intersections among the dendritic branches and spine density in the hippocampus. Results in FIG. 5B and FIG. 5D are mean±S.D. with presentation of value of individual mouse (n=6 for FIG. 5B, =8 for FIG. 5D). Exe: exercise, Sur: surgery.

FIGS. 6A-6F. Exercise attenuated surgery-induced changes in short chain fatty acids (SCFAs). Blood was harvested either 4 weeks after the onset of exercise protocol (for FIGS. 6A, 6C, 6E and 6F) or 7 days after surgery (FIGS. 6B and 6D) from 13 and 14-week old (young mice) or 19-month old mice (old mice). FIG. 6A blood SCFAs just before the surgery in young mice. For each set of bars, from left to right, Control, Exercise, (see also legend). FIG. 6B blood SCFAs 7 days after the surgery in young mice. For each set of bars, from left to right, Control, Surgery, Exercise plus Surgery (Exe+Sur) (see also legend). FIG. 6C blood SCFAs just before the surgery in old mice. For each set of bars, from left to right, Old Control, Old Exercise, (see also legend). FIG. 6D blood SCFAs 7 days after the surgery in old mice. For each set of bars, from left to right, Old Control, Old Surgery, Old Exercise plus Surgery (Exe+Sur) (see also legend). FIG. 6E correlation presentation between gut bacteria and blood SCFA concentrations in young adult mice. FIG. 6F correlation presentation between gut bacteria and fecal SCFA concentrations in young adult mice. Results in FIGS. 6A to 6D are median±interquartile range (for propionic acid, butyric acid, valeric acid and hexanic acid) with presentation of value of individual mouse or mean±S.D. (for other SCFAs) with presentation of value of individual mouse (n=7 for FIGS. 6A-6D). Exe: exercise, Sur: surgery. In FIGS. 6E and 6F, * P<0.05 for the correlation, red: positive correlation, blue: negative correlation, when shown in color.

FIGS. 7A-7F. Role of valeric acid and C3 signaling in exercise attenuation on surgery-induced learning and memory impairment. Nine-week old male mice with or without exercise for 4 weeks in the presence or absence of intraperitoneal injection of valeric acid (one injection per week) were subjected to left carotid artery exposure (surgery) under isoflurane anesthesia (FIGS. 7A to C). In another experiment, 9-week old mice with or without exercise for 4 weeks were subjected to surgery and received intracerebroventricular injection of a C3 agonist or SB290157, a C3ar antagonist, at 0, 24, 48 and 72 h after surgery (FIGS. 7D to 7F). FIG. 7A, D training sessions of Barnes maze test. FIG. 7B For each set of bars, from left to right, Surgery+NS, Exercise plus Surgery plus NS, (Exe+Sur+NS), Exercise plus Surgery plus Val, (Exe+Sur+Val) (see also legend), FIG. 7E memory assessment of Barnes maze test. FIGS. 7C and F novel object recognition test. Results are mean+S.D. in FIGS. 7A and 7D and median+interquartile range with presentation of value of individual mouse in FIGS. 7B, 7C, 7E and 7F (n=11-13 for FIGS. 7A to 7C, =13-15 for FIGS. 7D to 7F). Exe: exercise, Sur: surgery, NS: normal saline, DMSO: dimethylsulfoxide, Val: valeric acid, Anta-C3ar: C3ar antagonist, Agon-C3ar: C3ar agonist. * P<0.05 compared the two curves.

FIGS. 8A-8I. Exercise via regulating gut microbiota attenuated surgery-induced GDNF decrease and GDNF participated in regulation of immune and inflammatory responses after surgery. Nine-week old male mice with or without exercise for 4 weeks in the presence or absence of intraperitoneal injection of valeric acid (one injection per week) were subjected to left carotid artery exposure (surgery) under isoflurane anesthesia (FIGS. 8A and 8D). In second experiment, 9-week old mice were treated with antibiotics to eliminate their native gut microbiota and then transplanted with feces from control mice (Trans-Control) before the surgery (FIG. 8B). In third experiment, 9-week old mice were treated with antibiotics to eliminate their native gut microbiota and then transplanted with feces of exercise mice (Trans-Exe) or control mice (Trans-Control) before the surgery (FIG. 8C). In fourth experiment, 9-week old mice received intraperitoneal injection of valeric acid or normal saline (FIGS. 8E and 8F). In fifth experiments, 9-week old mice were subjected to surgery and received intracerebroventricular injection of GDNF immediately after surgery (FIGS. 8G to 8I). FIGS. 8A and 8D GDNF concentrations in the hippocampus. FIG. 8E valeric acid in the cerebral cortex. FIGS. 8F, 8G C3 concentrations in the hippocampus. FIG. 8H IL-6 concentrations in the hippocampus. FIG. 8I IL-1β concentrations in the hippocampus. Results in FIGS. 8A, 8B, 8D and 8G are median±interquartile range with presentation of value of individual mouse and results in other Figs. are mean±S.D. with presentation of value of individual mouse (n=10 for FIGS. 8A to 8D and FIG. 8F, =7-8 for FIG. 8E, =12 for FIG. 8G to 8I). Exe: exercise, Sur: surgery, NS: normal saline, Val: valeric acid.

FIGS. 9A-9J. Exercise via regulating gut microbiota and blood valeric acid attenuated surgery-induced learning and memory impairment in old mice. Nineteen-month old male mice with or without exercise for 4 weeks were subjected to left carotid artery exposure (surgery) under isoflurane anesthesia (FIGS. 9A to 9D). In second experiment, 19-month old mice were treated with antibiotics to eliminate their native gut microbiota and then transplanted with feces from exercise mice (Trans-Exe) or control mice (Trans-Control) before the surgery (FIGS. 9E to 9G). In third experiments, 19-month old male mice with or without exercise for 4 weeks in the presence or absence of intraperitoneal injection of valeric acid (one injection per week) were subjected to surgery (FIGS. 9H to 9J). FIGS. 9A, 9E, 9H training sessions of Barnes maze test.

FIGS. 9B, 9F, 9I memory assessment of Barnes maze test. FIGS. 9C, 9D, 9G, 9J novel object recognition test. Results are mean+S.D. with (FIGS. 9B and 9G) or without (FIGS. 9A, 9E and 9H) presentation of value of individual mouse or median+interquartile range (FIGS. 9F, 9I, and 9J) with presentation of value of individual mouse in FIGS. 9A-9J (n=11-12 for FIGS. 9A to 9D, =9-10 for FIGS. 9E to 9G, =9-11 for FIGS. 9H to 9J). Exe: exercise, Sur: surgery, NS: normal saline, Val: valeric acid. * P<0.05 compared the two curves.

FIGS. 10A-10H. Exercise stabilized gut microbiota of mice with surgery in old mice. Nineteen-month old male mice with or without exercise for 4 weeks were subjected to left carotid artery exposure (surgery) under isoflurane anesthesia. Presentation of a diversity is in FIGS. 10A, 10C, 10E and 10G. Presentation of β diversity is in FIGS. 10B, 10D, 10F and 10H (n=16 for FIGS. 10A and 10B, and =8 for FIGS. 10C-10H). OC-OS: old control mouse samples harvested before surgery, OE-OES: old exercise mouse samples harvested before surgery, OS: old surgery mouse samples harvested just before surgery, OSP: old surgery mouse samples harvested on post-surgery day 7, OES: old exercise mouse samples harvested before surgery, OESP: old exercise mouse samples harvested on post-surgery day 7.

FIGS. 11A-11I. Exercise attenuated surgery-induced GDNF decrease, immune and inflammatory responses and dendritic arborization impairment in old mice. Nineteen-month old male mice with or without exercise for 4 weeks were subjected to left carotid artery exposure (surgery) under isoflurane anesthesia. FIG. 11A GDNF expression. FIG. 11B C3 expression. FIG. 11C representative Iba-1 and C3ar immunostaining images. FIG. 11D quantitative data of Iba-1 and C3ar immunostaining. FIG. 11E IL-1ß and IL-6 expression. FIG. 11F representative GFAP and BrdU immunostaining images. FIG. 11G quantitative data of GFAP and BrdU immunostaining. FIG. 11H representative Golgi staining. FIG. 11I quantitative data of intersections among dendritic branches and spine density. Results in FIGS. 11A, 11D and 11E are median±interquartile range with presentation of value of individual mouse and results in FIGS. 11B, 11G and I are mean±S.D. with presentation of value of individual mouse (n=10 for FIG. 11A, =12 for FIG. 11B, =6 for FIGS. 11D and 11G, =8 for FIGS. 11E and 11I). Exe: exercise, Sur: surgery.

FIGS. 12A-12F. Transplantation of gut microbiota from surgery mice induced learning and memory dysfunction. Nine-week old mice received antibiotic treatment for 7 days (Antibiotic) or transplantation of feces from control mice (Trans-Control) (FIGS. 12A to 12C). In another experiment, nine-week old male mice received transplantation of feces from control mice (Trans-Control) or mice with surgery (Trans-Surgery) (FIGS. 12D to 12F). FIGS. 12A and 12D: training sessions of Barnes maze test. B and E: memory assessment of Barnes maze test. FIGS. 12C and 12F: novel object recognition test. Results in FIGS. 12A and 12D are mean±S.D. and results in FIGS. 12B, 12C, 12E and 12F are median±interquartile range with presentation of value of individual mouse (n=11-12 for FIGS. 12A to 12C, =16-17 for FIGS. 12D to 12F).

FIGS. 13A-13G. Diagrammatic presentation of experiments. O: old, Y: young, MCAO: middle cerebral artery occlusion, FMT: fecal microbiota transplant.

FIGS. 14A-14H. Age-dependent worsening of neurological outcome and heightened inflammatory response after brain ischemia. Old mice (18-months old) or young mice (8-weeks old) were subjected to 120-min MCAO. Their neurological outcome was evaluated and blood was collected 24 h after MCAO. FIG. 14A: representative brain slice images after 2,3,5-triphenyltetrazolium chloride staining. FIG. 14B: infarct volume. FIG. 14C: neurological deficit scores. FIG. 14D: performance on rotarod. FIG. 14E: IL-17 concentrations in the blood. FIG. 14F: IL-1β concentrations in the blood. FIG. 14G: IL-6 concentrations in the blood. FIG. 14H: IL-10 concentrations in the blood. Parametric results in normal distribution are in mean±S.E.M. and other results that are nonparametric data or parametric data in non-normal distribution are presented as median with interquartile range. Data of each individual animal is also presented (n=8-12).

FIGS. 15A-15J. Age-dependent gut microbiota changes. Feces were harvested from old mice (18-months old, n=11) or young mice (8-weeks old, n=9) and the gut microbiota in these two groups of mice was analyzed and compared. FIGS. 15A to 15E: a diversity difference between the two groups analyzed by chao1, observed_species, PD_whole_tree, Shannon or Simpson methods, respectively. FIG. 15F: heatmap presentation of the relative gut microbiota abundance in samples. FIG. 15G and FIG. 15H: β diversity difference between the two groups analyzed by the unweighted Unifrac methods Anosim and PCoA, respectively. I: venn map showing unique and shared Operational Taxonomic Unit (OUT) between the two groups of mice. J: boxplot of relative abundance of taxa within gut microbiota at genus level.

FIGS. 16A-16B. Age-dependent changes of short chain fatty acids in the blood. A: blood was collected from old mice (18-months old) or young mice (8-weeks old) without any treatment or brain ischemia and used to measure short chain fatty acids. B: blood was collected from young mice (8-weeks old) transplanted 14 days ago with old mouse (18-months old) (Young-oFMT) or young mouse (8-weeks old) (Young-yFMT) feces. Blood was then used to measure short chain fatty acids. Parametric results in normal distribution are in mean±S.E.M. and other results that are nonparametric data or parametric data in non-normal distribution are presented as median with interquartile range. Data of each individual animal is also presented (n=8 for FIG. 16A, =7 for FIG. 16B).

FIGS. 17A-17H. Worsening of neurological outcome and heightened inflammatory responses in the brain after brain ischemia in young mice transplanted with old mouse feces. Young mice (8-weeks old) received saline, cefazolin, cefazolin and then transplantation of old mouse feces or cefazolin and then transplantation of young mouse feces. They were subjected to 120-min MCAO 2 weeks after the fecal transplantation and named Young-saline, Young-antibiotic, Young-oFMT and Young-yFMT, respectively. Their neurological outcome was evaluated and brain was collected 24 h after MCAO. FIG. 17A: representative brain slice images after 2,3,5-triphenyltetrazolium chloride staining. FIG. 17B: infarct volume. FIG. 17C: neurological deficit scores. FIG. 17D: performance on rotarod. FIG. 17E: IL-17 concentrations in the brain. FIG. 17F: IL-1β concentrations in the brain. FIG. 17G: IL-6 concentrations in the brain. FIG. 17H: IL-10 concentrations in the brain. One cohort of mice was used to generate the data presented in FIGS. 17A to 17D. Another cohort was used for the data presented in FIGS. 17E to 17H. Parametric results in normal distribution are in mean±S.E.M. and other results that are nonparametric data or parametric data in non-normal distribution are presented as median with interquartile range. Data of each individual animal is also presented (n=14-19 for FIGS. 17B to 17D, =8 for FIGS. 17E to H).

FIGS. 18A-18D. Heightened inflammatory responses in the blood after brain ischemia in young mice transplanted with old mouse feces or treated with valeric acid. Blood was harvested 24 h after 120-min MCAO for cytokine analysis. FIG. 18A: young mice (8-weeks old) received saline, cefazolin, cefazolin and then transplantation of old mouse feces or cefazolin and then transplantation of young mouse feces. They were subjected to 120-min MCAO 2 weeks after the fecal transplantation and named Young-saline, Young-antibiotic, Young-oFMT and Young-yFMT, respectively. FIG. 18B: young mice received saline or valeric acid intraperitoneally and then subjected to 120-min MCAO. They were named Saline Stroke and Valeric Stroke, respectively. FIG. 18C: young mice received valeric acid and were subjected to 120-min MCAO. They were then received intravenous saline, mouse IgG1 or mouse monoclonal anti-IL-17 antibody and named saline, IgG isotype and anti-IL-17, respectively. FIG. 18D: young mice received intraperitoneal normal Saline, valeric acid, the combination of saline and GLPG-0974 or the combination of valeric acid and GLPG-0974. IL-17 in the blood was measured. Parametric results in normal distribution are in mean±S.E.M. and other results that are nonparametric data or parametric data in non-normal distribution are presented as median with interquartile range. Data of each individual animal is also presented (n=8 for FIG. 18A, =7-11 for FIG. 18B, =8 for FIG. 18C, n=9-10 for FIG. 18D).

FIGS. 19A-19D. Worsening of neurological outcome and heightened inflammatory responses in the brain after brain ischemia in young mice treated with valeric acid and improved outcome by an anti-IL-17 antibody. Young mice (8-week old) received intraperitoneal saline or valeric sodium. They were subjected to 120-min MCAO and named Saline Stroke and Valeric Stroke, respectively. Their neurological outcome was evaluated and brain was collected 24 h after MCAO for cytokine measurements (results are in FIGS. 19A and 19B). In another experiment, young mice (8-weeks old) were subjected to 120-min MCAO plus intraperitoneal valeric acid and then received intravenous saline, mouse IgG1 or mouse monoclonal anti-IL-17 antibody. They were named Saline, IgG isotype and anti-IL-17, respectively. Their neurological outcome was evaluated (results are in FIG. 19C). A: neurological outcome. FIG. 19B: inflammatory markers in the brain. FIG. 19C: neurological outcome. FIG. 19D: young mice received intraperitoneal normal saline, valeric acid, the combination of saline and GLPG-0974 or the combination of valeric acid and GLPG-0974. Cerebral cortex was harvested for measuring IL-17. Parametric results in normal distribution are in mean±S.E.M. and other results that are nonparametric data or parametric data in non-normal distribution are presented as median with interquartile range. Data of each individual animal is also presented (n=15 for FIG. 19A, =7 for FIGS. 19B, =8 for FIG. 19C, n=9-10 for FIG. 19D).

FIGS. 20A-20E. Worsened outcome in young mice transplanted with old mouse feces and improved outcome in old mice transplanted with young mouse feces. Young mice (8-weeks old) received saline, cefazolin, cefazolin and then transplantation of old mouse feces or cefazolin and then transplantation of young mouse feces. They were subjected to 120-min MCAO 2 weeks after the fecal transplantation and named Young-saline, Young-antibiotic, Young-oFMT and Young-yFMT, respectively. In another experiment, old mice (18-months old) received saline, cefazolin, cefazolin and then transplantation of old mouse feces or cefazolin and then transplantation of young mouse feces. They were subjected to 60-min MCAO 2 weeks after the fecal transplantation and named Old-saline, Old-antibiotic, Old-oFMT and Old-yFMT, respectively. Their body weights and survival curves were evaluated after MCAO. A: body weight changes of young mice. FIG. 20B: survival curve of young mice. FIG. 20C: body weight changes of old mice. FIG. 20D: survival curve of old mice. FIG. 20E: summary diagram for the mechanism of age-dependent microbiota changes-induced increase of ischemic brain injury and inflammatory responses. Results in FIGS. 20A and C are in mean±S.E.M. (n=4 to 9 for FIG. 20A and =6-7 for FIG. 20C). *F(1, 11)=8.635, P=0.013 compared with old mice receiving saline. \hat{ }F(1,12)=5.383, P=0.034 compared with old mice receiving old mouse feces. The survival curves of the four groups in FIGS. 20B and D were different when analyzed by the Mantel-Cox test (P=0.0014 for FIG. 20B and =0.0035 for FIG. 20D). & P<0.05, && P<0.01, &&& P<0.001 compared with young mice receiving old mouse feces by the Gehan-Breslow-Wilcoxon test; #P<0.05 compared with old mice receiving old mouse feces or old mice without fecal transplantation by the Gehan-Breslow-Wilcoxon test.

FIGS. 21A-21E. Transplanting old mouse gut microbiome worsened learning and memory dysfunction after surgery in young mice. Eight-week old male mice received transplant of gut microbiome from eight-week old male mice (Young-yFMT) or from eighteen-month old male mice (Young-oFMT). They were then subjected to surgery (carotid artery exposure) 11 days after the completion of fecal transplant. Mice were started to be tested 7 days after surgery. FIG. 21A: Train phase in Barnes maze. FIG. 21B: Memory phase at one day after training sessions in Barnes maze. FIG. 21C: Memory phase at eight days after training sessions in Barnes maze. FIG. 21D: Context-related fear conditioning behavior. FIG. 21E: Tone-related fear conditioning. Results are mean±S.D. (n=28-35). Data of individual mouse is included in the bar graphs. * P<0.05, ** P<0.01, *** P<0.005, **** P<0.001. In FIGS. 21B to 21E, the bars are in the following order from left to right: Control (circles); Surgery+Saline (squares); Surgery+Young-oFNT (triangles); Surgery+Young-yFMT (inverted triangles).

FIGS. 22A-22F. Transplanting old mouse gut microbiome increased proinflammatory cytokines after surgery in young mice. Eight-week old male mice received transplant of gut microbiome from eight-week old male mice (Young-yFMT) or from eighteen-month old male mice (Young-oFMT). Their blood, hippocampus and cerebral cortex were harvested at various times after surgery. FIG. 22A: IL-1ß in the serum. FIG. 22B: IL-6 in the serum. FIG. 22C: IL-1ß in the hippocampus. FIG. 22D: IL-6 in the hippocampus. FIG. 22E: IL-1ß in the cerebral cortex. FIG. 22F: IL-6 in the cerebral cortex. Results are mean±S.D. (n=7). Data of individual mouse is included in the bar graphs. * P<0.05, ** P<0.01, *** P<0.005, **** P<0.001. In FIGS. 22A to 22F, each set of bars are in the following order from left to right: Control (circles); Surgery+Saline (squares); Surgery+Young-oFNT (triangles); Surgery+Young-yFMT (inverted triangles).

FIGS. 23A-23G. Transplanting old mouse gut microbiome increased valeric acid after surgery in young mice. Eight-week old male mice received transplant of gut microbiome from eight-week old male mice (Young-yFMT) or from eighteen-month old male mice (Young-oFMT). Their blood was harvested at 24 h after surgery. FIG. 23Aa: Acetic acid. FIG. 23B: Propionic acid. FIG. 23C: Isobutyric acid. FIG. 23D: Butyric acid. FIG. 23E: Isovaleric acid. FIG. 23F: Valeric acid. FIG. 23G: Hexanic acid. Results are mean±S.D. (n=7). Data of individual mouse is included in the bar graphs. * P<0.05, ** P<0.01, *** P<0.005, **** P<0.001. In FIGS. 23A to 23G, the bars are in the following order from left to right: Control (circles); Surgery +Saline (squares); Surgery+Young-oFNT (triangles); Surgery+Young-yFMT (inverted triangles).

### DETAILED DESCRIPTION

The presently disclosed subject matter now will be described more fully hereinafter, in which some, but not all embodiments of the presently disclosed subject matter are described. Indeed, the presently disclosed subject matter can be embodied in many different forms and should not be construed as limited to the embodiments set forth herein; rather, these embodiments are provided so that this disclosure will satisfy applicable legal requirements.

### I. Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one skilled in the art. While the following terms are believed to be well understood by one of ordinary skill in the art, the following definitions are set forth to facilitate explanation of the presently disclosed subject matter.

Following long-standing patent law convention, the terms “a”, “an”, and “the” refer to “one or more” when used in this application, including the claims. Thus, for example, reference to “a cell” includes a plurality of such cells, and so forth.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

As used herein, the term “about,” when referring to a value or to an amount of a composition, mass, weight, temperature, time, volume, concentration, percentage, etc., is meant to encompass variations of in some embodiments±20%, in some embodiments±10%, in some embodiments±5%, in some embodiments±1%, in some embodiments±0.5%, and in some embodiments ±0.1% from the specified amount, as such variations are appropriate to perform the disclosed methods or employ the disclosed compositions. Numerical ranges recited herein by endpoints include all numbers and fractions subsumed within that range (e.g. 1 to 5 includes 1, 1.5, 2, 2.75, 3, 3.90, 4, 4.24, and 5). Similarly, numerical ranges recited herein by endpoints include subranges subsumed within that range (e.g. 1 to 5 includes 1-1.5, 1.5-2, 2-2.75, 2.75-3, 3-3.90, 3.90-4, 4-4.24, 4.24-5, 2-5, 3-5, 1-4, and 2-4). It is also to be understood that all numbers and fractions thereof are presumed to be modified by the term “about.”

The term “comprising”, which is synonymous with “including” “containing” or “characterized by” is inclusive or open-ended and does not exclude additional, unrecited elements or method steps. “Comprising” is a term of art used in claim language which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, the term “and/or” when used in the context of a listing of entities, refers to the entities being present singly or in combination. Thus, for example, the phrase “A, B, C, and/or D” includes A, B, C, and D individually, but also includes any and all combinations and subcombinations of A, B, C, and D.

A “subject” of analysis, diagnosis, or treatment is an animal. Such animals include mammals, in some embodiments, humans.

As used herein, a “subject in need thereof” is a patient, animal, mammal, or human, who will benefit from the methods and compositions of the presently disclosed subject matter.

As used herein, the term “subject” refers to any organism for which application of the presently disclosed subject matter would be desirable. The subject treated in the presently disclosed subject matter in its many embodiments is desirably a human subject, although it is to be understood that the principles of the presently disclosed subject matter indicate that the presently disclosed subject matter is effective with respect to all vertebrate species, including mammals, which are intended to be included in the term “subject”. Moreover, a mammal is understood to include any mammalian species in which treatment is desirable, particularly agricultural and domestic mammalian species.

The term “subject” as used herein refers to a member of species for which treatment using the compositions and methods of the presently disclosed subject matter might be desirable. Accordingly, the term “subject” is intended to encompass in some embodiments any member of the Kingdom Animalia including, but not limited to the phylum Chordata (e.g., members of Classes Osteichythyes (bony fish), Amphibia (amphibians), Reptilia (reptiles), Aves (birds), and Mammalia (mammals), and all Orders and Families encompassed therein.

In some embodiments, the subject can be a chronologically older or aged subject. By way of example and not limitation, a human subject that is aged, older or elderly can be a human subject that about 40 years of age or older, about 50 years of age or older, about 60 years of age or older, about 70 years of age or older, about 75 years of age or older, or about 80 years of age or older.

The compositions and methods of the presently disclosed subject matter are particularly useful for warm-blooded vertebrates. Thus, in some embodiments the presently disclosed subject matter concerns mammals and birds. More particularly provided are compositions and methods derived from and/or for use in mammals such as humans and other primates, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), rodents (such as mice, rats, and rabbits), marsupials, and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, also provided is the use of the disclosed methods and compositions on livestock, including but not limited to domesticated swine (pigs and hogs), ruminants, horses, poultry, and the like.

The terms “treat,” “treatment,” and “treating” as used herein refer to both therapeutic treatment and prophylactic or preventative measures, wherein the object is to prevent or slow down (lessen or reduce) the targeted condition, prevent the condition, pursue or obtain beneficial results, and/or lower the chances of the individual developing a condition, disease, or disorder, even if the treatment is ultimately unsuccessful. Those in need of treatment include those already with the condition as well as those prone to have or predisposed to having a condition, disease, or disorder, or those in whom the condition is to be prevented. Thus, in some embodiments, modulating a pathway as disclosed herein can treat the condition by preventing the condition in a subject prone to developing the condition and/or by improving the condition or outcome in a subject already suffering from the condition.

The term “isolated”, as used in the context of a nucleic acid molecule or polypeptide, indicates that the nucleic acid molecule or polypeptide exists apart from its native environment and is not a product of nature. An isolated nucleic acid molecule or polypeptide can exist in a purified form or can exist in a non-native environment such as a host cell.

The term “complementary” refers to two nucleotide sequences that comprise antiparallel nucleotide sequences capable of pairing with one another upon formation of hydrogen bonds between the complementary base residues in the antiparallel nucleotide sequences. As is known in the art, the nucleic acid sequences of two complementary strands are the reverse complement of each other when each is viewed in the 5′ to 3′ direction.

As used herein, the phrase “percent complementarity” refers to the percentage of contiguous residues in a nucleic acid molecule that can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). The terms “100% complementary”, “fully complementary”, and “perfectly complementary” indicate that all of the contiguous residues of a nucleic acid sequence can hydrogen bond with the same number of contiguous residues in a second nucleic acid sequence.

The term “subsequence” refers to a sequence of a nucleic acid or polypeptide that comprises a part of a longer nucleic acid or polypeptide sequence.

The term “elongated sequence” refers to an addition of nucleotides (or other analogous molecules) or amino acid residues incorporated into the nucleic acid or polypeptide. For example, a polymerase (e.g., a DNA polymerase) can add sequences at the 3′ terminus of the nucleic acid molecule. In addition, the nucleotide sequence can be combined with other DNA sequences, such as promoters, promoter regions, enhancers, polyadenylation signals, intronic sequences, additional restriction enzyme sites, multiple cloning sites, and other coding segments.

The terms “operatively linked” and “operably linked”, as used herein, refer to a nucleic acid molecule in which a promoter region is connected to a nucleotide sequence in such a way that the transcription of that nucleotide sequence is controlled and regulated by the promoter region. Similarly, a nucleotide sequence is said to be under the “transcriptional control” of a promoter to which it is operably linked. Techniques for operatively linking a promoter region to a nucleotide sequence are known in the art.

The terms “heterologous gene”, “heterologous DNA sequence”, “heterologous nucleotide sequence”, “exogenous nucleic acid molecule”, or “exogenous DNA segment”, as used herein, each refer to a sequence that originates from a source foreign to an intended host cell and/or, if from the same source, is modified from its original form. Thus, a heterologous gene in a host cell includes a gene that is endogenous to the particular host cell but has been modified, for example by mutagenesis and/or by isolation from native transcriptional regulatory sequences. The terms also include non-naturally occurring multiple copies of a naturally occurring nucleotide sequence. Thus, the terms refer in some embodiments to a DNA segment that is foreign or heterologous to the cell, or is homologous to the cell but in a position within the host cell nucleic acid wherein the element is not ordinarily found.

The term “expression vector” as used herein refers to a nucleotide sequence capable of directing expression of a particular nucleotide sequence (such as but not limited to a heterologous nucleotide sequence) in an appropriate host cell, comprising a promoter (such as but not limited to a minimal promoter or promoter as described herein) operatively linked to the nucleotide sequence of interest which is operatively linked to termination signals. It also typically comprises sequences required for proper translation of the nucleotide sequence. The construct comprising the nucleotide sequence of interest can be chimeric. The construct can also be one that is naturally occurring but has been obtained in a recombinant form useful for heterologous expression.

The term “promoter” or “promoter region” each refers to a nucleotide sequence within a gene that is positioned 5′ to a coding sequence and functions to direct transcription of the coding sequence. The promoter region comprises a transcriptional start site, and can additionally include one or more transcriptional regulatory elements.

A “minimal promoter” is a nucleotide sequence that has the minimal elements required to enable basal level transcription to occur. As such, minimal promoters are not complete promoters but rather are subsequences of promoters that are capable of directing a basal level of transcription of a reporter construct in an experimental system. Minimal promoters include but are not limited to the CMV minimal promoter, the HSV-tk minimal promoter, the simian virus 40 (SV40) minimal promoter, the human β-actin minimal promoter, the human EF2 minimal promoter, the adenovirus E1B minimal promoter, and the heat shock protein (hsp) 70 minimal promoter. Minimal promoters are often augmented with one or more transcriptional regulatory elements to influence the transcription of an operably linked gene. For example, cell-type-specific or tissue-specific transcriptional regulatory elements can be added to minimal promoters to create recombinant promoters that direct transcription of an operably linked nucleotide sequence in a cell-type-specific or tissue-specific manner

Different promoters have different combinations of transcriptional regulatory elements. Whether or not a gene is expressed in a cell is dependent on a combination of the particular transcriptional regulatory elements that make up the gene's promoter and the different transcription factors that are present within the nucleus of the cell. As such, promoters are often classified as “constitutive”, “tissue-specific”, “cell-type-specific”, or “inducible”, depending on their functional activities in vivo or in vitro. For example, a constitutive promoter is one that is capable of directing transcription of a gene in a variety of cell types.

The term “transcriptional regulatory sequence” or “transcriptional regulatory element”, as used herein, each refers to a nucleotide sequence within the promoter region that enables responsiveness to a regulatory transcription factor. Responsiveness can encompass a decrease or an increase in transcriptional output and is mediated by binding of the transcription factor to the DNA molecule comprising the transcriptional regulatory element.

The term “transcription factor” generally refers to a protein that modulates gene expression by interaction with the transcriptional regulatory element and cellular components for transcription, including RNA polymerase, Transcription Associated Factors (TAFs), chromatin-remodeling proteins, and any other relevant protein that impacts gene transcription.

The terms “reporter gene” or “marker gene” or “selectable marker” each refer to a heterologous gene encoding a product that is readily observed and/or quantitated. A reporter gene is heterologous in that it originates from a source foreign to an intended host cell or, if from the same source, is modified from its original form. Non-limiting examples of detectable reporter genes that can be operatively linked to a transcriptional regulatory region can be found in Alam & Cook, 1990 and PCT International Publication No. WO 97/47763. Exemplary reporter genes for transcriptional analyses include the lacZ gene, luciferase, and chloramphenicol acetyl transferase (CAT). Reporter genes for methods to produce transgenic animals include but are not limited to antibiotic resistance genes, for example the antibiotic resistance gene confers neomycin resistance. Any suitable reporter and detection method can be used, and it will be appreciated by one of skill in the art that no particular choice is essential to or a limitation of the presently disclosed subject matter.

An amount of reporter gene can be assayed by any method for qualitatively or quantitatively determining presence or activity of the reporter gene product. The amount of reporter gene expression directed by each test promoter region fragment is compared to an amount of reporter gene expression to a control construct comprising the reporter gene in the absence of a promoter region fragment. A promoter region fragment is identified as having promoter activity when there is significant increase in an amount of reporter gene expression in a test construct as compared to a control construct. The term “significant increase”, as used herein, refers to an quantified change in a measurable quality that is larger than the margin of error inherent in the measurement technique, in one example an increase by about 2-fold or greater relative to a control measurement, in another example an increase by about 5-fold or greater, and in yet another example an increase by about 10-fold or greater.

Nucleic acids of the presently disclosed subject matter can be cloned, synthesized, recombinantly altered, mutagenized, or combinations thereof. Standard recombinant DNA and molecular cloning techniques used to isolate nucleic acids are known in the art. Site-specific mutagenesis to create base pair changes, deletions, or small insertions is also known in the art.

As used herein, the term “cell” is used in its usual biological sense. In some embodiments, the cell is present in an organism, for example, mammals such as humans, cows, sheep, apes, monkeys, swine, dogs, cats, and rodents. In some embodiments, the cell is a eukaryotic cell (e.g., a mammalian cell, such as a human cell). The cell can be of somatic or germ line origin, totipotent or pluripotent, dividing or non-dividing. The cell can also be derived from or can comprise a gamete or embryo, a stem cell, or a fully differentiated cell.

As used herein, the term “RNA” refers to a molecule comprising at least one ribonucleotide residue. By “ribonucleotide” is meant a nucleotide with a hydroxyl group at the 2′ position of a β-D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

RNA interference (RNAi) is a natural process by which living cells can control which genes are expressed or suppressed. As used herein, the term “RNAi” refers to a mechanism that silences specific genes by inhibiting an RNA molecule and stopping or at least substantially reducing the expression of the protein encoded by this RNA molecule. If the target protein has a function in the cell, RNAi approaches can result in loss of that function. As such, RNAi technology is an attractive therapeutic tool to modulate the expression of genes in a way to suppress disease. RNAi can be mediated by several natural and synthetic constructs, including double stranded RNA (dsRNA), or smaller dsRNA known as small interfering RNAs (siRNA), short hairpin RNA (shRNA), microRNA (miRNA), or synthetic hammerhead ribozymes. These can be referred to as examples of RNAi molecules.

As used herein, the phrase “double stranded RNA” refers to an RNA molecule at least a part of which is in Watson-Crick base pairing forming a duplex. As such, the term is to be understood to encompass an RNA molecule that is either fully or only partially double stranded. Exemplary double stranded RNAs include, but are not limited to molecules comprising at least two distinct RNA strands that are either partially or fully duplexed by intermolecular hybridization. Additionally, the term is intended to include a single RNA molecule that by intramolecular hybridization can form a double stranded region (for example, a hairpin). Thus, as used herein the phrases “intermolecular hybridization” and “intramolecular hybridization” refer to double stranded molecules for which the nucleotides involved in the duplex formation are present on different molecules or the same molecule, respectively.

As used herein, the phrase “double stranded region” refers to any region of a nucleic acid molecule that is in a double stranded conformation via hydrogen bonding between the nucleotides including, but not limited to hydrogen bonding between cytosine and guanosine, adenosine and thymidine, adenosine and uracil, and any other nucleic acid duplex as would be understood by one of ordinary skill in the art. The length of the double stranded region can vary from about 15 consecutive basepairs to several thousand basepairs.

As used herein, the terms “corresponds to”, “corresponding to”, and grammatical variants thereof refer to a nucleotide sequence that is 100% identical to a preferred number of contiguous nucleotides of a nucleic acid sequence encoding a polypeptide of interest, e.g. IL-17. Thus, a first nucleic acid sequence that “corresponds to” a coding strand of a IL-17 subunit gene is a nucleic acid sequence that is 100% identical to at least 5, 10, 15, 20, 25, 50 or 100, for example, contiguous nucleotides of a IL-17 subunit gene, including, but not limited to 5′ untranslated sequences, exon sequences, intron sequences, and 3′ untranslated sequences.

As used herein, the terms “effective amount” and “therapeutically effective amount” are used interchangeably and mean a dosage sufficient to provide treatment for the disease state being treated. This can vary depending on the patient, the disease and the treatment being effected.

The term “gene” is used broadly to refer to any segment of DNA associated with a biological function. Thus, genes include, but are not limited to, coding sequences and/or the regulatory sequences required for their expression. Genes can also include non-expressed DNA segments that, for example, form recognition sequences for a polypeptide.

Nucleotide sequences are “substantially identical” where they have between about 70% and about 80% or more preferably, between about 81% and about 90%, or even more preferably, between about 91% and about 99%, sequence identity for nucleic acid residues which are identical to the nucleotide sequence of a desired subunit gene, e.g. IL-17.

The term “gene expression” generally refers to the cellular processes by which a biologically active polypeptide is produced from a DNA sequence and exhibits a biological activity in a cell. As such, gene expression involves the processes of transcription and translation, but also involves post-transcriptional and post-translational processes that can influence a biological activity of a gene or gene product. These processes include, but are not limited to RNA syntheses, processing, and transport, as well as polypeptide synthesis, transport, and post-translational modification of polypeptides. Additionally, processes that affect protein-protein interactions within the cell can also affect gene expression as defined herein.

The term “modulate”, when used in the context of gene expression, refers to a change in the expression level of a gene, or a level of RNA molecule or equivalent RNA molecules encoding one or more proteins or protein subunits, or activity of one or more proteins or protein subunits is up regulated or down regulated, such that expression, level, or activity is greater than or less than that observed in the absence of the modulator. For example, the term “modulate” can mean “inhibit” or “suppress”, but the use of the word “modulate” is not limited to this definition.

The term “modulate”, when used in the context of impacting a biological pathway in vivo in a subject, as described herein, refers to a change in a concentration and/or activity of one or more metabolites, compounds or components of a biological pathway such that the function of the biological pathway is substantially altered as compared. For example, the valeric acid-IL-17 pathway can be modulated by decreasing or increasing the concentration and/or activity of one or more compounds within the pathway such that a biological function or condition related to the pathway is affected. More particularly, and by way of example and not limitation, modulating the valeric acid-IL-17 pathway can result in a decrease in valeric acid and/or any downstream compounds in the pathway.

As used herein, the terms “silence”, “ablate”, “inhibit”, “suppress”, “downregulate”, “loss of function”, “block of function”, and grammatical variants thereof are used interchangeably and refer to an activity whereby gene expression (e.g., a level of an RNA encoding one or more gene products) is reduced below that observed in the absence of a composition of the presently disclosed subject matter. In some embodiments, inhibition results in a decrease in the steady state level of a target RNA. In some embodiments, inhibition results in an expression level of a gene product that is below that level observed in the absence of the modulator.

In some embodiments, the terms “inhibit”, “suppress”, “downregulate”, “block of function” and grammatical variants thereof refer to a biological activity of a polypeptide or polypeptide complex that is lower in the presence of a modulator than that which occurs in the absence of the modulator. For example, a modulator can inhibit expression or function of IL-17, or other valeric acid pathway target disclosed herein, thereby inhibiting the activity of IL-17. This can be accomplished by any mechanism, including but not limited to enhancing its existence in an inactive form and/or by enhancing the rate of degradation of IL-17.

As used herein, the terms “gene” and “target gene” refer to a nucleic acid that encodes an RNA, for example, nucleic acid sequences including, but not limited to, structural genes encoding a polypeptide. The target gene can be a gene derived from a cell, an endogenous gene, a transgene, etc. The cell containing the target gene can be derived from or contained in any organism, for example an animal. The term “gene” also refers broadly to any segment of DNA associated with a biological function. As such, the term “gene” encompasses sequences including but not limited to a coding sequence, a promoter region, a transcriptional regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

As is understood in the art, a gene comprises a coding strand and a non-coding strand. As used herein, the terms “coding strand” and “sense strand” are used interchangeably, and refer to a nucleic acid sequence that has the same sequence of nucleotides as an mRNA from which the gene product is translated. As is also understood in the art, when the coding strand and/or sense strand is used to refer to a DNA molecule, the coding/sense strand includes thymidine residues instead of the uridine residues found in the corresponding mRNA. Additionally, when used to refer to a DNA molecule, the coding/sense strand can also include additional elements not found in the mRNA including, but not limited to promoters, enhancers, and introns. Similarly, the terms “template strand” and “antisense strand” are used interchangeably and refer to a nucleic acid sequence that is complementary to the coding/sense strand.

The term “nucleic acid” refers to deoxyribonucleotides or ribonucleotides and polymers thereof in either single- or double-stranded form. Unless specifically limited, the term encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences and as well as the sequence explicitly indicated. Specifically, degenerate codon substitutions can be achieved by generating sequences in which the third position of one or more selected (or all) codons is substituted with mixed-base and/or deoxyinosine residues. The terms “nucleic acid” or “nucleic acid sequence” can also be used interchangeably with gene, open reading frame (ORF), cDNA, and mRNA encoded by a gene.

The term “RNA” refers to a molecule comprising at least one ribonucleotide residue. By “ribonucleotide” is meant a nucleotide with a hydroxyl group at the 2′ position of a β-D-ribofuranose moiety. The terms encompass double stranded RNA, single stranded RNA, RNAs with both double stranded and single stranded regions, isolated RNA such as partially purified RNA, essentially pure RNA, synthetic RNA, recombinantly produced RNA, as well as altered RNA, or analog RNA, that differs from naturally occurring RNA by the addition, deletion, substitution, and/or alteration of one or more nucleotides. Such alterations can include addition of non-nucleotide material, such as to the end(s) of an siRNA or internally, for example at one or more nucleotides of the RNA. Nucleotides in the RNA molecules of the presently disclosed subject matter can also comprise non-standard nucleotides, such as non-naturally occurring nucleotides or chemically synthesized nucleotides or deoxynucleotides. These altered RNAs can be referred to as analogs or analogs of a naturally occurring RNA.

The terms “short hairpin RNA” and “shRNA” are used interchangeably and refer to any nucleic acid molecule capable of generating siRNA. In one embodiment, the shRNA comprises a polynucleotide having one or more loop structures and a stem comprising self complementary sense and antisense regions, wherein the antisense region comprises a sequence complementary to a region of a target nucleic acid molecule, and wherein the polynucleotide can be processed either in vivo or in vitro to generate an active siRNA capable of mediating RNAi. In another embodiment, retroviral vectors, in some embodiments expression vectors, encode shRNA, which are processed intracellularly, to generate siRNA that silence the expression of a target gene.

The terms “small interfering RNA”, “short interfering RNA” and “siRNA” are used interchangeably and refer to any nucleic acid molecule capable of mediating RNA interference (RNAi) or gene silencing. See e.g., Bass, Nature 411:428-429, 2001; Elbashir et al., Nature 411:494-498, 2001a; and PCT International Publication Nos. WO 00/44895, WO 01/36646, WO 99/32619, WO 00/01846, WO 01/29058, WO 99/07409, and WO 00/44914. In one embodiment, the siRNA comprises a double stranded polynucleotide molecule comprising complementary sense and antisense regions, wherein the antisense region comprises a sequence complementary to a region of a target nucleic acid molecule (for example, an mRNA encoding IL-17). In another embodiment, the siRNA comprises a single stranded polynucleotide having self-complementary sense and antisense regions, wherein the antisense region comprises a sequence complementary to a region of a target nucleic acid molecule. As used herein, siRNA molecules need not be limited to those molecules containing only RNA, but further encompass chemically modified nucleotides and non-nucleotides.

The terms “microRNA” and “miRNA” refer are used interchangeably and refer to synthetic or single-stranded RNA molecules of 21-23 nucleotides in length, which regulate gene expression. The terms “miRNA” and “non-coding RNA” can be used interchangeably. Primary transcript (pri-miRNA) is processed to give rise to short-stem-loop pre-miRNA, which are further processed to produce miRNA, which are single-stranded RNA molecules of 21-23 nucleotides. The miRNA are partially complementary to one or several mRNA transcripts, and they downregulate expression of genes encoded by the transcripts with which they interact. Thus, by way of example and not limitation, synthetic miRNA that interact with IL-17 mRNA can be generated and used to effect the downregulation of IL-17 expression thereby inhibiting IL-17 protein expression and consequently IL-17 activity.

The term “Ribozyme”, also known as “RNA enzyme” or “catalytic RNA” refers to ribonucleotides or RNA molecules that can act as enzymes that catalyze covalent changes in the structure of RNA molecules and that can cleave the target RNA molecule. Synthetic hammerhead ribozymes can be generated that recognize and cleave IL-17 RNA, or any other target disclosed herein, thereby inhibiting IL-17 protein expression.

The terms “identical” or percent “identity” in the context of two or more nucleotide or polypeptide sequences refer to two or more sequences or subsequences that are the same or have a specified percentage of amino acid residues or nucleotides that are the same, when compared and aligned for maximum correspondence, as measured using one of the sequence comparison algorithms disclosed herein or by visual inspection.

The term “substantially identical”, in the context of two nucleotide sequences, refers to two or more sequences or subsequences that have in some embodiments at least 60%, in some embodiments about 70%, in some embodiments about 80%, in some embodiments about 90%, in some embodiments about 95%, in some embodiments, about 97%, and in some embodiments about 99% nucleotide identity, when compared and aligned for maximum correspondence, as measured using one of the following sequence comparison algorithms (described herein below) or by visual inspection. In some embodiments, the substantial identity exists in nucleotide sequences of at least 50 residues, in some embodiments in nucleotide sequence of at least about 100 residues, in some embodiments in nucleotide sequences of at least about 150 residues, and in some embodiments in nucleotide sequences comprising complete coding sequences.

In one aspect, polymorphic sequences can be substantially identical sequences. The terms “polymorphic”, “polymorphism”, and “polymorphic variants” refer to the occurrence of two or more genetically determined alternative sequences or alleles in a population. An allelic difference can be as small as one base pair. As used herein in regards to a nucleotide or polypeptide sequence, the term “substantially identical” also refers to a particular sequence that varies from another sequence by one or more deletions, substitutions, or additions, the net effect of which is to retain biological activity of a gene, gene product, or sequence of interest.

For sequence comparison, typically one sequence acts as a reference sequence to which test sequences are compared. When using a sequence comparison algorithm, test and reference sequences are entered into a computer program, subsequence coordinates are designated if necessary, and sequence algorithm program parameters are selected. The sequence comparison algorithm then calculates the percent sequence identity for the designated test sequence(s) relative to the reference sequence, based on the selected program parameters.

Optimal alignment of sequences for comparison can be conducted, e.g., by the local homology algorithm of Smith & Waterman, 1981, by the homology alignment algorithm of Needleman & Wunsch, 1970, by the search for similarity method for Pearson & Lipman, 1988, by computerized implementations of these algorithms (GAP, BESTFIT, FASTA, and TFASTA, in the Wisconsin Genetics Software Package, available from Accelrys Inc., San Diego, California, United States of America), or by visual inspection. See generally, Ausubel, 1995.

In some embodiments, an algorithm for determining percent sequence identity and sequence similarity is the BLAST algorithm, which is described by Altschul et al., 1990. Software for performing BLAST analyses is publicly available through the website of the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment. The BLASTN program (for nucleotide sequences) uses as defaults a wordlength W=11, an expectation E=10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See Henikoff & Henikoff, 1992.

In addition to calculating percent sequence identity, the BLAST algorithm also performs a statistical analysis of the similarity between two sequences. See e.g., Karlin & Altschul, 1993. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is in some embodiments less than about 0.1, in some embodiments less than about 0.01, and in some embodiments less than about 0.001.

Another indication that two nucleotide sequences are substantially identical is that the two molecules specifically or substantially hybridize to each other under stringent conditions. In the context of nucleic acid hybridization, two nucleic acid sequences being compared can be designated a “probe” and a “target”. A “probe” is a reference nucleic acid molecule, and a “′target” is a test nucleic acid molecule, often found within a heterogeneous population of nucleic acid molecules. A “target sequence” is synonymous with a “test sequence”.

The phrase “hybridizing substantially to” refers to complementary hybridization between a probe nucleic acid molecule and a target nucleic acid molecule and embraces minor mismatches that can be accommodated by reducing the stringency of the hybridization media to achieve the desired hybridization.

“Stringent hybridization conditions” and “stringent hybridization wash conditions” in the context of nucleic acid hybridization experiments such as Southern and Northern blot analysis are both sequence- and environment-dependent. Longer sequences hybridize specifically at higher temperatures. An extensive guide to the hybridization of nucleic acids is found in Tijssen, 1993. Generally, highly stringent hybridization and wash conditions are selected to be about 5° C. lower than the thermal melting point (Tm) for the specific sequence at a defined ionic strength and pH. Typically, under “stringent conditions” a probe will hybridize specifically to its target subsequence, but to no other sequences.

The Tm is the temperature (under defined ionic strength and pH) at which 50% of the target sequence hybridizes to a perfectly matched probe. Very stringent conditions are selected to be equal to the Tm for a particular probe. An example of highly stringent hybridization conditions for Southern or Northern Blot analysis of complementary nucleic acids having more than about 100 complementary residues is overnight hybridization in 50% formamide with 1 mg of heparin at 42° C. An example of highly stringent wash conditions is 15 minutes in 0.1× standard saline citrate (SSC), 0.1% (w/v) SDS at 65° C. Another example of highly stringent wash conditions is 15 minutes in 0.2×SSC buffer at 65° C. (see Sambrook & Russell, 2001 for a description of SSC buffer and other stringency conditions). Often, a high stringency wash is preceded by a lower stringency wash to remove background probe signal. An example of medium stringency wash conditions for a duplex of more than about 100 nucleotides is 15 minutes in 1×SSC at 45° C. Another example of medium stringency wash for a duplex of more than about 100 nucleotides is 15 minutes in 4-6×SSC at 40° C. For short probes (e.g., about 10 to 50 nucleotides), stringent conditions typically involve salt concentrations of less than about 1M Na+ ion, typically about 0.01 to 1M Na+ ion concentration (or other salts) at pH 7.0-8.3, and the temperature is typically at least about 30° C. Stringent conditions can also be achieved with the addition of destabilizing agents such as formamide. In general, a signal to noise ratio of 2-fold or higher than that observed for an unrelated probe in the particular hybridization assay indicates detection of a specific hybridization.

The following are examples of hybridization and wash conditions that can be used to clone homologous nucleotide sequences that are substantially identical to reference nucleotide sequences of the presently disclosed subject matter: a probe nucleotide sequence hybridizes in one example to a target nucleotide sequence in 7% sodium dodecyl sulfate (SDS), 0.5M NaPO4, 1 mm EDTA at 50° C. followed by washing in 2×SSC, 0.1% SDS at 50° C.; in another example, a probe and target sequence hybridize in 7% sodium dodecyl sulfate (SDS), 0.5M NaPO4, 1 mm EDTA at 50° C. followed by washing in 1×SSC, 0.1% SDS at 50° C.; in another example, a probe and target sequence hybridize in 7% sodium dodecyl sulfate (SDS), 0.5M NaPO4, 1 mm EDTA at 50° C. followed by washing in 0.5×SSC, 0.1% SDS at 50° C.; in another example, a probe and target sequence hybridize in 7% sodium dodecyl sulfate (SDS), 0.5M NaPO4, 1 mm EDTA at 50° C. followed by washing in 0.1×SSC, 0.1% SDS at 50° C.; in yet another example, a probe and target sequence hybridize in 7% sodium dodecyl sulfate (SDS), 0.5M NaPO4, 1 mm EDTA at 50° C. followed by washing in 0.1×SSC, 0.1% SDS at 65° C.

The term “similarity” is contrasted with the term “identity”. Similarity is defined as above; “identity”, however, means a nucleic acid or amino acid sequence having the same amino acid at the same relative position in a given family member of a gene family. Homology and similarity are generally viewed as broader terms than the term identity. Biochemically similar amino acids, for example leucine and isoleucine or glutamate/aspartate, can be present at the same position—these are not identical per se, but are biochemically “similar.” As disclosed herein, these are referred to as conservative differences or conservative substitutions. This differs from a conservative mutation at the DNA level, which changes the nucleotide sequence without making a change in the encoded amino acid, e.g. TCC to TCA, both of which encode serine.

As used herein, DNA analog sequences are “substantially identical” to specific DNA sequences disclosed herein if: (a) the DNA analog sequence is derived from coding regions of the nucleic acid sequence shown in any of odd numbered SEQ ID NOs: 1-14; or (b) the DNA analog sequence is capable of hybridization of DNA sequences of (a) under stringent conditions and which encode a biologically active gene product of the nucleic acid sequence shown in any of odd numbered SEQ ID NOs: 1-14; or (c) the DNA sequences are degenerate as a result of alternative genetic code to the DNA analog sequences defined in (a) and/or (b). Substantially identical analog proteins will be greater than about 60% identical to the corresponding sequence of the native protein. Sequences having lesser degrees of identity but comparable biological activity are considered to be equivalents.

Nucleotide sequences are “substantially identical” where they have between about 70% and about 80% or more preferably, between about 81% and about 90%, or even more preferably, between about 91% and about 99%, sequence identity for nucleic acid residues which are identical to the nucleotide sequence of a reference gene.

Peptide sequences which have about 35%, or 45%, or preferably from 45-55%, or more preferably 55-65%, or most preferably 65% or greater, or more preferably about 70%, or more preferably about 80%; or more preferably, between about 81% and about 90%; or even more preferably, between about 91% and about 99% (e.g., 91%, 92%, 93%, 94%, and 95%) amino acids which are identical or functionally equivalent or biologically functionally equivalent to the amino acids of a IL-17, FFAR2, C3ar1, C3, Iba-1, IL-1β, IL-6 polypeptide will be sequences which are “substantially similar”.

The term “homology” describes a mathematically based comparison of sequence similarities which is used to identify genes or proteins with similar functions or motifs. Accordingly, the term “homology” is synonymous with the term “similarity” and “percent similarity” as defined above. Thus, the phrases “substantial homology” or “substantial similarity” have similar meanings.

In certain embodiments, the presently disclosed subject matter concerns the use of IL-17, FFAR2, C3ar1, C3, Iba-1, IL-1β, IL-6 genes and gene products that include within their respective sequences a sequence which is essentially that of a IL-17, FFAR2, C3ar1, C3, Iba-1, IL-1β, IL-6 gene, or the corresponding protein. The presently disclosed subject matter also includes the use of shRNA, siRNA and miRNA molecules, as well as synthetic hammerhead ribozymes, capable of suppressing and/or inhibiting expression of IL-17, FFAR2, C3ar1, C3, Iba-1, IL-1β, IL-6 genes and gene products that include within their respective sequences a sequence which is essentially that of a IL-17, FFAR2, C3ar1, C3, Iba-1, IL-1β, IL-6 gene, or the corresponding protein. The term “a sequence essentially as that of a IL-17, FFAR2, C3ar1, C3, Iba-1, IL-1β, IL-6 gene”, means that the sequence substantially corresponds to a portion of a IL-17, FFAR2, C3ar1, C3, Iba-1, IL-1β, IL-6 gene and has relatively few bases or amino acids (whether DNA or protein) which are not identical to those of a IL-17, FFAR2, C3ar1, C3, Iba-1, IL-1β, IL-6 protein or IL-17, FFAR2, C3ar1, C3, Iba-1, IL-1β, IL-6 gene, (or a biologically functional equivalent of, when referring to proteins). The term “biologically functional equivalent” is well understood in the art and is further defined in detail herein. Accordingly, sequences which have between about 70% and about 80%; or more preferably, between about 81% and about 90%; or even more preferably, between about 91% and about 99% (e.g., 91%, 92%, 93%, 94%, and 95%); of amino acids which are identical or functionally equivalent to the amino acids of a IL-17, FFAR2, C3ar1, C3, Iba-1, IL-1β, IL-6 polypeptide or of nucleotides of IL-17, FFAR2, C3ar1, C3, Iba-1, IL-1β, IL-6 gene, will be sequences which are “essentially the same”.

Alternatively, functionally equivalent proteins or peptides can be created via the application of recombinant DNA technology, in which changes in the protein structure can be engineered, based on considerations of the properties of the amino acids being exchanged. Changes designed by man can be introduced through the application of site-directed mutagenesis techniques, e.g., to introduce improvements to the antigenicity of the protein or to test IL-17, FFAR2, C3ar1, C3, Iba-1, IL-1β, IL-6 mutants in order to examine IL-17, FFAR2, C3ar1, C3, Iba-1, IL-1β, IL-6 activity, or other activity at the molecular level.

The term “functionally equivalent codon” is also used herein to refer to codons that encode biologically equivalent amino acids (see Table 1). Thus, when referring to the sequence examples presented in SEQ ID NOs: 1-14 applicants provide substitution from Table 1 of codons that encode biologically equivalent amino acids as described herein into the sequence examples. Thus, applicants are in possession of amino acid and nucleic acids sequences which include such substitutions but which are not set forth herein in their entirety for convenience.

TABLE 1
Functionally Equivalent Codons.

| Amino Acids | | | Codons |
|---|---|---|---|
| Alanine | Ala | A | GCA GCC GCG GCU |
| Cysteine | Cys | C | UGC UGU |
| Aspartic Acid | Asp | D | GAC GAU |
| Glumatic acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCG CCU |
| Glutamine | Gln | Q | CAA CAG |
| Arginine | Arg | R | AGA AGG CGA CGC CGG CGU |
| Serine | Ser | S | ACG AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It will also be understood that amino acid and nucleic acid sequences can include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences which may, for example, include various non-coding sequences flanking either of the 5′ or 3′ portions of the coding region or can include various internal sequences, i.e., introns, which are known to occur within genes.

The presently disclosed subject matter also encompasses the use of DNA segments which are complementary, or essentially complementary, to the sequences set forth in the specification. Nucleic acid sequences which are “complementary” are those which are base-pairing according to the standard Watson-Crick complementarity rules. As used herein, the term “complementary sequences” means nucleic acid sequences which are substantially complementary, as can be assessed by the same nucleotide comparison set forth above, or as defined as being capable of hybridizing to the nucleic acid segment in question under relatively stringent conditions such as those described herein. A particular example of a provided complementary nucleic acid segment is an antisense oligonucleotide.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1,000 mM, typically less than 500 mM, and preferably less than 200 mM. However, the combination of parameters is much more important than the measure of any single parameter. (See, e.g., Wetmur & Davidson, 1968).

Probe sequences can also hybridize specifically to duplex DNA under certain conditions to form triplex or other higher order DNA complexes. The preparation of such probes and suitable hybridization conditions are well known in the art.

As used herein, the term “DNA segment” refers to a DNA molecule which has been isolated free of total genomic DNA of a particular species. Furthermore, a DNA segment encoding a IL-17, FFAR2, C3ar1, C3, Iba-1, IL-1β, or IL-6 polypeptide refers to a DNA segment which contains IL-17, FFAR2, C3ar1, C3, Iba-1, IL-1β, or IL-6 coding sequences, yet is isolated away from, or purified free from, total genomic DNA of a source species, such as Homo sapiens. Included within the term “DNA segment” are DNA segments and smaller fragments of such segments, and also recombinant vectors, including, for example, plasmids, cosmids, phages, viruses, and the like.

Similarly, a DNA segment comprising an isolated or purified IL-17, FFAR2, C3ar1, C3, Iba-1, IL-1β, or IL-6 gene refers to a DNA segment including IL-17, FFAR2, C3ar1, C3, Iba-1, IL-1β, or IL-6 coding sequences isolated substantially away from other naturally occurring genes or protein encoding sequences. In this respect, the term “gene” is used for simplicity to refer to a functional protein, polypeptide or peptide encoding unit. As will be understood by those in the art, this functional term includes both genomic sequences and cDNA sequences. “Isolated substantially away from other coding sequences” means that the gene of interest, in this case, the IL-17, FFAR2, C3ar1, C3, Iba-1, IL-1β, or IL-6 gene, forms the significant part of the coding region of the DNA segment, and that the DNA segment does not contain large portions of naturally-occurring coding DNA, such as large chromosomal fragments or other functional genes or cDNA coding regions. Of course, this refers to the DNA segment as originally isolated, and does not exclude genes or coding regions later added to the segment by the hand of man.

It will also be understood that the presently disclosed subject matter is not limited to the particular nucleic acid and amino acid sequences of SEQ ID NOs: 1-14. Recombinant vectors, in some embodiments expression vectors, and isolated DNA segments can therefore variously include the IL-17, FFAR2, C3ar1, C3, Iba-1, IL-1β, or IL-6 polypeptide-encoding region itself, or nucleic acids of shRNA, siRNA or miRNA molecules, or synthetic hammerhead ribozymes, as disclosed herein, include coding regions bearing selected alterations or modifications in the basic coding region, or include encoded larger polypeptides which nevertheless include IL-17, FFAR2, C3ar1, C3, Iba-1, IL-1β, or IL-6, shRNA or siRNA shRNA, siRNA or miRNA molecules, or synthetic hammerhead ribozymes, that inhibit IL-17, FFAR2, C3ar1, C3, Iba-1, IL-1β, or IL-6 expression or polypeptide-encoding regions or can encode biologically functional equivalent proteins or peptides which have variant amino acid sequences that interfere with the ability of IL-17, FFAR2, C3ar1, C3, Iba-1, IL-1β, or IL-6.

### II. Methods, Systems, and Compositions for Treating Neuroinflammation

Neuroinflammation has been implied in the development of POCD. A surgery on peripheral tissues or organs can induce systemic inflammation, which then is transmitted into the brain to cause neuroinflammation to induce POCD. Gut microbiota has been shown to be involved in regulating inflammation, however, direct evidence to suggest the involvement of gut microbiota in POCD has not been reported, nor have any mechanisms of action been discovered.

It is suggested that environmental enrichment reduces learning and memory impairment after surgery. Environment enrichment may enhance the physical activity of animals. Thus, in some embodiments, the present disclosure evaluates whether an appropriate level of exercise attenuates neuroinflammation, and whether impairment of learning and memory after surgery and that these effects are mediated by gut microbiota alterations. To test these hypotheses, and as discussed in detail in the Examples set forth herein below, adult and old mice were subjected to different levels of exercise. Fecal transplantation was performed to determine the role of gut microbiota in the effects of exercise on POCD development. Left carotid artery exposure was chosen to be the surgical procedure because this procedure is a component of carotid endarterectomy that is commonly performed in elderly patients. The presently disclosed results provide the first direct evidence for the involvement of gut microbiota in POCD. Valeric acid, a product of gut microbiota, was surprisingly discovered to play a role in mediating the effects of gut microbiota on learning and memory impairment after surgery.

Transplantation of feces from surgery mice but not from control mice led to learning and memory impairment in non-surgery mice. Low intensity exercise improved learning and memory in surgery mice. Exercise attenuated surgery-induced neuroinflammation and decrease of gut microbiota diversity. These exercise effects were present in non-exercise mice receiving feces from exercise mice. Exercise reduced valeric acid, a gut microbiota product, in the blood. Valeric acid worsened neuroinflammation, learning and memory in exercise mice with surgery. The downstream effects of exercise included attenuating growth factor decrease, maintaining astrocytes in the A2 phenotypical form possibly via decreasing C3 signaling and improving neuroplasticity. Similar to these results from adult mice, exercise attenuated learning and memory impairment in old mice with surgery. Old mice receiving feces from old exercise mice had better learning and memory than those receiving control old mouse feces. Surgery increased blood valeric acid. Valeric acid blocked exercise effects on learning and memory in old surgery mice. Exercise stabilized gut microbiota, reduced neuroinflammation, attenuated growth factor decrease and preserved neuroplasticity in old mice with surgery. These results provide the first direct evidence that gut microbiota alteration contributes to POCD development, valeric acid is a mediator for this effect and a potential target for brain health, and low intensity exercise stabilizes gut microbiota in the presence of insult, such as surgery.

Thus, in one aspect of the presently disclosed subject matter, exercise reduces post-surgery neuroinflammation and impairment of cognition and neuroplasticity. In another aspect, exercise decreases gut microbiota changes and valeric acid increase after surgery. In another aspect, the effects of exercise on surgery-induced changes are transferable by fecal transplantation. In another aspect, valeric acid blocks the beneficial effects of exercise.

Moreover, gut microbiota can produce multiple metabolites. Among them, short chain fatty acid (SCFA) metabolites may regulate inflammatory responses. As discussed further in the working Examples, studies disclosed herein tested whether SCFAs are involved in age-related changes in neuroinflammation tolerance, including for example brain ischemic tolerance, and how SCFAs affect stroke outcome.

Interleukin (IL)-17 is a proinflammatory cytokine. It is produced from a group of T helper cells and can induce the production of chemokines that recruit immune cells to the site of inflammation and facilitate the production of other proinflammatory cytokines, such as IL-6 and IL-1β. Thus, as disclosed herein, studies were designed to determine whether age-related changes in gut microbiota contribute to the worsened neurological outcome after brain ischemia and whether SCFAs and IL-17 mediate this gut microbiota effect. As disclosed herein for the first time, a novel pathway, gut microbiota-valeric acid-free fatty acid receptor (FFAR) 2-IL-17, mediates increased inflammatory response to neuroinflammatory events, including brain ischemia, and worsened neurological outcome after ischemic stroke, surgery and the like.

Thus, provided herein in some embodiments are methods of treating neuroinflammation in a subject. Such treatments methods can include administering a therapy for modulating, including disrupting, the valeric acid interleukin (IL)-17 pathway in the subject, and more specifically the gut microbiota-valeric acid-free fatty acid receptor (FFAR) 2-IL-17 pathway, such that neuroinflammation in the subject is treated. In some embodiments, the neuroinflammation is substantially prevented and/or reduced. As discussed herein, the neuroinflammation in the subject can include, but is not limited to post-surgery neuroinflammation, and/or impairment of cognition, and/or postoperative cognitive dysfunction (POCD), and/or neuroplasticity. In some embodiments, an effective amount of the therapy is administered. In some embodiments, the subject is an old or aged subject. In some embodiments, such methods further comprise measuring blood valeric acid concentration in the subject prior to, during and/or after administration of the therapy. Blood valeric acid can be measured as disclosed in the Examples herein, and/or using any acceptable method as would be appreciated by one of ordinary skill in the art upon a review of the instant disclosure.

By way of example, and without being bound by any particular theory or mechanism of action, the therapy can include one or more of: a) an agent for modulating valeric acid, including a concentration of valeric acid, such as an agent for modulating, including antagonizing and/or disrupting, the valeric acid interleukin (IL)-17 pathway in the subject, including the gut microbiota-valeric acid-free fatty acid receptor (FFAR) 2-IL-17 pathway, b) exercise, c) fecal transplantation and/or d) an agent to decrease bacteria producing valeric acid. For example, an appropriate exercise regimen can decrease gut microbiota changes in the subject and/or reduce valeric acid concentrations in the subject. Where the therapeutic approach administered to a subject comprises exercise, in some embodiments a low intensity exercise can be protective. In some aspects, a low intensity exercise is any exercise activity that is about 15 to 60%, about 25 to 50%, or about 35 to 40%, of the subject's maximal exercise capacity. In some embodiments, VO2 max is a good reflection of maximal exercise capacity, wherein VO2 max is the maximum (max) rate (V) of oxygen (O₂) a subject's body is able to use during exercise.

Likewise, a fecal transplantation can decrease bacteria producing valeric acid, including, for example, by decreasing Megasphaera massiliensis and other bacteria known to produce valeric acid. Fecal transplantation can act as an agent that can decrease bacteria producing valeric acid, and can particularly act as an agent that decreases Megasphaera massiliensis and other bacteria known to produce valeric acid.

With regard to fecal transplantation procedures and therapies, the human gut microbiome is unique to each individual and can include more than a thousand different species. Disruptions to the homeostatic of the gut microbiome can play a role in inflammatory bowel diseases. In some aspects reshaping the microbial contents can involve the use of prebiotics, probiotics, and antibiotics, but sometimes these approaches can fail to produce stable, long-term improvements in bacterial diversity. Fecal transplantation of gut microbiota from a healthy donor to an ill recipient, or a subject in need of therapy, in order to reconstitute the normal or desired flora of a patient can be an alternative.

Fecal microbiota transplants involves taking liquefied donor feces and transferring it to the patient such as during a colonoscopy. The patient receives a transplanted population of commensalistic bacteria, for example, that can combat the overgrowth of pathogenic bacteria. The donor is first tested for communicable infectious diseases. The donor then gives a stool sample which is mixed with a small amount of dilutant, such as sterile water or saline solution, which is blended by shaking the mixed material and then straining the blended material to separate some of the particulate material. A provider can then draw some of the liquid (e.g., an amount to provide or to initiating providing a desired microbiome or a component of a microbiome) into a syringe for administering into the intestinal tract of a patient (e.g., a recipient) which can be accomplished with an endoscope, colonoscopy or enema. By way of example and not limitation, a recipient is any subject that is in need of treatment as disclosed herein. By way of example and not limitation, a donor is any subject with a desired microbiome or component of a microbiome for which the recipient could benefit upon transplantation.

In some embodiments, the therapy administered to a subject can include an agent for reducing IL-17 in the subject, including for example, but not limited to, an antibody against IL-17. The agent could also include, in some aspects, an agent to decrease and/or block FFAR2 in mediating an increase of IL-17 caused by valeric acid, including for example an agent that comprises GLPG-0974, a FFAR2 antagonist. Another non-limiting example FFAR2 antagonist includes CATPB [(S)-3-(2-(3-Chlorophenyl) acetamido)-4-(4-(trifluoromethyl)phenyl) butanoic acid]. Moreover, such therapies can include an agent for reducing IL-17 downstream immune and neuroinflammatory targets selected from C3ar1, C3, Iba-1, IL-1β, IL-6, and combinations thereof, including antagonists and/or antibodies against C3ar1, C3, Iba-1, IL-1β, and/or IL-6. By way of example and not limitation, an agent and/or inhibitor for C3ar1 can comprise SB290157. By way of example and not limitation, an agent and/or inhibitor for C3 can comprise CR2-Crry. By way of example and not limitation, an agent and/or inhibitor for IL-1β can comprise interleukin-1 receptor antagonist (IL-1RA). By way of example and not limitation, an agent and/or inhibitor for IL-6 can comprise one or more, or combination of, tocilizumab, sarilumab, satralizumab and siltuximab (these are all monoclonal antibodies developed to antagonize IL-6 and are used in humans). In some embodiments, an effective amount of the agent is administered.

Each of IL-17, FFAR2, C3ar1, C3, Iba-1, IL-1β, and/or IL-6 represent therapeutic targets within the valeric acid IL-17 pathway disclosed herein for the treatment of neuroinflammation and related conditions. As discussed, further hereinbelow in the Examples, such targets can be modulated by way of administration of one or more antibodies with an affinity to the targets and/or RNAi constructs, e.g. dsRNA, siRNA, shRNA, miRNA, or synthetic hammerhead ribozymes, and/or vectors (including but not limited to expression vectors) as discussed hereinabove, directed to the targets.

Each of the IL-17, FFAR2, C3ar1, C3, Iba-1, IL-1β, and/or IL-6 targets can be further defined by their representative human nucleotide and polypeptide sequences as follows: IL-17 nucleotide (SEQ ID NO. 1), IL-17 polypeptide (SEQ ID NO. 2), FFAR2 nucleotide (SEQ ID NO. 3), FFAR2 polypeptide (SEQ ID NO. 4), C3ar1 nucleotide (SEQ ID NO. 5), C3ar1 polypeptide (SEQ ID NO. 6), C3 nucleotide (SEQ ID NO. 7), C3 polypeptide (SEQ ID NO. 8), Iba-1 nucleotide (SEQ ID NO. 9), Iba-1 polypeptide (SEQ ID NO. 10), IL-1ß nucleotide (SEQ ID NO. 11), IL-1β polypeptide (SEQ ID NO. 12), and/or IL-6 nucleotide (SEQ ID NO. 13), IL-6 polypeptide (SEQ ID NO. 14). These targets, their sequences and corresponding GENBANK® Accession and GenInfoIdentifier (GI) numbers are provided in Table 2.

TABLE 2

| | | Polypeptide | | GENBANK ® |
|---|---|---|---|---|
| | Nucleotide | (amino acid) | GENBANK ® | GenInfoIdentifier |
| Target | SEQ ID NO. | SEQ ID NO. | Accession No. | (GI) |
| IL-17 | 1 | 2 | NG_033021.1 | 429535870 |
| FFAR2 | 3 | 4 | EU432114.1 | 166714268 |
| C3ar1 | 5 | 6 | NG_050736.1 | 1032528813 |
| C3 | 7 | 8 | NG_009557.1 | 222831655 |
| Iba-1 | 9 | 10 | D86438.1 | 1596162 |
| IL-1β | 11 | 12 | NG_008851.1 | 210032106 |
| IL-6 | 13 | 14 | NG_011640.1 | 225543208 |

Such treatments can also include methods for improving neurological outcome and/or mediating inflammatory response in a subject suffering from ischemic stroke and/or surgery-induced neuroinflammation. As discussed above, such a subject can be administered a therapy for reducing valeric acid, including a) an agent for modulating valeric aid, including a concentration of valeric acid, such as an agent for modulating, including antagonizing and/or disrupting, the valeric acid interleukin (IL)-17 pathway in the subject, including a) the gut microbiota-valeric acid-free fatty acid receptor (FFAR) 2-IL-17 pathway, b) exercise, c) fecal transplantation and/or d) an agent to decrease bacteria producing valeric acid, as discussed herein.

### III. RNA Interference and Uses of Same for Targeting Valeric Acid-IL-17 Pathway

In some embodiments, the presently disclosed subject matter takes advantage of RNAi technology (for example shRNA, siRNA and miRNA molecules and ribozymes) to cause the down regulation of target genes, e.g., genes or nucleotide sequences for IL-17, FFAR2, C3ar1, C3, Iba-1, IL-1β, and/or IL-6, a process referred to as RNA interference (RNAi). As used herein, “RNA interference” (RNAi) refers to a process of sequence-specific post-transcriptional gene silencing mediated by a small interfering RNA (siRNA) or short hairpin RNA (shRNA) molecules, miRNA molecules or synthetic hammerhead ribozymes. See generally Fire et al., Nature 391:806-811, 1998, and U.S. Pat. No. 6,506,559. The process of RNA interference (RNAi) mediated post-transcriptional gene silencing is thought to be an evolutionarily conserved cellular defense mechanism that has evolved to prevent the expression of foreign genes (Fire, Trends Genet 15:358-363, 1999).

RNAi might have evolved to protect cells and organisms against the production of double stranded RNA (dsRNA) molecules resulting from infection by certain viruses (particularly the double stranded RNA viruses or those viruses for which the life cycle includes a double stranded RNA intermediate) or the random integration of transposon elements into the host genome via a mechanism that specifically degrades single stranded RNA or viral genomic RNA homologous to the double stranded RNA species.

The presence of long dsRNAs in cells stimulates the activity of the enzyme Dicer, a ribonuclease III. Dicer catalyzes the degradation of dsRNA into short stretches of dsRNA referred to as small interfering RNAs (siRNA) (Bernstein et al., Nature 409:363-366, 2001). The small interfering RNAs that result from Dicer-mediated degradation are typically about 21-23 nucleotides in length and contain about 19 base pair duplexes. After degradation, the siRNA is incorporated into an endonuclease complex referred to as an RNA-induced silencing complex (RISC). The RISC is capable of mediating cleavage of single stranded RNA present within the cell that is complementary to the antisense strand of the siRNA duplex. According to Elbashir et al., cleavage of the target RNA occurs near the middle of the region of the single stranded RNA that is complementary to the antisense strand of the siRNA duplex (Elbashir et al., Genes Dev 15:188-200, 2001b).

RNAi has been described in several cell type and organisms. Fire et al., 1998 described RNAi in C. elegans. Wianny & Zernicka-Goetz, Nature Cell Biol 2:70-75, 1999 disclose RNAi mediated by dsRNA in mouse embryos. Hammond et al., Nature 404:293-296, 2000 were able to induce RNAi in Drosophila cells by transfecting dsRNA into these cells. Elbashir et al. Nature 411:494-498, 2001a demonstrated the presence of RNAi in cultured mammalian cells including human embryonic kidney and HeLa cells by the introduction of duplexes of synthetic 21 nucleotide RNAs.

Other studies have indicated that a 5′-phosphate on the target-complementary strand of a siRNA duplex facilitate siRNA activity and that ATP is utilized to maintain the 5′-phosphate moiety on the siRNA (Nykanen et al., Cell 107:309-321, 2001). Other modifications that might be tolerated when introduced into an siRNA molecule include modifications of the sugar-phosphate backbone or the substitution of the nucleoside with at least one of a nitrogen or sulfur heteroatom (PCT International Publication Nos. WO 00/44914 and WO 01/68836) and certain nucleotide modifications that might inhibit the activation of double stranded RNA-dependent protein kinase (PKR), specifically 2′-amino or 2′-O-methyl nucleotides, and nucleotides containing a 2′-O or 4′-C methylene bridge (Canadian Patent Application No. 2,359,180).

Other references disclosing the use of dsRNA and RNAi include PCT International Publication Nos. WO 01/75164 (in vitro RNAi system using cells from Drosophila and the use of specific siRNA molecules for certain functional genomic and certain therapeutic applications); WO 01/36646 (methods for inhibiting the expression of particular genes in mammalian cells using dsRNA molecules); WO 99/32619 (methods for introducing dsRNA molecules into cells for use in inhibiting gene expression); WO 01/92513 (methods for mediating gene suppression by using factors that enhance RNAi); WO 02/44321 (synthetic siRNA constructs); WO 00/63364 and WO 01/04313 (methods and compositions for inhibiting the function of polynucleotide sequences); and WO 02/055692 and WO 02/055693 (methods for inhibiting gene expression using RNAi).

In some embodiments, the presently disclosed subject matter utilizes RNAi to at least partially inhibit expression of one or more targets of interest, including for example IL-17, FFAR2, C3ar1, C3, Iba-1, IL-1β, and/or IL-6. Inhibition is preferably at least about 10% of normal expression amounts, or at least about 20% of normal expression amounts, or at least about 30% of normal expression amounts, or at least about 40% of normal expression amounts, or at least about 50% or more of normal expression amounts, or at least about 60% or more of normal expression amounts, or at least about 70% or more of normal expression amounts, or at least about 80% or more of normal expression amounts, or at least about 90% or more of normal expression amounts. In some embodiments, the method comprises introducing an RNA into at least one of a plurality of cells, tissues, or organs in an amount sufficient to inhibit expression of IL-17, FFAR2, C3ar1, C3, Iba-1, IL-1β, and/or IL-6, wherein the RNA comprises a ribonucleotide sequence which corresponds to a coding strand of a gene encoding the target.

### IV. Antibodies, Dosages and Uses

A dosage regimen for treatment with the active agents is based on a variety of factors, including the type of injury, the age, weight, sex, medical condition of the individual, the severity of the condition, the route of administration, and the particular compound employed. Thus, the dosage regimen may vary, but can be determined routinely by a physician using standard methods.

In one aspect, an antibody of the presently disclosed subject matter can be administered at a dose of about 0.01 mg/kg to about 100 mg/kg body weight. In another aspect, an antibody of the presently disclosed subject matter can be administered at a dose of about 0.1 mg/kg to about 50 mg/kg. In yet another aspect, an antibody of the presently disclosed subject matter can be administered at a dose of about 1.0 mg/kg to about 25 mg/kg body weight. In another aspect, an antibody of the presently disclosed subject matter can be administered at a dose of about 0.1, 0.5, 0.75, 0.833, 1.0, 1.25, 1.5, 2.0, 2.5, 3.0, 3.5, 4.0, 4.5, 5.0, 5.5, 6.0, 6.5, 7.0, 7.5, 8.0, 8.5, 9.0, 9.5, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, and 20.0 mg/kg body weight. The presently disclosed subject matter further encompasses similar increments within each range of doses described herein.

In one embodiment, the agonist or additional therapeutic agent is administered at a dose of about 1 μg/kg body weight to about 1 g/kg body weight.

The treatment regimen will vary depending on the disease being treated, based on a variety of factors, including the type of injury, the age, weight, sex, medical condition of the individual, the severity of the condition, the route of administration, and the particular compound employed. The treatment can include administration of a pharmaceutical composition of the presently disclosed subject matter once or more than once. Other therapeutic drugs and agents can be administered as well.

In one embodiment, a dose can be administered once a week. In another embodiment, a dose can be administered at least once a week. In one embodiment, a dose is administered two or more times a week. In another embodiment, a dose is administered three or more times a week. In another embodiment, a dose is administered ever third day. In one embodiment, the duration of treatment can be for up to one year, or up to six months, or up to three months.

In one embodiment, an antibody of the presently disclosed subject matter is purified.

In one embodiment, an antibody of the presently disclosed subject matter is substantially pure.

The presently disclosed subject matter further provides for the use of the proteins or peptides where one or more conservative amino acid substitutions are made in the sequence and that the substitution has no effect on the desired biological activity, where such activity is desired. In one aspect, one conservative amino acid substitution is made. In one aspect, at least two conservative amino acid substitutions are made. When two or more substitutions are made, they do not have to be at adjacent amino acid residue positions.

TABLE 3
Exemplary Conservative Amino Acid Substitutions

| Group | Characteristics | Amino Acids |
|---|---|---|
| A. | Small aliphatic, nonpolar or slightly | Ala, Ser, Thr, Pro, Gly |
| | polar residues | |
| B. | Polar, negatively charged residues and | Asp, Asn, Glu, Gln |
| | their amides | |
| C. | Polar, positively charged residues | His, Arg, Lys |
| D. | Large, aliphatic, nonpolar residues | Met Leu, Ile, Val, Cys |
| E. | Large, aromatic residues | Phe, Tyr, Trp |

### V. Uses and Pharmaceutical Compositions

In some embodiments, provided are uses of a therapy for modulating a valeric acid IL-17 pathway for treating neuroinflammation in a subject, whereby neuroinflammation in the subject is treated. The therapies in such uses include the same methods and compositions for treating neuroinflammation as disclosed herein. The compositions can be for use in the preparation of a medicament for treating neuroinflammation.

In some embodiments, the presently disclosed subject matter provides a pharmaceutical composition, which can include a pharmaceutically acceptable carrier. In some embodiments, the compositions, such as but not limited to antibodies, RNAi constructs, fecal transplantation samples, and small molecule agents as mentioned herein above, of the presently disclosed subject matter are formulated for use in treating neuroinflammation. The compositions can be prepared for oral, parenteral, or other administration, such as using a formulation known in the art for preparing an agent for treating another indication known to be treated by the agent.

In some embodiments, compositions of the presently disclosed subject matter are provided for use in the treatments as disclosed herein, such as for use in the treatments in humans and in animals. In some embodiments, compositions can be provided for use in combination with each other.

In some embodiments, the method further comprises administering one or more additional therapeutic agents to the animal subject. The one or more additional therapeutic agents can be an agent use to treat or mitigate one or more symptoms in the subject. By way of example and not limitation, the additional therapeutic agent can be a therapeutic agent for treating fever or pain.

In some embodiments, the compositions of the presently disclosed subject matter can be provided as pharmaceutical compositions and be provided in pharmaceutically acceptable carriers. In some embodiments, the compositions can be provided as a pharmaceutically acceptable salt. Such salts include, but are not limited to, pharmaceutically acceptable acid addition salts, pharmaceutically acceptable base addition salts, pharmaceutically acceptable metal salts, ammonium and alkylated ammonium salts, and combinations thereof.

Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like.

Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris (hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like.

Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like.

In some embodiments, the presently disclosed compounds can further be provided as a solvate.

Liquid carriers suitable for use in the presently disclosed subject matter include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the presently disclosed subject matter include, but are not limited to, inert substances such as lactose, starch, glucose, methylcellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins.

Oral formulations can include standard carriers such as pharmaceutical grades of mannitol, lactose, starch, magnesium stearate, polyvinyl pyrollidone, sodium saccharine, cellulose, magnesium carbonate, etc.

### EXAMPLES

The following examples are included to further illustrate various embodiments of the presently disclosed subject matter. However, those of ordinary skill in the art should, in light of the present disclosure, appreciate that many changes can be made in the specific embodiments which are disclosed and still obtain a like or similar result without departing from the spirit and scope of the presently disclosed subject matter.

### Materials and Methods for Examples 1-4

The experimental protocols and procedures were approved by the Institutional Animal Care and Use Committee of the University of Virginia (Charlottesville, VA, USA; protocol number: 3114). All animal experiments were performed in accordance with the National Institutes of Health Guide for the Care and Use of Laboratory Animals (NIH publications number 80-23) revised in 2011.

The sources of key materials were listed in the Key Resources Table in the supplemental materials.

### Animals and Experimental Design

Eight-week old male C57BL/6J mice weighing 19-22 g were housed in a room maintained under constant environmental conditions (temperature 22-24° C., a 12 h light/dark cycle, and 50±10% humidity) with free access to food and water. All of the mice were allowed to acclimate for one week before experiments. The mice were randomly assigned to the following groups in the first experiment: 1) control group (not being exposed to anesthesia and surgery), 2) exercise group (exercised at 35-40% maximal capacity, but not being exposed to anesthesia and surgery), 3) surgery group (left carotid artery exposure for 15 min under isoflurane anesthesia for 2 h), and 4) to 6) Exe-1+Sur, Exe-m+Sur and Exe-h+Sur groups: exercised at 35-40%, 55-60% and 75-80% maximal capacity, respectively, and were subjected to anesthesia and surgery. The mice were used for learning and memory tests starting 4 days after the surgery (n=20) or their brains were harvested for biochemical assays at 6 h, 24 h, 48 h, 72 h, 96 h and 7 days after the surgery (n=9 or 14), for immunofluorescent staining at 48 h and 19 days after the surgery (n=6), and for Golgi staining at 19 days after the surgery (n=8). Blood and feces from mice of the first 4 groups were harvested for measuring short chain fatty acids (SCFAs) at 7 days after the surgery (n=7) and for 16S analyses at 3 and 7 days after the surgery (n=8) or at the end of 4-week exercise protocol (n=8) or at the corresponding time in the first 2 groups (n=16). Mice for tissue harvesting were different cohorts of mice that were used for learning and memory tests.

In the second experiment, mice were randomly assigned to: 1) Trans-control+Sur group and 2) Trans-exe+Sur group. Mice in the first group received 500 μl fecal solution from control group by gastric gavage and 600 μl by enema once a day for 7 consecutive days after antibiotic treatment to eliminate the native gut microbiota in the recipients. Mice in the second group received 500 μl fecal solution from exercise mice by gastric gavage and 600 μl by enema once a day for 7 consecutive days after the antibiotic treatment. Feces from the recipients were harvested 14 days after fecal transplantation for 16S analysis. The mice were then subjected to surgery. Learning and memory were evaluated from 4 days after surgery (n=15). Brain was harvested for biochemical studies as in the first experiment (n=10).

In the third experiment, mice were randomly assigned to: 1) Trans-control group, and 2) Trans-control+Sur group. Mice in both groups received transplantation of feces from control mice. The second group had surgery 14 days after fecal transplantation. Hippocampus was harvested 2 days after surgery to measure GDNF (n=10).

In the fourth experiment, mice were randomly assigned to: 1) control group (naïve mice that were not exposed to any experimental conditions described in this study), 2) antibiotic group that received antibiotics for 7 days, and 3) Trans-control group that received transplantation of feces from control mice. Learning and memory were assessed 19 days after the completion of fecal transplantation (26 days after the completion of antibiotic treatment in the antibiotic group) (n=12).

In the fifth experiment, mice were randomly assigned to: 1) control group, 2) Trans-control group that received transplantation of feces from control mice, and 3) Trans-surgery group that received transplantation of feces from surgery mice. Fecal transplantation was performed as described for the second experiment. Feces from the recipients were harvested 14 days after fecal transplantation for 16S analysis (n=10). Learning and memory were assessed 4 days after the fecal sample was harvested (n=17).

In the sixth experiment, mice were randomly assigned to: 1) surgery plus normal saline group that received 200 μl normal saline (NS) by intraperitoneal injection once a week for 4 weeks and then surgery, 2) exercise plus normal saline plus surgery group that received 200 μl NS by intraperitoneal injection once a week during the 4-week exercise at 35-40% maximal capacity and then surgery, 3) exercise plus valeric acid plus surgery group that received valeric acid (200 mg/kg in 200 μl) by intraperitoneal injection once a week during the 4-week exercise at 35-40% maximal capacity and then surgery. The injection was given in the morning of first exercise day of the week. One additional injection was performed on the surgery day. Behavioral (n=13) and biochemical (n=10) outcomes were assessed as in the second experiment.

In the seventh experiment, 13-week old (weighing 23-27 g) male C57BL/6J mice were randomly assigned to: 1) control group, 2) NS group that received 4 μl NS by intracerebroventricular injection once daily for 4 consecutive days, and 3) valeric acid group that received 4 mg/kg in 4 μl by intracerebroventricular injection once daily for 4 consecutive days. The left and right cerebroventricles were injected on an alternated schedule. Learning and memory were assessed 4 days after the injection (n=15).

In the eighth experiment, mice were randomly assigned to: 1) surgery plus dimethyl sulfoxide (DMSO) group that received DMSO by intracerebroventricular injection once daily for 4 consecutive days starting on the surgery day, 2) surgery plus C3ar antagonist group that received C3ar antagonist (SB290157) by intracerebroventricular injection once daily for 4 days starting on the surgery day, 3) exercise plus DMSO plus surgery group that received DMSO by intracerebroventricular injection once daily for 4 consecutive days starting on the surgery day after the 4-week exercise at 35-40% maximal capacity, and 4) exercise plus C3ar agonist plus surgery group that received a C3ar agonist by intracerebroventricular injection once daily for 4 consecutive days starting on the surgery day after the 4-week exercise at 35-40% maximal capacity. Behavior tests were started from 4 days after surgery as stated above (n=15).

In the ninth experiment, mice were randomly assigned to: 1) control group, 2) surgery group, 3) surgery plus GDNF group that received GDNF by intracerebroventricular injection when they had surgery, and 4) surgery plus heat-inactivated GDNF group that received heat-inactivated GDNF by intracerebroventricular injection when they had surgery. Hippocampi were harvested at 48 h after surgery for ELISA study (n=12).

After one-week acclimation, old mice (18-month old male C57BL/6J mice) weighing 30-36 g were used in three studies. In the first experiment, old mice were randomly assigned to: 1) control group, 2) exercise group in which mice had 4-week exercise at 35-40% maximal capacity, 3) surgery group, and 4) Exe+Sur group in which mice had 4-week exercise at 35-40% maximal capacity before surgery. The animals were used for learning and memory tests starting 4 days after the surgery (n=12) or their brains were harvested for biochemical assays at 48 h after the surgery (n=10 or 12), for immunofluorescent staining at 48 h and 19 days after the surgery (n=6), and for Golgi staining at 19 days after the surgery (n=8). Blood and feces from mice of the 4 groups were harvested for SCFA measurement at 7 days after the surgery (n=7) and for 16S analyses at 3 and 7 days after the surgery (n=8) or at the end of 4-week exercise protocol or at the corresponding time in the first 2 groups (n=16). Mice for tissue harvesting were different cohorts that were used for learning and memory tests. In second experiment, old mice were randomly assigned to: 1) Trans-Old Control+Sur group, and 2) Trans-Old Exe+Sur group. These mice were subjected to surgery 14 days after fecal transplantation as described in the second experiment of young adult mice. Learning and memory were evaluated from 4 days after surgery (n=10). In third experiment, old mice were randomly assigned to: 1) surgery plus NS group; 2) exercise plus NS plus surgery group; and 3) exercise plus valeric acid plus surgery group. These mice received exercise conditioning and valeric acid as described in sixth experiment of young adult mice. Behavioral outcomes were tested starting 4 days after surgery (n=11).

The sample sizes (n) described above represented the number of animals that were randomized into each group/condition. These numbers of animals were the sum of at least 3 replicates of each experiment. Animals were randomly distributed into groups based on computer-generated randomization tables in each experiment.

### Maximal Exercise Capacity Determination and Exercise Training

Before surgery, 8-week and 18-month old mice were exposed to a 4-week treadmill aerobic exercise after a determination of maximal exercise capacity of individual mouse. This determination was performed in a way similar to those described before. Briefly, the mice were acclimated to the treadmill in the first day with initial settings of shock grid at 25 V, 0.3 mA, and 2 Hz. and the speed and inclination at zero for 10 min. The treadmill speed was then increased to 10 cm/s with inclination set to 5° for 10 min. Next, the treadmill speed and inclination were increased to 15 cm/s and 100 for 5 min. The speed was then increased by 5 cm/s every 5 min to an average maximal speed of 70 cm/s in young mice and 60 cm/s in old mice (around 65-75 cm/s and 55-65 cm/s, respectively) as they reached to the criteria for exercise-induced exhaustion. These criteria were: 1) 10 consecutive seconds on the electric grid; 2) spending more than 50% of time on the grid; and/or 3) lack of motivation to manual prodding. The mouse was removed immediately from the respective lane once one or more of these criteria was met. On the next day, mice ran on the same treadmill at half of average maximal speed (35 cm/s in young mice or 30 cm/s in old mice) and 100 of inclination until they reached one of the exhaustion criteria again, and the duration of continuous exercise was recorded as the maximal exercise capacity of each mouse. After these two protocols, mice were housed separately for 30 min to avoid noticeable aggressive behavior following exercise. After resting for 2 days, mice assigned to exercise training groups were subjected to a 4-week protocol of forced treadmill running at 35-40%, 55-60% or 75-80% of the maximal capacity, respectively, for 5 days a week. Mice that were shocked for more than 5% of the total daily exercise time in 2 consecutive days or more than 3% of the total time in 3 consecutive days would be excluded. The non-exercise groups in the same set of experiments with the exercise groups were placed on a non-moving treadmill daily for about 30-40 min. They received 10 shocks for a total of 5 s, the average amount of shocks that mice in an exercise group received each day, during their stay in non-moving treadmill. If a set of experiments did not have an exercise group, no mice in the set of experiments received shocks.

### Antibiotic Treatment

To facilitate the colonization of the transplanted microbiota, recipient mice were given an antibiotic treatment once daily for 7 consecutive days to eliminate their original gut microbiota as described by us and others [36]. In brief, after purgation with 10% magnesium sulfate (200 μl/10 g, twice with 3 h interval) by gastric gavage in the first day, C57BL/6J mice received amoxicillin/clavulanic acid (200 mg/kg), metronidazole (200 mg/kg) and cefazolin (2 g/kg) by gastric gavage and enema (in 300 μl for gastric gavage and 400 μl NS for enema) in the morning once a day for 7 consecutive days, and amoxicillin/clavulanic acid (20 mg/kg), metronidazole (20 mg/kg) and cefazolin (300 mg/kg) in 200 μl NS daily by intraperitoneal injection for the same 7 days (at 4 μm to 5 pm). To prevent bacterial cross contamination, mouse handling and daily cage and water bottle changes were performed by technicians wearing a clean gown and gloves in a ventilated hood. Feces from these mice were harvested 24 h after the last treatment for 16S analyses to determine the effect.

### Fecal Transplantation

Fresh fecal pellets collected within 30 min after bowel movement from control mice, mice immediately after the completion of 4-week exercise or mice 2 to 8 days after surgery were diluted with 100 mg/ml sterile saline. All fecal pellets of 7 to 10 donors with each experimental condition were mixed and re-suspended together in saline. Briefly, the fecal matter was vortex-mixed for 5 min and then passed through 70 μm nylon cell strainer to remove undigested food and particulate materials. The recipient mice received 500 μl corresponding fecal solution by gastric gavage in the morning and 600 μl by enema in the afternoon from 24 h after the last dose of antibiotics for 7 consecutive days. Transplanted mice were maintained for 2 weeks after fecal transplantation before they were subjected to fecal collection for 16S analyses and surgery.

### Anesthesia and Surgery

The surgery was left carotid artery exposure as described before. Briefly, mice were anesthetized by 2% isoflurane, and kept spontaneous respirations with a facemask supplied with 100% oxygen during the procedure. Rectal temperature was monitored and maintained at 37° C. with the aid of a heating blanket. A 2-cm midline neck incision and soft tissue dissection with 1-cm long common artery exposure were performed without any damage to the vagus nerve after the mouse was anesthetized by isoflurane for at least 20 min. The wound was then irrigated and closed by using 4-0 surgical suture. The surgical procedure was performed under sterile conditions and lasted around 15 min in young mice and 12 min in old mice. The total duration of general anesthesia was 2 h. After the surgery, all animals received a subcutaneous injection of 3 mg/kg bupivacaine. No response to toe pinching was observed during the anesthesia.

### Intracerebroventricular Injections

As described above, some groups of mice received intracerebroventricular injection. Briefly, these mice were placed in a stereotactic head frame in the prone position. The injection site was located as: 1.00 mm mediolateral, −0.3 mm anteroposterior from Bregma, and −2.5 mm dorsoventral depth. Mice in surgery plus GDNF group received intracerebroventricular injection of 10 μg/kg recombinant mouse GDNF in 3 μl phosphate-buffered saline (PBS) as described in previous studies [6, 26], and mice in surgery plus heat-inactivated GDNF group received injection of heat-denatured (5 min at 100° C.) GDNF solution. Mice in surgery plus C3ar antagonist group received 10 μg/kg SB290157 in 3 μl PBS containing 5% DMSO at 0 h, 24 h, 48 h and 72 h after surgery. Mice in exercise plus C3ar agonist and surgery group received 1 μg/kg C3ar agonist in 3 μl PBS containing 5% DMSO at the same time as for SB290157. Mice in valeric acid group of the sixth experiment received 4 mg/kg valeric acid in 4 μl for 4 consecutive days.

### Behavioral Testing

Learning and memory were evaluated by novel object recognition and Barnes maze test. All behavioral tests were conducted at 10:00 am-5:00 pm in a sound-isolated room. Mice used in the learning and memory tests were not used for any biochemistry studies to avoid the effects from these tests.

### Novel Object Recognition Test

As described before [20, 49, 50], mice were put in an open-field chamber for 5 min for habituation 4 days after surgery. The test was performed in the following way. Two of the same objects were placed at adjacent angles of the chamber on the learning day. Mice were put into the chamber with their backs turned towards the objects and allowed to explore the chamber freely for 5 min. The animal was eliminated if the total exploration time on two objects was less than 5 s. One of the objects was replaced by a novel object 30 s or 24 h later. The mouse was put into the chamber with their backs turned towards the objects and allowed to explore for 5 min. Animal behavior was recorded by ANY-maze behavioral tracking software (Stoelting Co., IL). Exploratory time of new (T2) and old (T1) objects within 5 min was recorded and the memorization ability of the mouse was quantified by discrimination index: DI=T2/(T1+T2). The DIs at 30 s and 24 h after the training reflected the instant and long-term memory, respectively. The field was always provided with even light, and the objects and fields were cleaned with 70% ethanol after each test.

### Barnes Maze

Seven days after surgery, animals were subjected to Barnes maze to test their spatial learning and memory as previously described [6, 49]. Barnes maze is a circular platform with 20 equally spaced holes (SD Instruments, San Diego, CA). One of the holes was connected to a dark chamber that was called target box. The test started by placing animals in the middle of the Barnes maze. Aversive noise (85 dB) and bright light (200 W) shed on the platform were used to encourage mice to find this box. After training for 4 days, their reference memory was tested on day 5 and day 12. No test was performed during the period from day 5 to day 12. The latency to enter the target box during each trial was recorded by an ANY-Maze video tracking system (SD Instruments).

### Gut Microbiota Profiling

Fresh feces from mice were collected for gut microbiota profiling. Mice were placed individually in an autoclaved cage for collecting feces and allowed to defecate freely. Feces were collected immediately in the sterile Eppendorf tubes on dry-ice and then stored at −80° C. until further processing. Bacteria DNA was extracted with Power Lyzer Power soil DNA isolation kit according to previous protocols [11]. Sequencing libraries of the hypervariable V3-V4 region were prepared according to the Illumina MiSeq system instructions. Briefly, 12.5 ng DNA was used as DNA template for the first 16S rRNA PCR. Primers used were as follows: F: 5′-TCGTCGGCAGCGTCAGATGTGTATAAGAGACAGCCTACGGGNGGCWGCAG-3′ (SEQ ID NO. 15) and R: 5′-GTCTCGTGGGCTCGGAGATGTGTATAAGAGACAGGACTACHVGGGTATCTAATCC-3′ (SEQ ID NO. 16). The amplicons were then cleaned up with AMPure XP magnetic beads and then used for Index PCR by using the Nextera XT Index Kit. Qubit dsDNA HS assay kit and TapeStation high sensitivity D1000 ScreenTape (Agilent, Blacksburg, VA, USA) were used to measure the concentrations of PCR products and normalize the quantity for library preparation. Sequencing was operated on an Illumina MiSeq instrument by MiSeq reagent kit v2 (500 cycles). Data was analyzed with the MiSeq Reporter software Metagenomics workflow v2.5.1.3 (Illumina, San Diego, CA, USA).

The paired reads obtained by double-terminal sequencing were spliced into a sequence through Pandaseq software. The long reads with high variable region were obtained. The reads whose average phred score in the window (5 bp in size, 1 bp step length) was less than 20 were trimmed. Reads containing ambiguous “N” or with length <220 bp were discarded. After quality control of the original data, the high-quality sequences without chimeras were arranged according to abundance from large to small and then clustered with 97% similarity into an Operational Taxonomic Units (OTU). Each OTU was considered to represent a species. To avoid the deviation of analysis caused by the different sizes of sample sequencing data, the number of Reads to OTU was entered according to the minimum sequence number matched to OTU when the sequencing depth was sufficient. Alpha diversity was analyzed by random leveling. A Read was extracted from each OTU as a representative sequence. The representative sequence was compared with ribosomal database project database. Species were classified for each OTU, and species abundance tables were obtained for subsequent analysis. OTU picking was performed using Uclust on the software platform QIIME v1.9.1. Alpha diversity including Chaol index and goods coverage index was used for the analysis of species diversity in a sample. Beta diversity analysis was used to compare differences in species diversity among samples and characterized by principal coordinate analysis (PCoA) based on Bray-Curtis distance. The differences among the groups were tested by analysis of similarity (ANOSIM).

### Plasma SCFA Profiling

Plasma SCFA concentrations were quantified using a 7890-5977 gas chromatography-mass spectrometry (GC-MS, Agilent, Blacksburg, VA, USA). Plasma samples were collected from mice at time points described above, stored at −80° C. quickly and then thawed to room temperature for processing. Briefly, 100 μl plasma sample was vortex-mixed with 900 μl ethanol (containing 0.5% HCl, V/V). Ultrasonic treatment was applied for 40 min and then the samples were centrifuged at 14000 RPM for 10 min. The supernatant of samples were measured by GC-MS analysis on an Agilent 7890-5977 GC-MS with electron impact ionization and a DB-FFAP capillary column (30 m×0.25 mm×0.25 μm).

### Cortical Valeric Acid Quantification

The amount of valeric acid in the cerebral cortex harvested 24 h after the last intraperitoneal injection of valeric acid was determined by a method similar to that reported before [51, 52]. Briefly, 0.1 g cerebral cortex was homogenized in 500 μl aqueous acetonitrile. Supernatant was extracted with 8 ml extraction buffer (hexane: diethyl ether=1:1) and centrifuged at 1800 g for 5 min. 7.5 ml supernatant was collected, mixed with 93 μl 20 mM KOH in methanol and dried at 40° C. under nitrogen gas. Dried residue was reconstituted in 50 μl 2.5% 18-Crown-6 in acetonitrile and was further derivatized with 9-chloromethylanthracene in acetonitrile with addition of tetramethylammonium hydroxide. Finally, 30 μl derivatization solution was loaded to Acclaim C18 column (3 μm, 4.6×100 mm, Thermo Scientific) and separated in Ultimate 3000 high performance liquid chromatograph (HPLC, Thermo Scientific) equipped with UV-visible detector. The peak of derivatized valeric acid was detected at a wavelength of 254 nm. 2-Ethylbutyric acid (2-EA) was added as an internal reference control. The peak area of valeric acid was measured as mAU*min and its peak area of each sample was normalized with that of 2-EA in the sample. Final results were presented as ratios of valeric acid group to NS group.

### Brain Tissue Harvesting

Mice were weighed and anesthetized with isoflurane at 6 h, 24 h, 48 h, 72 h, 96 h and 7 days after surgery and perfused with 4° C. NS for brain tissue harvesting. These hippocampi were used for ELISA analysis or RNA assay. Whole brain at Bregma −3 to −6 mm was used for immunofluorescent staining. All dissection procedures were performed on ice.

mRNA Library Preparation and Illumina Hiseq Sequencing

The hippocampi harvested at 24 h after surgery from control group, surgery group, and Exe+Sur group in the first experiment were placed in liquid nitrogen for 1 h, and then stored in-80° C. freezer quickly for no more than 1 week. RNA purification, reverse transcription, library construction and sequencing were performed at WuXi NextCODE in Shanghai according to the manufacturer's instructions (Illumina). The mRNA-focused sequencing libraries from total RNA were prepared using Illumina TruSeq® RNA sample preparation Kit. PolyA mRNA was purified from total RNA using oligo-dT-attached magnetic beads and then fragmented by fragmentation buffer. Taking these short fragments as templates, first strand cDNA was synthesized using reverse transcriptase and random primers, followed by second strand cDNA synthesis. Then the synthesized cDNA was subjected to end-repair, phosphorylation and ‘A’ base addition according to Illumina's library construction protocol. Illumina sequencing adapters were added to the cDNA fragments. After PCR amplification for DNA enrichment, the AMPure XP Beads (Beckmen) were used to clean up the target fragments of 200-300 bp.

After library construction, Qubit 3.0 fluorometer dsDNA HS Assay (Thermo Fisher Scientific) was used to quantify concentrations of the resulting sequencing libraries, while the size distribution was analyzed using Agilent BioAnalyzer 2100 (Agilent).

Sequencing was performed using an Illumina system following Illumina-provided protocols for 2×150 paired-end sequencing in WuXi NextCODE in Shanghai, China.

Hippocampal mRNA Expression by Quantitative Polymerase Chain Reaction (q-PCR)

The hippocampus harvested at 24 h after surgery from the control group, exercise group, surgery group, Exe+Sur group, Trans-Control+Sur group, Trans-Exe+Sur group, Exe+Sur+NS group, and Exe+Sur+Val group was placed in liquid nitrogen for 1 h, and then stored in −80° C. freezer for no more than 3 days. RNA extraction, RNA-to-cDNA reverse transcription, and q-PCR were performed as previously described [49, 53]. Briefly, RNA was extracted by RNeasy Micro kit. Reverse transcription was finished by using 5× All in-One MasterMix. Finally, CFX connect real time system (Bio-Rad) was used for quantitative quantification. Primers for qPCR were: C3ar1-F: TGGGCTGGTGCTGTGGGTAG (SEQ ID NO. 17) and C3ar1-R: GATGGCGAAGGCGGTTCTCAC (SEQ ID NO. 18). Relative gene expression for C3ar1 was calculated using the comparative threshold cycle AACt and housekeeping gene β-actin for normalization of gene expression. The mRNA levels for target genes are expressed as fold increases relative to relevant group, respectively.

### Cytokines and GDNF Determination in Hippocampus

IL-1β, IL-6, C3 and GDNF in the hippocampus were detected by using ELISA kits. For testing the IL-1β, IL-6 and GDNF, brain tissues were homogenized on ice in the RIPA buffer containing 25 mM Tris-HCl with pH 7.6, 150 mM NaCl, 1% sodium deoxycholate, 0.1% SDS and a protease inhibitor cocktail containing 10 mg/ml aproteinin, 5 mg/ml peptastin, 5 mg/ml leupetin and 1 mM phenylmethane sulfonylfluoride. For C3 testing, brain tissues were homogenized on ice in the PTR buffer (supplied in the kit). The supernatant was collected for ELISA detection after the homogenate was centrifuged at 13,000 g for 20 min at 4° C. and the protein concentration was determined by the BCA protein assay (Bio-Rad, Hemel Hempstead, Herts, UK). IL-1β, IL-6, C3 and GDNF in supernatant of brain were detected according to the manufacturer's instruction for ELISA, and the amount of them in each sample in the brain was normalized by its total protein content and was expressed as pg/mg protein.

### Western Blotting

Western blotting was performed as previously described [49]. In brief, protein concentrations of samples were determined using the BCA protein assay. Twenty micrograms of each sample were subjected to Western blotting analysis using the following primary antibodies: rabbit polyclonal anti-postsynaptic density protein 95 (PSD95) at 1:1000 dilution, rabbit polyclonal anti-synapsin 1 at 1:1000 dilution and rabbit anti-α-tubulin antibody at 1:1000 dilution. Images were scanned by an Image Master II scanner (GE Healthcare, Milwaukee, WI, USA) and analyzed using ImageQuant TL software v2003.03 (GE Healthcare). The band signals of interested proteins were normalized to those of the corresponding α-tubulin and expressed as fractions of control sample from the same gels.

### Immunofluorescent Staining

The immunofluorescent labeling and quantification of the staining were performed as described before [6, 54]. Briefly, brain was fixed in 4% paraformaldehyde at 4° C. for 24 h and then incubated in 30% sucrose over night at 4° C. before being frozen in optimal cutting temperature compound. Coronal 20-μm thick sections were cut sequentially from Bregma −3 to −6 mm by using a cryostat and mounted on microscope slides. After being washed in Tris-buffered saline (TBS), sections were blocked in 10% donkey serum plus 1% bovine serum albumin (BSA) in TBS containing 0. 3% triton-X 100 for 2 h at room temperature and then incubated at 4° C. overnight with the following primary antibodies: rabbit monoclonal anti-C3 antibody (1:50), goat monoclonal anti-GFAP antibody (1:200), rat monoclonal anti-C3ar antibody (1:50) and rabbit monoclonal anti-Iba-1 antibody (1:200). Sections were rinsed in TBS with 0.1% Triton-x 100. The donkey anti-rabbit IgG antibody conjugated with Alexa Fluor 594 (1:200), donkey anti-goat IgG antibody conjugated with Alexa Fluor 488 (1:200), donkey anti-rat IgG antibody conjugated with Alexa Fluor 594 (1:200) or donkey anti-rabbit IgG antibody conjugated with Alexa Fluor 647 (1:200) were incubated with the sections for 1 h at room temperature in the dark. The sections were washed in TBS, incubated with Hoechst 33342 (1:1000) for nuclear staining, rinsed and mounted with Vectashield mounting medium (H-1000; Vector Labs, Burlingame, CA). Images of immunostaining were acquired by z-stack with an LSM710 microscopy system (ZEISS), and a negative control omitting the incubation with the primary antibody was included in all experiments. The quantification was performed as described previously [6, 54]. Briefly, the whole dentate gyrus region that was covered by 2-3 non-overlapping fields from each of six sequential hippocampus sections of one mouse was imaged. The number of pixels per image with intensity above a predetermined threshold level was considered as a positively stained area for an interested marker and quantified using the Image-pro plus 6.0 (Media Cybernetics, Inc., Rockville, MD, USA) and presented as percentage of positive area in the total area. Six measurements per mouse were then averaged to reflect the level of positive staining. All quantitative analyses were performed in a blinded manner.

### BrdU Administration and Immunofluorescent Staining

Seven days after surgery, mice were given 7 consecutive intraperitoneal injections of 80 mg/kg 5′-bromo-2′-deoxyuridine (BrdU) at 12:00 once daily as previously described [14]. Mice were sacrificed 5 days later for harvesting hippocampus. Hippocampus was fixed in 4% paraformaldehyde in 0.1 M phosphate-buffered saline at 4° C. for 24 h and embedded in paraffin. Five-micron thick coronal brain sections were cut sequentially from Bregma −2 to −4 mm. Antigen retrieval was performed by incubating sections with sodium citrate buffer containing 10 mM sodium citrate, 0.05% Tween 20 (pH 6.0) at 95-100° C. for 20 min. DNA denaturation was done by incubating with 1 N HCl on ice for 3 min, 2 N HCL at room temperature for 3 min, and at 37° C. for 6 min. Sections were blocked with 5% donkey serum in phosphate-buffered saline (PBS) containing 0.5% triton-X 100 for 2 h at room temperature. The sections were then incubated overnight at 4° C. with the following primary antibodies: rat monoclonal anti-BrdU antibody (1:100), and goat monoclonal anti-GFAP antibody (1:200). The sections were incubated with donkey anti-rat IgG antibody conjugated with Alexa Fluor 594 (1:200) or donkey anti-goat IgG antibody conjugated with Alexa Fluor 488 (1:200) for 1 h at room temperature in a dark room. After being washed in PBS, sections were counterstained with Hoechst 33342 (1:1000), rinsed and mounted with Vectashield mounting medium (Vector Labs). Images were acquired with a fluorescence microscope with a charge-coupled device camera (High Mag Olympus BX51) and a LSM710 confocal microscopy system (ZEISS). A negative control omitting the incubation with the primary antibody was included in all experiments. For each mouse brain, six sequential hippocampal sections were used for cell counting. The number of all cells positively stained for an interested marker or the combination of two markers in the sub-granular zone of the dentate gyrus of each section was counted. The quantitative analyses were performed in a blinded manner.

### Golgi Stain

Golgi staining was performed using FD Rapid GolgiStain™ Kit. Nineteen days after surgery, brains of 6 mice in each group were immersed in the impregnation solution (solutions A and B: 1:1) for 2 weeks and then transferred to solution C for 3 days. Coronal brain sections at a thickness of 100 μm and around-2.7 mm from bregma were cut on a vibratome (Microslicer® 10110, Ted Pella, Inc. California, USA). As described before [55, 56], more than 10 well individualized neurons in the dentate gyrus (DG) region of the hippocampus were randomly selected from each mouse, and sequential optical multiple-layers scanning images were taken at an interval 2.0 μm along the z-axis (3DHISTECH, Pannoramic MIDI, Hungary) with 20× objective. The multiple-layer scanning files were superposed to become one clear scanning file that can be viewed with Caseviewer (3DHISTECH, Hungary) on a computer. The total branch number and dendritic length were measured by Fiji software (Fiji-win64, NIH, USA). The complexity of total dendritic trees (intersections) was estimated using Sholl analysis. For spine density measurement, 5 neurons were selected from each animal. Five randomly selected microscopic fields at the apical or basal dendrites were photographed from the scanning files. The spine numbers in 40 μm segments were counted by an observer who was blind to group assignment. The results were expressed as the number of spines/10 μm segments. The data of dendritic branch numbers, length and intersections as well as spine density from one mouse were averaged to reflect the level of the mouse.

### Statistical Analysis

Sample size calculation for each experiment was not performed. The determination of sample size was mostly based on experience: larger sample sizes were used for experiments testing learning and memory than those for biochemical studies. All data that were collected were included in analyses. Missing data included the animals that met pre-determined exclusion criteria for exercise training and animals that died before the intended observation period. The number of animals that contributed data for analysis for each experimental condition is stated in figure legends. Results are presented as means+S.D. (normal distribution data) or median+interquartile range (not normal distribution data) with the presentation of individual animal data in the bar graph and means+S.D in the line plots. Data from the training sessions of Barnes Maze were analyzed by two-way repeated analysis of variance (ANOVA) followed by Tukey's test. Gut microbiota data were analyzed as described in section Gut microbiota profiling. The other data were analyzed by one-way ANOVA followed by Tukey test if the data were normally distributed, by one-way ANOVA on rank followed by Tukey test or Student-Newman-Keuls Method if the data were not normally distributed, by two-way ANOVA, by t-test or rank sum test. Significant difference was defined as P<0.05 based on two-tailed hypothesis testing. Adjustments for multiple comparisons were not performed. All statistical analyses were performed with SigmaStat (Systat Software, Inc., Point Richmond, CA, USA).

### Example 1

### Low Intensity Exercise Prevented Surgery-Induced Cognitive Dysfunction, Neuroinflammation, and Impairment of Brain Cell Generation and Dendritic Arborization Possibly Via Stabilizing Gut Microbiota in Adult Mice

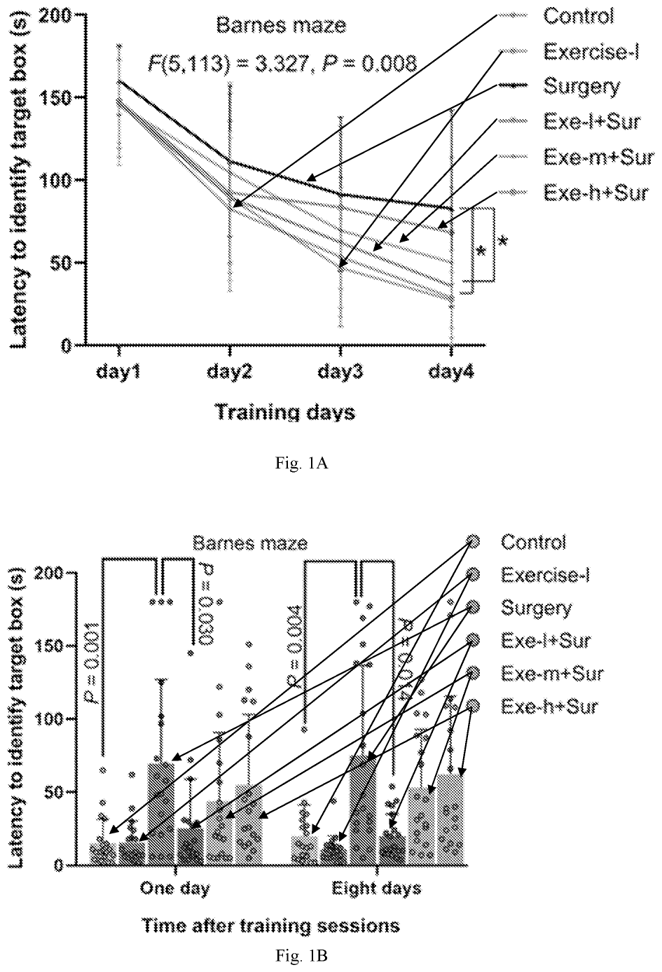

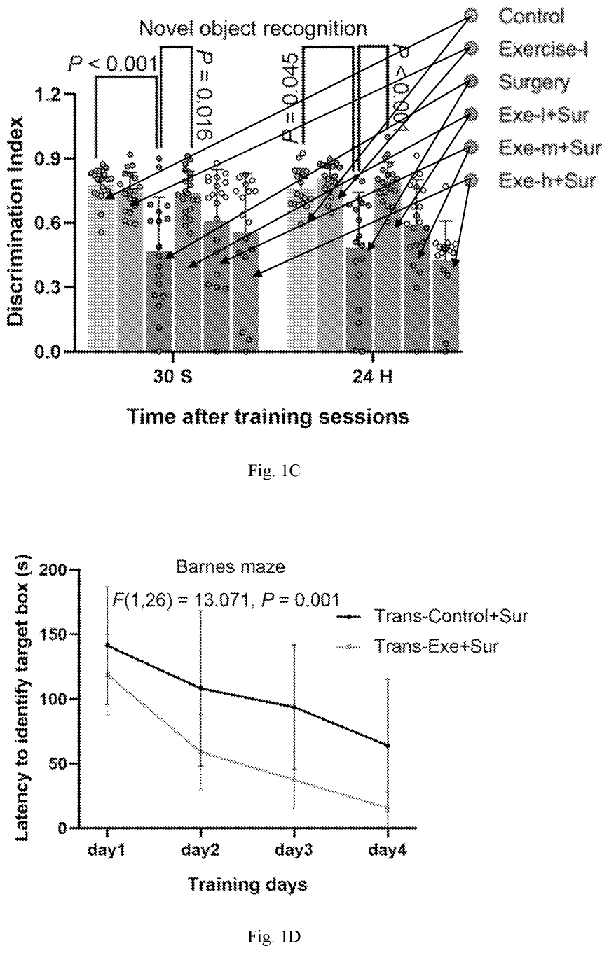

To determine the effect of physical activity on the function and structure of brain, 8-week old mice were subjected to forced mouse treadmill running to 35-40% (low intensity), 55-60% (middle intensity), or 75-80% (high intensity) of their individual maximal exercise capacity, respectively, 5 days a week for 4 weeks before they had left carotid artery exposure for 15 min under isoflurane anesthesia (surgery and anesthesia). Mice were assessed by novel object recognition and Barnes maze tests from one week after the surgery. Mice in all groups took less time to find the target box with more training sessions in the Barnes maze test (FIG. 1A). Surgery was a significant factor to affect the animals to find the target box [F (1,113)=7.360, P=0.008] and low intensity exercise reversed this surgery effect in the training sessions of the Barnes maze test [F (1,76)=5.278, P=0.024]. Mice with surgery took longer than control mice to find the target box, one or eight days after the training sessions. This surgery effect was attenuated by low intensity exercise but not by middle or high intensity exercise (FIG. 1B). Mice with surgery spent less time with novel object than control mice in the novel object recognition test and this decreased time was reversed by low intensity exercise but not by middle or high intensity exercise no matter when the test was performed either 30 s or 24 h after the initial exploration with two objects (familiarization phase) (FIG. 1C). Various lengths of delay between the familiarization and test phases (from 10 s to 24 h) have been used previously. The memorization after short and long delays may involve perirhinal cortex and hippocampus, respectively [20]. The presently disclosed results suggest that surgery induces learning and memory dysfunction and that low intensity exercise but not middle and high intensity exercise prevents these surgery effects.

As an initial step to determine whether gut microbiota played a role in the effects of exercise on learning and memory, mice transplanted with feces from control mice or from mice with low intensity exercise were subjected to surgery. Transplantation with feces from exercise mice reduced the amount of time to identify the target box in the training sessions [F (1,26)=13.071, P=0.001] and one or eight days after training sessions (FIGS. 1D, 1E). Mice transplanted with feces from exercise mice spent more time with novel object than mice transplanted with feces from control mice (FIG. 1F). These results suggest that gut microbiota mediates the beneficial effects of low intensity exercise. This level of exercise was used for mice receiving exercise conditioning in the following experiments and was referred as exercise for simplicity.

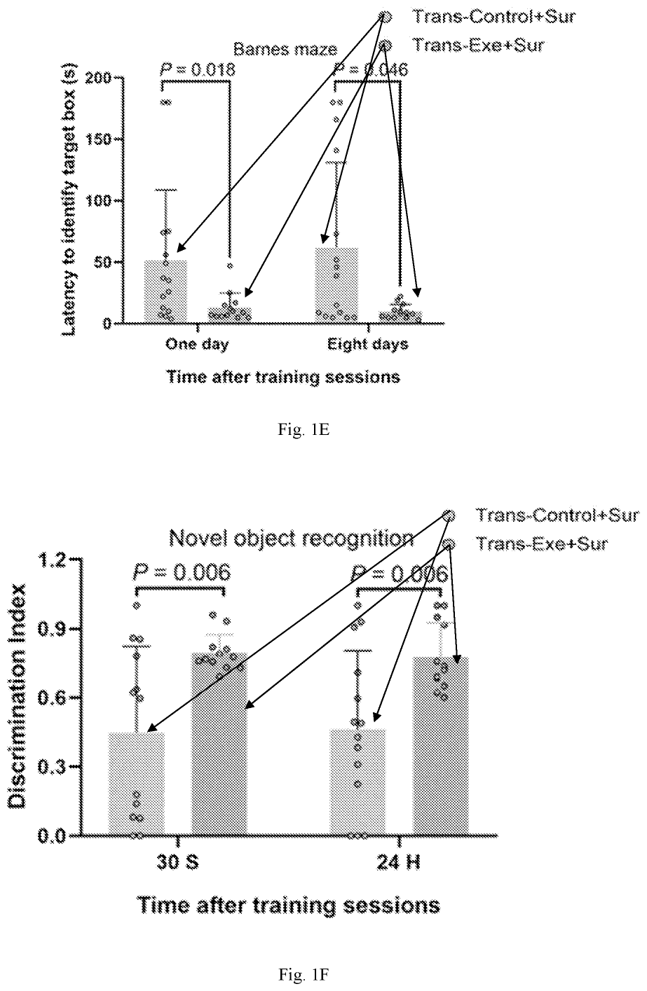

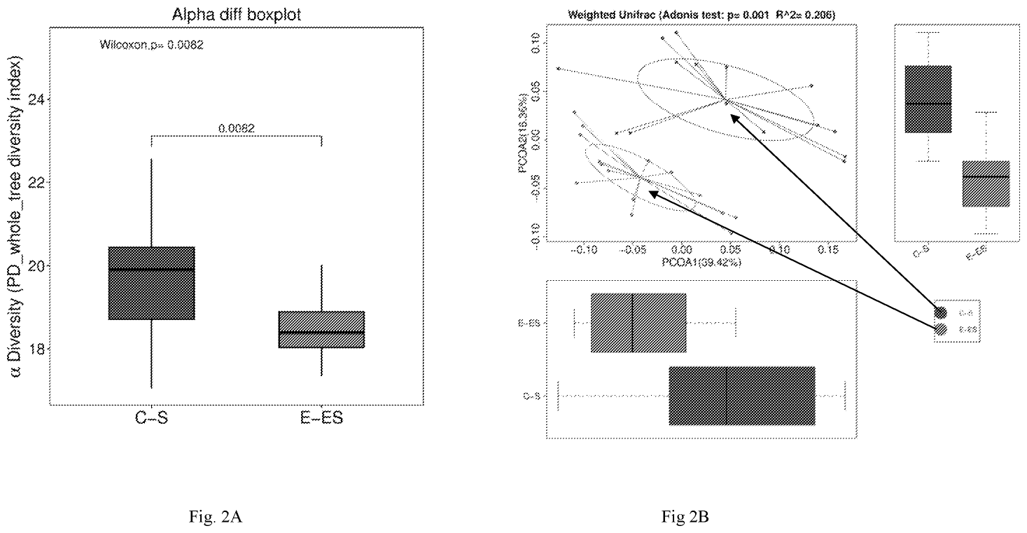

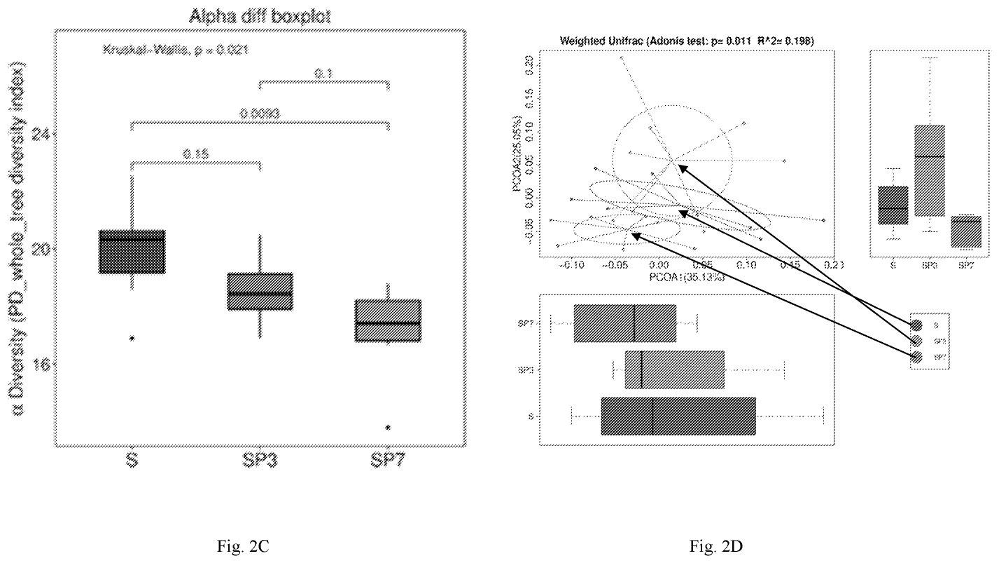

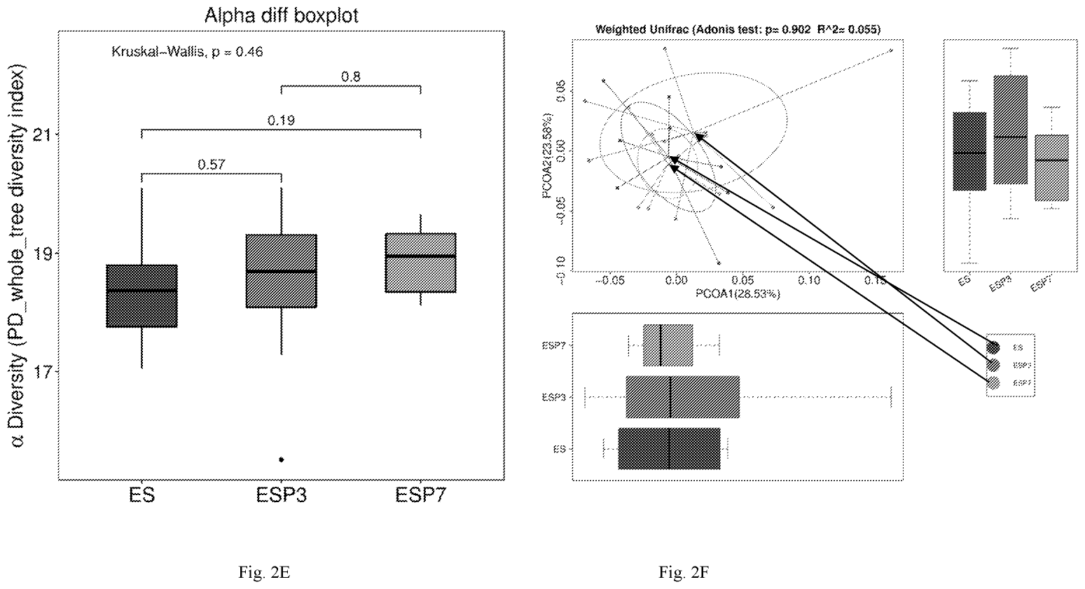

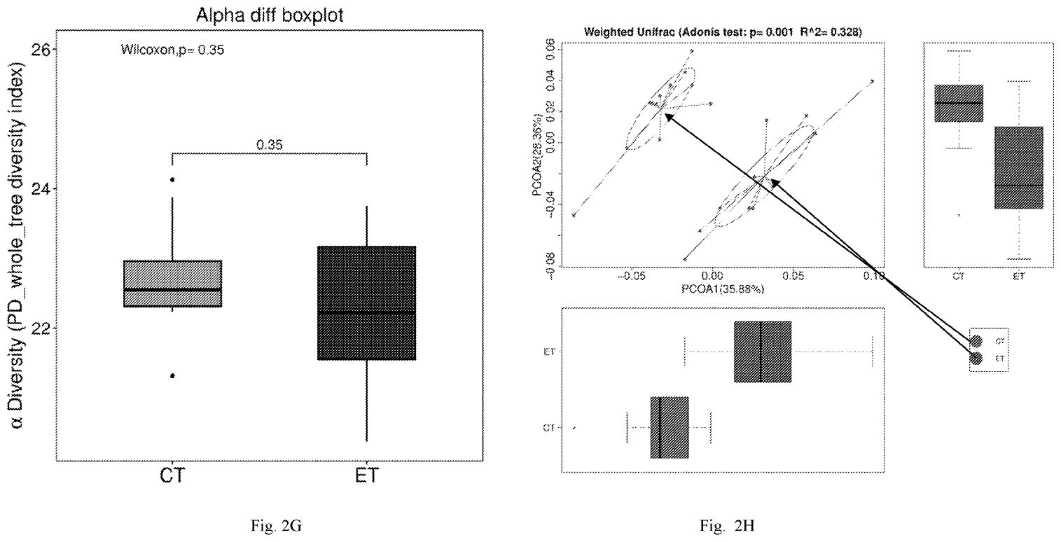

Exercise modified the diversity of gut microbiota within one sample (a diversity) and the difference in diversity among samples (B diversity) (FIGS. 2A, 2B). Exercise increased the abundance of some bacteria, such as Bacteroidales and Alistipes. Control mice had abundant lactobacilliaceae and lactobacillus because the linear discriminant analysis (LDA) scores for the comparisons of these bacteria between control and exercise mice were all more than 3.5 (the direction can be negative or positive), a threshold indicating difference in the abundance of bacteria in samples from different experimental conditions [21]. LDA was performed after the difference among samples from different experimental conditions was determined to be significant by Kruskal-Wallis test by rank per LDA effect size analysis protocol. Mice had a reduced diversity of gut microbiota and the difference in diversity among samples 7 days after surgery. This reduction was attenuated in exercise mice (FIGS. 2C-2F). Mice receiving fecal transplantation had treatment of the mixture of antibiotics to eliminate their native gut microbiota prior to the transplantation. This treatment nearly abolished the gut microbiota in these mice because the bacterial DNA in the feces was barely detectable. Mice transplanted with feces from exercise mice also had a reduced difference in diversity among samples (FIG. 2g, h). Gut microbiota of mice transplanted with feces from exercise mice contained an abundant amount of Alistipes. Mice transplanted with feces of control mice contained a large amount of lactobacilliaceae and lactobacillus. These results suggest that exercise changed gut microbiota with increased abundance in phylum Bacteroidetes, such as Bacteroidales and Alistipes, and decreased abundance in phylum Firmicutes, such as lactobacilliaceae and lactobacillus. Exercise also stabilizes gut microbiota after surgery.

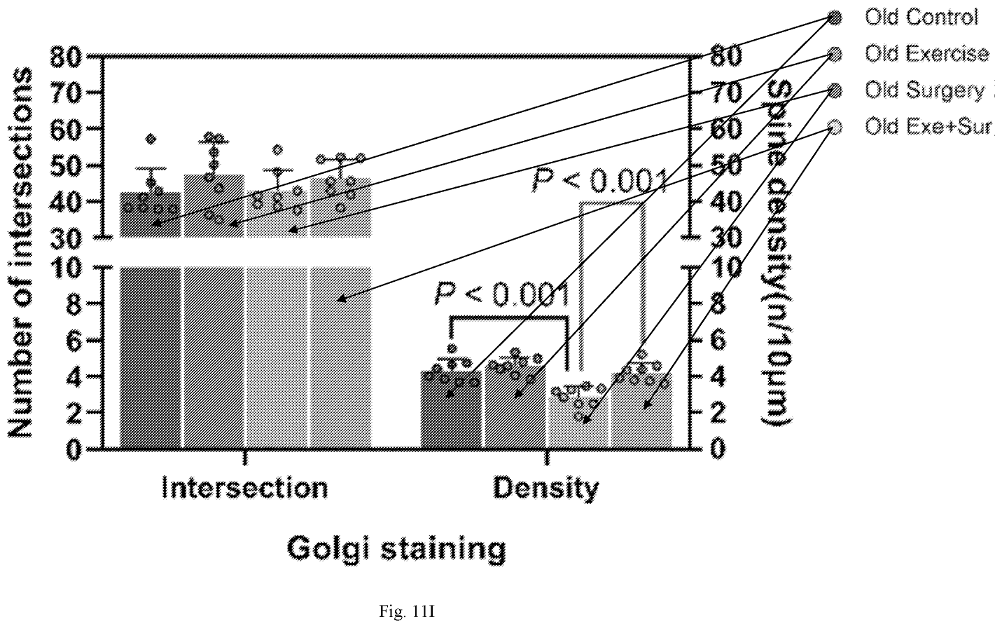

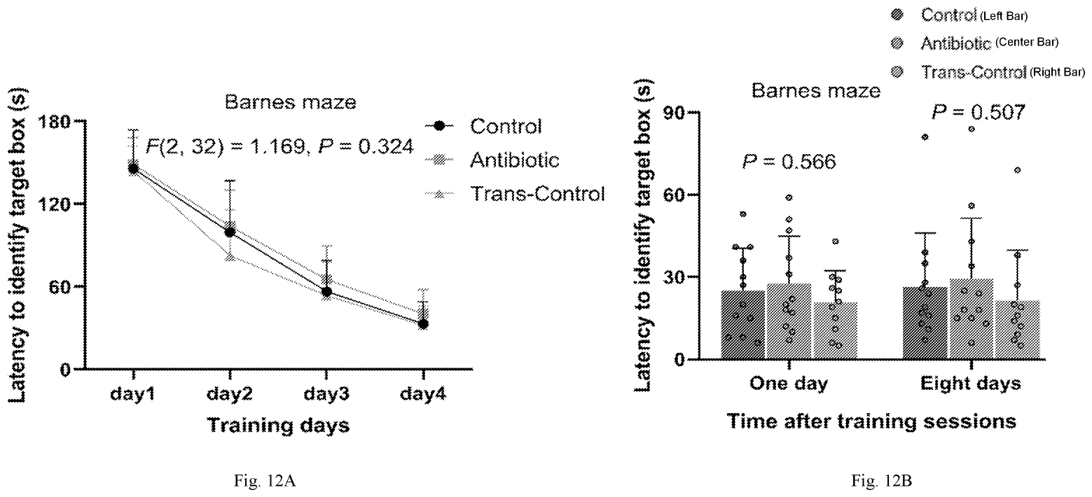

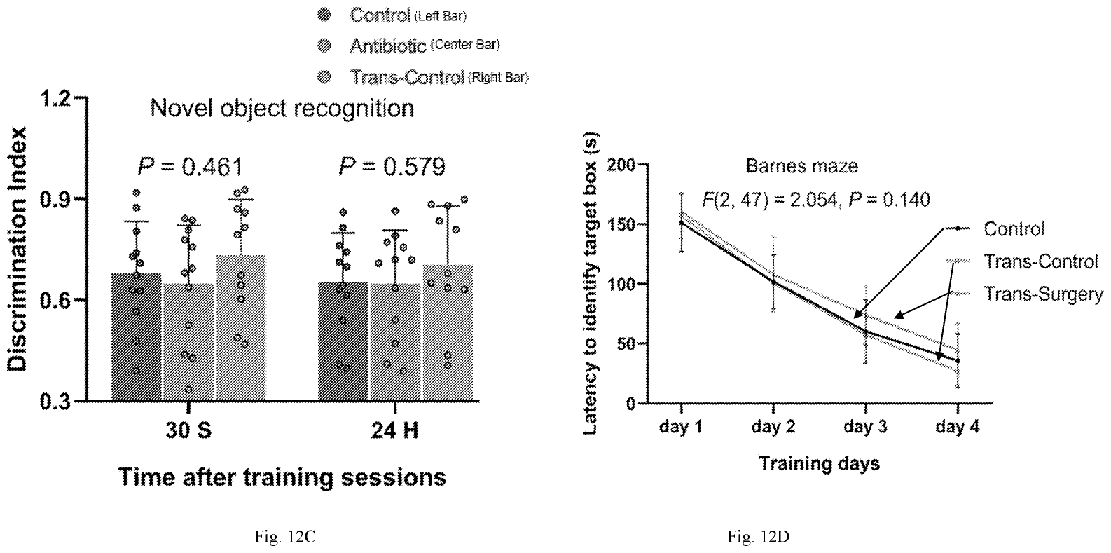

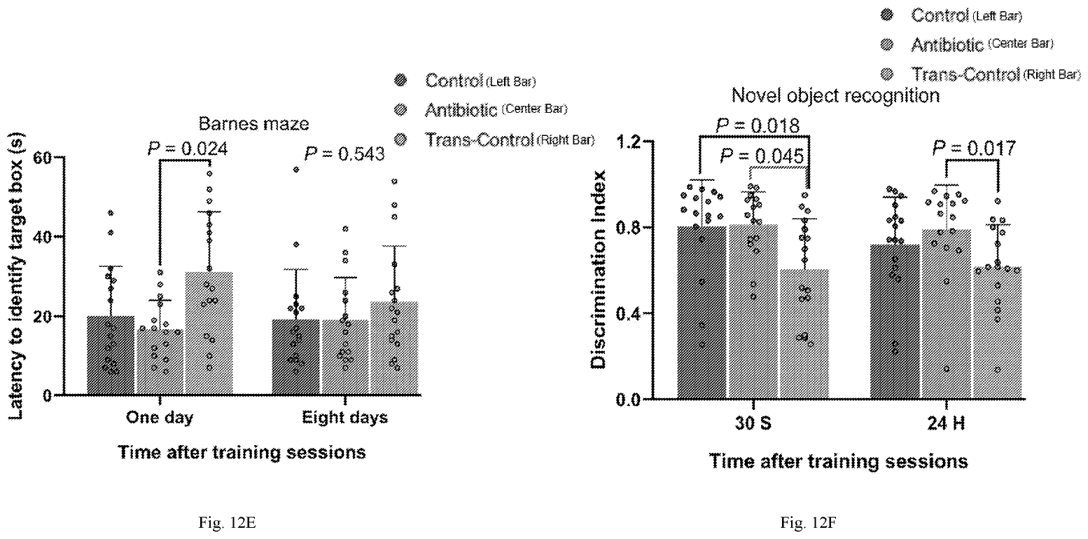

Consistent with the pattern of microbiota in mice with surgery, mice transplanted with feces of surgery mice had reduced diversity of gut microbiota compared with mice transplanted with feces of control mice. Mice receiving surgery mouse feces had decreased abundance in Bacteroidales and Alistipes compared with mice receiving control mouse feces. These results suggest that altered microbiota in surgery mice is successfully transferred to non-surgery mice. Mice receiving surgery mouse feces took longer than mice receiving control mouse feces to identify the target box in Barnes maze one day after the training sessions. The mice receiving surgery mouse feces also spent less time with novel object than mice receiving control mouse feces in the novel object recognition test. However, there was no difference among control mice (naïve mice), mice receiving antibiotics and mice receiving control mouse feces in the performance of Barnes maze and novel object recognition tests (FIG. 12). These results indicate a role of microbiota alteration in learning and memory dysfunction after surgery.

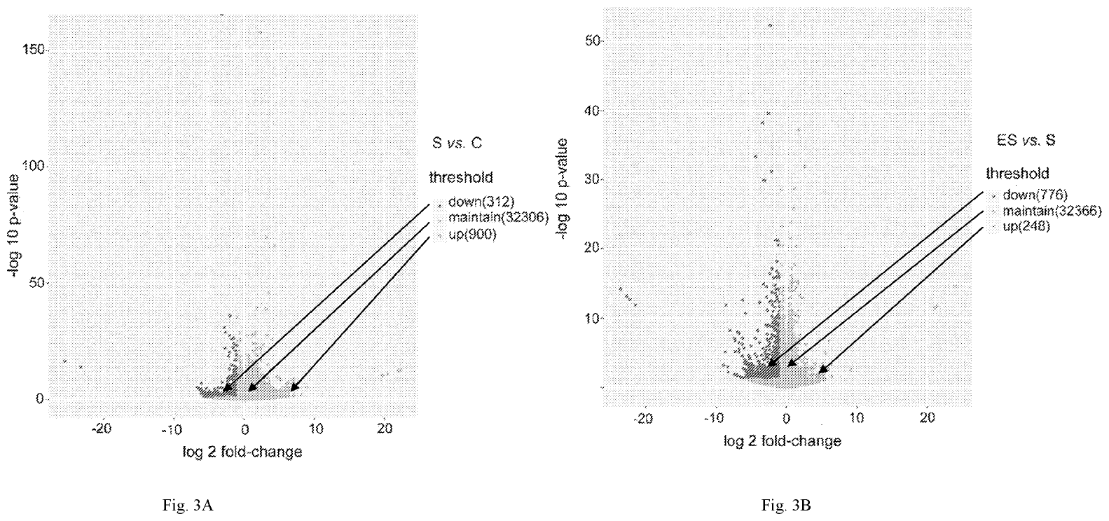

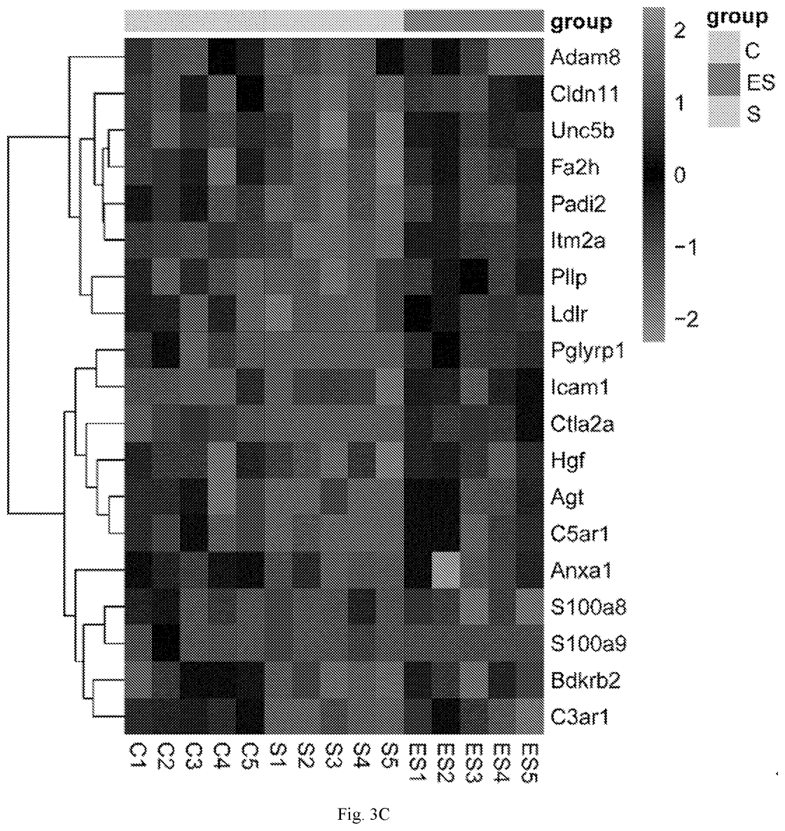

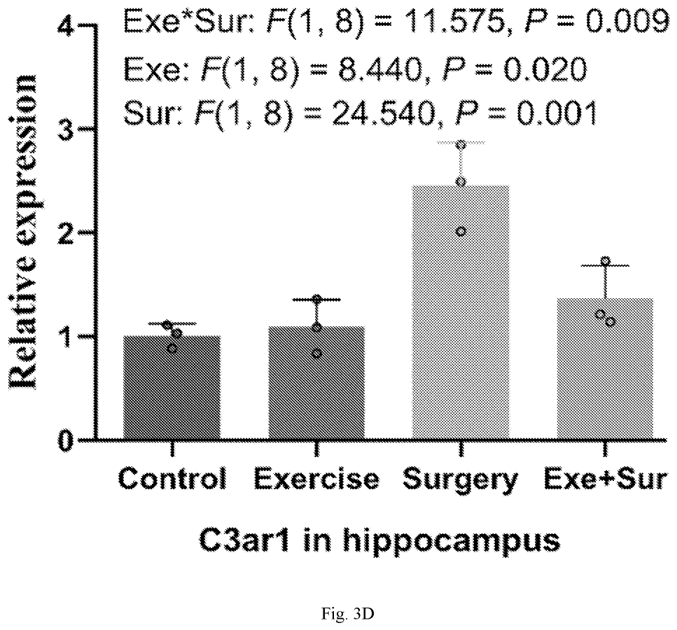

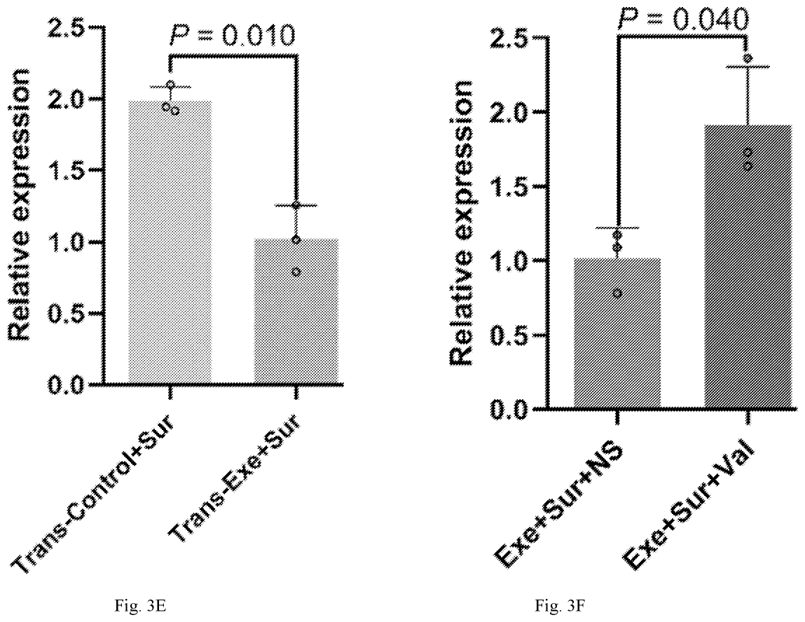

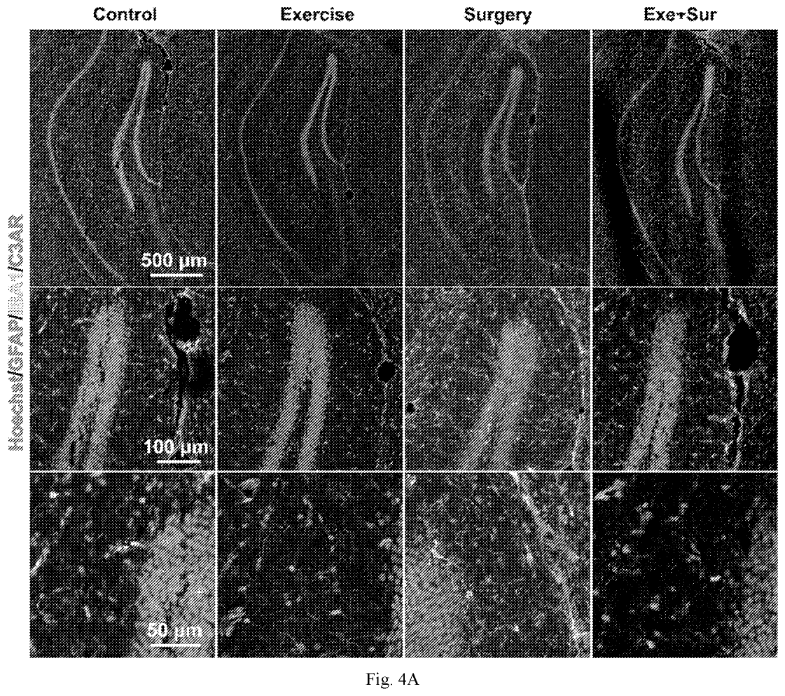

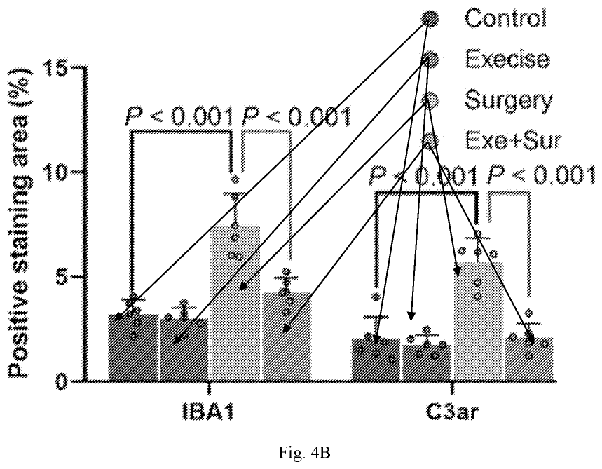

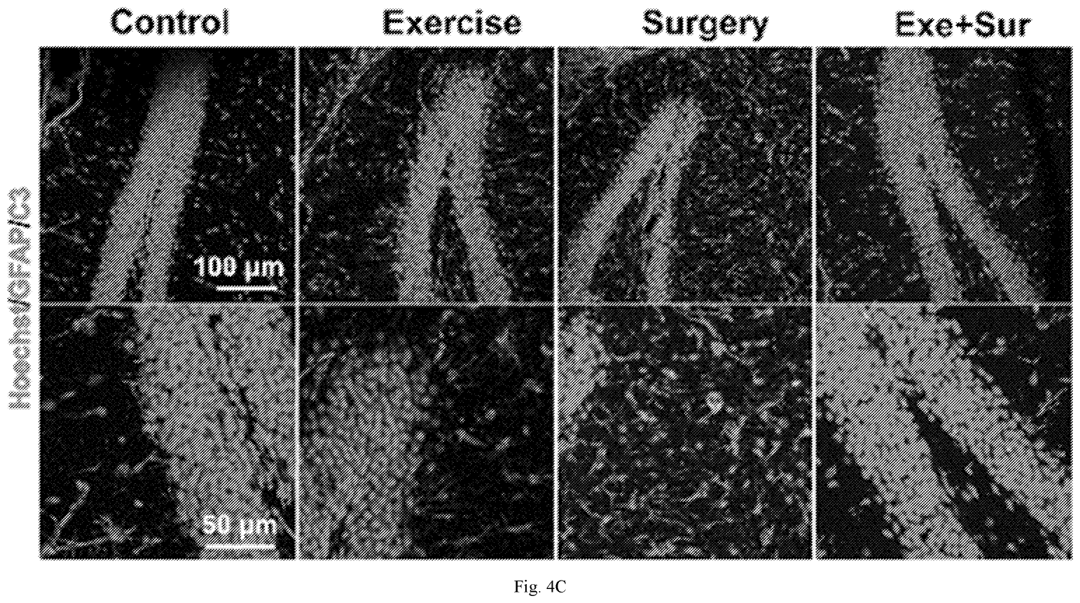

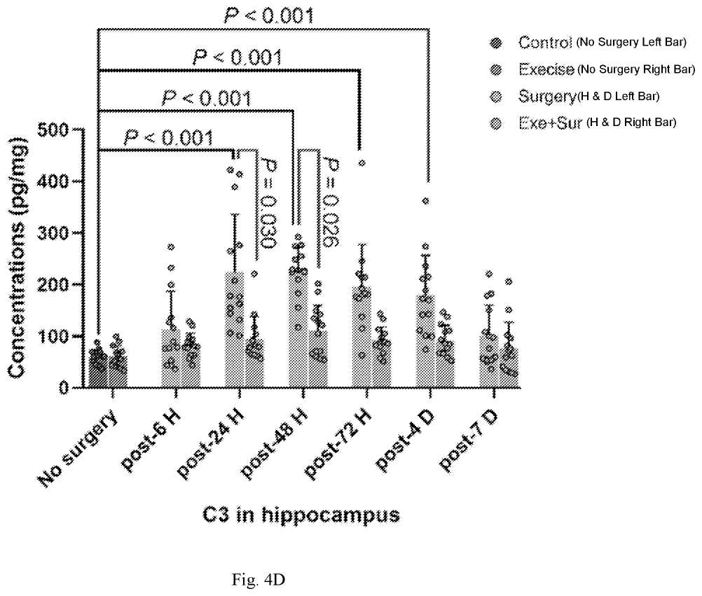

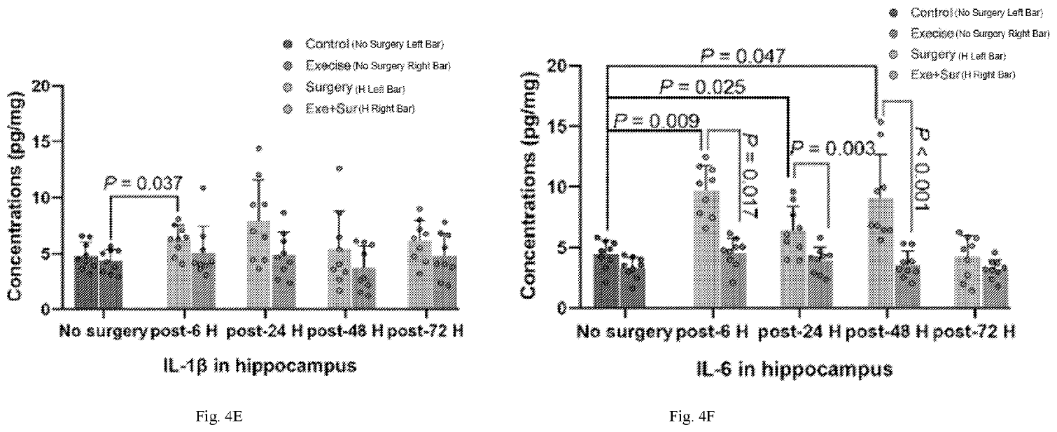

As an initial step to identify possible molecules downstream of low intensity exercise and gut microbiota for regulating learning and memory, RNA-seq analysis was performed. Surgery and exercise altered the profiles of mRNA expression in the hippocampus (FIG. 3A, 3B), a brain region involved in learning and memory [6, 22]. The genes with most changes by surgery and exercise were displayed in FIG. 3C. Among them, the mRNA of complement 3a receptor 1 (C3ar1) was increased by surgery and this increase was attenuated by exercise based on a two-way analysis of variance with surgery and exercise as two independent factors (FIGS. 3C, 3D). The mRNA expression of C3ar1 after surgery was decreased in mice transplanted with feces from exercise mice (FIG. 3E). Consistent with this mRNA result, surgery increased C3ar protein (FIGS. 4A, 4B). C3, a ligand for C3ar [23], was also increased by surgery and this increase was reduced by exercise (FIGS. 4C, 4D). Consistent with the ideas that C3 signaling is important in immunomodulation and that surgery can induce neuroinflammation [8], surgery increased ionized calcium binding adaptor molecule 1 (Iba-1) and interleukin (IL)-6 expression in the hippocampus and exercise attenuated this increase (FIGS. 4A, 4B, 4F). However, surgery and exercise did not significantly change the expression of IL-1β (FIG. 4E). These results suggest that surgery induces immune and inflammatory responses and that exercise blocks these responses.

Learning and memory often require brain structural modification, such as brain cell genesis and dendritic arborization [14, 22]. Surgery reduced brain cell genesis assessed by 5′-bromo-2′-deoxyuridine (BrdU) incorporation. The decreased newly-generated cells included glial fibrillary acidic protein (GFAP)-positive cells. Exercise attenuated this surgery effect (FIGS. 5A, 5B). Surgery also reduced intersections among dendritic branches and spine densities. These surgical effects were attenuated by exercise (FIGS. 5C, 5D). These results suggest that surgery impairs dendritic arborization and that exercise blocks this impairment.

### Example 2

### Role of Valeric Acid, C3 Signaling and GDNF in Mediating the Effects of Low Intensity Exercise on POCD Via Gut Microbiota in Adult Mice

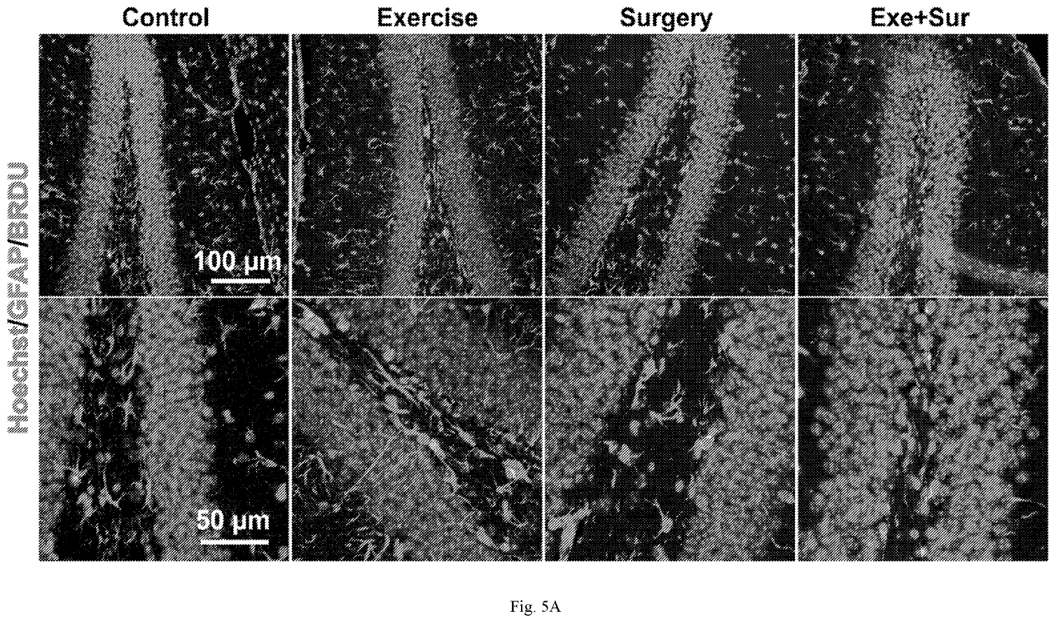

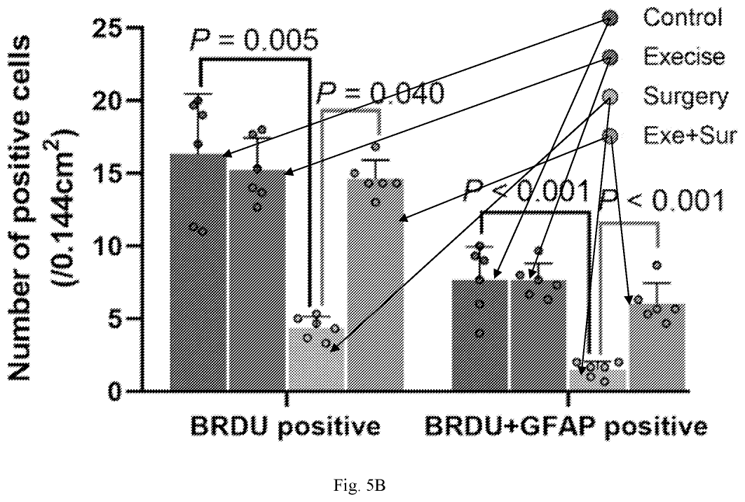

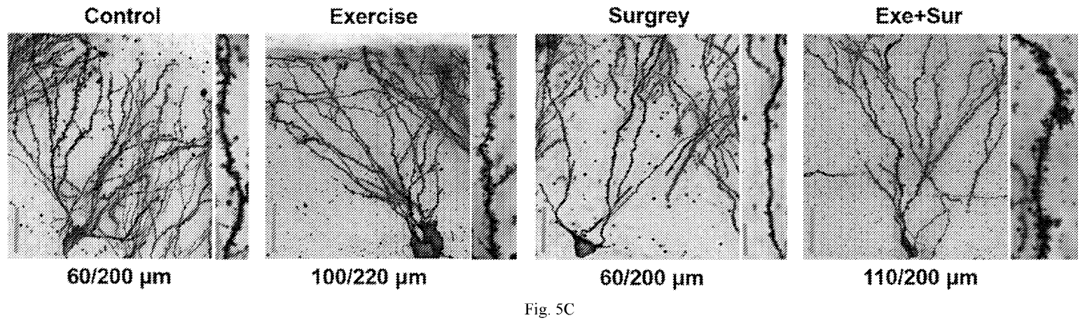

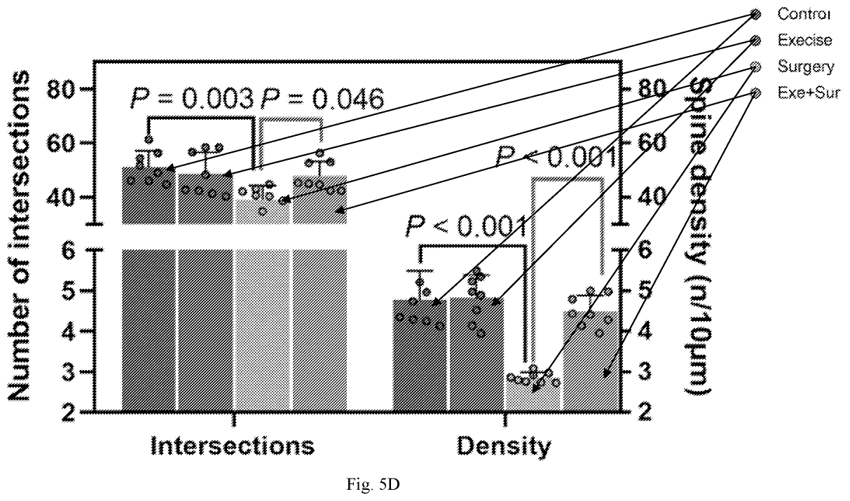

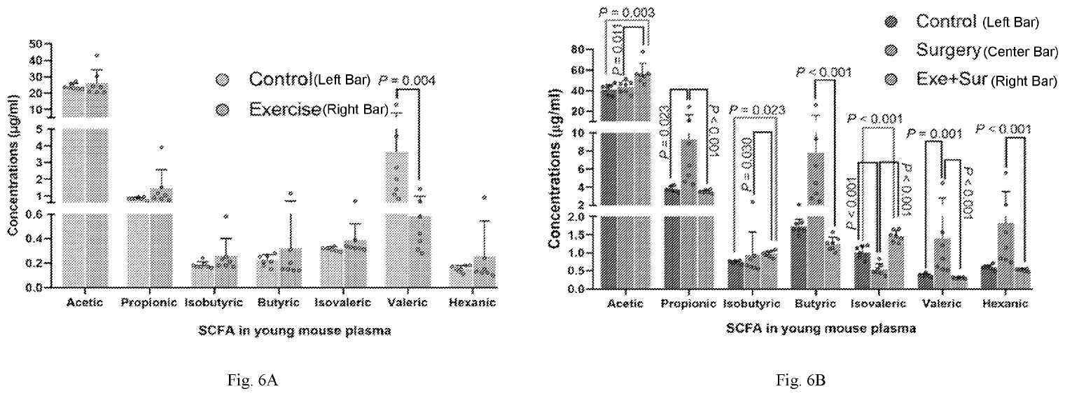

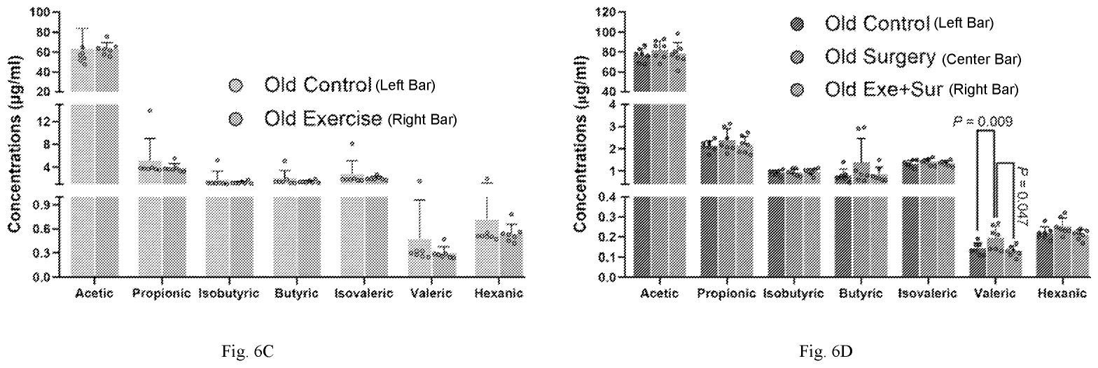

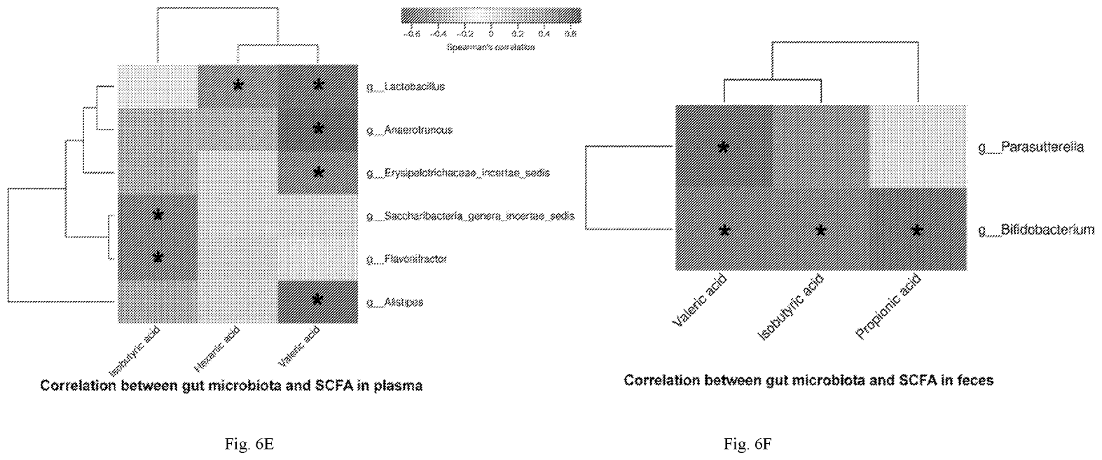

To identify possible mediators for the effects of gut microbiota on learning and memory after surgery and to determine whether gut microbiota mediates the effects of exercise on surgery-induced neuroinflammatory responses and impaired dendritic arborization, short chain fatty acids (SCFAs) in the blood were measured. SCFAs are products of gut microbiota. Valeric acid was the only SCFA whose concentrations in the blood were decreased in mice with exercise prior to surgery (FIG. 6A). Valeric acid was increased by surgery and this increase was attenuated by exercise in mice with surgery (FIG. 6B). In addition to valeric acid, exercise decreased propionic acid, butyric acid and hexanic acid and increased acetic acid, isobutyric acid and isovaleric acid in surgery mice. The increase of valeric acid in the blood was positively associated with lactobacillus and anaerotruncus and was negatively associated with Alistipes when the results of control mice and exercise mice were analyzed together (FIG. 6E). Consistent with this correlation and as described above, exercise reduced lactobacillus and increased Alistipes. Interestingly, the bacteria that were associated with decreased valeric acid in the feces (FIG. 6F) did not include the bacterium that was associated with decreased valeric acid in the blood, suggesting additional mechanisms for regulating the absorption of valeric acid in the gut into the blood or the metabolism of valeric acid in the feces during the transporting of feces to rectum.

Mice transplanted with feces from exercise mice had lower blood valeric acid than mice transplanted with feces from control mice. In another set of experiments, the findings were reproduced that exercise reduced valeric acid, butyric acid and hexanic acid in mice with surgery. Intraperitoneal injection of valeric acid attenuated this reduction caused by exercise. Interestingly, exercise did not change the concentrations of SCFAs in the feces. SCFAs in the feces of mice transplanted with feces from exercise mice were not different from those of mice transplanted with feces from control mice. These results support the idea that measuring fecal SCFAs may not be useful to reflect the concentrations of SCFAs in the blood.

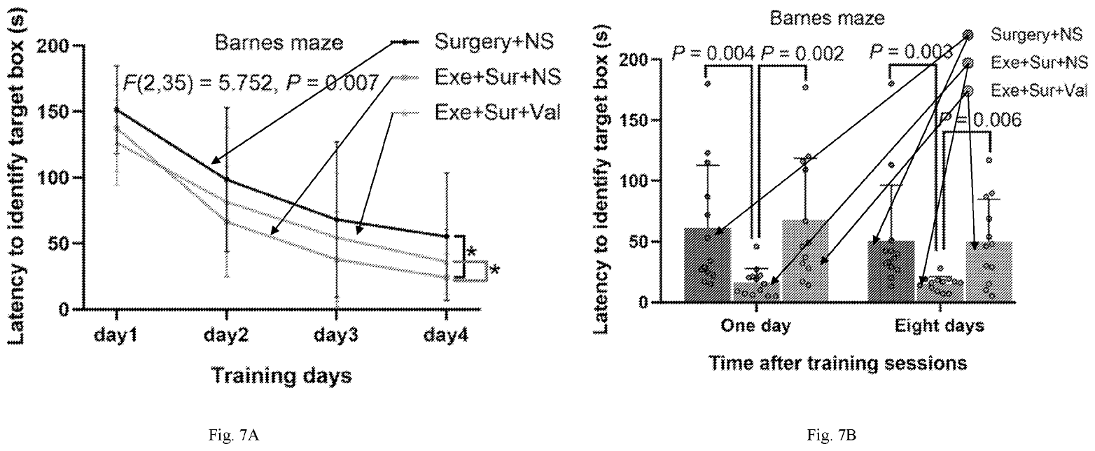

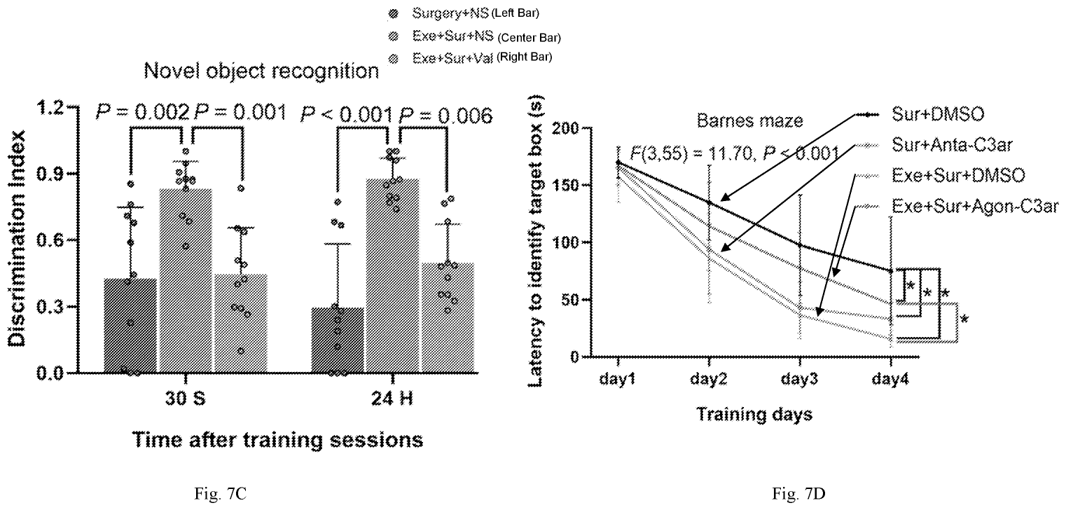

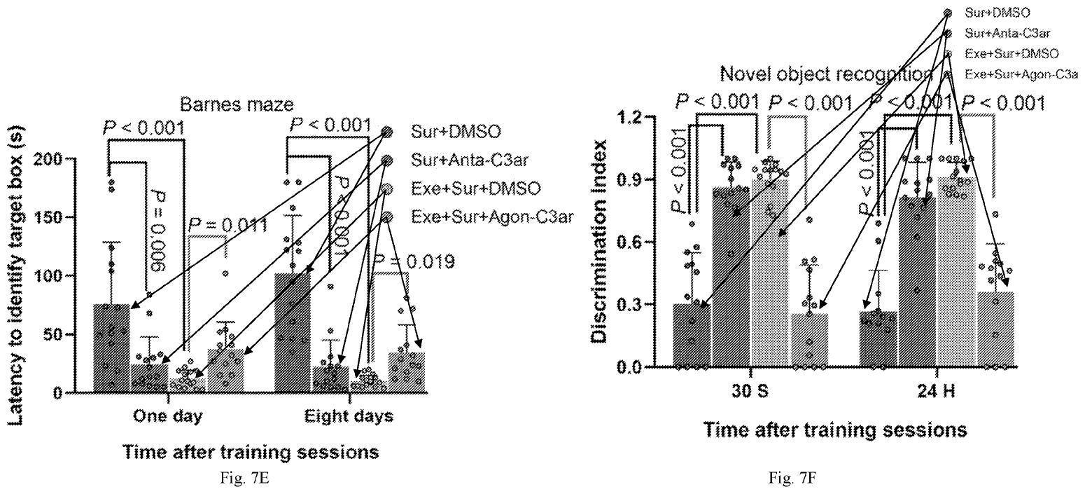

It was decided to study the potential role of valeric acid in participating in exercise effects because its concentration was decreased by exercise under baseline condition or after surgery. Consistent with the findings described above, mice with surgery took longer than control mice to identify target box in the Barnes maze test and this effect was attenuated by exercise. Intraperitoneal injection of valeric acid reversed the effects of exercise (FIGS. 7A, 7B). Similarly, exercise blocked the impairment of surgical mice in novel object recognition test and this protection of exercise was abolished by intraperitoneal injection of valeric acid (FIG. 7C). However, intracerebroventricular injection of valeric acid at 1/50 dose of intraperitoneal injection did not affect the performance of mice in the Barnes maze and novel object recognition tests. Taking together, these results suggest that reducing blood valeric acid provides a basis for exercise-induced protection against learning and memory impairment after surgery.

Consistent with the results of learning and memory, mice transplanted with feces from exercise mice had reduced C3ar1, C3, Iba-1, IL-1β and IL-6 in the hippocampus compared with mice transplanted with feces from control mice after surgery. Exercise reduced C3, C3ar, Iba-1 and IL-6 in the hippocampus and this reduction was blocked by intraperitoneal injection of valeric acid in mice with surgery (FIG. 3G). These results suggest that reducing blood valeric acid provides a mechanism for exercise-induced protection against immune and neuroinflammatory responses in the brain after surgery.

To determine whether C3 signaling played a role in the learning and memory impairment after surgery, mice received intracerebroventricular injection of a C₃ar antagonist or agonist. Surgery mice received SB290157, a C3ar antagonist [25], took less time than surgery mice received solvent injection to identify target box in Barnes maze. C3ar agonist increased the time for surgery mice with exercise to identify the target box (FIGS. 7D, 7E). SB290157 also improved the performance of surgery mice in novel object recognition test. C3ar agonist worsened the performance of exercise mice with surgery in novel object recognition test (FIG. 7F). These results suggest the role of C3 signaling in the development of POCD.

Mice transplanted with feces from exercise mice after surgery had more newly generated brain cells, intersections among dendritic branches and spine density than mice transplanted with feces from control mice. Exercise attenuated the decrease of newly generated brain cells, intersections among dendritic branches and spine density in the hippocampus and this attenuation was blocked by intraperitoneal injection of valeric acid in mice with surgery. In addition, surgery reduced the expression of postsynaptic density protein 95 (PSD95) and synapsin-1, two synaptic proteins. This decrease was attenuated by exercise. Mice transplanted with feces from exercise mice after surgery had increased PSD95 and synapsin-1 compared with mice transplanted with feces from control mice. Together, these results suggest that reducing blood valeric acid via altering gut microbiota provides a mechanism for exercise-induced protection against the reduction of brain cell genesis and dendritic arborization after surgery.

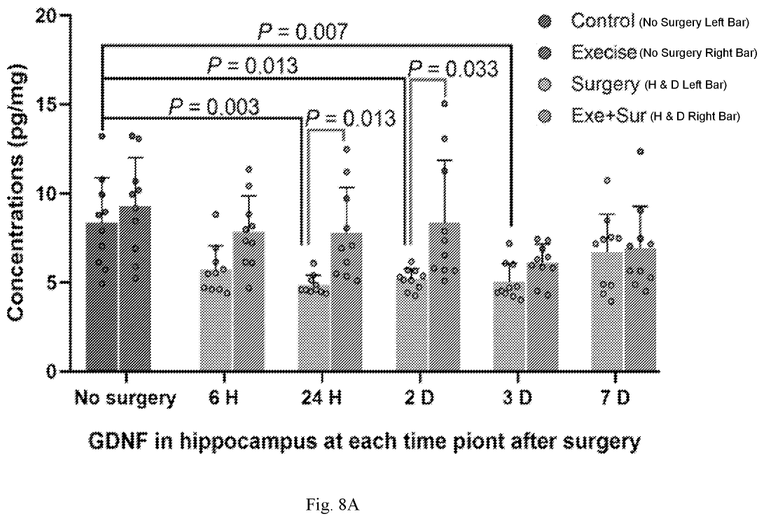

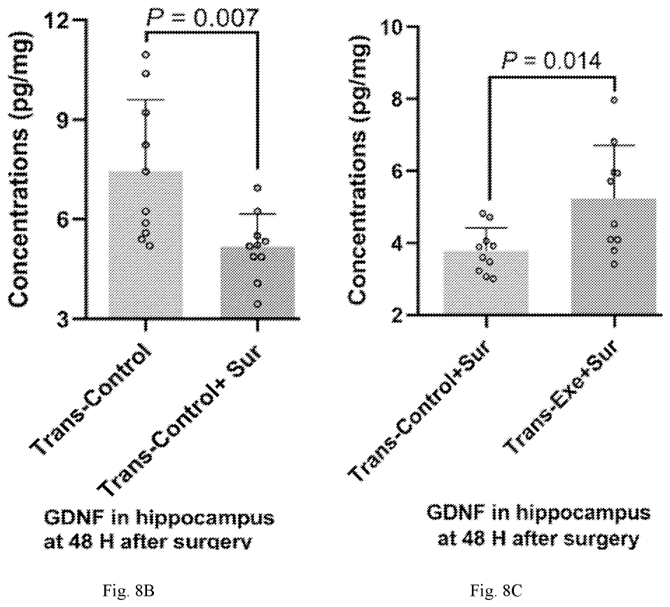

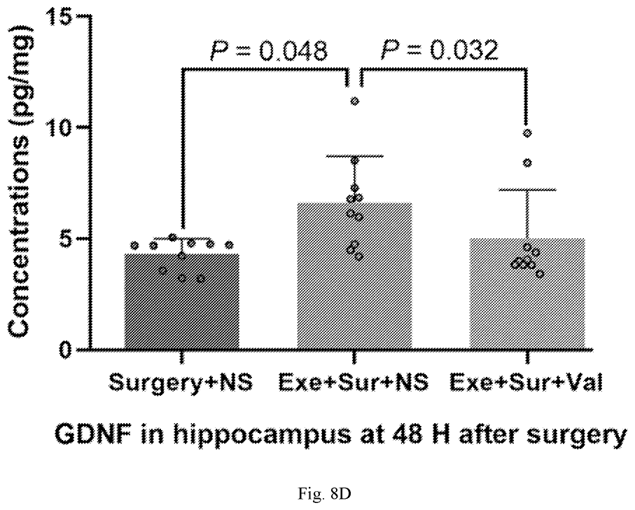

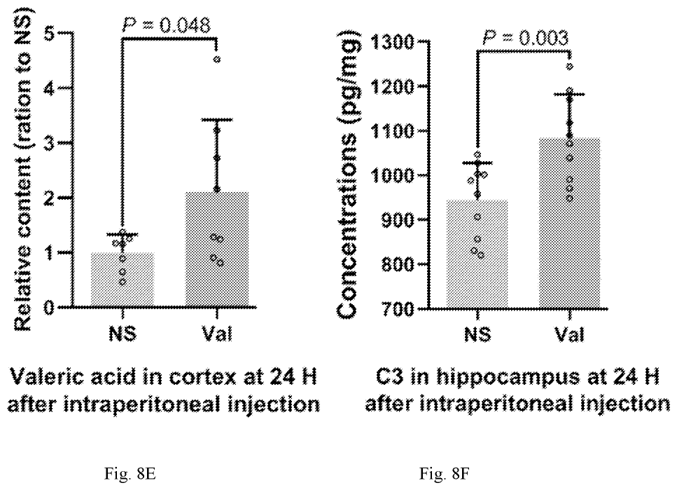

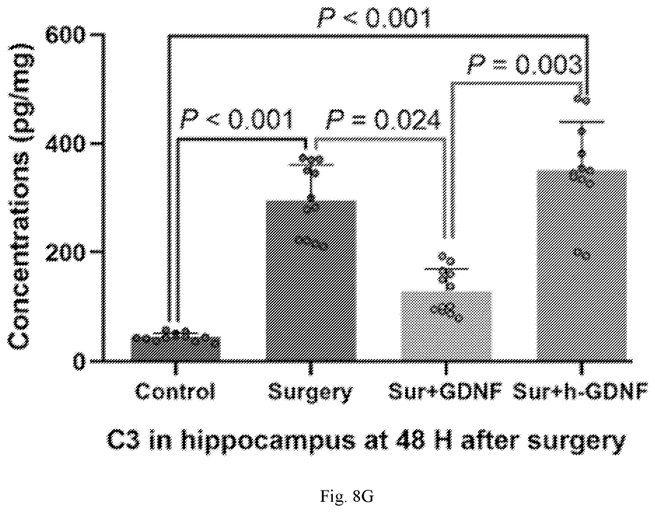

Previous studies have shown that the reduction of glia-derived nerve factor (GDNF) plays an important role in POCD [6,26]. Surgery decreased GDNF in the hippocampus. Exercise blocked this decrease (FIG. 8A). Surgery reduced GDNF in the hippocampus of mice transplanted with feces from control mice (FIG. 8B). Mice transplanted with feces from exercise mice had higher GDNF concentrations in the hippocampus than mice transplanted with feces from control mice after surgery (FIG. 8C). Intraperitoneal injection of valeric acid decreased GDNF levels in the hippocampus of exercise mice with surgery (FIG. 8D). This injection increased the amount of valeric acid and C3 in the brain (FIGS. 8E, 8F). These results suggest that GDNF can play a role as a molecule downstream of the alteration of gut microbiota for the effects of exercise to regulate the development of POCD. To support this idea, GDNF but not the heat-inactivated GDNF reduced C3, IL-6 and IL-1β levels in the hippocampus after surgery (FIGS. 8G-8I), suggesting that GDNF attenuates surgery-induced immune and inflammatory responses, which are considered to be important for POCD development.

### Example 3

### Gut Microbiota and Valeric Acid Played an Important Role in Exercise-Induced Attenuation of Neuroinflammation and Impairment of Learning, Memory, Brain Cell Generation and Dendritic Arborization after Surgery in Old Mice

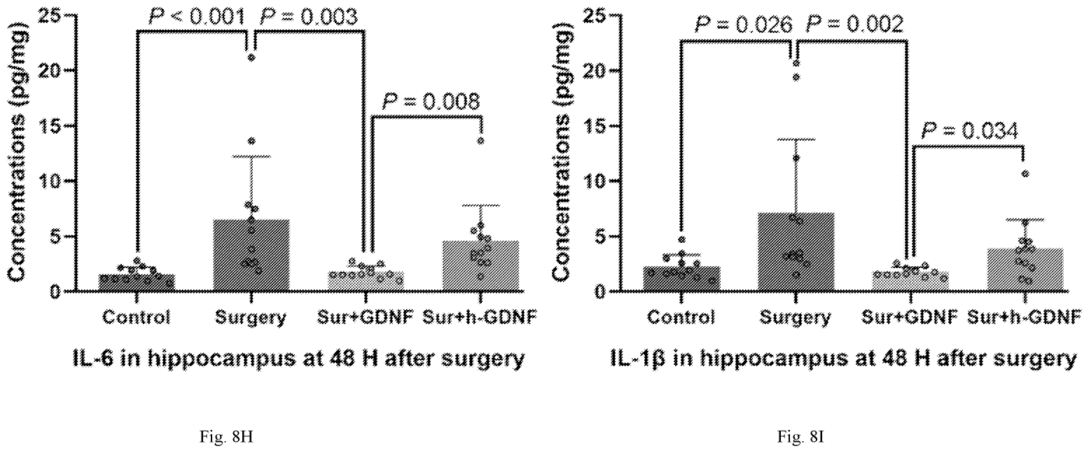

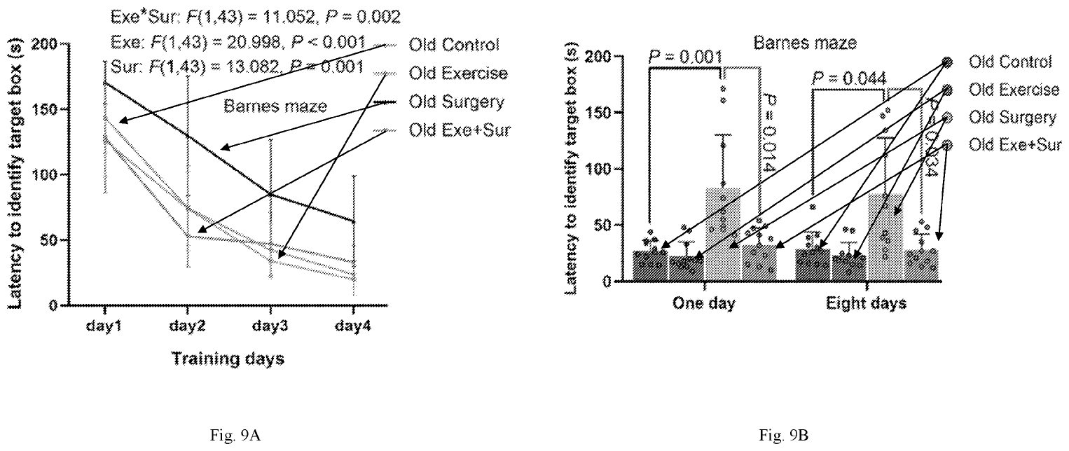

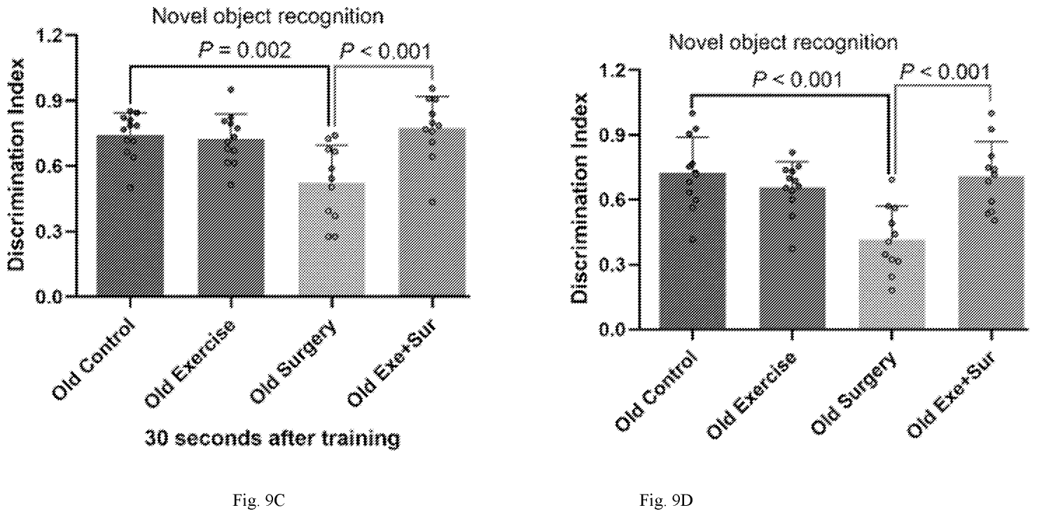

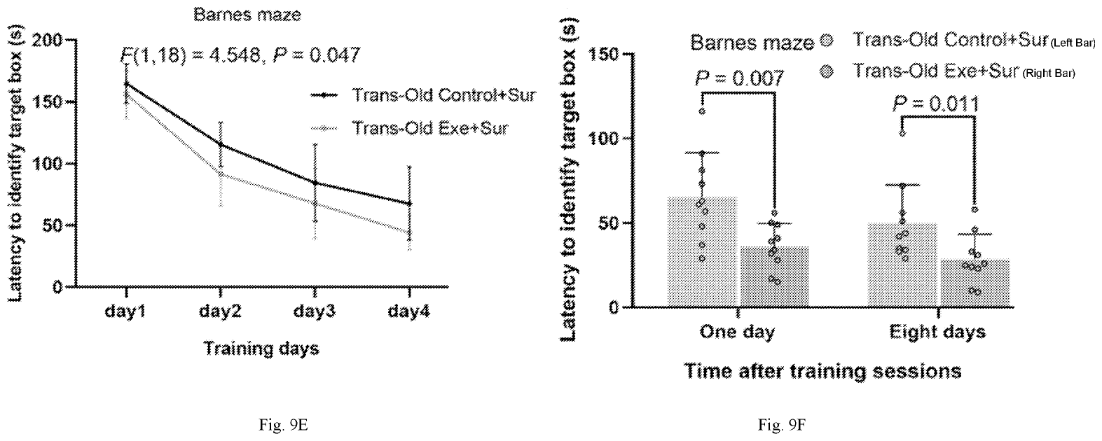

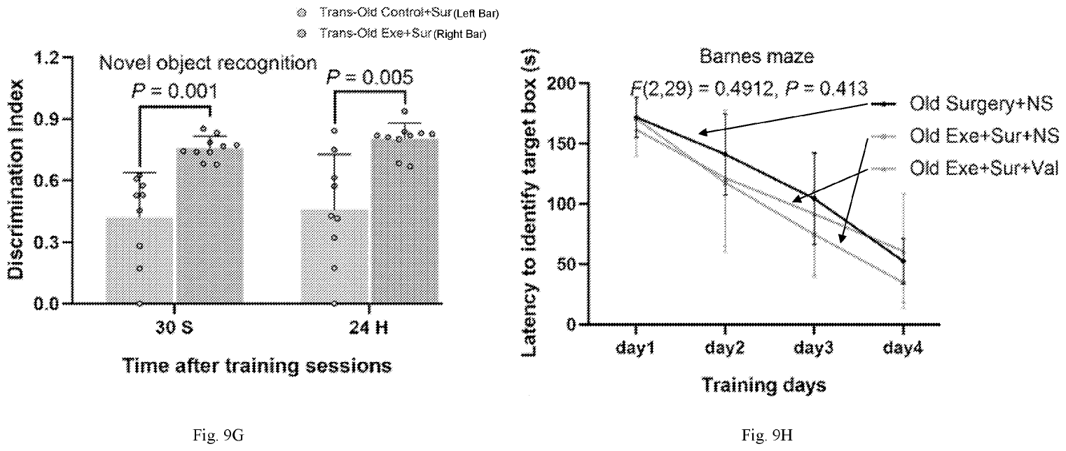

Since age is a risk factor for POCD [1, 2, 5], it was determined whether those mechanisms that identified in the young adult mice operated in old mice for POCD. Nineteen-month old mice with surgery took longer than control mice to identify the target box in Barnes maze test. This increase was attenuated by exercise (FIG. 9A, 9B). Similarly, surgery impaired the performance of old mice in novel object recognition test and this impairment was attenuated by exercise (FIGS. 9C, 9D). Surgery mice transplanted with feces from exercise mice performed better than surgery mice transplanted with feces from control mice in Barnes maze and novel object recognition tests (FIGS. 9E-9G). Also, intraperitoneal injection of valeric acid impaired the performance of surgery mice with exercise in Barnes maze and novel object recognition tests (FIGS. 9H-9J). These results suggest that exercise reduces the development of POCD via altering gut microbiota and that valeric acid is a mediator for this effect in old mice.

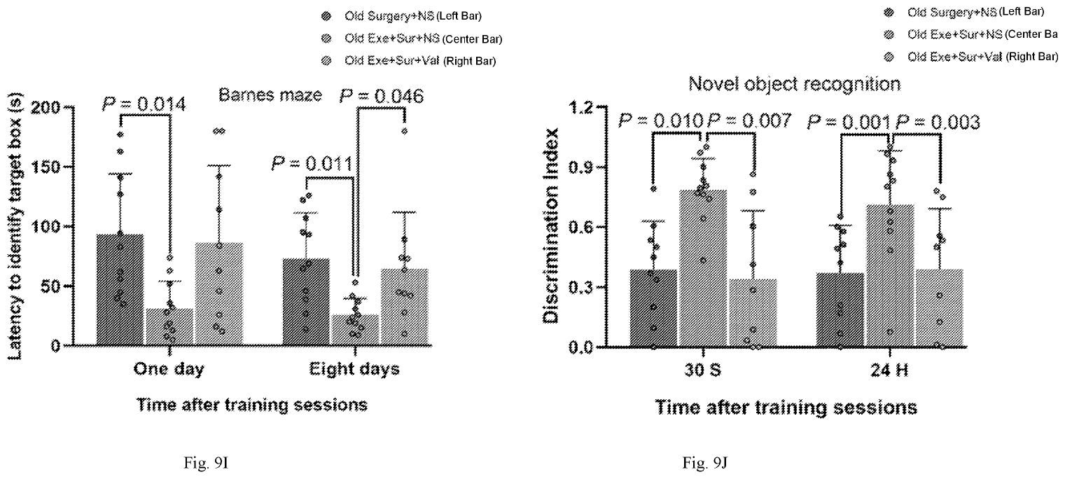

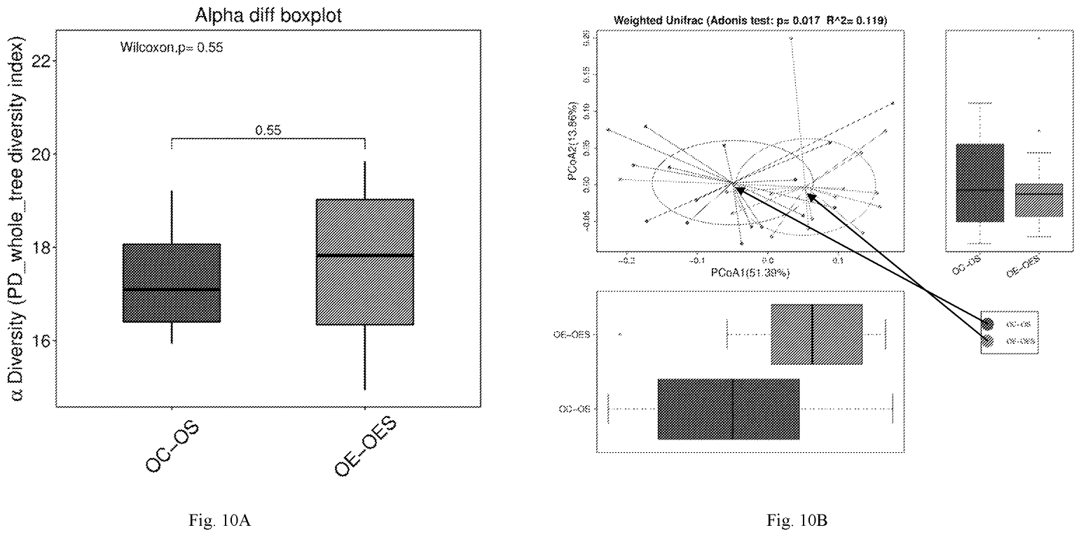

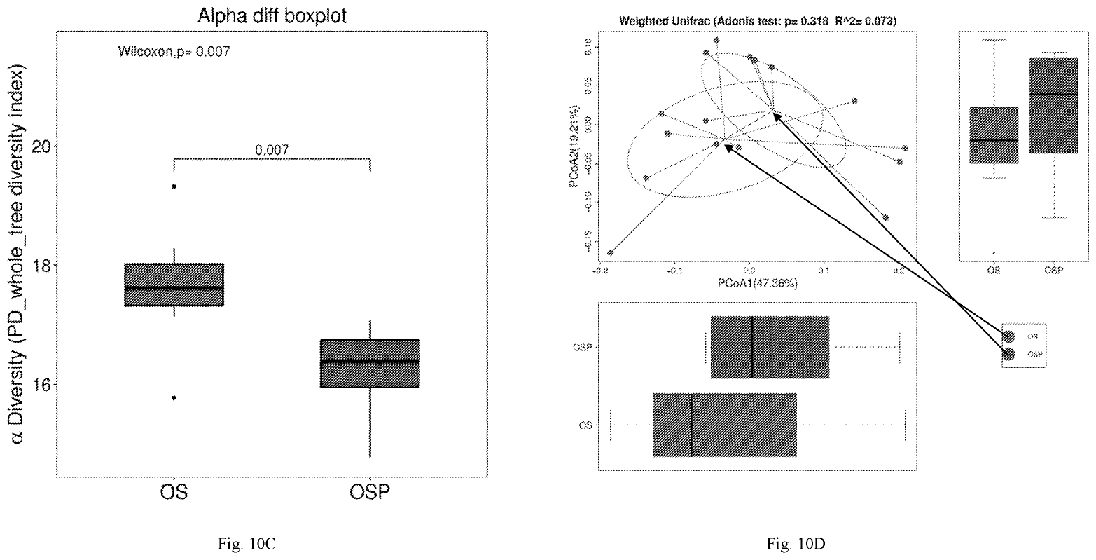

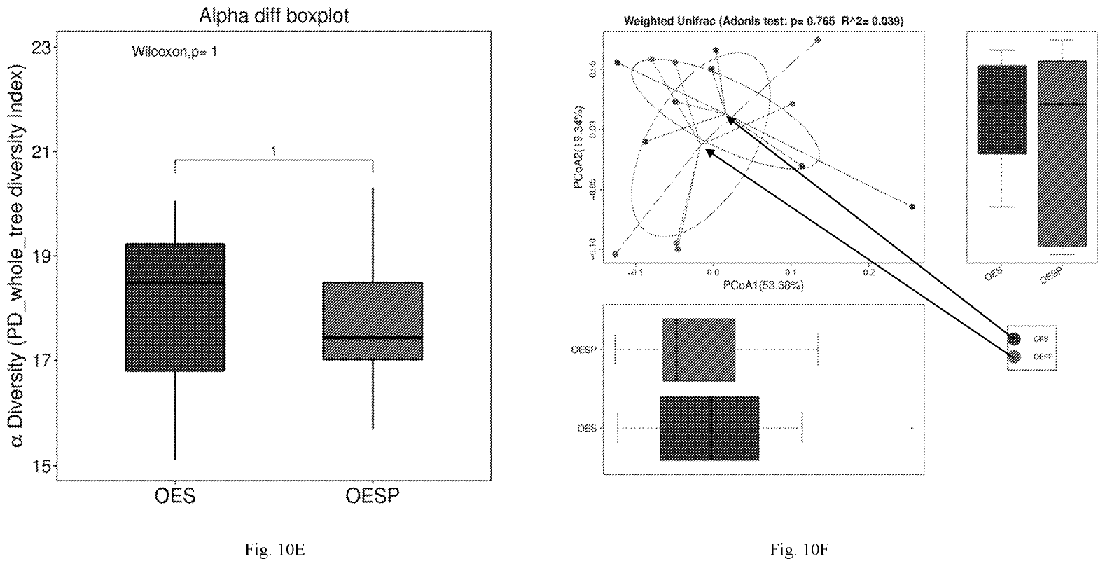

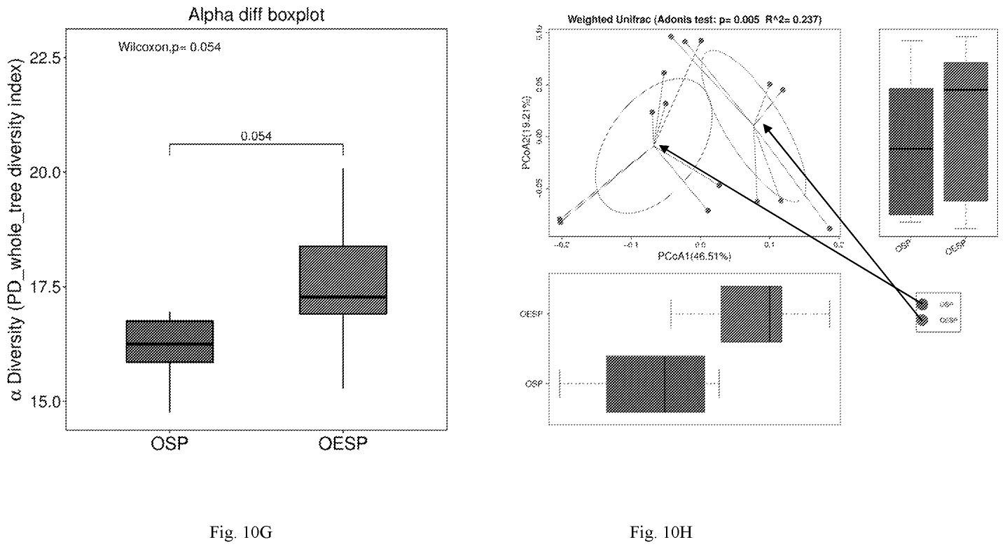

Exercise increased the β diversity among samples of old mice (FIG. 10A, 10B). Surgery decreased the diversity of gut microbiota in old control mice and this decrease was attenuated by exercise (FIG. 10C-10H). Gut microbiota of exercise mice or surgery mice transplanted with feces from exercise mice had an abundant amount of Bacteroidetes and Bacteroidales, while gut microbiota of control mice or surgery mice transplanted with feces from control mice contained a large amount of Clostridiales, Clostridia and Firmicutes. These results suggest that exercise increases the diversity of gut microbiota and stabilizes the gut microbiota of surgery mice.

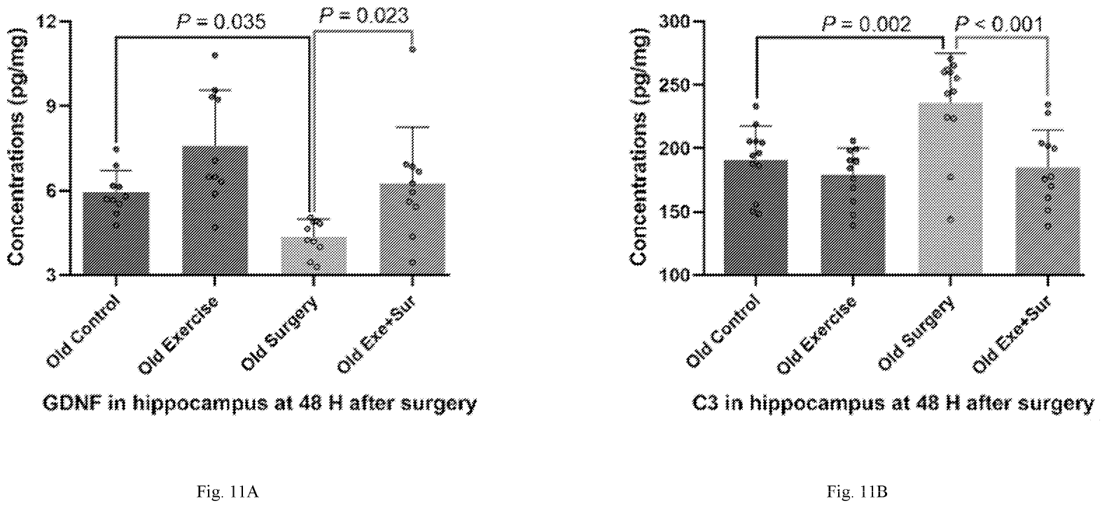

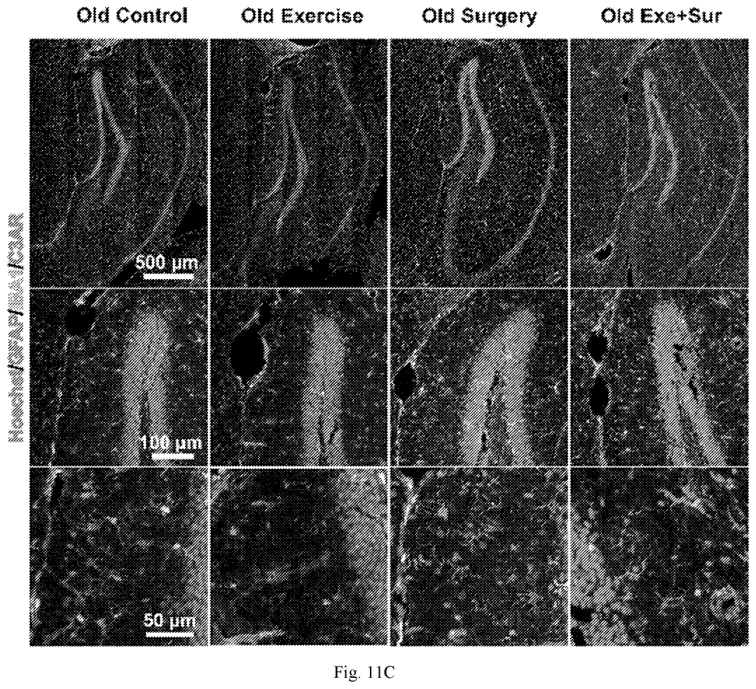

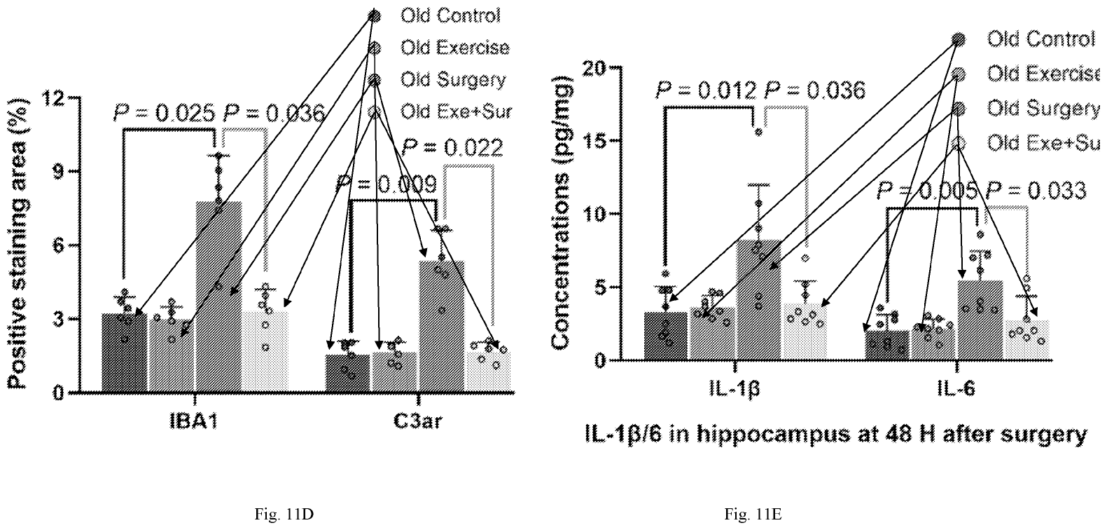

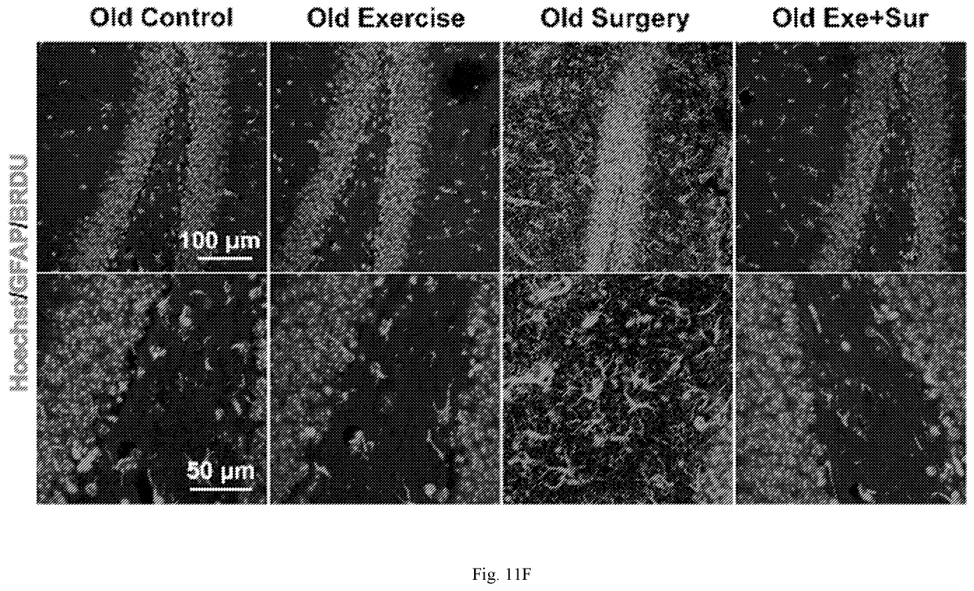

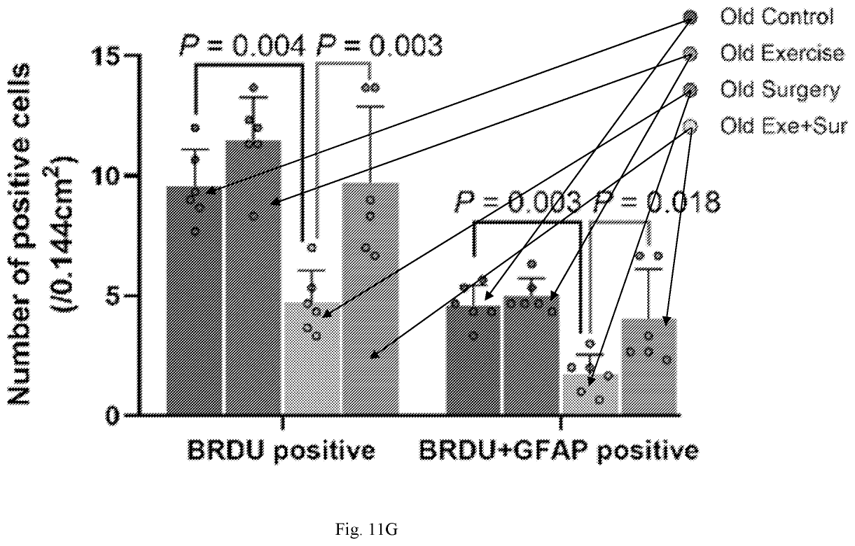

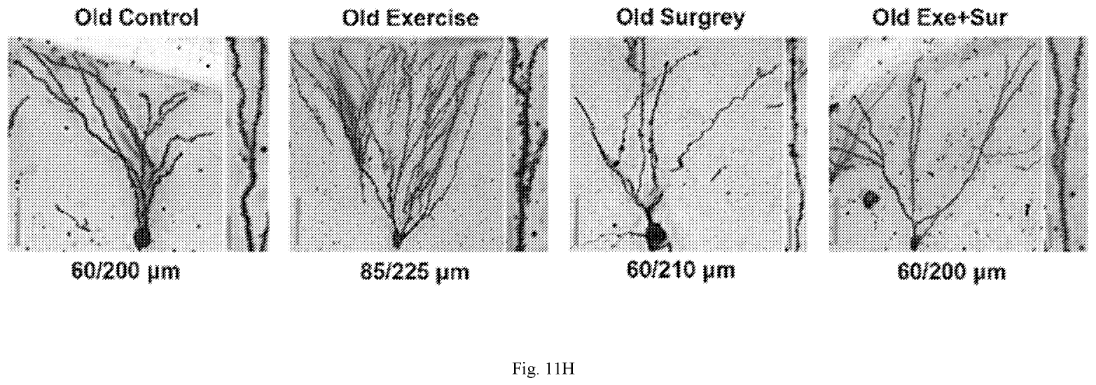

Although exercise did not alter valeric acid before surgery, exercise reduced valeric acid in surgery mice (FIGS. 6C, 6E). Similar to the findings in young adult mice, surgery reduced GDNF in the hippocampus. This reduction was attenuated by exercise (FIG. 11A). Surgery increased the expression of C3, C3ar, Iba-1, IL-1β and IL-6. These increases were attenuated by exercise (FIGS. 11B-11E). In addition, surgery decreased brain cell generation and spine density and exercise reserved brain cell generation and spine density in surgery mice (FIGS. 11F-11I). Finally, surgery reduced the expression of PSD95 and synapsin-1. Exercise blocked this decrease. These results suggest that pathological processes similar to those in young adult mice occur in old mice after surgery and that these processes including neuroinflammation, decrease of growth factor expression and dendritic arborization impairment can also be inhibited by exercise.

### Example 4

### Discussion of Examples 1-3

The presently disclosed results suggest that low intensity exercise attenuates surgery-induced learning and memory impairment as assessed by Barnes maze and novel object recognition tests. These beneficial effects occurred in both young adult and old mice. Physical activity ability has been associated with better preservation of mental status after surgery in humans [18]. The effects of exercise on POCD in general population have not been reported. Interestingly, the presently disclosed results showed that middle and high intensity exercise did not protect mice against learning and memory impairment after surgery, suggesting that only an appropriate level of exercise is protective. Consistent with this functional data, this level of exercise attenuated neuroinflammatory responses to surgery. Neuroinflammation is considered a critical pathological process for POCD [6, 7]. In addition, exercise attenuated surgery-induced decrease of brain cell genesis and dendritic arborization, structural bases for learning and memory [14, 22].

Previous studies have suggested an association between learning and memory dysfunction and gut dysbiosis after surgery [11-13]. The presently disclosed studies showed that mice transplanted with feces from exercise mice had better performance in Barnes maze and novel object recognition tests than mice transplanted with feces from control mice after surgery. Transplanting feces from control mice or mice with surgery established their corresponding gut microbiota in control mice. The recipients of feces from surgery mice but not those receiving transplantation of feces from control mice developed learning and memory dysfunction compared with control mice without fecal transplantation. Mice transplanted with feces from exercise mice had decreased neuroinflammatory responses and increased brain cell genesis and dendritic arborization. In addition, surgery decreased gut microbiota diversity and exercise stabilized the gut microbiota after surgery. These results provide direct evidence that gut microbiota alteration after surgery may contribute to the learning and memory dysfunction after surgery and that exercise may work on gut microbiota to reduce neuroinflammatory responses and to preserve learning, memory, brain cell genesis and dendritic arborization.

Gut microbiota produces SCFAs that can affect various brain functions including learning and memory. However, previous studies have only consider using a few SCFAs as a group for the effects. The presently disclosed results surprisingly suggest the contribution of decreasing valeric acid to the effects of exercise because exercise reduced valeric acid in the blood under baseline condition and after surgery. Valeric acid was decreased in the blood of mice transplanted with feces of exercise mice. Although exercise altered the concentrations of other SCFAs, such as propionic acid, in young adult mice with surgery, valeric acid was the only SCFA whose concentration in the blood was decreased by exercise in old mice with surgery. In supporting the role of valeric acid in the exercise effects, intraperitoneal injection of valeric acid worsened the learning and memory of young adult and old mice with exercise and surgery. Valeric acid also increased the inflammatory responses and impaired brain cell genesis and dendritic arborization. Together, these results suggest a detrimental effect of valeric acid on the brain. However, valeric acid injected intracerebroventricularly at 1/50 dose injected intraperitoneally did not impair learning and memory. This dose was selected because the brain accounts for about 2% total body weight. The failure to induce learning and memory impairment by this approach may be expected because valeric acid can cross blood-brain barrier [27, 32]. Consistent with this idea, valeric acid was detected in the brain tissues and intraperitoneal injection of valeric acid increased valeric acid in the brain in the presently disclosed study. Thus, the small amount of valeric acid injected into the brain may cross blood-brain barrier to be redistributed to other tissues and organs, which decreases the concentrations of valeric acid in the brain. Additional high doses of valeric acid injected into the brain were not performed because it does not appear that this approach will provide clear-cut evidence on whether valeric acid works directly on the brain to have its effects on the brain. Of note, intraperitoneal injection of valeric acid did not increase valeric acid in the blood of mice with exercise and surgery. This result may be anticipated because the blood was harvested 7 days after the last injection of valeric acid (7 days after surgery). However, the amount of valeric acid in the cerebral cortex was increased 24 h after the last intraperitoneal injection of valeric acid, suggesting that these injections increase valeric acid in tissues and organs. Also, it is not uncommon that previous studies measure SCFAs in the feces in the investigation of determining their effects on various organs [33, 34]. The presently disclosed results showed that the concentrations of SCFAs in the feces were much higher than those in the blood, consistent with a previous finding that only a small fraction of SCFAs in the feces can get into the circulation due to the gut absorption regulation and liver metabolism [28]. In addition, the presently disclosed study showed that surgery and exercise altered the concentrations of various SCFAs in the blood, they did not alter SCFAs in the feces. Thus, it is important to measure blood SCFAs when investigating their role in various organs.

Relatively limited information is known on how SCFAs affect cell activity and function. Recent studies suggest that SCFAs via working on their receptors or target proteins to activate intracellular signaling pathways, such as nuclear factor KB, to regulate the immune and inflammatory responses. SCFAs can also induce epigenetic regulation of gene expression including the expression of growth factors [27, 28]. The presently disclosed results suggest that these downstream events may contribute to the effects of exercise and valeric acid on learning and memory after surgery. Exercise attenuated surgery-induced neuroinflammation and GDNF reduction. Valeric acid blocked these exercise effects. Consistent with this GDNF role and the knowledge that neuroinflammation is critical for POCD [6, 7], it was shown in this study that GDNF reduced neuroinflammation after surgery.

An important molecule downstream of valeric acid to induce the effect on learning and memory can be C3 signaling. Surgery increased C3 and C3ar. Exercise attenuated this increase. Intraperitoneal valeric acid blocked the effects of exercise on C3ar and C3 expression. Intraperitoneal valeric acid increased C3 in the brain of control mice. More importantly, the C3 antagonist, SB290157 [25], blocked surgery-induced learning and memory dysfunction. A C3 agonist worsened learning and memory of mice with exercise and surgery. These results suggest that C3 signaling is important for learning and memory dysfunction after surgery. Compstatin is a peptide inhibitor and its blood-brain barrier permeability is not known [24]. Compstatin was given systemically in the previous study [35]. It is unclear whether the effects of compstatin on the brain were through its systemic effect or working on the brain. C3 agonist and antagonist was given directly into the brain, providing evidence to suggest the effects of C3 signaling in the brain on the learning and memory dysfunction after surgery. More importantly, the presently disclosed study has provided initial evidence to suggest that exercise and SCFAs, such as valeric acid, can regulate C3 signaling in the brain. Considering the broad effects of C3 signaling in the brain including regulating immune and inflammatory responses and neuronal activity [24], the presently disclosed study has identified a molecular mechanism for exercise to provide beneficial effects on the brain and for gut microbiota to affect the brain. In particular, the presently disclosed results suggest that exercise maintains astrocytes in an A2 phenotypic form because it reduced C3 and increased GDNF expression, a possible cellular mechanism for the beneficial effects of exercise.

Feces from 7 to 10 donors were pooled to prepare fecal solution for transplantation as described previously [36]. This method does not capture well the effects of individual differences in gut microbiota [37] but shall provide excellent representations of gut microbiota of the specific condition to all recipients. Individual variation in recipient response shall be preserved in this approach. In addition, this method does not require arbitrary match between recipients and donors. The fecal solution was applied by gastric gavage and enema to the recipients. This method of application quickly and effectively established the transplanted gut microbiota in the recipients in the presently disclosed study, possibly because microbiota applied by enema will not be affected by acidic solution and solution containing various enzymes or chemicals in the upper gastrointestinal tract.

The presently disclosed studies showed that exercise prior to surgery attenuated learning and memory impairment of mice with surgery. However, exercise did not affect the learning and memory of control mice. Exercise has been shown to improve learning and memory of animals modeled for various diseases [38, 39]. However, the effects of exercise on control animals are not consistent: both beneficial and no effects on learning and memory have been reported [40-42]. Specifically, four studies that used Barnes maze and/or novel object recognition tests to assess learning and memory in control mice with or without exercise were identified. One study showed that adult control rats with one episode of exercise that was at 60-70% maximal indirect oxygen uptake for 30 min immediately after the training session of novel object recognition test had better memory performance assessed 7 days but not at 1 day or 14 days after the training session [43]. The second study showed that voluntary exercise for 4 weeks did not affect the learning and memory assessed by Barnes maze and novel object recognition tests in control rats [44]. Two other studies had the data that swimming exercise improved memory of old rats assessed by novel object recognition test [454, 46]. A possible explanation for this inconsistency is that it requires appropriate levels of exercise to provide beneficial effects in control mice, as in the case shown in this study to prevent surgery-induced learning and memory dysfunction. This level of exercise was not specifically searched for because determining whether exercise improves learning and memory in control mice is not the focus of this study.

The presently disclosed findings have significant clinical implications. First, the study provided evidence to suggest that appropriate exercise levels are beneficial. It may not be difficult to understand that over-exhaustion may not be good for health. Since each individual has a different exercise capacity, it is wise to determine the maximal exercise capacity of the individual and have an individualized exercise protocol as was done in this study. This method shall be used in clinical situations if the beneficial effects of exercise are shown in humans. Second, the presently disclosed findings have suggested multiple potential interventions for reducing POCD, such as fecal transplantation and antagonizing C3 signaling. Third, the presently disclosed study suggests a detrimental role of valeric acid. Although there is no antagonist for valeric acid, these antagonists or methods to reduce blood valeric acid concentrations may be developed.

The possible detrimental effects of valeric acid on the brain are suggested by the presently disclosed study. Detailed mechanisms for valeric acid to induce these effects are to be further determined, in addition to providing evidence to suggest that C3 signaling activation can be a downstream event. Also, there was a focus on investigating the mechanisms for the beneficial effects of low intensity exercise but it was not determined why higher intensity exercises lack beneficial effects on surgery-induced learning and memory dysfunction. Determining the mechanisms for this failure may provide insights on exercise physiology and pathophysiology and identify possible targets to be eliminated for the higher intensity exercises to be protective. However, it appears the need for this determination is not high since low intensity exercise is protective and easy to be achieved.

In summary, the presently disclosed study suggests that low intensity exercise can stabilize gut microbiota after surgery. This effect reduces valeric acid in the blood, which then maintains the productions of growth factors, such as GDNF, and inhibits neuroinflammation (FIG. 12). Transplantation of healthy gut microbiota and antagonizing C3 signaling provide bases for interventions to reduce learning and memory dysfunction after surgery.

### Methods and Materials for Examples 5-9

### Animals and Experimental Design

Eight-week C57BL/6J male mice (328 mice in total) were purchased from Charles River (Wilmington, MA, USA) and 18- to 21-month old male C57BL/6J mice (64 mice in total) were provided by the National Institutes of Health (Bethesda, MD). They were maintained in the vivarium under pathogen-free conditions (23±2° C.; 12-h light/dark cycle) with free access to food and water and housed 3 to 5 mice per cage before and during the experiments. All experimental procedures were approved by the Institutional Animal Care and Use Committee of the University of Virginia (Charlottesville, VA, USA). All animal experiments were performed in accordance with the National Institutes of Health Guide for the Care and Use of Laboratory Animals (NIH publications number 80-23) revised in 2011.

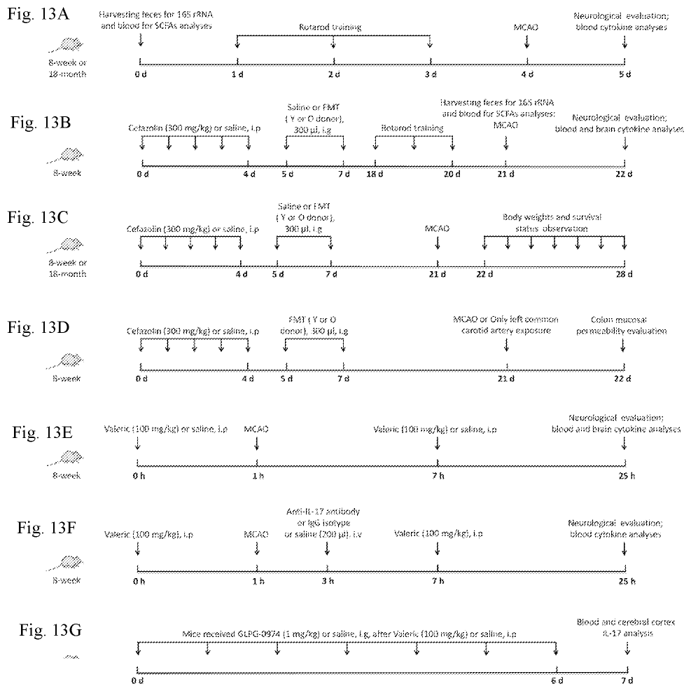

In the first experiment (FIG. 13A), 8-week and 18-month old C57BL/6J male mice were subjected to the left middle cerebral artery occlusion (MCAO). Blood and feces from mice of these two ages without MCAO were harvested for analyses of SCFAs in the blood and 16S rRNA in the feces. Neurological outcome and cytokines in the blood were evaluated 24 h after MCAO.

To explore the effects of gut microbiota of young (8 weeks) or old mice (18˜ 20 months) on stroke, 8-week old C57BL/6J male mice were randomly divided into four groups (FIG. 13B): control group that was treated with normal saline by intraperitoneal injection daily for 5 days and then normal saline by gastric gavage once a day for two days; antibiotic group that was treated with cefazolin 300 mg/kg daily by intraperitoneal injection for 5 consecutive days and then normal saline by gastric gavage once a day for two days; old mouse fecal transplantation (Young-oFMT) group that was treated with cefazolin 300 mg/kg daily by intraperitoneal injection for 5 consecutive days and then 300 μl fecal solution from old mice by gastric gavage once a day for two days, and young mouse fecal transplantation (Young-yFMT) group that was treated with cefazolin 300 mg/kg daily by intraperitoneal injection for 5 consecutive days and then 300 μl fecal solution from young mice by gastric gavage once a day for two days. Gastric gavage occurred on day 1 and day 3 after the last dosage of cefazolin. Feces and blood were harvested 14 days after fecal transplantation for 16S rRNA and SCFA analyses. Mice were subjected to MCAO 2 weeks after gastric gavage. Neurological outcome was evaluated 24 h after MCAO. Blood and brain were harvested for cytokine analyses 24 h after MCAO in another cohort of mice. The third cohort of the same 4 groups of 8-week old C57BL/6J male mice (control group, antibiotic group, Young-oFMT and Young-yFMT) were used to determine the body weights and survival status after MCAO (FIG. 13C). Similarly, 18-month-old C57BL/6J male mice were randomly divided into four groups: control group, antibiotic group, old mouse fecal transplantation group (Old-oFMT) and young mouse fecal transplantation group (Old-yFMT). The treatments of these four groups were the same as described above for 8-week old C57BL/6J mice. Body weights and survival status were observed after MCAO (FIG. 13C).

To determine the effects of gut microbiota of young or old mice on the colon mucosal permeability after stroke, 8-week-old C57BL/6J male mice were randomly divided into four groups (FIG. 13D): old mouse fecal transplantation-sham group that received old mouse fecal transplantation and left common carotid artery exposure but without the induction of MCAO; young mouse fecal transplantation-sham group that received young mouse fecal transplantation and left common carotid artery exposure but without the induction of MCAO; old mouse fecal transplantation-MCAO group and young mouse fecal transplantation-MCAO group. Colon mucosal permeability was tested 24 h after ischemic reperfusion.

To investigate the effects of valeric acid on stroke, 8-week old C57BL/6J male mice were randomly divided into two groups (FIG. 13E): saline-stroke group that received intraperitoneal 100 μl normal saline 1 h before MCAO and 6 h after MCAO, and valeric-stroke group that received intraperitoneal 100 mg/kg in 100 μl valeric sodium (catalog number: V091420, LOT number: 1-SHE-30-1, Toronto Research Chemicals, Inc., Toronto, ON, Canada) 1 h before MCAO and 6 h after MCAO. Neurological outcome was evaluated 24 h after MCAO. Blood and brain were harvested for cytokine analyses. A pilot experiment was performed to determine the dosage of valeric acid used in this study. Eight-week old mice were divided into two groups: Y-Saline group that received two intraperitoneal 100 μl normal saline injections at an interval of 7 h, and Y-Valeric group that received two intraperitoneal 100 mg/kg in 100 μl valeric sodium injections at an interval of 7 h. One group of 18-month old mice that was labeled O-Saline received two intraperitoneal 100 μl normal saline injections at an interval of 7 h. Blood was harvested 3 h after the second injection of valeric sodium or normal saline to measure valeric acid.

To test the role of IL-17 in valeric acid-worsened neurological outcome after MCAO, 8-week old C57BL/6J male mice that received 2 doses of valeric acid as described above were randomly divided into three groups (FIG. 13F): saline control group that received intravenous 200 μl normal saline 2 h after the initiation of MCAO; IgG isotype control group that received intravenous 100 μg in 200 μl mouse IgG1 kappa isotype control (catalog number: 14-4714-85, LOT number: 2087674, Thermo Scientific, Rockford, IL, USA) 2 h after the initiation of MCAO; and anti-IL-17 antibody group that received intravenous 100 μg in 200 μl mouse monoclonal anti-IL-17 antibody (catalog number: 16-7173-85, LOT number: E06909, Thermo Scientific, Rockford, IL, USA) 2 h after the initiation of MCAO. Neurological outcome was evaluated 24 h after MCAO. Blood was harvested for cytokine analyses.

To determine whether valeric acid increased IL-17 via SCFA receptors, 8-week-old male mice were randomly divided into four groups (FIG. 13G): saline control group that received intraperitoneal 100 μl normal saline for 7 days, valeric acid group that received intraperitoneal 100 mg/kg in 100 μl valeric sodium for 7 days, valeric acid plus antagonist group that received 1 mg/kg GLPG-0974 (catalog number: SML2443, Sigma-Aldrich, Inc. St. Louis, MO, USA), a FFAR2 antagonist [19], by gastric gavage immediately after each of the valeric acid intraperitoneal injections for 7 days, and antagonist control group that received 1 mg/kg GLPG-0974 by gastric gavage, once a day, for 7 days. Blood and cerebral cortex were collected for IL-17 analysis.

### Fecal Transplantation

Fresh fecal pellets collected within 30 min after bowel movement from old (18 to 21 months old) or young (8 weeks old) mice were diluted with 100 mg/ml sterile saline. All fecal pellets of old mice were from the same eleven old mice and re-suspended together in saline. These donors also provided feces individually for gut microbiome profiling as the old mouse group. However, the feces of different sets of young mice (10 mice for each set) were harvested for transplantation to keep the donor age at 8 to 10 weeks. These donors (old mice or young mice) were housed together at 3 to 5 mice per cage. Briefly, the fecal matter was vortex-mixed for 5 min and then passed through 70 μm nylon cell strainer (catalog number: 08-771-2, Fisher Scientific, San Diego, CA, USA) to remove undigested food and smaller particulate materials. To facilitate the colonization of the transplanted microbiota, recipient mice were given an intraperitoneal injection of 300 mg/kg cefazolin once daily for 5 consecutive days to eliminate the original gut microbiota in the recipients. This regimen reduced the gut microbiota in the colon to an almost undetectable level by 16S rRNA analyses in our previous study [20]. The recipient mice received 300 μl corresponding fecal solution by gastric gavage at 24 h and 72 h after the last dose of cefazolin. A volume at 10 ml/kg and 20 ml/kg as an ideal and maximal volume, respectively, for gastric gavage has been recommended for mice and a volume at 500 μl was used in our previous study [22]. An interval of 24 h between the last dose of cefazolin and receiving fecal solution was used because an interval of 1 to 2 days is commonly used in gut microbiota transplant studies [22-24]. A very small amount of cefazolin may remain in the mice 24 h later because its plasma half-life in rodents is 0.5 h [24]. Transplanted mice were maintained for 2 weeks after fecal transplantation before they were subjected to MCAO.

### Middle Cerebral Artery Occlusion

As we described previously [25, 26], left MCAO with intravascular suture technique was performed to mice. Briefly, mice were anesthetized with isoflurane and the rectal temperature was maintained at 37±0.5° C. by the feedback-controlled heating system. A monofilament nylon suture (catalog number: 1622-A1, Beijing CiNontech Co. Ltd., Beijing, China) was used to induce the MCAO via the external carotid artery to the left internal carotid artery until slight resistance was felt. Mice were re-anesthetized by isoflurane and the sutures were removed at 60 min (only for the set of experiments of old mice with fecal transplantation; this selection was because of a high mortality rate after a longer MCAO) or 120 min (for all other sets of experiments) after the onset of MCAO.

### Evaluation of Motor Co-Ordination, Neurological Deficit Scores, and Infarct Volumes

The infarct volumes were assessed 24 h after MCAO by a blinded investigator after 2,3,5-triphenyltetrazolium chloride staining [25, 26]. Briefly, mice were anaesthetized deeply with 5% isoflurane and transcardiacally perfused by saline. Brains were cut into 6 2-mm thick coronal slices to evaluate the infarct volume. The average infarct area of the rostral and caudal sides of each slice was calculated. The infarct volume of the brain slice was calculated by multiplying the average infarct area of the slice by the thickness of the slice. The sum of infarct volumes of all brain slices from the mouse was considered as the total infarct volume of the brain. To correct the cerebral edema and differential shrinkage resulting from brain ischemia and tissue processing, the corrected brain infarct volume was calculated: corrected brain infarct volume in percentage=[right hemisphere volume-(left hemisphere volume-left infarction volume)]×100/right hemisphere volume.

Neurological deficit scores were tested 24 h after the stroke by a blinded investigator [25, 26]. Briefly, mice were scored as follows: zero, no apparent deficits; one, failure to extend right forepaw fully; two, decreased grip of the right forelimb; three, spontaneous movement in all directions, contralateral circling only if pulled by the tail; four, circling or walking to the right; five, walking only if stimulated; six, unresponsiveness to stimulation and with depressed level of consciousness; and seven, dead.

Rotarod test was performed to evaluate the motor coordination as we described before [25, 26]. All mice received the training for three continuous days before the induction of brain ischemia. The formal test was performed just 24 h before and 24 h after the MCAO. Mice were placed on a rotarod with the speed accelerated from 4 to 40 rpm within 5 min. The latency and the speed were recorded when the tested mice fell off the rod. Each mouse was tested five times. The speed-latency index, which is latency(s)×speed (rpm), was calculated. The ratio of mean index of the five trials obtained after MCAO and before MCAO was calculated to reflect the change in coordinate function of each mouse.

### In Vivo Colon Mucosal Permeability Assay

Colon mucosal permeability was measured under in vivo condition using Evans blue as described before [27]. Mice were placed in a supine position on a heating pad and a laparotomy was performed under isoflurane anesthesia with spontaneous breathing. The cecum was dissociated and placed outside the abdominal cavity. A small hole was cut in the cecum. A small polyethylene tube (G18) was inserted into rectum through the anus and secured by a ligature. The colon was gently flushed with phosphate buffered saline (PBS) via this tube until all feces were rinsed out. The proximal and distal colon were ligated and 1 ml of 1.5% Evans blue (catalog number: E2129-10G, Sigma-Aldrich, St. Louis, MO, USA) in PBS was instilled into the colon. The colon was rinsed 30 min later with PBS until the perianal washout was clear. Then the mice were sacrificed under deep anesthesia and the colon was removed rapidly. The dissected colon was opened and rinsed again with 3 ml PBS, followed by 5 ml 6 mM N-acetylcysteine (catalog number: PHR1098-1G, Sigma-Aldrich, St. Louis, MO, USA) to eliminate any unabsorbed Evans blue in the colonic mucus. The colon was weighed and then placed in 1 ml formamide (catalog number: F9037-100ML, Sigma-Aldrich, St. Louis, MO, USA) at 50° C. for 24 h to extract the Evans blue dye. The dye concentration in the supernatant was measured spectrophotometrically at 655 nm and given as microgram per gram colonic tissue.

### Gut Microbiota Profiling

Fresh feces from mice were collected for gut microbiota profiling. Mice were placed individually in an autoclaved cage for collecting feces and allowed to defecate freely. Feces were collected immediately in the sterile Eppendorf tubes on ice and then stored at −80° C. until further processing. Bacteria DNA was extracted with Power Lyzer Power soil DNA isolation kit (catalog number: 12855-100, QIAGEN, Germantown, MD, USA) according to our previous protocols [20]. Sequencing libraries of the hypervariable V3-V4 region were prepared according to the Illumina MiSeq system instructions [20]. Briefly, 12.5 ng DNA was used as DNA template for the first 16S rRNA PCR. Primers used were as follows: F: 5′-TCGTCGGCAGCGTCAGATGTGTATAAGAGACAGCCTACGGGNGGCWGCAG-3′ (SEQ ID NO. 15) and R: 5′-GTCTCGTGGGCTCGGAGATGTGTATAAGAGACAGGACTACHVGGGTATCTAATCC-3′ (SEQ ID NO. 16). The amplicons were then cleaned up with AMPure XP magnetic beads (catalogue number: A63881, Beckman Coulter, Brea, CA, USA). The barcode primer sets N701-704 and S502-511 (the Nextera XT Index Kit, catalogue number: FC-131-2001, Illumina, San Diego, CA, USA) were used for pair-ended sequencing. Library preparation and sequencing were performed by Genome Analysis Technology Core at the University of Virginia. Qubit dsDNA HS assay kit (catalogue number: Q33230, Thermo Scientific, Rockford, IL, USA) and TapeStation high sensitivity D1000 ScreenTape (Agilent, Blacksburg, VA, USA) were used to measure the concentration of PCR products and normalize the quantity for library preparation. Sequencing was operated on an Illumina MiSeq instrument by MiSeq reagent kit v2 (500 cycles). Data was analyzed with the MiSeq Reporter software Metagenomics workflow v2.5.1.3 (Illumina, San Diego, CA, USA).

The paired reads obtained by double-terminal sequencing were spliced into a sequence through Pandaseq software. The long reads with high variable region were obtained. The reads whose average phred score in the window (5 bp in size, 1 bp step length) was less than 20 were trimmed. Reads containing ambiguous “N” or with length <220 bp were discarded. After quality control of the original data, the high-quality sequences without chimeras were arranged according to abundance from large to small and then clustered with 97% similarity into an Operational Taxonomic Units (OTU). Each OTU was considered to represent a species. To avoid the deviation of analysis caused by the different sizes of sequencing data in samples, the number of Reads to OTU was entered at a level of the sequencing depth that was minimal among the samples. Alpha diversity was analyzed by random leveling. A Read was extracted from each OTU as a representative sequence. The representative sequence was compared with the database of the Ribosomal Database Project. Species were classified for each OTU, and species abundance tables were obtained for subsequent analysis. OTU picking was performed using Uclust on the software platform QIIME v1.9.1. Alpha diversity including Chaol index, observed_species index, PD_whole_tree index, Shannon index and Simpson index was used for the analysis of species diversity in a single sample. Unweighted Unifrac principal coordinate analysis (PCoA) and analysis of similarity (ANOSIM) were used for Beta diversity analysis to compare microbial compositions among samples. The relative abundance of taxa within gut microbiota at genus level was compared between groups. The gut microbiota data analysis was performed by Realgene (Shanghai, China).

### Plasma SCFA Profiling

Plasma SCFA concentrations were quantified using an Agilent 7890-5977 gas chromatography-mass spectrometry (GC-MS, Agilent, Blacksburg, VA, USA). Plasma samples were collected from old and young mice or 2 weeks after the mice were transplanted with young or old mouse fecal solution, stored at −80° C. and thawed to room temperature for processing. Briefly, 100 μl plasma sample was mixed with 900 μl ethanol (containing 0.5% HCl, V/V) and vortex-mixed. Ultrasonic treatment was applied for 40 min and then the samples were centrifuged at 14000 RPM for 10 min. The supernatant of samples was measured by GC-MS analysis on an Agilent 7890-5977 GC-MS with electron impact ionization and a DB-FFAP capillary column (30 mx0.25 mm×0.25 μm).

Valeric acid in the blood harvested from the experiment to determine the dosage of valeric acid injected intraperitoneally was measured by using an ELISA kit (catalog number: JM-12270M1, Jingmei Biotechnology, Jiangsu, China) following the manufacturer's instructions.

### Serum and Brain Tissue Harvesting

Mice were weighed and anesthetized with isoflurane at 24 h after MCAO. Blood samples were obtained from the right heart after thoracotomy. Blood was centrifuged (13,000 g, 4° C.) after it had been placed at 4° C. for 2 h for the collection of serum. After the collection of blood, mice were perfused with 4° C. saline for brain tissue harvesting. The cerebral cortex (anteroposterior: −1.5 to 2.5 mm, mediolateral: 1.0 to 3.0 mm) was dissected out. All dissection procedures were performed on ice.

### ELISA of Interleukins in the Serum and Brain

IL-10, IL-17, IL-1β and IL-6 in the serum and brain were detected by using ELISA kits (catalog number: M1000B, M1700, MLB00C and D6050, R&D SYSTEM, Inc., Minneapolis, MN, USA) as we described before [8]. The brain tissues were homogenized on ice in the RIPA buffer containing 25 mM Tris-HCl with pH 7.6, 150 mM NaCl, 1% sodium deoxycholate and 0.1% SDS (catalog number: 89901, Thermo Scientific, Rockford, IL, USA) and a protease inhibitor cocktail containing 10 mg/ml aproteinin, 5 mg/ml peptastin, 5 mg/ml leupetin and 1 mM phenylmethane sulfonylfluoride (catalog number: SRE0055, Sigma-Aldrich, St. Louis, MO, USA). The supernatant was collected for ELISA detection after being centrifuged for 20 min (13,000 g, 4° C.) and the protein concentration was determined by the BCA method. Cytokines in the serum and supernatant of brain were detected according to the manufacturer's instruction. The amount of IL-10, IL-17, IL-1β and IL-6 in each sample in the brain was normalized by its protein content.

### Statistical Analysis

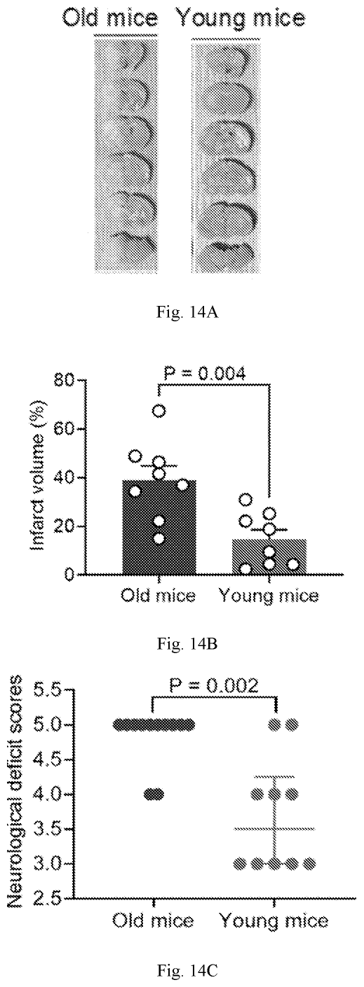

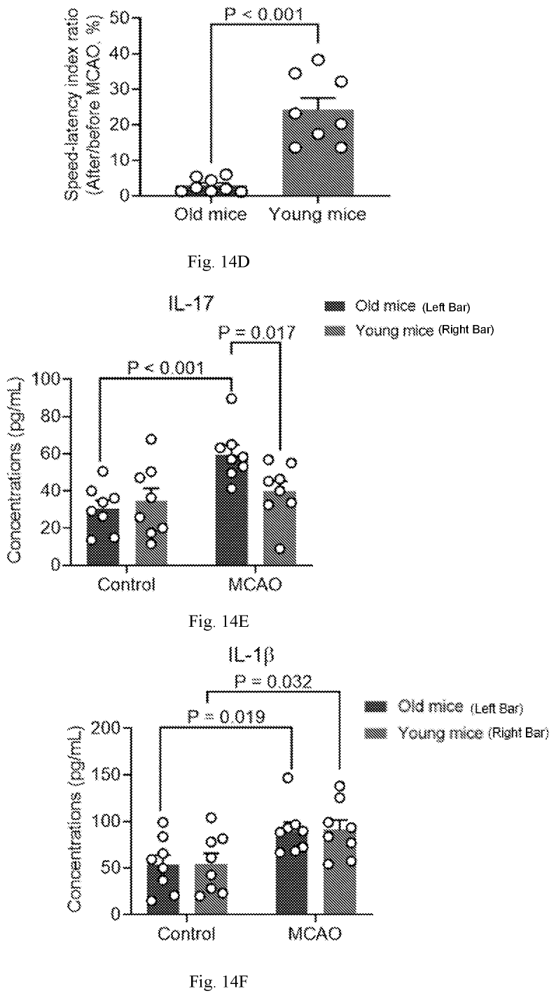

Parametric results in normal distribution are presented as means±S.E.M. Data are presented as median with interquartile range when they are not in normal distribution or are not parametric. Data of each individual animal is also presented in the figures. Two-way ANOVA was used to determine whether age and brain ischemia were significant factors in determining the cytokine concentrations in the blood (data in FIGS. 14E-14H). Gut microbiota data were analyzed as described in section Gut microbiota profiling. The other data were analyzed by one-way ANOVA followed by Tukey's test if the data were normally distributed, by one-way analysis of variance on rank followed by Tukey test if the data were not normally distributed, by t-test or rank sum test. Body weight data were analyzed by two-way repeated measures ANOVA. Survival curves were analyzed by Mantel-Cox test followed by the Gehan-Breslow-Wilcoxon test. Significant difference was defined as P<0.05. All statistical analyses were performed with SigmaStat (Systat Software, Inc., Point Richmond, CA, USA).

### Example 5

### Old Mice had a Worsened Neurological Outcome and Enhanced Proinflammatory Cytokine Production after Brain Ischemia and Changed Composition in Gut Microbiota and Increased SCFAs in the Blood Before Brain Ischemia

To determine whether aging affected neurological outcome after brain ischemia, 8-week and 18-month old C57BL/6J male mice were subjected to 120-min left MCAO. They were called young and old mice, respectively, in this study. Old mice had larger infarct brain volumes, higher neurological deficit scores and worse performance on rotarod than young mice (FIGS. 14A-D).

These results suggest that old mice have worse neurological outcome after focal brain ischemia than young mice. Brain ischemia and reperfusion increased IL-1β and IL-6 in the blood of young mice and IL-17, IL-1β and IL6 in the blood of old mice (FIGS. 14E-14G). Consistent with the finding of neurological outcome, old mice had higher IL-17 and IL-6 concentrations in their blood after brain ischemia and reperfusion [age was a factor to influence the effects of brain ischemia on IL-17 and IL-6 concentration: F (1,28)=4.673, P=0.039, for IL-17; F (1,28)=4.559, P=0.042, for IL-6], although there was no difference in these cytokines at baseline between young and old mice (FIGS. 14E-14G). Brain ischemia and reperfusion did not affect the expression of IL-10 (FIG. 14H), an anti-inflammatory cytokine [8], in the blood of old and young mice. These results suggest that brain ischemia induces a heightened inflammatory response in old mice.

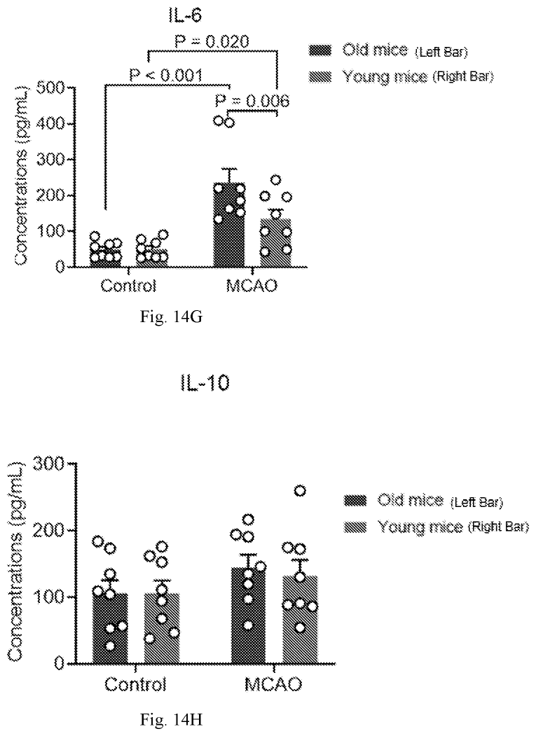

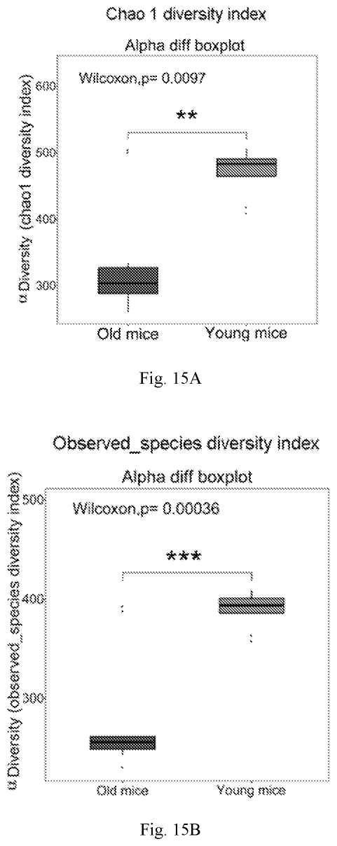

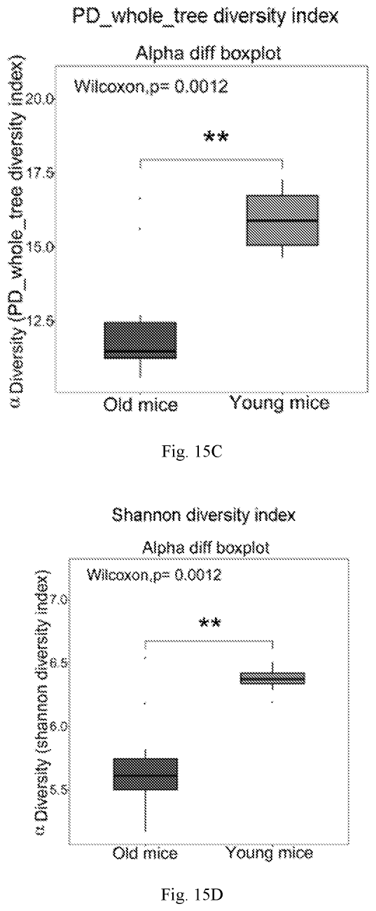

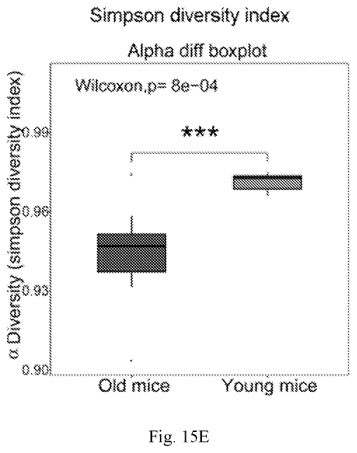

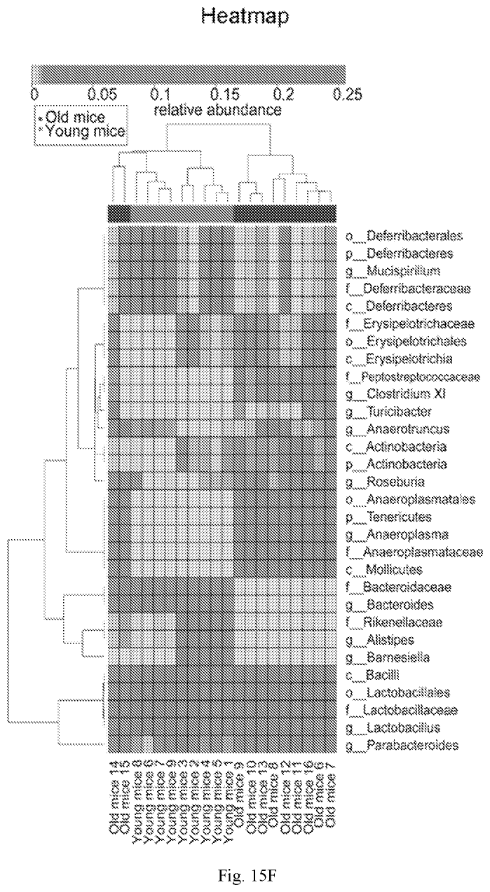

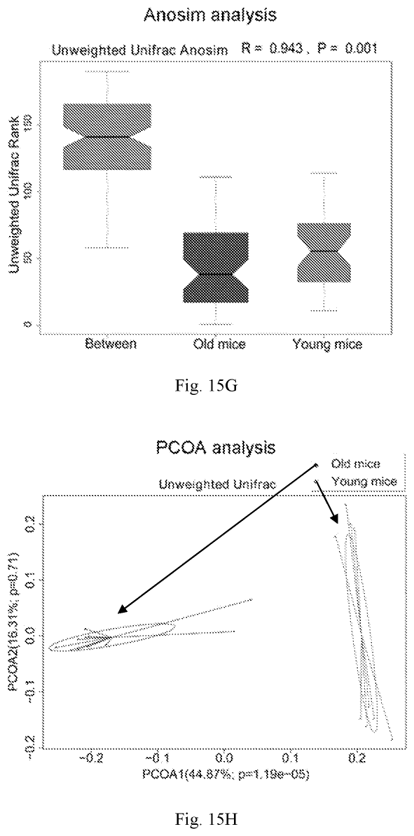

As an initial step to determine the role of gut microbiota in the age-related changes in ischemic brain injury and inflammation response, gut microbiota was investigated. Gut microbiota in young mice was more diverse than that in old mice as indicated by a diversity parameters and had a taxonomic composition different from that of old mice as indicated by β diversity parameters (FIG. 15). The Anosim analysis (R=0.943, P=0.001) showed that the intergroup difference in the taxonomic composition between young and old mouse fecal microbiota was greater than intragroup variation. Young mice had an increased relative abundance of certain bacteria, such as g_Bacteroides, g_Alistipes and g_Barnesiella. The relative abundance of some bacteria, such as g_Parabacteroides and g_Lactobacillus, was increased in old mice (FIG. 15). These results indicate that gut microbiota in the old mice is different from that in the young mice. Since SCFAs are mainly produced from gut microbiota and gut microbiota can affect outcome after brain ischemia [9-11], blood concentrations of SCFAs were measured in mice without brain ischemia. Old mice had an increase of propionic acid, butyric acid, isovaleric acid, valeric acid and hexanic acid (FIG. 16A), suggesting that age-related changes in gut microbiota composition leads to changes of SCFAs in the host blood.

### Example 6

### Transplanting Old Mouse Feces Worsened Neurological Outcome, Heightened Proinflammatory Cytokine Production and Increased Valeric Acid in the Blood of Young Mice after Brain Ischemia

To determine whether different gut microbiota plays a role in the worsened neurological outcome after brain ischemia, 4 groups of 8-week old C57BL/6J male mice were studied: control mice, antibiotic group, Young-oFMT group and Young-yFMT group. Cefazolin was used to eliminate gut microbiota [20], which could facilitate the growth of transplanted microbiota in the host gut. These mice were subjected to 120-min left MCAO 2 weeks after fecal transplantation. The gut microbiota was examined prior to MCAO to determine the success of fecal transplant. Similar to the young and old mice without fecal transplantation, Young-oFMT mice had a decreased richness in gut microbiota at 2 weeks after the transplantation compared to Young-yFMT mice. There was a difference in the taxonomic composition of gut microbiota between these two types of mice receiving fecal transplant. Young-yFMT mice had an increased relative abundance of g_Alistipes compared with Young-oFMT mice. As stated above, the relative abundance of g_Alistipes was increased in young mice when compared with old mice. These results suggest the success of transplanting corresponding gut microbiota into the young mouse gut. However, the taxa composition of gut microbiota of recipients was not identical to that of the donors because the β diversity of gut microbiota was different between young mice and Young-yFMT mice or old mice and Young-oFMT mice. The relative abundance of gut microbiota species was also different between young mice and Young-yFMT mice or old mice and Young-oFMT mice.

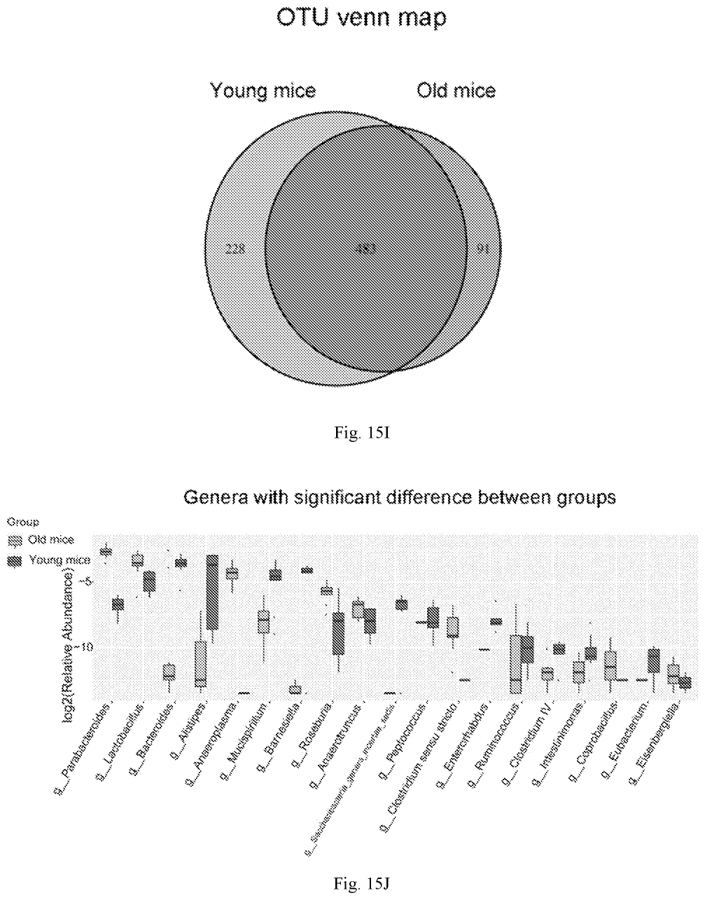

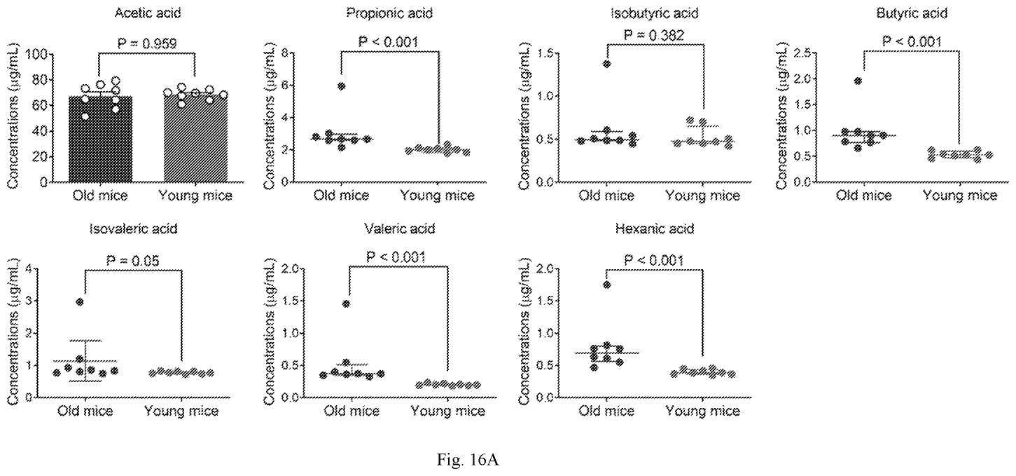

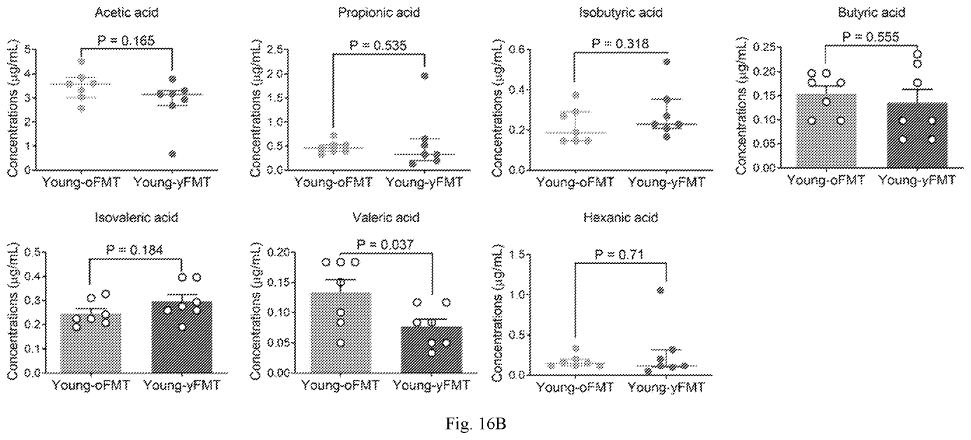

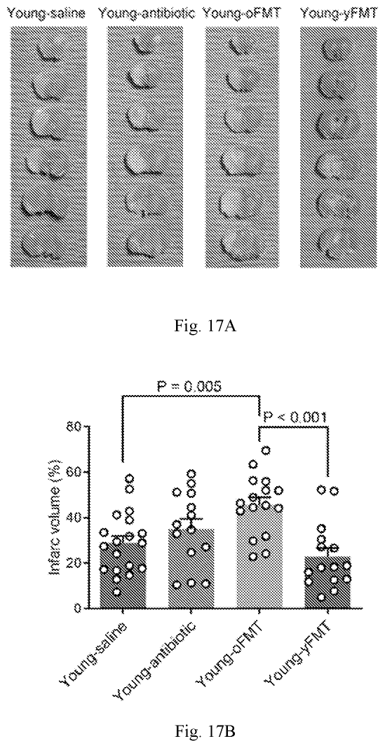

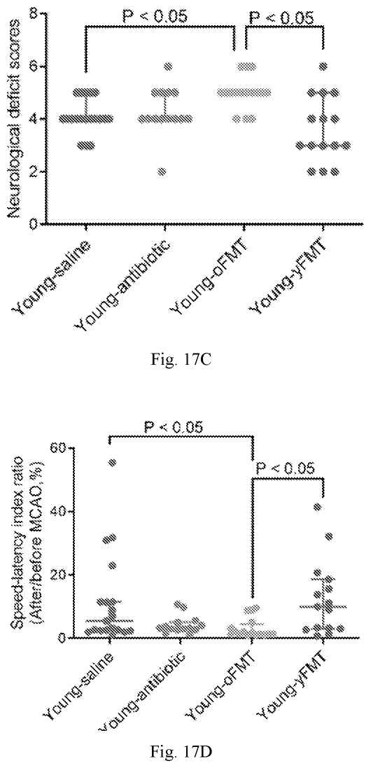

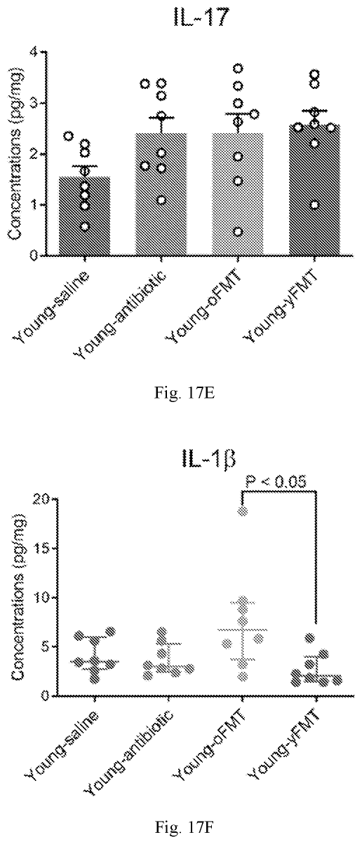

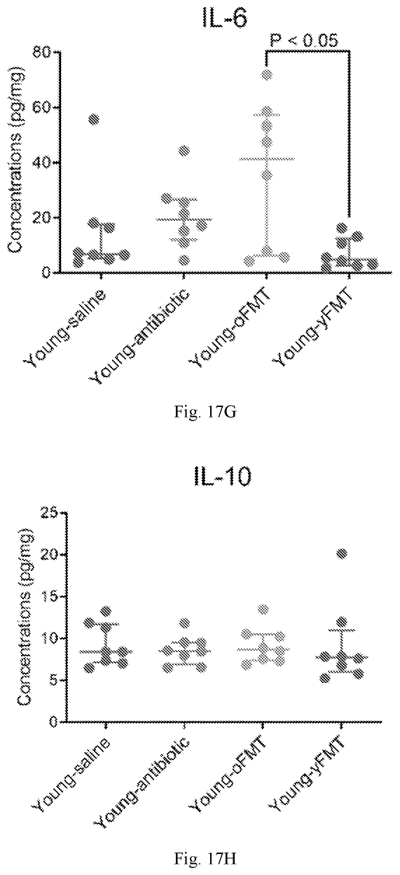

Young-oFMT mice had larger brain infarct volumes, higher neurological deficit scores and worse performance on rotarod than young mice without fecal transplantation or Young-yFMT mice (FIGS. 17A-17D). These results suggest that old mouse microbiota may contribute to the worse neurological outcome after brain ischemia. Young-oFMT mice had higher IL-1ß and IL-6 concentrations in the left frontal cerebral cortex area 1 (Fr1), an ischemic penumbral region [8, 28]. Transplantation of young or old mouse feces did not change the levels of IL-17 or IL-10 in the Fr1 (FIGS. 17E-17H). These results suggest that young mice with transplantation of old mouse feces had heightened inflammatory response, a condition similar to that of old mice. However, the body weights of mice in the four groups before brain ischemia were not different, suggesting that the general conditions of these mice with fecal transplantation were similar to those without fecal transplantation. Antibiotic treatment that was necessary before fecal transplant did not affect the weight increase curve. Also, the magnitude of cerebral blood flow decrease was similar between mice transplanted with young mouse or old mouse feces. These results suggest that the worsened neurological outcome of mice transplanted with old mouse feces is not due to different levels of cerebral blood flow decrease or poor general condition.

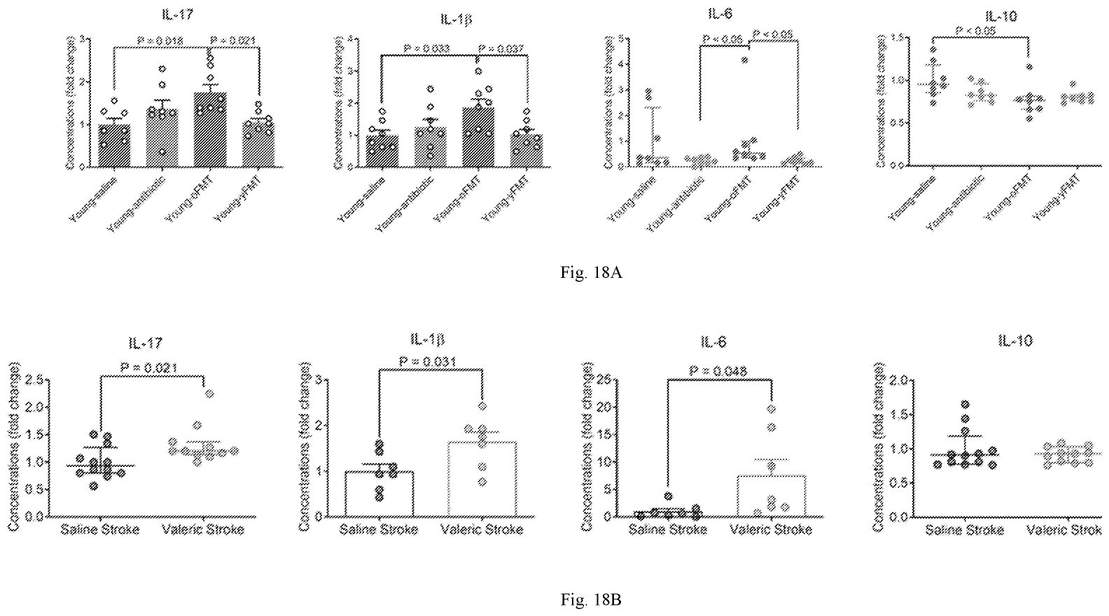

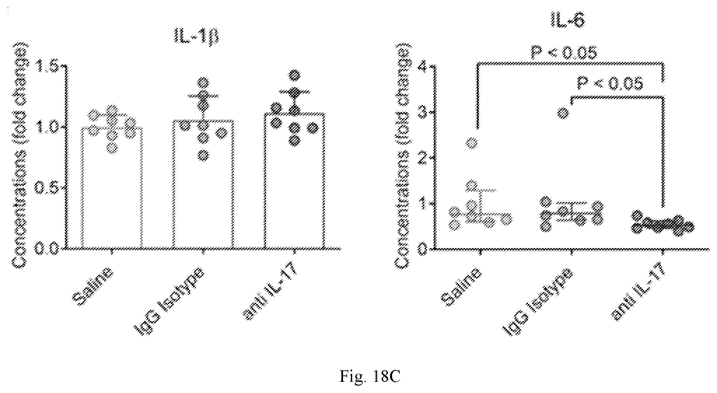

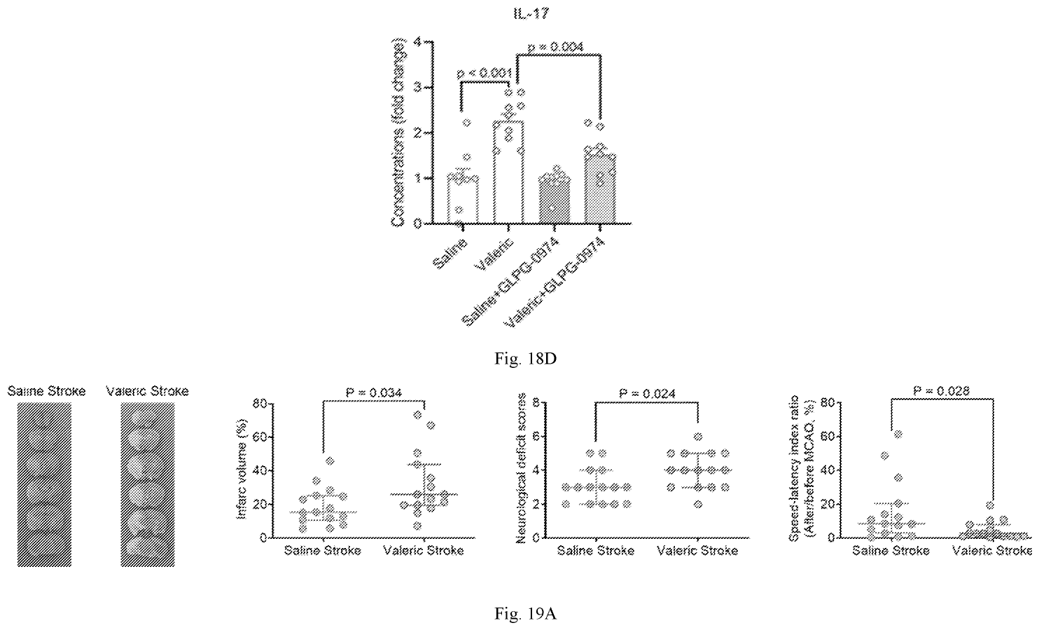

Similar to the inflammatory cytokine changes in the ischemic penumbral region, Young-oFMT mice had increased IL-17, IL-1β and IL-6 in the blood compared with young mice without fecal transplantation or Young-yFMT mice. In addition, Young-oFMT mice had decreased IL-10 compared to young mice without fecal transplantation (FIG. 18A). These results suggest that brain ischemia and reperfusion induced a heightened inflammatory response in Young-oFMT mice.

Since there was a significant difference in the neurological outcome and inflammatory response in the blood and ischemic penumbral brain tissues between young mice transplanted with young and old mouse feces, we determined whether there was a difference in SCFAs between these mice. Young-oFMT mice had increased valeric acid in the blood compared with young mice transplanted with young mouse feces. The other SCFAs were not different between these two types of mice (FIG. 16B). These results suggest a potential role of valeric acid in mediating the effects of old mouse gut microbiota. Interestingly, the colon mucosal permeability of Young-oFMT mice was higher than that of young mice transplanted with young mouse feces. MCAO increased this permeability in those two types of mice but the colon mucosal permeability remained higher in the Young-oFMT mice. These results suggest that gut microbiota of old mice may increase colon mucosal permeability and, therefore, may facilitate the absorption of toxic agents from colon into the circulation.

### Example 7

### Valeric Acid Worsened Neurological Outcome and Heightened Proinflammatory Cytokines Production in Young Mice after Brain Ischemia

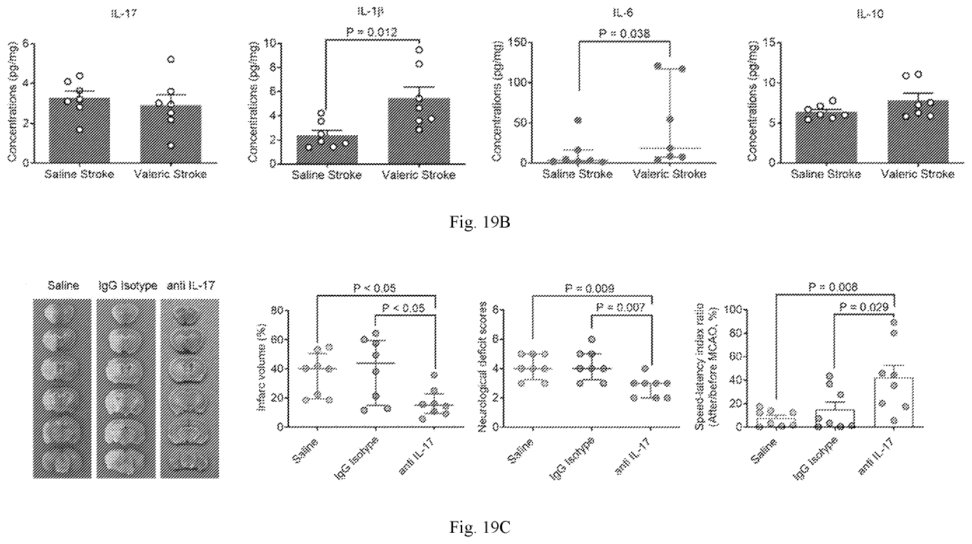

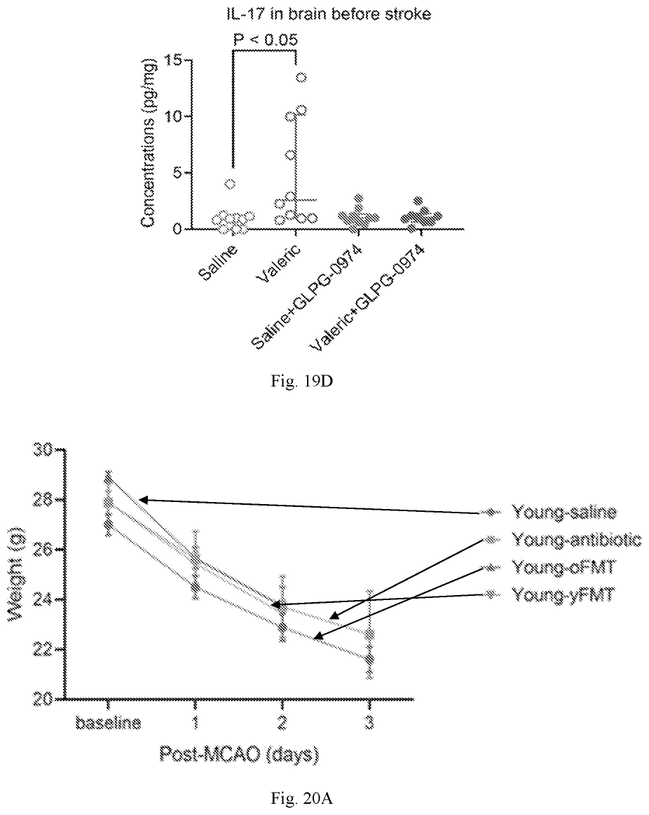

To determine the role of valeric acid in brain ischemic injury, young mice received 100 mg/kg valeric sodium intraperitoneally at 1 h before and 6 h after the MCAO. This pilot study showed that this regimen increased the concentrations of valeric acid in the blood of young mice to a level similar to that in the old mice. Valeric acid increased the concentrations of IL-17, IL-1β and IL-6 in the blood of mice with experimental stroke (FIG. 18B). These results suggest that valeric acid enhances the inflammatory response after stroke. Young mice that received valeric sodium had a larger infarct volume, worse neurological deficit scores and poorer performance on rotarod than young mice that received saline injection (FIG. 19A). Valeric sodium also increased IL-1β and IL-6 in the ischemic penumbral brain tissues (FIG. 19B). These results suggest that valeric acid may enhance inflammatory response and increase brain ischemic injury.

### Example 8

### Blocking IL-17 in the Blood Attenuated Valeric Acid-Induced Worsening of Neurological Outcome and Heightening of Proinflammatory Cytokine Increase

IL-17 can regulate the production of chemokines and other proinflammatory cytokines, such as IL-1β and IL-6 [17, 18]. Our results described above showed that transplantation of old mouse feces or intraperitoneal injection of valeric acid did not increase IL-17 in the brain but increased IL-17 in the blood of mice with brain ischemia (FIGS. 17E, 18A, 18B and 19B). On the other hand, IL-1β and IL-6 were increased in the blood and brain in stroke mice that received valeric acid or transplantation of old mouse feces (FIGS. 17F, 17G, 18A, 18B and 19B). Thus, IL-17 in the blood could be a molecule that can easily be targeted without the concern of additional production sites, such as brain, in the case of IL-1ß and IL-6.

To determine the role of IL-17 in valeric acid effects on brain ischemic injury, young mice that received intraperitoneal injection of valeric sodium as described above had intravenous injection of 200 μl saline, 100 μg mouse IgG1 in 200 μl or 100 μg mouse monoclonal anti-IL-17 antibody 200 μl 2 h after the initiation of MCAO. The antibody but not the mouse IgG1 reduced IL-6 concentrations in the blood (FIG. 18C). The anti-IL-17 antibody but not the mouse IgG1 reduced brain infarct volume and improved neurological function of mice that received valeric acid (FIG. 19C). These results suggest that IL-17 neutralization reduced valeric acid-induced worsening of brain ischemic injury and inflammatory response.

As presented above, IL-17 appeared to be a molecule downstream of valeric acid to mediate the worsened neurologic outcome with aging after brain ischemia. Four FFARs have been identified. FFAR1 and FFAR4 are for long chain fatty acids. FFAR2 and FFAR3 are for SCFAs. FFAR2 are expressed in adipocytes, enteroendocrine cells, immune cells and white blood cells. FFAR3 are expressed in adipocytes, enteroendocrine cells and peripheral nervous tissues [29]. Thus, GLPG-0974, a FFAR2 antagonist [19], was used. GLPG-0974 blocked the increase of IL-17 in the blood and brain caused by valeric acid in mice without MCAO (FIGS. 18D, 19D). These results suggest that valeric acid activates FFAR2 to increase IL-17.

### Example 9

### Transplanting with Old Mouse Feces Worsened General Outcome of Young Mice and Transplanting with Young Mouse Feces Improved General Outcome of Old Mice

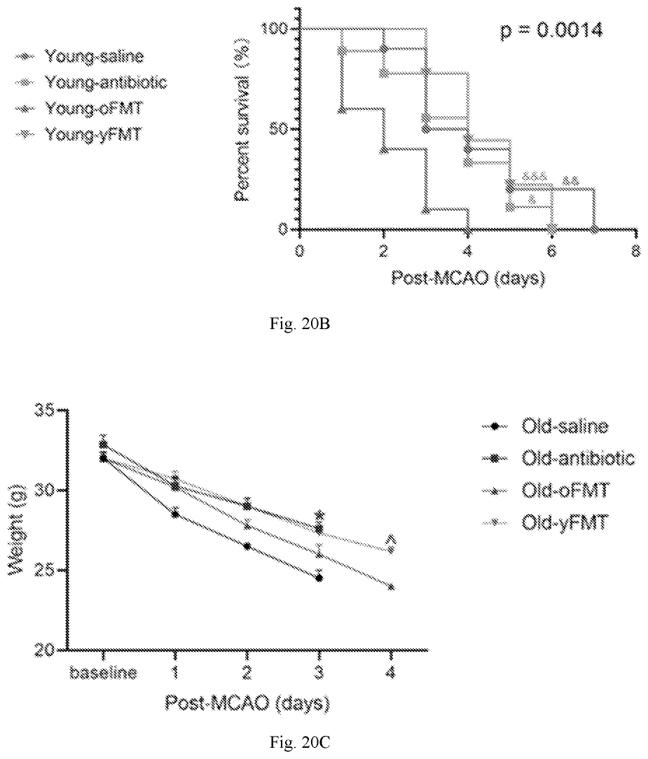

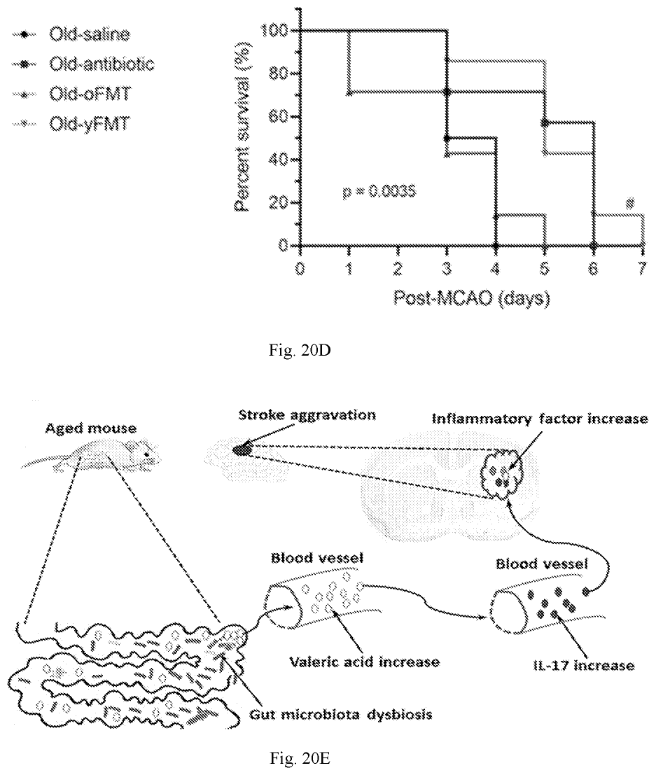

To provide additional evidence for the role of gut microbiome in determining the outcome after brain ischemia, body weights and mortality were recorded after brain ischemia. Young mice lost body weights after MCAO whether they had fecal transplant (FIG. 20A). Young-oFMT mice had a worse survival curve than control young mice, young mice receiving antibiotic treatment or Young-yFMT mice (FIG. 20B). These results suggest that transplanting young mice with old mouse feces worsened the outcome of young mice after brain ischemia. In another experiment, Old-yFMT or Old-oFMT mice were subjected to a 60-min left MCAO. Old-yFMT mice had less body weight loss over time after the MCAO than Old-oFMT mice [F (1,12]=5.383, P=0.034] (FIG. 20C). In addition, Old-yFMT mice had a better survival curve than Old-oFMT mice or old mice without fecal transplantation (FIG. 20D). These results suggest that transplantation with young mouse feces improves the outcome of old mice after brain ischemia.

### Example 10

### Discussion of Examples 5-9

The results herein clearly show that the gut microbiota in young mice is more diverse than that in old mice. Old mice had a relatively abundant amount of g_parabacteroides and g_lactobacillus. Gut microbiota in young mice was rich in g_alistipes and g_bacteroides. Both g_alistipes and g_bacteroides are genus in the phylum Bacteroidetes. While g_parabacteroides is in the phylum Bacteroidota, g_lactobacillus is in the phylum Firmicutes. Increased Firmicutes is considered a marker of age-related changes in gut microbiota. These results suggest that old mice have gut dysbiosis.

To determine whether age-related changes in gut microbiota contributes to the worsened neurological outcome in old mice, cefazolin was used to eliminate the existing gut microbiota of young mice [20]. These mice were then transplanted with old or young mouse feces. Young-oFMT mice were rich in g_anaerotruncus, a genus of Firmicutes, while Young-yFMT mice had relatively abundant g_alistipes, a genus that was rich in young mouse gut microbiota. These results suggest that transplantation renders young mice to have gut microbiota similar to that transplanted. Young-oFMT mice had worsened neurological outcome and heightened inflammatory response in the blood and ischemic penumbral brain tissues, a situation similar to that of old mice. Young-oFMT mice also had a higher mortality rate than Young-yFMT mice. In addition, this study showed that Old-yFMT mice lost less body weight and survived better than Old-oFMT mice. These results suggest that difference in gut microbiota contributes to the severity of ischemic brain injury, the levels of inflammatory response and general outcome worsening after brain ischemia.

Gut microbiota produces various metabolites. Among them, SCFAs in humans are mostly produced by gut microbiota. To determine whether SCFAs mediate the effects of gut microbiota on the outcome of ischemic stroke, blood levels of SCFAs and gut mucosal permeability were measured. Gut mucosal permeability was higher in old mice than that in young mice no matter whether the mice had MCAO. These results suggest that mucosa in the old mice may let more chemicals including SCFAs get into circulation. This situation was worsened when mice had ischemic stroke. Old mice had an increased blood level of 5 SCFAs among the 7 SCFAs measured in the study. However, valeric acid was the only one that was increased in the mice transplanted with old mouse feces. Since these mice also had worse neurological outcome and heightened inflammatory response in the blood and brain tissues, these results suggest that valeric acid in the blood plays a role as a mediator for these effects of old mouse gut microbiota. In supporting this idea, intravenous valeric acid worsened neurological outcome and heightened the inflammatory response in the blood and ischemic penumbral brain tissues. Thus, these results strongly and surprisingly show that valeric acid mediates the effects of old mouse gut microbiota on neurological outcome and inflammatory response.

To determine a mediator downstream of valeric acid, an IL-17 neutralizing antibody was used. IL-17 in the blood was increased by intravenous valeric acid. IL-17 in the ischemic brain tissues was not affected by valeric acid. Thus, neutralizing IL-17 in the blood could have an effect if IL-17 is a downstream mediator for valeric acid. The mouse monoclonal anti-IL-17 antibody improved neurological outcome and reduced inflammation response of young mice after the combination of ischemic stroke and valeric acid. IL-17 in the blood of young mice after ischemic stroke was not changed compared to young mice without ischemic stroke, suggesting that IL-17 is a downstream mediator for old mouse gut microbiota- and valeric acid-induced worsening of neurological outcome and increase of inflammatory response. Since the increase of IL-17 caused by valeric acid was inhibited by an FFAR2 inhibitor, it is suggested that valeric acid-induced increase of IL-17 is mediated by FFAR2. Thus, the novel pathway, gut microbiota-valeric acid-FFAR2-IL-17, is suggested to be an underlying mechanism for the worsened neurological outcome and heightened inflammatory response in old mice (FIG. 20E).

Importantly, these results clearly showed that SCFAs were increased in the blood of old mice, as opposed to simply measuring fecal SCFAs. Old mice had an increase in colon mucosal permeability, which may facilitate the absorption of SCFAs. Thus, part of the decrease of SCFAs in feces with aging as shown in the previous studies may be due to the absorption of SCFAs into the blood. These results suggest the importance to measure SCFAs in the blood to know the possible role of SCFAs in ischemic brain injury. A human study has shown that stroke patients have increased valeric acid in their feces and this increase was associated with increased white blood cells and highly sensitive C-reactive protein in the blood. However, the role of this increased valeric acid in feces in stroke and inflammatory response has not previously been investigated. The instant results suggest a detrimental effect of valeric acid in ischemic stroke. Thus, different SCFAs may have different effects on neurological outcome after brain ischemia.

The present disclosure further shows that the age-related worsening of neurological functional and structural outcome, heightened inflammatory response in the brain and this suggests that poor general outcomes are due to age-related gut microbiota changes.

The presently disclosed results suggest that IL-17 is a mediator downstream of valeric acid for the worsened neurological outcome with aging. Thus, reducing IL-17 blood concentrations, either by mediating the upstream valeric acid, IL-17 activity, and/or combinations thereof, can improve neurological outcome with aging after brain ischemia or other neuroinflammation.

The results herein pertaining to gut microbiota profiling indicate the success of gut microbiota transplantation. In supporting this success, Young-oFMT mice had worse neurological outcome and neuroinflammation after brain ischemia than Young-yFMT mice, which is similar to the situation that old mice had worse neurological outcome and neuroinflammation than young mice after brain ischemia. However, the recipients of fecal transplantation did not have a gut microbiota identical to that of the corresponding controls (young mice vs. Young-yFMT mice and old mice vs. Young-oFMT mice). This situation may be anticipated because of the difficulty to completely eliminate the original gut microbiota of recipients by antibiotic treatment, changes of gut microbiota during the harvest and transplant processes and impossibility of all transplanted bacteria to survive and grow in the same proportion as in the donors. Thus, transplantation may not fully replace the gut microbiota of the recipients. In addition, the feces used for transplantation were pooled from donors and gut microbiota was profiled individually for comparison.

This study has significant implications. The detrimental role of valeric acid in the experimental ischemic stroke was identified, suggesting that not all SCFAs are beneficial and that it is necessary to measure individual SCFA. Our study has illustrated that it is necessary to measure the concentrations of SCFAs in the blood to fully know the effects of SCFAs. More importantly, these results suggest that valeric acid and IL-17 are targets for improving neurological outcome after ischemic stroke. However, Megasphaera massiliensis is known to produce valeric acid [41]. Methods to inhibit the production of valeric acid from this bacterium or other bacteria can reduce valeric acid in the blood. Fecal transplantation with healthy gut microbiota can also be a way to decrease Megasphaera massiliensis and to reduce valeric acid. Changing diet is another method to alter the composition of gut microbiota. Also showed herein is the importance of IL-17 in age-related worsening of ischemic brain injury. IL-17 in the blood can be neutralized by its antibody as shown in this study. Finally, the studies disclosed herein suggest a role of FFAR2 in mediating the increase of IL-17 caused by valeric acid and that inhibiting FFAR2 provides a basis for an intervention for improving the outcome after brain ischemia in old population.

### Example 11

### Differences in Gut Microbiome with Aging Contributes to the Increased Inflammatory Responses

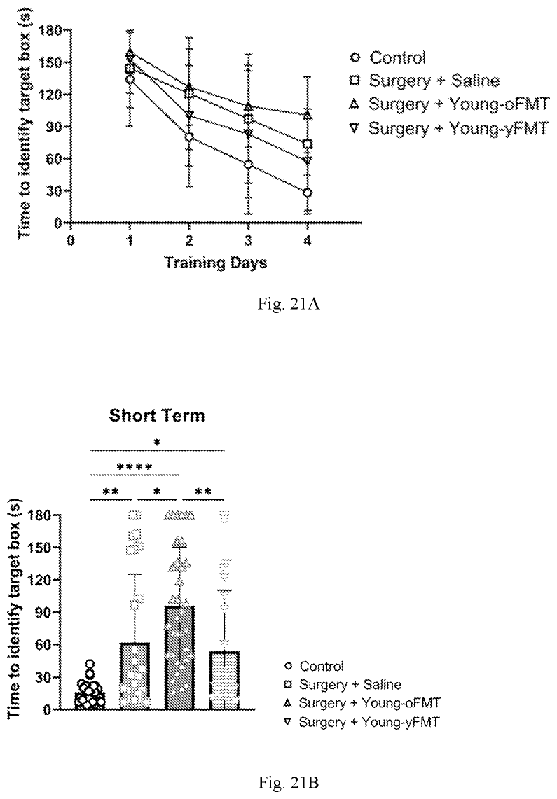

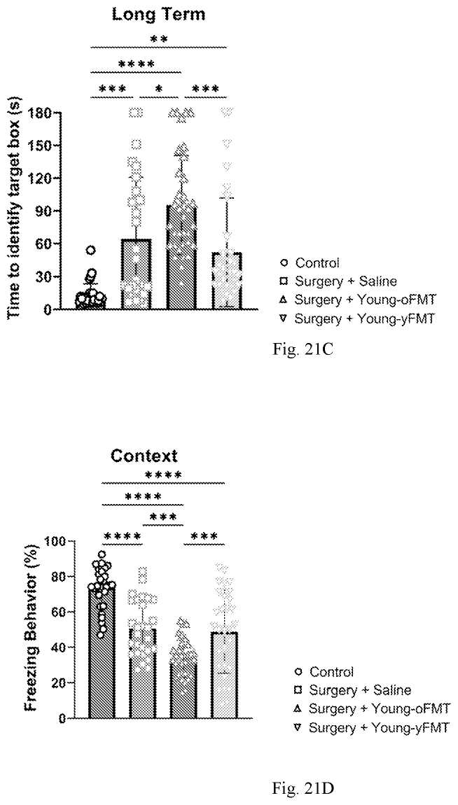

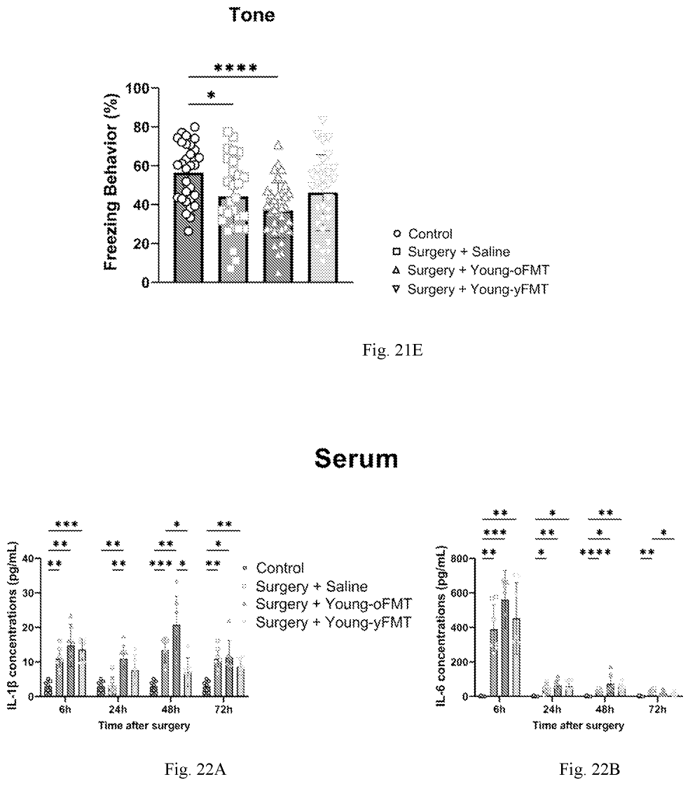

Transplanting old mouse gut microbiome worsened learning and memory dysfunction after surgery in young mice. Eight-week old male mice received transplant of gut microbiome from eight-week old male mice (Young-yFMT) or from eighteen-month old male mice (Young-oFMT). They were then subjected to surgery (carotid artery exposure) 11 days after the completion of fecal transplant. Mice were started to be tested 7 days after surgery. Results are shown in FIGS. 21A-21E as follows: FIG. 21A: Train phase in Barnes maze. FIG. 21B: Memory phase at one day after training sessions in Barnes maze. FIG. 21C: Memory phase at eight days after training sessions in Barnes maze. FIG. 21E: Context-related fear conditioning behavior. FIG. 21E: Tone-related fear conditioning. Results are mean±S.D. (n=28-35). Data of individual mouse is included in the bar graphs. * P<0.05, ** P<0.01, *** P<0.005, **** P<0.001.

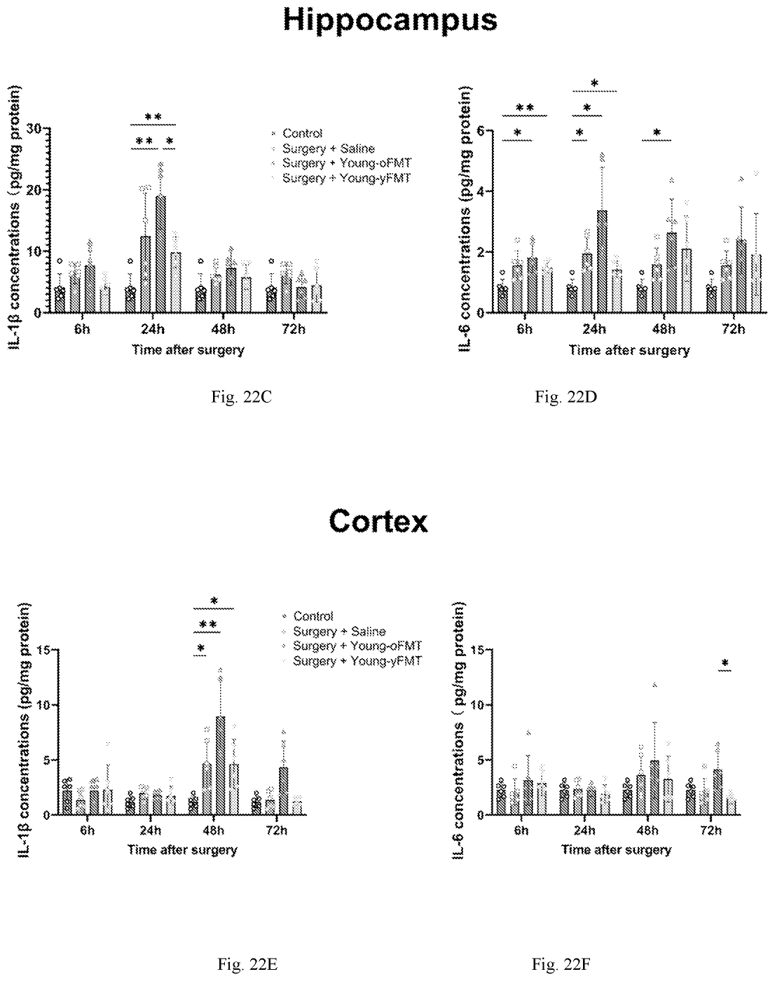

Transplanting old mouse gut microbiome increased proinflammatory cytokines after surgery in young mice. Eight-week old male mice received transplant of gut microbiome from eight-week old male mice (Young-yFMT) or from eighteen-month old male mice (Young-oFMT). Their blood, hippocampus and cerebral cortex were harvested at various times after surgery. Results are shown in FIGS. 22A-22F as follows: FIG. 22A: IL-1ß in the serum. FIG. 22B: IL-6 in the serum. FIG. 22C: IL-1β in the hippocampus. FIG. 22D: IL-6 in the hippocampus. FIG. 22E: IL-1β in the cerebral cortex. FIG. 22F: IL-6 in the cerebral cortex. Results are mean+S.D. (n=7). Data of individual mouse is included in the bar graphs. * P<0.05, ** P<0.01, *** P<0.005, **** P<0.001.

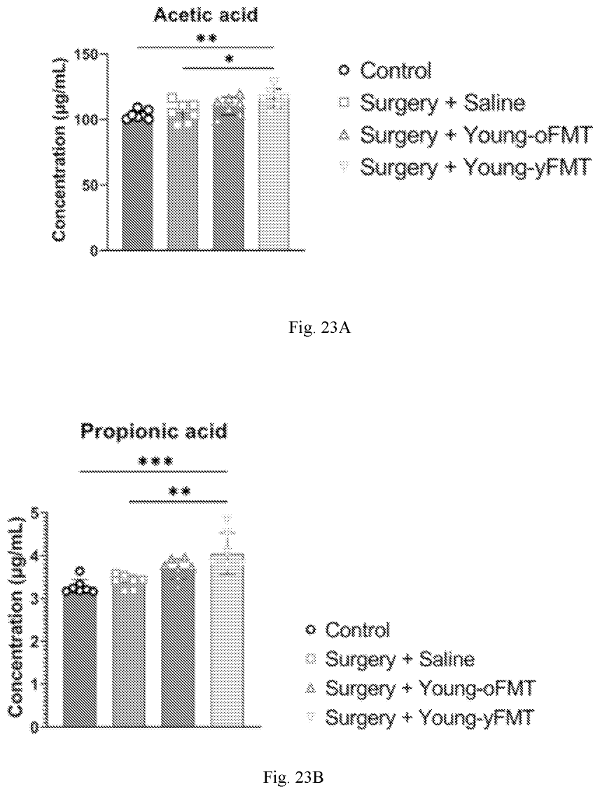

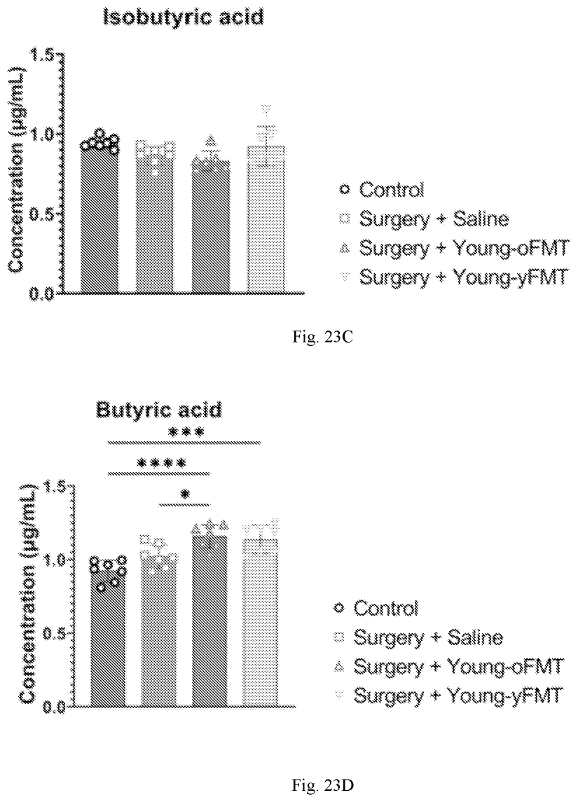

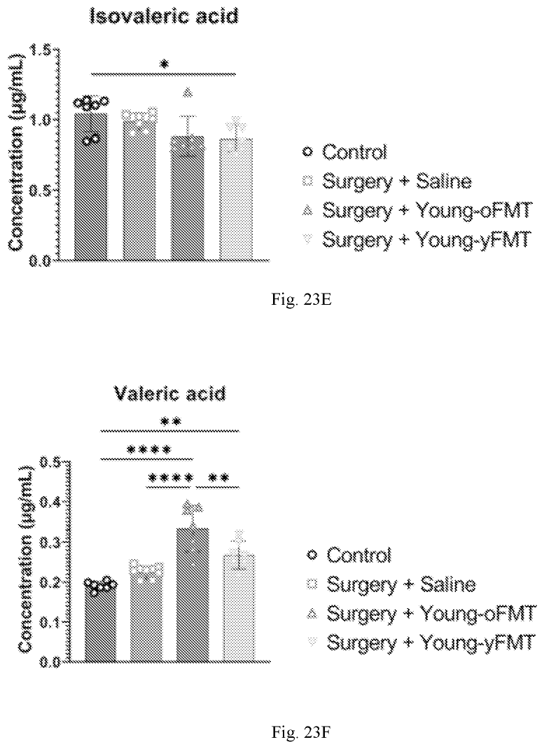

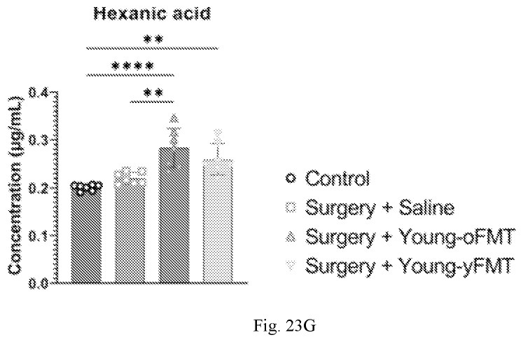

Transplanting old mouse gut microbiome increased valeric acid after surgery in young mice. Eight-week old male mice received transplant of gut microbiome from eight-week old male mice (Young-yFMT) or from eighteen-month old male mice (Young-oFMT). Their blood was harvested at 24 h after surgery. Results are shown in FIGS. 23A-23G as follows: FIG. 23A: Acetic acid. FIG. 23B: Propionic acid. FIG. 23C: Isobutyric acid. FIG. 23D: Butyric acid. FIG. 23E: Isovaleric acid. FIG. 23F: Valeric acid. FIG. 23G: Hexanic acid. Results are mean±S.D. (n=7). Data of individual mouse is included in the bar graphs. * P<0.05, ** P<0.01, *** P<0.005, P<0.001.

These data demonstrate that difference in gut microbiome with aging contributes to the increased inflammatory responses and worsened dysfunction of learning and memory after surgery. Valeric acid can play a role in these worsened effects with aging.

### REFERENCES

All references cited and/or listed herein including but not limited to all patents, patent applications and publications thereof, scientific journal articles, and database entries (e.g., GENBANK® database entries and all annotations available therein) are incorporated herein by reference in their entireties to the extent that they supplement, explain, provide a background for, or teach methodology, techniques, and/or compositions employed herein.
1. Moller J T, Cluitmans P, Rasmussen L S, Houx P, Rasmussen H, Canet J et al. Long-term postoperative cognitive dysfunction in the elderly ISPOCD1 study. ISPOCD investigators. International Study of Post-Operative Cognitive Dysfunction. Lancet 1998; 351:857-61.
2. Monk T G, Weldon B C, Garvan C W, Dede D E, van der Aa M T, Heilman K M et al.

Predictors of cognitive dysfunction after major noncardiac surgery. Anesthesiology 2008; 108:18-30.
3. Newman M F, Kirchner J L, Phillips-Bute B, Gaver V, Grocott H, Jones R H et al.

Longitudinal assessment of neurocognitive function after coronary-artery bypass surgery. N Engl J Med 2001; 344:395-402.
4. Hall M, Schwartzman A, Zhang J, Liu X. Ambulatory Surgery Data from Hospitals and Ambulatory Surgery Centers: 2010. National Health Statistics Report 2017.
5. Li Y, Chen D, Wang H, Wang Z, Song F, Li H et al. Intravenous versus Volatile Anesthetic Effects on Postoperative Cognition in Elderly Patients Undergoing Laparoscopic Abdominal Surgery. Anesthesiology 2021; 134:381-94.
6. Zhang J, Tan H, Jiang W, Zuo Z. Amantadine alleviates postoperative cognitive dysfunction possibly by increasing glial cell line-derived neurotrophic factor in rats. Anesthesiology 2014; 121:773-85.
7. Lai Z, Min J, Li J, Shan W, Yu W, Zuo Z. Surgery Trauma Severity but not Anesthesia Length Contributes to Postoperative Cognitive Dysfunction in Mice. J Alzheimers Dis 2021; 80:245-57.
8. Zhang J, Tan H, Jiang W, Zuo Z. The choice of general anesthetics may not affect neuroinflammation and impairment of learning and memory after surgery in elderly rats. J Neuroimmune Pharmacol 2015; 10:179-89.
9. Terrando N, Monaco C, Ma D, Foxwell B M, Feldmann M, Maze M. Tumor necrosis factor-alpha triggers a cytokine cascade yielding postoperative cognitive decline. Proc Natl Acad Sci USA 2010; 107:20518-22.
10. Blander J M, Longman R S, Iliev I D, Sonnenberg G F, Artis D. Regulation of inflammation by microbiota interactions with the host. Nat Immunol 2017; 18:851-60.
11. Liang P, Shan W, Zuo Z. Perioperative use of cefazolin ameliorates postoperative cognitive dysfunction but induces gut inflammation in mice. J Neuroinflammation 2018; 15:235.
12. Zhan G, Hua D, Huang N, Wang Y, Li S, Zhou Z et al. Anesthesia and surgery induce cognitive dysfunction in elderly male mice: the role of gut microbiota. Aging (Albany NY) 2019; 11:1778-90.
13. Jiang X L, Gu X Y, Zhou X X, Chen X M, Zhang X, Yang Y T et al. Intestinal dysbacteriosis mediates the reference memory deficit induced by anaesthesia/surgery in aged mice. Brain Behav Immun 2019; 80:605-15.
14. Fan D, Li J, Zheng B, Hua L, Zuo Z. Enriched Environment Attenuates Surgery-Induced Impairment of Learning, Memory, and Neurogenesis Possibly by Preserving BDNF Expression. Mol Neurobiol 2016; 53:344-54.
15. Kawano T, Eguchi S, Iwata H, Tamura T, Kumagai N, Yokoyama M. Impact of Preoperative Environmental Enrichment on Prevention of Development of Cognitive Impairment following Abdominal Surgery in a Rat Model. Anesthesiology 2015; 123:160-70.
16. Liu Y, Wang Y, Ni Y, Cheung C K Y, Lam K S L, Wang Y et al. Gut Microbiome Fermentation Determines the Efficacy of Exercise for Diabetes Prevention. Cell Metab 2020; 31:77-91 e5.
17. Pyke W, Ifram F, Coventry L, Sung Y, Champion I, Javadi A H. The effects of different protocols of physical exercise and rest on long-term memory. Neurobiol Learn Mem 2020; 167:107128.
18. Hayashi K, Oshima H, Shimizu M, Kobayashi K, Matsui S, Nishida Y et al. Preoperative 6-Minute Walk Distance Is Associated With Postoperative Cognitive Dysfunction. Ann Thorac Surg 2018; 106:505-12.
19. Feng X, Uchida Y, Koch L, Britton S, Hu J, Lutrin D et al. Exercise Prevents Enhanced Postoperative Neuroinflammation and Cognitive Decline and Rectifies the Gut Microbiome in a Rat Model of Metabolic Syndrome. Front Immunol 2017; 8:1768.
20. Antunes M, Biala G. The novel object recognition memory: neurobiology, test procedure, and its modifications. Cogn Process 2012; 13:93-110.
21. Pandit R J, Hinsu A T, Patel N V, Koringa P G, Jakhesara S J, Thakkar J R et al. Microbial diversity and community composition of caecal microbiota in commercial and indigenous Indian chickens determined using 16s rDNA amplicon sequencing. Microbiome 2018; 6:115.
22. Guan J S, Haggarty S J, Giacometti E, Dannenberg J H, Joseph N, Gao J et al. HDAC2 negatively regulates memory formation and synaptic plasticity. Nature 2009; 459:55-60.
23. Litvinchuk A, Wan Y W, Swartzlander D B, Chen F, Cole A, Propson N E et al.

Complement C3aR Inactivation Attenuates Tau Pathology and Reverses an Immune Network Deregulated in Tauopathy Models and Alzheimer's Disease. Neuron 2018; 100:1337-53 e5.
24. Barnum S R. Inhibition of complement as a therapeutic approach in inflammatory central nervous system (CNS) disease. Mol Med 1999; 5:569-82.
25. Hutamekalin P, Takeda K, Tani M, Tsuga Y, Ogawa N, Mizutani N et al. Effect of the C3a-receptor antagonist SB 290157 on anti-OVA polyclonal antibody-induced arthritis. J Pharmacol Sci 2010; 112:56-63.
26. Gui L, Lei X, Zuo Z. Decrease of glial cell-derived neurotrophic factor contributes to anesthesia- and surgery-induced learning and memory dysfunction in neonatal rats. J Mol Med (Berl) 2017; 95:369-79.
27. Silva Y P, Bernardi A, Frozza R L. The Role of Short-Chain Fatty Acids From Gut Microbiota in Gut-Brain Communication. Front Endocrinol (Lausanne) 2020; 11:25.
28. Dalile B, Van Oudenhove L, Vervliet B, Verbeke K. The role of short-chain fatty acids in microbiota-gut-brain communication. Nat Rev Gastroenterol Hepatol 2019; 16:461-78.
29. Sadler R, Cramer J V, Heindl S, Kostidis S, Betz D, Zuurbier K R et al. Short-Chain Fatty Acids Improve Poststroke Recovery via Immunological Mechanisms. J Neurosci 2020; 40:1162-73.
30. Magrone T, Jirillo E. The interaction between gut microbiota and age-related changes in immune function and inflammation. Immun Ageing 2013; 10:31.
31. Parada Venegas D, De la Fuente M K, Landskron G, Gonzalez M J, Quera R, Dijkstra G et al. Short Chain Fatty Acids (SCFAs)-Mediated Gut Epithelial and Immune Regulation and Its Relevance for Inflammatory Bowel Diseases. Front Immunol 2019; 10:277.
32. Frost G, Sleeth M L, Sahuri-Arisoylu M, Lizarbe B, Cerdan S, Brody L et al. The short-chain fatty acid acetate reduces appetite via a central homeostatic mechanism. Nat Commun 2014; 5:3611.
33. Spychala M S, Venna V R, Jandzinski M, Doran S J, Durgan D J, Ganesh B P et al. Age-related changes in the gut microbiota influence systemic inflammation and stroke outcome. Ann Neurol 2018; 84:23-36.
34. Biagi E, Nylund L, Candela M, Ostan R, Bucci L, Pini E et al. Through ageing, and beyond: gut microbiota and inflammatory status in seniors and centenarians. PLOS ONE 2010; 5: e10667.
35. Ji M H, Qiu L L, Mao M J, Zhang L, Yang J J. Hippocampal complement C3 might contribute to cognitive impairment induced by anesthesia and surgery. Neuroreport 2020; 31:507-14.
36. Benakis C, Brea D, Caballero S, Faraco G, Moore J, Murphy M et al. Commensal microbiota affects ischemic stroke outcome by regulating intestinal gammadelta T cells. Nat Med 2016; 22:516-23.
37. Walter J, Armet A M, Finlay B B, Shanahan F. Establishing or Exaggerating Causality for the Gut Microbiome: Lessons from Human Microbiota-Associated Rodents. Cell 2020; 180:221-32.
38. Klein C, Jonas W, Iggena D, Empl L, Rivalan M, Wiedmer P et al. Exercise prevents high-fat diet-induced impairment of flexible memory expression in the water maze and modulates adult hippocampal neurogenesis in mice. Neurobiol Learn Mem 2016; 131:26-35.
39. Prado Lima M G, Schimidt H L, Garcia A, Dare L R, Carpes F P, Izquierdo I et al. Environmental enrichment and exercise are better than social enrichment to reduce memory deficits in amyloid beta neurotoxicity. Proc Natl Acad Sci USA 2018; 115: E2403-E09.
40. Butler C W, Keiser A A, Kwapis J L, Berchtold N C, Wall V L, Wood M A et al. Exercise opens a temporal window for enhanced cognitive improvement from subsequent physical activity. Learn Mem 2019; 26:485-92.
41. Kitahara M, Inoue T, Mani H, Takamatsu Y, Ikegami R, Tohyama H et al. Exercise and pharmacological inhibition of histone deacetylase improves cognitive function accompanied by an increase of gene expressions crucial for neuronal plasticity in the hippocampus. Neuroscience letters 2021; 749:135749.
42. Singhal G, Morgan J, Jawahar M C, Corrigan F, Jaehne E J, Toben C et al. Short-term environmental enrichment, and not physical exercise, alleviate cognitive decline and anxiety from middle age onwards without affecting hippocampal gene expression. Cogn Affect Behav Neurosci 2019; 19:1143-69.
43. Ramires Lima K, de Souza da Rosa A C, Severo Picua S, Souza E S S, Marks Soares N, Billig Mello-Carpes P. One single physical exercise session improves memory persistence by hippocampal activation of D1 dopamine receptors and PKA signaling in rats. Brain Res 2021; 1762:147439.
44. Rajizadeh M A, Esmaeilpour K, Haghparast E, Ebrahimi M N, Sheibani V. Voluntary exercise modulates learning & memory and synaptic plasticity impairments in sleep deprived female rats. Brain Res 2020; 1729:146598.
45. Cechella J L, Leite M R, Rosario A R, Sampaio T B, Zeni G. Diphenyl diselenide-supplemented diet and swimming exercise enhance novel object recognition memory in old rats. Age (Dordr) 2014; 36:9666.
46. Amirazodi F, Mehrabi A, Amirazodi M, Parsania S, Rajizadeh M A, Esmaeilpour K. The Combination Effects of Resveratrol and Swimming HIIT Exercise on Novel Object Recognition and Open-field Tasks in Aged Rats. Exp Aging Res 2020; 46:336-58.
47. Gibb A A, McNally L A, Riggs D W, Conklin D J, Bhatnagar A, Hill B G. Corrigendum: FVB/NJ Mice Are a Useful Model for Examining Cardiac Adaptations to Treadmill Exercise. Front Physiol 2017; 8:800.
48. Koch L G, Britton S L. Divergent selection for aerobic capacity in rats as a model for complex disease. Integr Comp Biol 2005; 45:405-15.
49. Shan W, Li J, Xu W, Li H, Zuo Z. Critical role of UQCRC1 in embryo survival, brain ischemic tolerance and normal cognition in mice. Cell Mol Life Sci 2019; 76:1381-96.
50. Leger M, Quiedeville A, Bouet V, Haelewyn B, Boulouard M, Schumann-Bard P et al. Object recognition test in mice. Nature protocols 2013; 8:2531-7.
51. Torii T, Kanemitsu K, Hagiwara A. Simultaneous Assay of Fecal Short-Chain Fatty and Bile Acids and Ratio of Total Bile Acids to Butyrate in Colon Cancer. Chromatography 2019; 40:49-57.
52. Lee J, d′Aigle J, Atadja L, Quaicoe V, Honarpisheh P, Ganesh B P et al. Gut Microbiota-Derived Short-Chain Fatty Acids Promote Poststroke Recovery in Aged Mice. Circulation research 2020; 127:453-65.
53. Feng C, Zhang Y, Yin J, Li J, Abounader R, Zuo Z. Regulatory factor X1 is a new tumor suppressive transcription factor that acts via direct downregulation of CD44 in glioblastoma. Neuro-oncology 2014; 16:1078-85.
54. Zheng B, Lai R, Li J, Zuo Z. Critical role of P2X7 receptors in the neuroinflammation and cognitive dysfunction after surgery. Brain Behav Immun 2017; 61:365-74.
55. Sholl D A. Dendritic organization in the neurons of the visual and motor cortices of the cat. J Anat 1953; 87:387-406.
56. Ferreira T A, Blackman A V, Oyrer J, Jayabal S, Chung A J, Watt A J et al. Neuronal morphometry directly from bitmap images. Nat Methods 2014; 11:982-4.

The below references correspond to the citations in Examples 5-10.
1. Feigin V L, Lawes C M, Bennett D A and Anderson C S. Stroke epidemiology: a review of population-based studies of incidence, prevalence, and case-fatality in the late 20th century. The Lancet. Neurology 2003: 2 (1): 43-53.
2. Benjamin E J, Blaha M J, Chiuve S E, Cushman M, Das S R, Deo R, et al. Heart Disease and Stroke Statistics-2017 Update: A Report From the American Heart Association. Circulation 2017: 135 (10): e146-e603.
3. Pohjasvaara T, Erkinjuntti T, Vataja R and Kaste M. Comparison of stroke features and disability in daily life in patients with ischemic stroke aged 55 to 70 and 71 to 85 years. Stroke 1997: 28 (4): 729-35.
4. Dennis M S, Burn J P, Sandercock P A, Bamford J M, Wade D T and Warlow C P. Long-term survival after first-ever stroke: the Oxfordshire Community Stroke Project. Stroke 1993: 24 (6): 796-800.
5. Manwani B, Liu F, Xu Y, Persky R, Li J and Mccullough L D. Functional recovery in aging mice after experimental stroke. Brain Behav Immun 2011: 25 (8): 1689-700.
6. Roy-O'Reilly M and Mccullough L D. Age and Sex Are Critical Factors in Ischemic Stroke Pathology. Endocrinology 2018: 159 (8): 3120-31.
7. Wang Z, Meng S, Cao L, Chen Y, Zuo Z and Peng S. Critical role of NLRP3-caspase-1 pathway in age-dependent isoflurane-induced microglial inflammatory response and cognitive impairment. J Neuroinflammation 2018: 15 (1): 109.
8. Li H, Yin J, Li L, Deng J, Feng C and Zuo Z. Isoflurane postconditioning reduces ischemia-induced nuclear factor-kappaB activation and interleukin 1beta production to provide neuroprotection in rats and mice. Neurobiol Dis 2013: 54 (216-24.
9. Benakis C, Brea D, Caballero S, Faraco G, Moore J, Murphy M, et al. Commensal microbiota affects ischemic stroke outcome by regulating intestinal gammadelta T cells. Nat Med 2016: 22 (5): 516-23.
10. Singh V, Roth S, Llovera G, Sadler R, Garzetti D, Stecher B, et al. Microbiota Dysbiosis Controls the Neuroinflammatory Response after Stroke. J Neurosci 2016: 36 (28): 7428-40.
11. Spychala M S, Venna V R, Jandzinski M, Doran S J, Durgan D J, Ganesh B P, et al. Age-related changes in the gut microbiota influence systemic inflammation and stroke outcome. Ann Neurol 2018: 84 (1): 23-36.
12. Xia G H, You C, Gao X X, Zeng X L, Zhu J J, Xu K Y, et al. Stroke Dysbiosis Index (SDI) in Gut Microbiome Are Associated With Brain Injury and Prognosis of Stroke. Front Neurol 2019: 10 (397.
13. Sadler R, Cramer J V, Heindl S, Kostidis S, Betz D, Zuurbier K R, et al. Short-Chain Fatty Acids Improve Poststroke Recovery via Immunological Mechanisms. J Neurosci 2020: 40 (5): 1162-73.
14. Magrone T and Jirillo E. The interaction between gut microbiota and age-related changes in immune function and inflammation. Immun Ageing 2013: 10 (1): 31.
15. Biagi E, Nylund L, Candela M, Ostan R, Bucci L, Pini E, et al. Through ageing, and beyond: gut microbiota and inflammatory status in seniors and centenarians. PLOS ONE 2010: 5 (5): e10667.
16. Yamashiro K, Tanaka R, Urabe T, Ueno Y, Yamashiro Y, Nomoto K, et al. Gut dysbiosis is associated with metabolism and systemic inflammation in patients with ischemic stroke. PLOS ONE 2017: 12 (2): e0171521.
17. Miossec P, Korn T and Kuchroo V K. Interleukin-17 and type 17 helper T cells. N Engl J Med 2009: 361 (9): 888-98.
18. Kolls J K and Linden A. Interleukin-17 family members and inflammation. Immunity 2004: 21 (4): 467-76.
19. Pizzonero M, Dupont S, Babel M, Beaumont S, Bienvenu N, Blanque R, et al. Discovery and optimization of an azetidine chemical series as a free fatty acid receptor 2 (FFA2) antagonist: from hit to clinic. J Med Chem 2014: 57 (23): 10044-57.
20. Liang P, Shan W and Zuo Z. Perioperative use of cefazolin ameliorates postoperative cognitive dysfunction but induces gut inflammation in mice. J Neuroinflammation 2018: 15 (1): 235.
21. Gad S C, Spainhour C B, Shoemake C, Pallman D R, Stricker-Krongrad A, Downing P A, et al. Tolerable Levels of Nonclinical Vehicles and Formulations Used in Studies by Multiple Routes in Multiple Species With Notes on Methods to Improve Utility. Int J Toxicol 2016: 35 (2): 95-178.
22. Lai Z, Shan W, Li J, Min J, Zeng X and Zuo Z. Appropriate exercise level attenuates gut dysbiosis and valeric acid increase to improve neuroplasticity and cognitive function after surgery in mice. Mol Psychiatry 2021:26:7167
23. Bokoliya S C, Dorsett Y, Panier H and Zhou Y. Procedures for Fecal Microbiota Transplantation in Murine Microbiome Studies. Front Cell Infect Microbiol 2021: 11 (711055.
24. Lee F H, Pfeffer M, Van Harken D R, Smyth R D and Hottendorf G H. Comparative pharmacokinetics of ceforanide (BL-S786R) and cefazolin in laboratory animals and humans. Antimicrob Agents Chemother 1980: 17 (2): 188-92.
25. Deng J, Zhang J, Feng C, Xiong L and Zuo Z. Critical role of matrix metalloprotease-9 in chronic high fat diet-induced cerebral vascular remodelling and increase of ischaemic brain injury in mice. Cardiovasc Res 2014: 103 (4): 473-84.
26. Li J, Shan W and Zuo Z. Age-Related Upregulation of Carboxyl Terminal Modulator Protein Contributes to the Decreased Brain Ischemic Tolerance in Older Rats. Mol Neurobiol 2018: 55 (7): 6145-54.
27. Liu X C, Mei Q, Xu J M and Hu J. Balsalazine decreases intestinal mucosal permeability of dextran sulfate sodium-induced colitis in mice. Acta Pharmacol Sin 2009: 30 (7): 987-93.
28. Zheng S and Zuo Z. Isoflurane preconditioning induces neuroprotection against ischemia via activation of p38 mitogen-activated protein kinase. Mol Pharmacol 2004: 65 (1172-80.
29. Kimura I, Ichimura A, Ohue-Kitano R and Igarashi M. Free Fatty Acid Receptors in Health and Disease. Physiol Rev 2020: 100 (1): 171-210.
30. Singh V, Sadler R, Heindl S, Llovera G, Roth S, Benakis C, et al. The gut microbiome primes a cerebroprotective immune response after stroke. J Cereb Blood Flow Metab 2018: 38 (8): 1293-98.
31. Parada Venegas D, De la Fuente M K, Landskron G, Gonzalez M J, Quera R, Dijkstra G, et al. Short Chain Fatty Acids (SCFAs)-Mediated Gut Epithelial and Immune Regulation and Its Relevance for Inflammatory Bowel Diseases. Front Immunol 2019: 10 (277.
32. Stanley D, Mason L J, Mackin K E, Srikhanta Y N, Lyras D, Prakash M D, et al. Translocation and dissemination of commensal bacteria in post-stroke infection. Nat Med 2016: 22 (11): 1277-84.
33. Brennan C A, Clay S L, Lavoie S L, Bae S, Lang J K, Fonseca-Pereira D, et al. Fusobacterium nucleatum drives a pro-inflammatory intestinal microenvironment through metabolite receptor-dependent modulation of IL-17 expression. Gut Microbes 2021: 13 (1): 1987780.
34. Chen R, Xu Y, Wu P, Zhou H, Lasanajak Y, Fang Y, et al. Transplantation of fecal microbiota rich in short chain fatty acids and butyric acid treat cerebral ischemic stroke by regulating gut microbiota. Pharmacol Res 2019: 148 (104403.
35. Winek K, Engel O, Koduah P, Heimesaat M M, Fischer A, Bereswill S, et al. Depletion of Cultivatable Gut Microbiota by Broad-Spectrum Antibiotic Pretreatment Worsens Outcome After Murine Stroke. Stroke 2016: 47 (5): 1354-63.
36. Gelderblom M, Weymar A, Bernreuther C, Velden J, Arunachalam P, Steinbach K, et al. Neutralization of the IL-17 axis diminishes neutrophil invasion and protects from ischemic stroke. Blood 2012: 120 (18): 3793-802.
37. Huppert J, Closhen D, Croxford A, White R, Kulig P, Pietrowski E, et al. Cellular mechanisms of IL-17-induced blood-brain barrier disruption. FASEB J 2010: 24 (4): 1023-34.
38. Liberale L, Diaz-Canestro C, Bonetti N R, Paneni F, Akhmedov A, Beer J H, et al. Post-ischaemic administration of the murine Canakinumab-surrogate antibody improves outcome in experimental stroke. Eur Heart J 2018: 39 (38): 3511-17.
39. Diaz-Canestro C, Reiner M F, Bonetti N R, Liberale L, Merlini M, Wust P, et al. AP-1 (Activated Protein-1) Transcription Factor JunD Regulates Ischemia/Reperfusion Brain Damage via IL-1beta (Interleukin-1beta). Stroke 2019: 50 (2): 469-77.
40. Waje-Andreassen U, Krakenes J, Ulvestad E, Thomassen L, Myhr K M, Aarseth J, et al. IL-6: an early marker for outcome in acute ischemic stroke. Acta Neurol Scand 2005: 111 (6): 360-5.
41. Yuille S, Reichardt N, Panda S, Dunbar H and Mulder I E. Human gut bacteria as potent class I histone deacetylase inhibitors in vitro through production of butyric acid and valeric acid. PLOS ONE 2018: 13 (7): e0201073.
42. Baxter N T, Schmidt A W, Venkataraman A, Kim K S, Waldron C and Schmidt T M. Dynamics of Human Gut Microbiota and Short-Chain Fatty Acids in Response to Dietary Interventions with Three Fermentable Fibers. mBio 2019: 10 (1) doi: 10.1128/mBio.02566-18
43. Kostulas N, Pelidou S H, Kivisakk P, Kostulas V and Link H. Increased IL-1beta, IL-8, and IL-17 mRNA expression in blood mononuclear cells observed in a prospective ischemic stroke study. Stroke 1999: 30 (10): 2174-9.

It will be understood that various details of the presently disclosed subject matter may be changed without departing from the scope of the presently disclosed subject matter. Furthermore, the foregoing description is for the purpose of illustration only, and not for the purpose of limitation.

1. A method of treating neuroinflammation in a subject, the method comprising administering to a subject a therapy for modulating a valeric acid-interleukin (IL)-17 pathway in the subject, whereby neuroinflammation in the subject is treated.

2. The method of claim 1, wherein the neuroinflammation in the subject comprises post-surgery neuroinflammation, and/or impairment of cognition, and/or postoperative cognitive dysfunction (POCD), and/or neuroplasticity, optionally wherein the subject is a human subject.

3. The method of claim 1, wherein the therapy administered to the subject comprises exercise, optionally wherein the exercise decreases gut microbiota changes in the subject and/or reduces valeric acid concentrations in the subject.

4. The method of claim 1, wherein the therapy administered to the subject comprises fecal transplantation, optionally wherein the fecal transplantation decreases bacteria producing valeric acid, optionally wherein the fecal transplantation decreases Megasphaera massiliensis.

5. The method of claim 1, wherein the therapy administered to the subject comprises an agent for reducing IL-17 in the subject, optionally wherein the agent comprises an antibody targeting IL-17, optionally wherein the antibody targets a polypeptide of IL-17 comprising an amino acid sequence of SEQ ID NO. 2.

6. The method of claim 1, wherein the therapy administered to the subject comprises a RNA interference (RNAi) construct targeting an IL-17 gene in the subject, optionally wherein the RNAi construct targets an IL-17 gene comprising a nucleic acid sequence of SEQ ID NO. 1.

7. The method of claim 1, wherein the therapy administered to the subject interrupts the gut microbiota-valeric acid-free fatty acid receptor (FFAR) 2-IL-17 pathway, whereby the concentration of valeric acid and/or IL-17 in the subject is reduced.

8. The method of claim 7, wherein the therapy administered to the subject comprises an agent to decrease and/or block FFAR2 in mediating an increase of IL-17 caused by valeric acid, optionally wherein the agent comprises a FFAR2 antagonist, optionally wherein the FFAR2 antagonist is GLPG-0974.

9. The method of claim 5, wherein the therapy comprises an agent for reducing IL-17 downstream immune and neuroinflammatory targets selected from the group consisting of FFAR2, C3ar1, C3, Iba-1, IL-1β, IL-6, and combinations thereof, optionally wherein the agent comprises an antagonist and/or antibody against FFAR2, C3ar1, C3, Iba-1, IL-1β, and/or IL-6, optionally wherein the antibody targets a polypeptide of FFAR2 comprising an amino acid sequence of SEQ ID NO. 4, C3ar1 comprising an amino acid sequence of SEQ ID NO. 6, C3 comprising an amino acid sequence of SEQ ID NO. 8, Iba-1 comprising an amino acid sequence of SEQ ID NO. 10, IL-13 comprising an amino acid sequence of SEQ ID NO. 12, or IL-6 comprising an amino acid sequence of SEQ ID NO. 14.

10. The method of claim 6, wherein the therapy administered to the subject comprises a RNA interference (RNAi) construct targeting an FFAR2, C3ar1, C3, Iba-1, IL-1β, and/or IL-6 gene in the subject, optionally wherein the RNAi construct targets a gene encoding FFAR2 comprising an nucleic acid sequence of SEQ ID NO. 3, C3ar1 comprising a nucleic acid sequence of SEQ ID NO. 5, C3 comprising a nucleic acid sequence of SEQ ID NO. 7, Iba-1 comprising a nucleic acid sequence of SEQ ID NO. 9, IL-1β comprising a nucleic acid sequence of SEQ ID NO. 11, or IL-6 comprising a nucleic acid sequence of SEQ ID NO. 13.

11. The method of claim 1, further comprising measuring blood valeric acid concentration in the subject prior to, during and/or after administration of the therapy.

12. A method for improving neurological outcome and/or mediating inflammatory response in a subject suffering from ischemic stroke and/or surgery-induced neuroinflammation, the method comprising:
providing a subject suffering from ischemic stroke and/or surgery-induced neuroinflammation; and
administering to the subject a therapy for reducing valeric acid in a subject, the therapy comprising: a) an agent for modulating, including antagonizing and/or disrupting, the valeric acid interleukin (IL)-17 pathway in the subject, including a) the gut microbiota-valeric acid-free fatty acid receptor (FFAR) 2-IL-17 pathway, b) exercise, c) fecal transplantation and/or d) an agent to decrease bacteria producing valeric acid, whereby the neurological outcome in the subject is improved and/or the inflammatory response in the subject is reduced.

13. The method of claim 12, wherein the exercise decreases gut microbiota changes in the subject and/or reduces valeric acid concentrations in the subject.

14. The method of claim 12, wherein the fecal transplantation decreases bacteria producing valeric acid, optionally wherein the fecal transplantation decreases Megasphaera massiliensis to reduce valeric acid.

15. The method of claim 12, further comprising modulating a valeric acid interleukin (IL)-17 pathway in the subject.

16. The method of claim 12, further comprising measuring blood valeric acid concentration in the subject prior to, during and/or after administration of the therapy.

17.-27. (canceled)


SEQUENCE LISTING (18 sequences)

SEQ ID NO: 1 - DNA, 11252 bp, Homo sapiens
  source 1..11252: mol_type = genomic DNA
ctagtaaagaggatgtgttaaaggcaagccccatctactcaggcgataaaggcactacacgttgtttggaagtcaccagacacagaatgcctttctatcaaacacttcacatgaataatggctgttcaatgttcttttgaccatacagattgggtttgtttttcacagatgtccaaacagttttgtgatctggttccatcagatgaaaacagcctggtgtgcactaggtggaggaaacatcaaaaaccaatagtaaaaaaagaaaattagtcactcaaaggagctgcacagatggtcttcatccgggcaaaatgcctaccaagagttagaatgaccaatggtgaggaaacaggacccaaatgttgaaaaaggattctgcctgccatctctcagcactcaccattaacaatgactctataaaaacatcctcattgttaatcatgcctttaattcatcatgtcatataaaaagagaattttctccagcagggattccagcacaggatgaagcctgtactcacccccaaagtgttctcatccatcagctcactgttcaggacggccctcaggagcacagccatgcggatacaaacaagcattatgctggagaggaacaatgatgctatcaggttggtaccaagactgatacttcctatttaagaaatcatgttaattatataggtattaagttctggtttttgcaagacaacatcggtaacctcagagatgaacttctagtatgtaatagtattgatatcttcaaattcagggtagatgatctagagcttgataaactgggtctaaatctgagtctcaattaaatcctgattttatcaactccccaaggccttccagatggctctctattgcaaatcttctcctctctaaacatcacgtcttccttttaaatgtctgtttgacattgctccttgtgtgctggcccatttgcaaagtatcagcccctacatttaaaatccaagagaattctctccaacgactcatttattctctctatattaagtgaagatcaagagagtagtcttgcctggtgatgtgcacagcattaatcaaaatggctccaatctttttgaacagccaccatcttttattaactttaagcagaaacaatggcatgatgcctcctcctatactgagacctttgttacttttataaaatggctatagaatataaatttaaaatatattaatctctcaatggtaaccttactacctaagcaaaaattaaaatttatttattttatcttttagcaagccttaattttccaccacactgagacaaaagactcaaaaatactctctccaggacacaaaaggaaaaatttttctttccatcccttacattattctctattgcaagtagaaagaaatactgtttaatggcctaataatttcaaggagccttgaaaatcatttctctaaatcaagaagggcagaaacattttaccactatcatcccttcaaatgcatgtttgtctcctggaaatctttccctgtctccagttgcatacttgcccccacccattgaagctctgaactcacaacatgcagttccccattgcttatggtgatctgtttcaagccctcagagaagcaagattgataaacctggaagacaatcactaacaaaataatcatccctaatttactactcccatttgttctttactatattcatctccagaaaatcatagatgatcaaaagattacctggtgcaaaactcattttataagacaggaaactaagcctcaaagaggtgaggtacccaagggcatgggtagttagtggcagattcacagtgaaaacaaaggtgtcctctcattcagtgttctaggcactctaatgcccagtccagcatgcactccacctcaacggtctcggattgaaacctgagtcagctggctctgcagacaaatgcagaggaagagccctgcccactgtgaacacactcttacacttccagctgctcctccaggagccacaggcttccagctcaagcaaacgtctgggaaatcagccatactcaaggctgcacacacccagctccaaccaccatgtcaaaatgatccttttaacacttctgtgaaaacccattttgtttccccattttattttaaagcatttcaaagcatagcacacaggctgaattacataggcacaggaggcaagactagggaaaacagctagagtcagcttctccccctgcaacttcaatcaaaatagttcacagtaagcacattctcccctccttcttccttgccagaaagcaacaacaaggaatcctccactccagggtgatcctgaataggctatagcctcatcacactaagttcaaaaggatcagaatgcagagccaaggcactagacaactcagtagggttattgagatgcaggtaggttctaagaagaatatagaaaattctgaacttctcaaataacattactcatactgtcaattaatcaaaatgttgaacagctattacttactagacactatgctaagtgctacaaagaccaataagactacttgtcttcaggtagcttgtagtctagcaagaaatgagctacaaagaaaacaagaaagtcagtatgtaaatgaatggcatgcaatacactagaaatatgcatgaacagccctgtatcagagaagaacaagcaacacaaaagcagtctgtgaagacccatgttgtataaagggccacattaggcaccataggagtcacaaagttgaagaacatactagccttgaaggctgacagtaaaatctaaacatggaaataaacacatatcttacccatagcttttaaagccaaatgaaacacattacaaaatgaaagaaaagttactagagaggtaggtgtaagctacatgtgatggatggaactatttatttcaactgaggtaatcaggccagacctgtgagatacatttaaacaggattttgaaggacaagtagcattttgataacctgaggatggggaagggcatttcagaaacaataccttaacaaagggtgggtaagctttgaaagaaggcctggagaaaaatcaggaccccatcatcccaggtcctggagcatggtggggggtaaggctagaaaggaagttgtacgaactcaaatattaattcaaatgctaagaggcttacccttcattctgtatgcaagctaatggcagaagaaaaggcacaattagagccatgctttgagaatcattattgaaagcatgtcgaagatggtctgaaggaagcaattgggaaaagcaagcatagctcatccaagtgggtgagagtgtgagttagaggaagcttggaaattggtgatgtgagagatgctgcagcttctgggattgctgcctggtcgtgtgtagaggaggggcagtagggctcattctgaatcttgtcttgaaaagcacatagatagtgatgccaaaaccaggactacggaaatcacttgaagctgtatcctacctcctcctccatctgtatctgcttcacctatcaaggatatctactattgccactaaaattcaggtgcttatggcctccctcattcatcagccagggtttatctggccaggaaagagaagccccttcaggcatttgcaacagagggagtttaagtcagagaactagtcaccctggtagttgagattgccatcagcaagaagctgttaccatttgaaggctgcagggacaaagggagtgagcagtcctctgggagactgaggaaggaagctcctggcttctccccactttccactttccacttcccacttttactcatctgtcctccaattcccttttggctgagcctagctgaaacccagctgacaggggagtttgagcaagcagcctccagggtcagccctctgagttacaggtagagcaggacagggaggaatggatctcaggacaaacaggttcaggatccggcaaactaattttacactccagccattgagttggaactactggccagcctccccccgagttagcatgtagaatatgggataccagctgagtgcctgagagttatcattcacctcagtgggggtaggggcggagaagggtgacatatagccagccacatctatatccactggcccttccttgtcctagtcctctgtattcctgagaaggaactattctcaaggacctgagtccaagttcatcttacttagagtacagagaaaagaaccgctaactccttctctctttcccccatcatgtctcctctcctttctagttctcatcactctctactcccccctgccccccttttctccatctccatcacctttgtccagtctctatccccattttcaattccttcctcaaaacaccaagttgcttggtagcatgcagggttggaacatgcctttaacagaaaatctcgtgtctcttgaacctagttatttattccttgagcagagtagatattcaacaaaagaattgttaaattcaattaaataggatatatcttattattaaatatttttttcattttttgtttacttatatgatgggaacttgagtagtttccggaattgtctccacaacacctggccaaggaatctgtgaggaaaagaaagatcaaatggaaaatcaaggtacatgacaccagaagacctacatgttacttcaaactttttcttcctcatgaaccattaaaatagagcataactcttctggcagctgtacatatgttcataaatacatgatattgacccatagcatagcagctctgctcagcttctaacaagtaagaatgaaaagaggacatggtctttaggaacatgaatttctgcccttcccattttccttcagaaggagagattcttctatgacctcattgggggcggaaattttaaccaaaatggtgtcacccctgaacccactgcgacacgccacgtaagtgaccacagaaggagaaaagccctataaaaagagagacgatagcgctacattttgtccatctcatagcaggcacaaactcatccatccccagttgattggaagaaacaacgatgactcctgggaagacctcattggtggtgagtcctgcactaacgtgcgatgctcttgctgatttggaccagatagtatttctggaccgtgggcatgaaacgctgggttctgactatggagatccaggaatactgtatatgtaggataggaaatgaaagctttggtaggtatttaagtcattgtgcagcattttcaagaactgatacacagcagtttgaaagataagattaaaactgaaagatagctatattggggctaaaccacacaagaagtgtcacatgatgctgtgcagtaagaaagaaaatttattgaaagtctgtttttctgagtacaaaggatttaatataattctcccacggcatttttctttaaaatgggtcactatccttgagattttgaaagccgtagcagcaacaacctttgtttccattatctcgtaccatattctcagtacattgaaactatgtattctaactaaacataggtataactgtgttttagaataagtggggtttatattttttaaatatttaacttcaagtatcttttttgaaatctgattttattacagaatcaatacatgttaaatttagaacaactggaaaatatacctaagaaaacatgaaggagatcgagtttttagttggatgcctgccagtagcaccaacagcacttctagcatgaatattgataccacatagattttctatagctctttcttccaatgtgaatgtttgacttcacgatgagtttcacagaatatgggactgagaacaatggtgcaggaggatatttctacctagaaaatcaaggttattattccttcccagacctgacaatgatgcatgtgctgataggctaatgacatgccatgacttgacatttttattaaaattattgccaaccaatggataacatgtctttcctaagtcaaaaggagaatgttgaaactagtttttttaaaaaaattttaaagccatggtgttaacattatgttggtcatctacctagatttttctctagctgatctgaaaaatgtagtatagattgtcctggaacattgtgtgttctctatgattagcaatgcatcatcatcacaattaatttgtcaaaaagaaccacatagtaatctaatctccaacctctctctcctttcccattcaattctagtcactgctactgctgctgagcctggaggccatagtgaaggcaggaatcacaatcccacgaaatccaggatgcccaaattctgaggacaagaacttcccccggactgtgatggtcaacctgaacatccataaccggaataccaataccaatcccaaaaggtcctcagattactacaaccgatccacctcaccttggaatctccagtacgtaaagcttccagataaaaatgctatattcttcatccctcttatgcatcagactgccagttaaatctccctgaggatgattttattcatttagaattaccagtcaaacctggaaggaccactgtgaagagcaattctcaaactttctacagatttctttaaccaagcacaggacagcctccaataatccctatcctgttagatctaattgtcactgacaccaataatcaacccaaattaattataatcattattctaatatttatgagaccccaagtctattctttatttattcaaagaatagacatttatcaaagaggattaatgcttttattatcttaaccagagctgccattgagaagatttattgcaaataattaataattagggttttttacttttattcttttgcttatttttgtttttgaatcccagtggaataagtatcactggggtatttctacccctttgtgtgttaaatagtcttgatctacttcctaacatacctatgcttgctgtatccttagtatacccagtatttagaccccatcaagggttaaataccaaatgtattttgatcatttgacttcatacaaataagtctctgttctgtggagcctacagattggtctgattgtaggatttcttctcttcttcccattactaggaagagtcaaaataaatcaattcaaaaatgcaagcaaatcattcactgatctaaaagagagagggaagagaaggtcatagagacacttaaccttttgtttccagccctttatctcagctctgggctctgtcccacgaatgtgatctcagataaaattttgatgtattccctcttcaaagacagacttcatcaagtcaaataaacagctatcttattctagatggttccaagtctactcttcctttggtcttcttctgtctgtcaaatgtaccctaaaaaagctatcatttgtgtcaaacttaaattttttctgtggcctcagtctatcttattttattcattcttcaaataaattggagaaaaactgatcactgtcttcttttctataacaattcacgtgcttgaaaaaaaaatccaatttgtccccaaagttcttcttcaaactaacatcatttaaagaatttgcaatgcctataatttgtcatcctgtgaacttgcctctcttcatgtattcctgttttatttctttcccactttaccaggaattcactttcctcctgatttttctcccctctgcagccgcaatgaggaccctgagagatatccctctgtgatctgggaggcaaagtgccgccacttgggctgcatcaacgctgatgggaacgtggactaccacatgaactctgtccccatccagcaagagatcctggtcctgcgcagggagcctccacactgccccaactccttccggctggagaagatactggtgtccgtgggctgcacctgtgtcaccccgattgtccaccatgtggcctaagagctctggggagcccacactccccaaagcagttagactatggagagccgacccagcccctcaggaaccctcatccttcaaagacagcctcatttcggactaaactcattagagttcttaaggcagtttgtccaattaaagcttcagaggtaacacttggccaagatatgagatctgaattacctttccctctttccaagaaggaaggtttgactgagtaccaatttgcttcttgtttacttttttaagggctttaagttatttatgtatttaatatgccctgagataactttggggtataagattccattttaatgaattacctactttattttgtttgtctttttaaagaagataagattctgggcttgggaattttattatttaaaaggtaaaacctgtatttatttgagctatttaaggatctatttatgtttaagtatttagaaaaaggtgaaaaagcactattatcagttctgcctaggtaaatgtaagatagaattaaatggcagtgcaaaatttctgagtctttacaacatacggatatagtatttcctcctctttgtttttaaaagttataacatggctgaaaagaaagattaaacctactttcatatgtattaatttaaattttgcaatttgttgaggttttacaagagatacagcaagtctaactctctgttccattaaacccttataataaaatccttctgtaataataaagtttcaaaagaaaatgtttatttgttctcattaaatgtattttagcaaactcagctcttccctattgggaagagttatgcaaattctcctataagcaaaacaaagcatgtctttgagtaacaatgacctggaaatacccaaaattccaagttctcgatttcacatgccttcaagactgaacaccgactaaggttttcatactattagccaatgctgtagacagaagcattttgataggaatagagcaaataagataatggccctgaggaatggcatgtcattattaaagatcatatggggaaaatgaaaccctccccaaaatacaagaagttctgggaggagacattgtcttcagactacaatgtccagtttctcccctagactcaggcttcctttggagattaaggcccctcagagatcaacagaccaacatttttctcttcctcaagcaacactcctagggcctggcttctgtctgatcaaggcaccacacaacccagaaaggagctgatggggcagaacgaactttaagtatgagaaaagttcagcccaagtaaaataaaaactcaatcacattcaattccagagtagtttcaagtttcacatcgtaaccattttcgcccccatggcccatgtgctgtcttgccctacttctgaaggcctctagatattctcaggccactctgcaggctccctgcttcttgaaagaccttcctcttcactccatcctgctccatccagtgtgctccagcccacccagaggcccatggcttttctaggcttctttccctattccaaatccaggggttggcctctccagccacctcctctcagtcattttgtgctcacatcttgattcctgtgaccatccctgtcaccattttcctacaggaaatccccaggcagcttccagggacagttcctcacagcctttacgctttcaagcttctcactaaaaacctgctgctcgttttcaagctttctctgaacggtgatgatgaaaagaaaaaattgtttttgtttttgttgggagagggagttgtaaaggatgtatatgtctatatgtgtgtgtgtgtgtgtgtgtgtggtcctgaaccattatttcccactagattttgggtggggggtatgcggatgggaagaggcagttgctttccatgtactcctcccctgatacactttgcttttattctctcttcattttccccagttagcactaccatgtacctagtcatccaagctagaaatctatgtggcttccctgaatctttttccttctgcctctccctcattcctaacatccacatatcaccaaatactatattctcacttccaaaagttctccaaggaacccccacctccccaccaataccatactacccaggttcaggactgcatctattctcatttctctcctccatgattgctacaaccttccacctgttcttgctgccattgttcccttctctcaatccatcctccgacctgtacctatgacacaaattcaaatataacaaaaagcgccccccagaaatctggccttgctccctctcctgttctatcccagggtgcctcatccttgacgttttaccctcgagtcgtggtgaagttgttgcggagaccaccatgctattcttggccttgatgcctatgctatgtggtcccttctgcgtgaatgcccttgccaccttccccctgctatccctccagtcacacgctgccctccccactctcccccatttctatggctctactcacatattaaaactttctttaggtctcatcttctctaaggagactcctggacccatcactgcctgttatcctagattaggtccctctctttcttacgggctccaaaagcactgtgcagatcctttatcactgaccttacatactatatcagctttacctatttagccagtttgtggttcctcaagagcagaaacatcagcacctgacatggcaggtagtagacagtcaatactcagctgaactttgaaccaatagtccacagacacctcaaaatcaacagagacaaaactaagctcgctagctttactgccaaactggctcctcctactgcttttttatctactggaatggcatcgtctactcatctgagcaagaaacctagctttctctccatccctcattcccctcctccaatcagtgaccaactcttcatcaatgtctcacatgtcctccccttccttgctaactccaagtttgctattacagttgcattatattatgacactaactggcctctctgccttgcttcttatatttaataattcggtatctctcaaacttgtaagggagtctagtcagagtaggtaattaatagcttctgcaacagacaaagttcaaataaacaaaggtttgtttcttgtgcatgttacaatccaacaggatcattagggagctttgctctacttggccactgagagacccaggctgacacagaggttctgacttctacaacaccatctagcactgcaatgtctaacacagtagccacgaggcatttgtaactattgagcgcttggaatgtggctagtcccagttgagttgtgttataagtgtaaaatgcatactagatttaaaatacttaagtttt

SEQ ID NO: 2 - protein, 155 aa, Homo sapiens
MTPGKTSLVSLLLLLSLEAIVKAGITIPRNPGCPNSEDKNFPRTVMVNLNIHNRNTNTNPKRSSDYYNRSTSPWNLHRNEDPERYPSVIWEAKCRHLGCINADGNVDYHMNSVPIQQEILVLRREPPHCPNSFRLEKILVSVGCTCVTPIVHHVA

SEQ ID NO: 3 - DNA, 993 bp, Homo sapiens
  source 1..993: mol_type = genomic DNA
atgctgccggactggaagagctccttgatcctcatggcttacatcatcatcttcctcactggcctccctgccaacctcctggccctgcgggcctttgtggggcggatccgccagccccagcctgcacctgtgcacatcctcctgctgagcctgacgctggccgacctcctcctgctgctgctgctgcccttcaagatcatcgaggctgcgtcgaacttccgctggtacctgcccaaggtcgtctgcgccctcacgagttttggcttctacagcagcatctactgcagcacgtggctcctggcgggcatcagcatcgagcgctacctgggagtggctttccccgtgcagtacaagctctcccgccggcctctgtatggagtgattgcagctctggtggcctgggttatgtcctttggtcactgcaccatcgtgatcatcgttcaatacttgaacacgactgagcaggtcagaagtggcaatgaaattacctgctacgagaacttcaccgataaccagttggacgtggtgctgcccgtgcggctggagctgtgcctggtgctcttcttcatccccatggcagtcaccatcttctgctactggcgttttgtgtggatcatgctctcccagccccttgtgggggcccagaggcggcgccgagccgtggggctggctgtggtgacgctgctcaatttcctggtgtgcttcggaccttacaacgtgtcccacctggtggggtatcaccagagaaaaagcccctggtggcggtcaatagccgtggtgttcagttcactcaacgccagtctggaccccctgctcttctatttctcttcttcagtggtgcgcagggcatttgggagagggctgcaggtgctgcggaatcagggctcctccctgttgggacgcagaggcaaagacacagcagaggggacaaatgaggacaggggtgtgggtcaaggagaagggatgccaagttcggacttcactacagagtag

SEQ ID NO: 4 - protein, 330 aa, Homo sapiens
MLPDWKSSLILMAYIIIFLTGLPANLLALRAFVGRIRQPQPAPVHILLLSLTLADLLLLLLLPFKIIEAASNFRWYLPKVVCALTSFGFYSSIYCSTWLLAGISIERYLGVAFPVQYKLSRRPLYGVIAALVAWVMSFGHCTIVIIVQYLNTTEQVRSGNEITCYENFTDNQLDVVLPVRLELCLVLFFIPMAVTIFCYWRFVWIMLSQPLVGAQRRRRAVGLAVVTLLNFLVCFGPYNVSHLVGYHQRKSPWWRSIAVVFSSLNASLDPLLFYFSSSVVRRAFGRGLQVLRNQGSSLLGRRGKDTAEGTNEDRGVGQGEGMPSSDFTTE

SEQ ID NO: 5 - DNA, 16628 bp, Homo sapiens
  source 1..16628: mol_type = genomic DNA
acagtggcactatcatagctccctacagcctcaacctcccaggctcaagccatcctcccgcctcagcctcccaagtagctgggtctacaggtgtgcaccaccatgcccagctgattttttgtatttttagtagaggggggttttgccatattacccaggctggtcttgaactcctgggctcaggcaatctgcctgccttggcctcccaaaatgctggggttacaggcgtgagtgaccgctttcttttactttttttaaaatttttttatttttatttttttgagacggagtatcggtctgtcacccaggctggagtgcagtggcgcgatctcggctcactgcaagctccgcctgccgggttcacgccattctcctgccttagcctccagagtagctgggactacaggcgcccaccaccacgcctggcgaatttctttatgtatttttagtagagacggggttttactgtgttagccaggatggtctcgatctcctgacctcatgatgcgcccgcctcggcctcccaaagtgctgggacacaggcatgagccaccgcgcccagcctcttttacttttaaaatagatttgcaatctctgtgcacaaccaagttatgaaattaaaagcaatagttcccctttctccttactttccattgacacatgaacatgtccttccggaagatcctattcagaacccagctggagagaaggtagaggagacatagtccataggtttctaatagataataaggaagggtgtgggcggcttgagtatcgatagatgatatctgacacagtccctgttttgcagataagaaaacgagaacatacagtgtgagaagcttattttcagtaacacaattagtaagtggcaaagttgagacttaaacccaggctttctacctgccctcccttagtccttagaataccctttttactcccaccaacgccatgtacctgtgccatttagttgtgttgggctcacctcccccttaatatgtgctcatgcttttcccttgaaatgccctctcagcttcaatctccctgctcaaatgagagaattatagattgttagatgtaaaagaaacctgaaagaccatttaatccaacctttaattttccacctaagaaaacaacaacaaatttctaggtcaagtatctagcccaaggtcgagtagttattacaaaactgaaagtcaaatgtatgtctccacattcctggtctagtgctctattcacctcatgaatcttttcttccataaaaattttccagctaattctagccaccatcaatcaatcttaccctttgctgaccagccattgcatttgtctgtgtcatccacagtttttggcacttagttatatttaatttagagcataggttactacaaattatctaataacggatttaatcattttttgttgaagtctttaaggacagtaactttataatgtatttaactctctcagagaaaacaagctagagatctgtgattctaacatttttattttggccaaacaagctttaatacagatctccatcaattacagtgtagaattagtcaagtgactggtaaaaggttttaatatggctgcctttgcaacatacattcaaattttcttttttccaaagcatctgatctctgcttttagaaatttatatttagatattcagaatagaacaaaacagaagtgaatcacataacttcctcagagctttccacaatacaccacaaaacaaccagtactttttaagaagagagagaggtcatccagaaacaggagcactattcacaacaagtttttagtaattttcttcctgaaaaatccaaagaacacagctgggagctcttgaaccagtttaaattgctgtataacatccagctaaatttttatatttttagtagagatagggtttcatcacgttggccaggctggtctcgaactcctaacctcaggtgatccgcccgccctggcctcccaaagtgctgggattacaggcatgagccaccacacccagccacattgctgtgtaatctttagagagagagaaattagtaaaagtgaattcatgccagaaacaactctaaggactcaagttaattatttgaattatattcagatgtcaacatccttttcaaacaaacattcattcacttattggtgccacagacctgttataccaggcaaggtagcagccactggagacagacaagaaattcatggtaggggagagagagagacaaggtattattttacagtactattgcctgacaaatttctacccatccttatggccccaatgctacctcttcagtgcatcatcgtgggtttgcttccatgagaattagtctcttacggtttcatattcccagggtgtattgtgccctcctaacagcccctactgtattacagttatatacatctctcattcctcctccatcttccgcagtgcaacacactgtatcaggagcaggagcagaagcagagtatatgtttatggtactgaacttagtgtgacaatttgtgaagtctctttagtggaaaattctgtatctttcaaaatgtaaaagtgtgtgctcttagacttagcaattccaattctaaaactctattctacagaaatacatgtaaatactatagtgctcattgagttttgtttataatcatttaaaattggataaaaacaatattccacgaaaaataattaagtaagtggaataatatggagcaacttaaaaagtataatttgggaccgcacgcagtggctcacgcctgtaatcccaggactttgggaggccgaggtgggcagatcacctgagagcaggagttcgagaccagcctggccagcatggtgaaaccccgtctctactaaaaatacaaaaaattagccaggtgtggttgtgggcacctgtaatcccagctacttgggaggctgaggcaggagaatcgattgaagccaggaggcagaagttgcagtgagccgagatcgcaccattgcactccaacctgggtgacaagagcaaaactctgtctcaggaaaaaaaaaaaaaaaagtgtaatttgggccaagatgtcctgatataaaaagataagtggaaaaaatagaacaaaacaatatgcgtagtagaatatgcgtatttaagagagagactacatctggaaagtggaaagaggatagctaggtgtttaggaaattttggtttttactttaatcttctcatatttaatttttaccatggggaattaattgtttttataaaatatattttaattagatatatatgtacatatatgataatcctacattttatatgctttctggatacctatattattcaatatttccaaatattggaggaaagaaaacccaaaataatcaataggattatatgtactaacatggaagtatgttttgatatattgttaagtaaaaaagcaagacataaaatcacacataaattaccatcccatttatgttcaaaactaccttttagaaaaatgtgtgttaggccgggtggggtggctcacaccggtaatcacagcactttgggaggctgaggcaggtggatcacctgagctcaggagttcaagaccagcctgcccaacatggcaaaaccccatctctaccgaaaaaatataaaaattagccggttgtggtggcacatgcctgtagtcccagctactaaggaggatgaggtgggagaattgcttgaacctaggaggcagaggttgcagtgagccaagatggtggcactgcactccagcctgggcgacagactgaggccctgtctccaacaaacaaaatgtgtgctatattcagaaaaaaaaaaatcaggaagaatgaacactaacctattaaaaatagttacttcgagcagtggaattgaaaagaaaatctttcattttttttacccattataattctgtattatttcaatttatctcttctagaacaaagtatgttctatttttgtaattagatgatgacaatggaaataaactaataaatactactcaactcttggcatttttcttttgatctttctgcctcaaaaatattccgtggtgacttgactgcaaacaagcagaggagcagatattatggtttggacaagttgattaatatagactgaagcctatatcacagacaacagtgttcaaatacatttgacccaaataaactttattttatatgcatatatctaaaagagtatatgcatatatctaaaagaataatataagaattatttacagcatagtattcattaagcaggaattaccttcagtcttctgtactccttcctgatgaaacagcacctagactccagcctgttctccagtgaccactaagctctcccacctacctccccacctacctgccctctaccacctacctgcctttgactaggctccacttatagcctaagcagcttttcacctttttctgtctctagaattcctacaggtacttaattaagtacttgcccacatcttatttctatcttaagactctcctagattgccctgggttatatatacccctggctagagtgttctattattttacttctattgttacttctattattatagcctagtttgtattacagctttgtgtgtatatgtatgggtacatattcccactcgattgtgaatctctggagattagaatatagcttaattcatcctgattttgacatggcaccgtttttcacatatatcaagcatttcagatatgttatttgagattaattaaatgagtttattcaggtcattcctccccaaccactccccactccaaccctcacatctcatagagtcttttttttttcatttctaaacaaaacctataaagttgaaccctatgtacagaggtaccctctctctaaccattctctgtgtgctgcagaaaatgtctcaggattataaactcctacttctgttttcctgctttgcctttctaagccatttagaaatgactcactttgaaactcttcctgtgaggtcagatagtggtctagagcataagacttaacttattgccggaaacagagagagaacagaagaagagaaagctcagcaaattttcttgccatacttcatgacttcactgtggctaagtgtggggaccagacaggactcgtggagacatccaggtgctgaagccttcagctactgtctcagttttttggtaagaaaacctagacctacctgatgacttgtgcttagcctgttttgctcattataggtaatgagtttgagtgtcactatcttctatttcccctattccttctctgactttgaattcttttatcttatctctatgtctaacttcctttttctaaatattccacattgcatttgtctttgcttttaatattttcatagtggaattaatagtgaatttttacatcatttctttcctttctttgaattggctatgtatttcaccctgaactttgattccagctgtccccattaatttgtttttcaaataattgattgcactttttttccctttccatttcctctttcttttttttattcttttgcccattccttattgctactgactctgttatcctattactacaatttgattctggggtccacttttctttctctatcacagtggaattttagtttcaggatattaattacctttaaattacggctggtcaacttagtaataattttttttaatcctctactaatcttaaacacataaaggtatggtatttttagccaaattaacttgaagaaatgtaaaaagtaatcttgctctgcaggactttttttttttttttttttttttgagacagagtctcactctgttgcccaggctggagtacagtagcatgatctcagctcactgcaacctccgcctcccaggttcaagcgattctcctgcctcagcctcctgagtaggtgggattgcaggcacgtgctaccacacccagttaaatttttttgtatttttagtagagacagggttccaccatgttggccaggttggtctcaaactcctgacctcaggtgatctacccgcctctgcctcccaaagtgctgggattacaggcgtgagccaccacacccagccttgttctaaaggacttttaatccctgactcctacatactttcatttcaaaacagataataacaatatttaacatatagctcatgacagataactctatttttattaaaattttgctgtttgcagtccctgctacttcagttcatgcagttctcggcagcttcccctttattagcaataccatatatctttttttttttaatgtgatttttttttttttttggtagtaaaaacagcatttgcctaacagtcctcggacctgaaatccaagaacctccctagtaatgattatatgcttgtaatctaatttgctgagtttcactgtcaaacttgagaaataaaagcagagaaaacgtaggctgggcacagtggctcatgtctgtaaccccagcacactgtgaggccaacatgggaggattgcttgagtccaggagtttgagagcagcttgggcaacatagcaagaccctatctctacaaacaaacaaacaaacaaacaacaacaacaacaacaaaatgaggagaggagagatgattaccaagttttctttcagccctagcatcccatgactctattcttctctcaatattttaggggggtaccgtgatagtatttaaatatctgagtagacaaggccatggaaaggggaatgagaataatttcttcttctttttttttttttgagatggagttttgcttttgttgcccaggctggagtgtagtggcgcaacctcggctcaccacaacctctgcctcccaggttcaagcgattctcctgcctcagcctcctgagtagctgggattacaggcatgtgccaccatgcccacctaattttgtatttttagtagagacagagtttctccatgttggataggctggtctcaaactgacctcaggtgatctgcctgccttggcttcccaaagtgctgggataacaggtgtgagccactgtgcccagcccatgtcttcttttttattattttgttgacttgctattttaacttctgctaatcatatgaggccctatggcaatatttggctgactcagcagaactactttcaagtcacaaaaatattttgagcctctataaaagtaaaatgttattttatccagtaaaaattaggaatttcacaaaaagaaagttaaaagggacagcatgggaattaaggaagaggcctgggtaaggattacatggatacaaattagaattttagatgtaattgcaaaagaaaaaaaaagtcaacccccaaaatgggcatccatctattcaagtaattttttttttctttttttttcttttgagacagagtctctttgtcatgcaggctggagtgcagtggtgcaatctcagctcactgcaacctccacctctccagttcaagcgattctcgtgcctcagcctcccaagtagctgggattacaagtgtgagctaccacacccagctaatttttgtatttttggtagagatgggattttgccatgttagccaggctggtcttgaactcctagccccaagcgatcttctcccctcggccccccaaagtgctgcgattacaggcatgagccactgcgcccagcctttccacataatctttaaccttggtgtctcataaggcattatgttaaattatgtgaaatgagcatttatgaataagactcctttttaccatcataaagtttaaatccagaataatagattagacagccattataattattgtacaagataaaatgtgtcattgcatatagaatatgaaaaaaaggttcaaacatgcgcacacacacaaattaagaagctgaagacttggtgaagggcataattccagatagaagtaaacagcattagccatggaacggaaaatggcattaagttggaatagtgaattgttcaggaaagctataaagcagggtacatttacgagcatgttcacagttaggggaaggtaatatcacagaggccaagagaagagagtgttaagaagtcagtgtatctaatgacacatattgtggaaggtgactgagaaacaaacgtttggatttggtttttagaagtaattttagcagaattatggaagcagaagatacattacaaagaattaagaagttggtggccggccgggcctggtggctcacatctgtaatcccgggactttgggaggccgaggcgggcggatcaccaggtcaggaaatcaagatcatcctggctaacatggtgaaaccccgtctctactaaaaatacaaaaattagccgggcgtggtggtacacgcctgtaatcccagctcctcgggaggctgaggcagaagaattgcttaaacccgggatgcggaggctgtagtgagccgagatcgcaccattgcactccagcctgggagacagagcgagactccatcacaaaaaaaaaaaaaaaaaaaaaaaaaagggccgggcgcggtggctcacgcctgtaatcccaacactttgggaggccgaggcgggcggatcgcctgaggtcgggagatcaacatcatcctggccaacatggagaaaccccgttctctactaaaaatacaaaaaaaaattagccgggcatggtggcgcatgcctgtaatcccagctactgggaggctgaggcaggagaatcacttgaacccgggaggaggaggttgcagtgagccaagatcgcgccactgcactccagcgtgggcaacaagagcgaaactccatctcaagaaaaaagaaaaaaagaagaagttagtgttcagaacagtaggcgtaggccccaaaacaaagcagtatccttgaaaaagagaaattatgctaaattaagagacttaagaagaaagtgcgatctgaagtagatattgtcgtggacaagccagctataaaagatgtcttagggacagttgaaaaataatcatataaaagggggggcatggtggttcgtgcctgtaatctcagcacttcgggaggccgaggaagaatcagtagagcccaggagttggagaccagcctgagcaacatagcaagaccccatctttacaaacagaaacaaaacagataaaggtcgggtattccttatggtacatattgtataatgtggagactgctaactgaaaaaagaaaaaaatgtataaaaaatatgtatttacacccatgttcattgatgcataattcacaatagtcaaaaggtgaaagcaatccagatgtcctctgtggaatgactggataaacaaaatgaagtatagacctacaatggaatattattcagccttaaaaagaaagaaaattctgacccatgccacaacgcggaggaagcttgaagacattatgctaagtgaaatacaccagacacaaaaagacaaatactgtgtgattccacttagatcagatatctaaagtagtcgaattcatagaaattgaaagtacaatggtgattgccaggggctgagaaaaggagaaaatggggaattctttcctgagcacattttcagttttgtaagatgaaaaagttctgaaaattggttgtacaacgtgaatatggtaaacactactgaactgtgtacttaaaaatggtgaagatggtaaatgttatgatacatgtgtttggcaattaaaatttttttgttagggccaggcgcggtggctcgcacctgtaatcccagcactttgggagggagaggagggaggatcactcgagcctagcagttcaagagtagcctggccaacatggcaaaacctgtctctgctaaaaatacaaaaattagttggtacgttggtgggcaactgcagtcccagctactcaggaggctgaggcacgataattgcttgaacccgggaggcagaggctgcagtgagccgagattgtatcactgcactccagcctgaatgacggagggagattctgtctcaaaaataataataataataataataaataaataaatgaagcactgtcccacatattagaaggcttctagccatcacagcccctgctgtctaaagatacgcatgtgtatacctaaatgcacacacacacataaaaaaaggtcaagaggatataaattcaggtgctaaaataataatcactgactagtgagtatatttttattttcttttttgtttgtctatattttccaattttcttcatgcatattttttgcttttgtaataataaagctcttttcccaagttacggtcataaaacacaaataaataagaaagaaatgataggtagtgaggaagtcaatgcagagggccaacaactcttggaaaatttgaaagcaaaaggagatggagttgtatctaaaagacatcgctgagtctagagtacctctttctgtggcggcgagtcctctgaaaatctggtggggagagtggatgaagcttctgccctcagagaaatgagaatatgtaaagttgaagttttgcatatcattttaggtggtgatggaacttcctaaaaccctttcgtgacctcaggttggagacctccagtccagatatttttgtgtgtttacttatttagcttatttgtttatttttaaacacactgggtgaagaaaggagccagtggaaaaaccaagattgaaagtacaagaaagaggagaaatttacactaatatggacttccagatgaggctgtgattttgatacacacataaatcaatacagtagattttaaattgtctatcataggatgggcatggtggctcatgcctataatcccagcactttgggaggccaaggcaggcagatcacccgaggtcaggagttcaagaccagcctggccaacatggcaaaaccccgtctctactaaaaatacaaaaattagccaggcgtggtggtgcacgcctgtaatcccagctactctggaggctgaggcaggagaatcgcttgaactcgggaggcggagcttacagtgagctgaaatcaatccactgcactccagcctgcgcgacagaggaagactctgtctgaaaaataattaataaattaataaataaatataattgtctatcagagaatgcttttatgtggtcccgtgtgaggtgaaggaaggcaaactaaaacagcgtgaggaccttctggtttcatgatcccacatctttatgtgggaagattagaatcctaagaatatgtatgcattttcaaaaagatactgtttgttttaacatttttttcatctttttgcagaagtttagcaatggcgtctttctctgctgagaccaattcaactgacctactctcacagccatggaatgagcccccagtaattctctccatggtcattctcagccttacttttttactgggattgccaggcaatgggctggtgctgtgggtggctggcctgaagatgcagcggacagtgaacacaatttggttcctccacctcaccttggcggacctcctctgctgcctctccttgcccttctcgctggctcacttggctctccagggacagtggccctacggcaggttcctatgcaagctcatcccctccatcattgtcctcaacatgtttgccagtgtcttcctgcttactgccattagcctggatcgctgtcttgtggtattcaagccaatctggtgtcagaatcatcgcaatgtagggatggcctgctctatctgtggatgtatctgggtggtggcttttgtgatgtgcattcctgtgttcgtgtaccgggaaatcttcactacagacaaccataatagatgtggctacaaatttggtctctccagctcattagattatccagacttttatggagatccactagaaaacaggtctcttgaaaacattgttcagccgcctggagaaatgaatgataggttagatccttcctctttccaaacaaatgatcatccttggacagtccccactgtcttccaacctcaaacatttcaaagaccttctgcagattcactccctaggggttctgctaggttaacaagtcaaaatctgtattctaatgtatttaaacctgctgatgtggtctcacctaaaatccccagtgggtttcctattgaagatcacgaaaccagcccactggataactctgatgcttttctctctactcatttaaagctgttccctagcgcttctagcaattccttctacgagtctgagctaccacaaggtttccaggattattacaatttaggccaattcacagatgacgatcaagtgccaacacccctcgtggcaataacgatcactaggctagtggtgggtttcctgctgccctctgttatcatgatagcctgttacagcttcattgtcttccgaatgcaaaggggccgcttcgccaagtctcagagcaaaacctttcgagtggccgtggtggtggtggctgtctttcttgtctgctggactccataccacatttttggagtcctgtcattgcttactgacccagaaactcccttggggaaaactctgatgtcctgggatcatgtatgcattgctctagcatctgccaatagttgctttaatcccttcctttatgccctcttggggaaagattttaggaagaaagcaaggcagtccattcagggaattctggaggcagccttcagtgaggagctcacacgttccacccactgtccctcaaacaatgtcatttcagaaagaaatagtacaactgtgtgaaaatgtggagcagccaacaagcaggggctcttaggcaatcacatagtgaaagtttataagaggatgaagtgatatggtgagcagcggacttcaaaaactgtcaaagaatcaatccagcggttctcaaacggtacacagactattgacatcagcatcacctagaaacttgttagaaatgcaaattctcaagccgcatcccagacttgctgaatcggaatctctgggggttgggacccagcaagggcacttaacaaaccctcgtttctgattaatgctaaatgtaagaatcattgtaaacattagttctatttctatcccaaactaagctatgtgaaataagagaagctactttgtttttaaatgatgttgaatatttgtcgatatttccatcattaaatttttccttagcattgtctaagtcttccagattccatttaaaaccatttcttgttctcctacgtgagtgaaagatgatcatatatcctaatgctttgttgtcgtgtggtgttgatggttttaaacgaaaagaaagtgcaaaaagaaaatgcctgtgaagacaagaagccatgagactgagtctggagcatagggttatgcaatgatgcctgtccctgggaacacccctgggtacaggatatagaaatttccactattacatagagtttccactattacaactaaataagcatctattgtgtgaaaactgactcatgaaatgttatgaaagctgtggtttggggagttctgtttcttctaactgcctaccggttgggcacctattttccactcctcttcctaagctccttaatttccttattactccccagcctccaaatcttccacatcagactttgtgcctcaaacaacctctaatttcgtaagattctagttactcccttcctcttgctccaaatgaatactttctaagaaagtatttcaagtggaaggagaaagagggtggaggatggagcagcaattcttctactctctgcaactgagtaccctaccaggcttgccatcacattttaaaacatgacgacaggcaacttacatgccaaaattaccaaatatatcttctgggttttttaaatccttttctttgccaaagtaatacatgcacatagttttaaaataatttaataaggtatataatgaaatatgaggtctcctacctcactgtgcccaaaagttccctcctcccactctcatttcccagagataatccttgcacaattttagatgtttcctttgataattatcatgatgtttctaaatcatgtgcttatgctgctcttttctggaggcatgataaaacgacttcttgttttgaaagatgaagatgtttatccaagcaccccatatttttaatttgtttatccagcatcccaacattcattaataaccatattttaattcattcatgaccacatatttttcttctactttgtctatacactccaaccatttatatagctttccttctgtccctttttcatttaaaacaaaattacctaactccctaccaccttctcatttttctgtatatataaatgtttgtgtcaaacgtctgaaatttctggcttgtttgtatcacaacgtggcctcatctaaaccaaatacaatgatgtagtctaaaaacagaaaatgacatgtgttttagacctgcaagacactatctgttcaatggctgaggtgagggtctggactacagattttttataaagtatatgcagaaaaattacaaatcactaggaattctttcagttgtgaagaatgtctgacataagatttgaagtgctacctttccagcttatatattaatttgcttatatatttgatatgaataaatgctttttttctcatgggtccttgcgaggctcagagatttatgaatccttttgtttcacattcagaatatatcacatctctaaatatgcagcaaataagtctcctagtgagtgactatagacttgctataatagcaattagccaggcttccagaaaccccagcacatcccataagaccctgggaataagacctaaactcctggggttagtgtataggatatcaaggtgaataatcttgacgcagttataaagatttaatgtacaacgagtaatcagcatttctggactccttttcttttccatttgctttggaaatgtattctgaagaaattcctacaattagagctccctcatttctgatgaaaacctggtgagtacctgtaactattttgtactgtatcttctccaggaagtcccagatgggaacacagctcctgtccaaagtataatatcttttctttttttttttgagatgaagtatcactctgtagcccaggctagagcacagtggcacacttggcacgatctcagctcactgcaacctccacctctcgggttcaagcaattcttctgcctcagcctctcaagtagctgggattacaggcgcctgccaccacacccggctaatttttgtatttatagtagagatgaggtttcaccatgttggccaggctggtctcgaactcctgaccttgtgatctgcccacctcggcctcccaaagtgctgggattacaggtgtgacccaccgcgcccaaccaatatcttttctttttgagagagagtttcgttcttgtcacccaggctggagtgcaacagtgcagtcttggcttactataacctccgccttccaggttcaaacgattctcctgcctcagcctccagagtagctgggataacaggcatgcaccaccacgcccggctaattttgtgtttttagtagagacggggttttaccatgttggtcaggctgagctcgaactcctgacctcaggtgatctgcctgcctcggcctcccaaactgctgggattataggcatgagccaccacgcccagccccaaagtataatatcttaaatctttaaactatcctaaaatgcaagcaaggtgggcctactccatataccaacctacaaggggtgactgactctcccgcaatatgtcatcatacttgatagcatttgaaaagaggcacgttaaggacgtgcctttgattcttctttcccaccaaggtcacaaaacgtgtcagggtcaactaattcaacccagtggtggaaggtggaagaggaataaagacgaactgagactaaattctttcacgtttctcttttccttccctggccaaaagaaacttgagaccactaccagtaagataaaactgtaaacattctttttttttttttattaagaaagcatgcaaaaaaacaatgacaacaacttgtgagaccatatacccccttcccaggatgcagcaatgcaaagcattatgggtggtggcacaaaggactcaccaaaccagaaacatgaggagcagggactgaggagagaaaggttgggatgggtggatctataccaaataattactccttttgtccctctcctccactttctctcttatttcctcttataggcagtgagcttctgcagttttgcacacatcacccaatacaacaataatgactacactttgtgtccagcaatgctagaccaacaagaaaaagtagtattcttttcatttcatgaaggggaggggaaactgaggcccaaagggagtcaggtttgccctgtgccacgtagcaagactgggcttggtggaagggagctggaatctcaggaggcctaatggccaatctccagtcccagacccaaacttcagctaccctggctgcacgccagaattcttcatgagactagtttgcagaagacacagatgaacaaggggcatttgcatgtatgatcctacccttgcaaatcctgtatggatctgaacaggaaaagggtttagaaaagagaagtgatcatttacaaatggggaatattgaggaga

SEQ ID NO: 6 - protein, 482 aa, Homo sapiens
MASFSAETNSTDLLSQPWNEPPVILSMVILSLTFLLGLPGNGLVLWVAGLKMQRTVNTIWFLHLTLADLLCCLSLPFSLAHLALQGQWPYGRFLCKLIPSIIVLNMFASVFLLTAISLDRCLVVFKPIWCQNHRNVGMACSICGCIWVVAFVMCIPVFVYREIFTTDNHNRCGYKFGLSSSLDYPDFYGDPLENRSLENIVQPPGEMNDRLDPSSFQTNDHPWTVPTVFQPQTFQRPSADSLPRGSARLTSQNLYSNVFKPADVVSPKIPSGFPIEDHETSPLDNSDAFLSTHLKLFPSASSNSFYESELPQGFQDYYNLGQFTDDDQVPTPLVAITITRLVVGFLLPSVIMIACYSFIVFRMQRGRFAKSQSKTFRVAVVVVAVFLVCWTPYHIFGVLSLLTDPETPLGKTLMSWDHVCIALASANSCFNPFLYALLGKDFRKKARQSIQGILEAAFSEELTRSTHCPSNNVISERNSTTV

SEQ ID NO: 7 - DNA, 49817 bp, Homo sapiens
  source 1..49817: mol_type = genomic DNA
gcagatagattgattcagtcagtcaggtcaaggttaacttgaattaatcagtaatagggtggaagaaggggatggccttgctgtgggttctggagaaaaattctaggaaagcagccacctcagcctggaattagacgatgggataggggtttcccagctgctcccaggcctggctgcccctttgttggggaaggggagggatgggatataggggacagtgagtgaactcaggcaggtgtgagccgggggcatctgggtcccccacccagaaatcattcccacttccttcctcttattttctttctttttcctgtcttgctctgtcattcaggctggggggcagtggtgcagtcatagctcagtgcagcctctaactcctcctgcctcagcctcccgaggagctgggactgcaggcacgccaccatgccctgctaatttttttttttttttcaattgtagagacgaagtctcactgtatttctcaggctggtctcgaactcctggactcaaacaatgctctcacctcggcctcccgaaagtgctgggattacaagcacgagccaccgcaccctggccccttctcattttccccttgcaccccagctaggattgccaaacagaatacaggacgctcagttacatttgaatttcagataaataacaactactttttcagtatatgtagcttccagataacccacgaatggtcagcccggttggccacactctccctccttgattccgggaatgctgggctgggtgggcctcaaaatggaaagtaccccaacacacacccagacctccttctctccctcccctgctggctcatccttgtgcactatccccctcccaaacctctggacaccaatgcacatctcccagaaaaaagtcacgaggttctgaagaattcccggtctcatctccctccctccttccctcccagtaggctaccatctgctccagcctccaaccccctcacttctcatcctgcccctcccctctggtcacttcttggaggtcagggtagggccagaccctttccaggttcaagtgattctcctgcttcagcctcccgagtagctgggattataggcacctgccaccatgctcagctaattctttgttgttgttgtttgttttgttttgttttgagacagagtctcgctcttgtcgcccaggctagagtgcagtggcacgatcttggctcactgcaacctccgcctcccaggttcaagtaattctcctgcctcggcctccccagtagctgggattacaggtgcccgccaccaatcctagttaatttttgtatttttagtagagatggggtttcaccatgttggccaggctggtcttgaactcctgacctcaggtgatccacccatctcggccttccaaagtgctgggatgacaggtgtgagccaccatgcctagccagctaatttttgtattttttagtagaaacagggtttcaccatgttagccaggctggtctcgaacccccgacctccagcgatcccccagcctcagcctcccaaagtgctgggatgacaggcgtgagccaccacacctggcccctctgagcctggtggcttctaggcatcctggtttctttaattgtcacaacaaccagaactatcttcagtcgcattgtttagttggattaaccgaggctcagagaaaagaggaacccaggcttgccgctagacagaggccagacaggaattccttctcaaggttgtcaaaccacagtgccgaatgcttgagtctagaatgaaaccaggaaatggggtggcttgaggagaaagtgggggatagaagatggaatggggcaattgggagatccagtttctttcctttttttaatttttttttttttttttggcaacagggtctctctctgtcacccaggctggagtgcagtggtgcaatctcagctcactgcaacctctgcctcccggcttcaagcgattctcctgcctcagcctcctgagtagctgggattacaggcacccaccaccacgcctggctaatttttgtactttcagtaaagacggggtttcaccatgttggccaggctggtctccaactcctggcctcaagtgatctgcctgcctcggcctcccaaagtgctgggattacagacgtgagccactgcgcctggcaaggggatgcagtttcaaaagctgaaccccaattctggagagcaagcaggtattttcattctctctcctcctcctcctcctcttccaaagagtgtgtcgcaatcagtgcagacagacgccaggtttgttctcatgctccacgcctccccctacccctggcacggaaaagaatgtggtttacaggaaatcagagaaaactccccattaaccccttcagtggggtttcagaaaccgcctctccagggataagggggccccacccacagacccttctcctgccctcaccatccacctcgtatgcctgggcagcaatgctgcagaacgtcagaggaatgccagttaaaatgacaccggctgccggggtgtggtggctcacttctataatcccagcactctgggaggccgaggtgggcggatcacctgaggtcaggagtttgagaccagcctggccaacatggcgaaaccctttctctactaaaaatacaaaaaataaaaaataaaagaaaaaaaaaattagccaggtgtagtggcgcatgcctgtgatcccagctctttgggaggctgaggcaggagaatcacttgaacccaggaggcagaggttgcagtgagctgagatggcgccactgcactccaccctgggtgacagcacaagactccatttaaaaaaacaaaacaaaacaaaaaaaatgacaccagggtaccagttttcacccataaggctggcaaaaatcttcaagttcatcaacatgcccttgtgatgaggctgtggaagaaactgacaattcatttcatgcagggctcataagtgtgtaaatcaatacaacttctgtgcaggggaatttggcaatatctagcaagattaccagtgcattcagagattgacccaacatatttcctttcattgcaacgacaactctatgaagcaggtggtaagggtttccttttccatgaacaaactgaggctcagggcggtaatcagtagcttacccaaagatcacagctagtttcagagctagaaaataacgcaggttcaagcttattcactgcagagagcctggtgtgaagccacagatgtcagtctctccatcaagaagaggctggtggctggacacagcggctcacgcctgtaattccaacactttgggaggccaaggtaggtgggtcacttgaagtcaggagttcaagaccagcctggccaacatggtgaaaccccttgtccactaaaaatacaaaaattgccagacgtggtggtgctcacctataatttcagccattccggaggctgaggcaggagaattgtttgaacccaggaggtggagggtgcagtgagctgagatagcgccactgccctccagcctgggtgacagggcaagactctaaaaaaaaaacactcaaacaaacaaaatatcccccaaaaagtaggaggctggttactttctcacaatataacaagaggcctgtaacctgtaagaatgaggcagttctttgctcactgaggtgaaatagcctctgaggtatattgttcatgaaaaaacgaaacaaaacgaaacccaagatttaactgaagagaccaggaagaatagtatgtgctatgtgctgtccacagggcacagtagttcacaccagcactttgtgaggctgctgcgggaggatcacttgagcccaggagttcaagactggactgggcaacatcgtgggacccccatctccacaaaaataaaaaaattatccgggcatggtggcggccacccgtagtcccggctacttgggtggttgagccaggatgatcacttgaccccaggaggttgaggctgcagtgagctgtgattgcaccactgcaatttagcctgagtgacagaatgaaaaaaaaatttttttaaaggaaaacacaaaaagaatatgctgtcaacagggatgggaggaagaccacctttactgctatacacatttgtaccttttagatgttgatcaatatgaatatattatacacacagacacacacacagacacacacacacacacaaacaatacaatttaatatcctaagaggatattgacattagacaggtacaaaagctctagaaatgaggactttcctcagtgatgacttttttcaccaccaaagtcactcaggcatcctgacaagggtaagtgaggggagcctccttggaaaataaactcacttggatagtgaactcctgcacatacctcaaagcccatctgaaatgtcccctcctacaggaagttttccctgaccctccaagaagcagagttctatttcactggggaaaacatttcttcttcttcttttttttccctgccctgcacatgagctagaaaacatttcatgaaactgggagtttctgtgctgggctctgtccctcccccattctacttcccctccctcagcatggaagcctctggaagtggggctctgactcccagcctacagagagattcctaggaagtgttcgactgataaacgcatggccaaaagtgaactggggatgaggtccaagacatctgcggtggggggttctccagaccttagtgttcttccactacaaagtgggtccaacagagaaaggtctgtgttcaccaggtggccctgaccctgggagagtccagggcagggtgcagctgcattcatgctgctggggaacatgccctcaggttactcaccccatggacatgttggccccagggactgaaaagcttaggaaatggtattgagaaatctggggcagccccaaaaggggagaggccatggggagaagggggggctgagtgggggaaaggcaggagccagataaaaagccagctccagcaggcgctgctcactcctccccatcctctccctctgtccctctgtccctctgaccctgcactgtcccagcaccatgggacccacctcaggtcccagcctgctgctcctgctactaacccacctccccctggctctggggagtcccatgtgagtggttatgactctacccacaaacagggctggttctggggtggaagcagacatttgggggtccaggtccctgtagaattcagggtgcatttgggtgtttgtggattcaggggttagcaggttgggaatgattatatatatttgggctgcctgtgagtttgggtgtttgtggttgggtgtttgtggaatccaggtatcatggaattggagtttatatacatttgggctgcctgtgagtttgggtgtttgtggttgggtgtttgtggaatccaggtatcgtggaattggagtttatatacatttgggctgcctgagagtttgggtgtttatgggttgggtgtttgtggaatccaggtatggtggaattggagtttgggatgtttctagaattgaggtcatctgttggtttagggtgtatgtggtgttcattgatggtgcggttgggggtgtttggagactcggaggtttggactttacaagatttgggagtttgcagcttggggacttgcaattttcagtgtgggtttaaagattggctacttcgggttcatgtatagttggggcatttggaattgattgtatttattaggactggggtgttggaggtttaggctgggtttggggtgctctaagatttgaggtttagaggttttggcgtatgtgggtttgggtaggtagagttgagggtgtccgggagtttgagtgtttacatatttggagtgtttagagaggtagaggtttagggtttggggcatgtgtgggtttaggcgattgtgggtctggaagtccagagacttggaggagttgctgacgctggttggaaggttcagggtttggtgggatgtgtggccccctcgttgcccaggctttcaaaggccaggcccagctggctgagagtgggagtcatggtggctgctgtcctgcccatgtggttgagacggtggcagtgcccagagaagataatggcattggcaagtgcgccggcagtcactggatcctctccaggaccagaggctggggcacacagcctgccaggcgctgactccagtgaggactggcgtctcacatccgtggaatgacaagcccactcccgtgccccactccgacaggtactctatcatcacccccaacatcttgcggctggagagcgaggagaccatggtgctggaggcccacgacgcgcaaggggatgttccagtcactgttactgtccacgacttcccaggcaaaaaactagtgctgtccagtgagaagactgtgctgacccctgccaccaaccacatgggcaacgtcaccttcacggtgagtgcagactggcgcaggacccggctgacacccacagccacgcccactccccccctcctcctgagcccctccccttctgtcttctccctttctaagccctgcccttccctgagactccaccccttcggagtcgcctctccttctaagcccctcccttctctgagactccaccccttctgagtctcctccccttataagcccctcccttttctgagacccccccccaccccttctgaatctcctccccttctaagccctgaccttccctgagaccccaccccttctgagactcctccccttctgagtccctcccttccctgagaccccaccccttctgaggttcctccccttctctgagactccaccccttctgagtctcctccccctctaagtccctcccactgaattccttttccaagcccctccccctcgaagtctcctcttctgaactcctcccctcttagtctccatcactttctaagttccctcacctgagtccctccccctttctgagcccctcccatgtcagccccttccctttctgagtccccgccccttctgagcccctcctcctataagctctctcctccttgtgagctcttctttttgagttccctccctggtcccccctctcccctcgcacctccttcacatgcccctccctccccaaaacggccacctcggaagaccaagaataatgggcaggcaaggagggacccagcccaagatccggaagctggaccgtgggcatggggccttggaacagacccctgacaatgccctgcccacgcctagatcccagccaacagggagttcaagtcagaaaaggggcgcaacaagttcgtgaccgtgcaggccaccttcgggacccaagtggtggagaaggtggtgctggtcagcctgcagagcgggtacctcttcatccagacagacaagaccatctacacccctggctccacaggtgaggctgggggcggctggagagggcggggcaccggcgtgggcgggctagggtctcacgaggcctctttgtctctccccagttctctatcggatcttcaccgtcaaccacaagctgctacccgtgggccggacggtcatggtcaacattgaggtgccagccagagggggccccaggggaagcaggggcacaggcttaggagaggcaaagagatcgagagagacagagaaagacacaccggaaggggtgcagtggcagagacacagaggcaaagagatatgcagacacacacccacacaacacacacacatacagcacacaacatgcacacacacagcacacaatacacacacagaggcaaagagatatgcagacacatgtgcacacacaatgcacacacacaatgcaacacacacaaacacacaacatacacgaccacacaacacacacaacacaacacacaacacaatacacacagcacaacgtgcatgaccacacacacaacacacaacacacacaacacaatacacaacatacacaaccacgcaatacacacaaaacacacacaacacaacacaacatacataaccacaccacacacaacacacaaccacacaacactatcacacaacacacacaaacacacacaacacacaacacacacaacacacacaaaacacaacacacacacaacatacacaaccacacaacacacaaccacacaacatacacgaccacacaacacagtgcacacaaacatagcacacacaacacacaacccaacacacaaccacacaatacaccatatggcgcgcacacacacacacacacacacacaggctgagagacaaggtggagatccagggagaccccagggagcagtgcaggtgtccgtggattctgctttcagttaaacccctgatcacttcacctccctgagcctcagttaccttatctgaatatcgggatcatgacggataattgtatgtcatctattctaccgacggcagccagaggacgcctgtgagcacctgagtcagggcccatccctgctctgcctacagccctccatggctcccaccttcctatgcgtcaaagcccaagtcctccctgcagtccacaaggccctgcacaccttgccctgtcccttccctgccctcccctcctccctctctccccctcgttcactcttctggagccacacgggccatcctccctgttcctccaacacccaggtgcagtcctgccttggcgccttggcacgggctgtgccctcttctcaagaaaaccctcttcttccaaatatccacacagcttgttctctctcctcctttaagtctttgctcaaatgtcaccaatgtctcaattttacaatgaggtctctctgagtaacctataaagtcgcaaatacccaccctgagcgtcccccctccccgctacacacactcctccttcctgccatgtcctgcaaatgagatttattcatttgataattgcttctcccatcgcctcgccctctattgaacctaaatccctccaggaaggaattgttatgtttgttgagggttttgtcacctgaactcagcacaatgctggtatatagttgggtttaataaaaaacttactggaagaagcgagaaggatgggaggagagaaggggaaggagggtgttctcatagaattatcatgaggatgtgttgaaatcatacaaggctaggtgcagtggctcacacttgtaatcccagctgtttgggaggccaaggcgggaggatcgcttgagcccaagagtccaagaccagcctgggcaacacagccagaccctgtctctacaaaaaagaaaagttaaaaacaaacaaaaaaacagctgtgtgtggtggtgcttgcttgtggttgcagctaccccaggaggctgaggcaggaggatcacttgagcccaggaattccaggctgcagtgagccgtgatcgcaccactgcactccagcctgggtggcagagtgagaccctgtctcaaaaaataattggggcaaatgcaatggctcaagcctgtaattccaacatttcgggaggcagaggtgggaagactgctcgaggccaagagttcaagaccagcctgggaaagctagggagactacatctctacaaaaaaaatgtaaaaattatctagatttagggattgatgtggtctgtggggaacagagaccacacatctcttgtaaaggcacaacagttgcccagctccaattagatgtctcctgctaaccagagtacactatccacagaaatttccttgtttccaacagaagctagaaaaacagatttttggccaggtgcagtggctcactcctataatcccagcactttgggaggtggaggcgggcagatcacgaggtcaggagatcgagaccatcctggctaacacggtgaaaccccgtctttattaaaagtacaaaaaaaaaattagctgggcgtggtggcgggcacctgtagtcccagctactcgagaggctgaggcaggagaatggtgtgaacccgggaggcggagcctgcagtgagccgagatctcgccattacactccagcctgggcgacagagcaagactccgtctcaaaaaaaaaaacaaaaaaaacaaaaaaaaaacagatttttatatgttttaattcctaaagccagctcacggccttcagatatgccacttgcctgatccctgttacctctgtacaatttcttttaaacttatttattcattcattcattcattattattatttttgagacagggtctcattctgttgcccaggctagagtgcagtggcacaatcacagctcactgcagcattgacctcctgggcccaagctgtcctcctgtctcagcctcctgggtagctgggaccacagacgtgcgccaccacatccagctaattttaaaaaatttttgtagagatggagtctccctacatttcccaggctggtcttgaacccttgaccttgagcaatcttcccacttctgcctctcaaagtgctgggattacaggcttgagccattgcgctcgccctaatacattattttttgagatggggtctcgctctttcacccagactggagtgcagtggtgcaatgatgtctcatgatgtttaaatgttggcagcaaatgaaatgacactactagttattagtattcagagagacactgaaaaaatgagcccctactcatatgaactatgtcccaagccaacacagtaggtgccattataatctcctgtttcaagatttgcacattgagcacagagaggttaggtaacttgcccagggtcacacagcttgtaagtggcacagtagagattgaaacctaaggttgactgactccggtccttgttctttttttcgagacagactctcactctgtctcccaggctggagtgcagtggagtgatcttggctctctgcaatctccgcctcccgggttcaagcgattctcccgcctcagcctcctgagtagctgggattacgggtgcctaccaccatgcctggctaatttttgtatttttagtagagacagggtttcatcacgttggccaggctggtcttgaactcctgacctcaggtgatctgcccgcctcagcctcccagagtgctgggatgacaggcgtgagccgctgcgcccacctgggtccctgttcttaaccacagtagacactgtgcacagagaatgtccagacacaggtcggggagagctgagaggctaagcccagcctccgaagagccactttatcctctatccttccctcctgcctcccacagaacccggaaggcatcccggtcaagcaggactccttgtcttctcagaaccagcttggcgtcttgcccttgtcttgggacattccggaactcgtcaagtatgtcaggttcttgaggagggggctcagggctcccctatccccggagagggagcaggggggctccgaggcctgagagaccactcatccgccctcctcacagcatgggccagtggaagatccgagcctactatgaaaactcaccacagcaggtcttctccactgagtttgaggtgaaggagtacggtaagaggaggaggggctggggggagtcagtgcccagaacgcctggcccagcgccggccccaccaacgccatctctcccccagtgctgcccagtttcgaggtcatagtggagcctacagagaaattctactacatctataacgagaagggcctggaggtcaccatcaccgccaggtgagggactgggggtggggccaggtaagagccaggtgagggaccaggtgaagaccaggtgggggactgggggtggagtcaggtggggggctggagatgggaccaggtggggggctgggggtggagtcaggtggggggctgggggtggggaaggtggggggctgggggtggggcaaggtgaggggctgggggtgggaccaggtggggggctggggggtggagtcaggtgggggctgggagtggggaaggtggggggctgggggtggggccaggtgaggggctggaggtgggaccatgtggggggtgggagtggggcaaggtggggggctgggggtggggccaggtgaggggctggaggtggggccaggtgagaggccagcagtgggttgggggctccagtcttcagcacaggcaggagaagctgggggagatcccattctccaggagggatggacctgaagccctccttgtctgtcccgtaggttcctctacgggaagaaagtggagggaactgcctttgtcatcttcgggatccaggatggcgaacagaggatttccctgcctgaatccctcaagcgcattccggtaccatagacggaggccgctttgatccctgccccagtccccgccacctctgagcccgctcccctctctgagccctcctctcccttctcagattgaggatggctcgggggaggttgtgctgagccggaaggtactgctggacggggtgcagaacccccgagcagaagacctggtggggaagtctttgtacgtgtctgccaccgtcatcttgcactcaggtgaggcccagtctgaaggccaggctcaggaccaccaagtgggccggtctgagaggggagaccaggtcagaagagaaagcctagtctaaggagggaggctcagagtgaaagtggggttcagtctgatggggtaggcccagtctgagaggggaggccgagtatgaagatggattccagcctgatggggggaggcagggccagtataaaggtggggtccgggctgatgggggcacaggcccagtatgaagtctgtgtccagtctgatgagggaggcagggccagtataaagatgggtccagtctgatgggggaggcagggccagtataaaggtggggtccggtctgatgggggtcacaggcccagtatgaagtctgtgccagtctgatggaggaggcaaggccagtataaaggtggagtccagtctgatggggggcacaggcccagtatgaaagtggactctactctgagggaggaggtctagtctgaagttggggtccattctgagggaggaggtctaatcctgaggggtggcccagaagcctacactcacagctggtcccctcaggcagtgacatggtgcaggcagagcgcagcgggatccccatcgtgacctctccctaccagatccacttcaccaagacacccaagtacttcaaaccaggaatgccctttgacctcatggtgagacccggggcgggaaggggtcccactcctcccttcggggacaccggccacagccctgagcctgcctgaacttcccccacctgcaccccacatcacaggtgttcgtgacgaaccctgatggctctccagcctaccgagtccccgtggcagtccagggcgaggacactgtgcagtctctaacccagggagatggcgtggccaaactcagcatcaacacacaccccagccagaagcccttgagcatcacggtgcgtctgggcccagcctcggaaccccatcactgggaagacggtacaggggttctggtgtttgcacagtggggtcctgtcatttgcatacagatattctcatctgcatagagaggttctctcctgcgcagaggggtcctgccatttgcatagagatactctcatctgcatagaggggttctgtcctgcacagtggggtcctgccatttgcatagacattctcatttgcctagaggggttctgtcctgcacagtggggtcctgccgtctgcatggaggggtccgcagtttgaggaaacaggaatcttcctcttgcatgccctgctccttccacttacacggagaggcgctccatccacgcacagtctttccactcccatgggggaaggagcctgaatctcacaaggagggttgtgtagtgtttgggacaggcccattgttgtgaggtggtctcagttctcctggcttctgtgcacgtggctctgttgcccctcactgggagggaagcaagtctcatgacagctgcggaggttgcagatggcctcccagtccctctgcagctcccaggctgcgcaccccacttacccctccctgtgctcagcatgtgcgtgaatttccggtggctaccatgagaaatggccacagcctagtgatctaaagcaacacacatttatgggtctatagtttgagaggtcagaagtcctggctctgggggaaagttcgctcccttgctttttccagtgtcgccagggcaccctaaaggcctggctcatggccccttcctccacctttaaaggcagcagcatagcatcttccagtgtctctctttctctctgtctctgtctctcctttctcccctgcccctgcttaataaagacccttatgattacattagctccacctacataatccaggataatgattccatctccagatccctaacttaatcccatctgcaaagccccttttgttaagaaaggccaccaattcccaggtctcagggattcgggtgtgggtatcctcgggcggcgaccagcaggcatccctctttccccacccaggtgcgcacgaagaagcaggagctctcggaggcagagcaggctaccaggaccatgcaggctctgccctacagcaccgtgggcaactccaacaattacctgcatctctcagtgctacgtacagagctcagacccggggagaccctcaacgtcaacttcctcctgcgaatggaccgcgcccacgaggccaagatccgctactacacctacctggtccgtggccacctggaaacctcagcccccgcctcctccttgtttcttccgcacccctgggactccttcccccatcccggatccctccctgcgttccctgccactcaccctccccagcctgatgccagcctgtccccccagatcatgaacaagggcaggctgttgaaggcgggacgccaggtgcgagagcccggccaggacctggtggtgctgcccctgtccatcaccaccgacttcatcccttccttccgcctggtggcgtactacacgctgatcggtgccagcggccagagggaggtggtggccgactccgtgtgggtggacgtcaaggactcctgcgtgggctcggtaagtgtgccctgggctcgctcgccccctctccctctccctactcctctctctctctctctctccctgtctcctctctctctctctctccctttctccttttctctctcctttctctctcttctcttcctctccctttctctcctccctctctgtctctcaactgtctctctttttatctctctttccctctctctacatctctctttccctctctctttatttctctttccttctctctctccctctctcgatctctctttctctccatctctctccttttctctctccctctctctctccttttctctctccctgtctctttccctttccctctctctcccctctctttctctccctctctctttccctctccctctctctctccctttctctctctccctctctctccttctctctccctctttctctccttctctctttccctctctctctccctctctctttccctctctctccctctccctttctctccctctttccctttccctctctcccccctcactctccctctctctgtctctccgtctctctccctctctccctgtctctccgtctctctccctgtctctccctttctctctctctcccgccctctctccctctctctccctccctctctccctttctctctctctccctctctctccccctccccagccccacggctccccccaacctttctgtctttccactctagcccagcacccactccatcccaggcactcctctctcccagggctgacttctttcggcgtctccaccctccccacagctggtggtaaaaagcggccagtcagaagaccggcagcctgtacctgggcagcagatgaccctgaagatagagggtgaccacggggcccgggtggtactggtggccgtggacaagggcgtgttcgtgctgaataagaagaacaaactgacgcagagtaaggtaagggccagtgacccaaggctgctgagaagaggcggaggcacggagctggggctgggggaggtgggtgggactggagagggcagtgcagtggggggcatgcgctgaaagcagagatcggagcagaccagacacagggatggttgaagctgaagatgggaatgaggttggacatgggttccaattggggatggtcctgagaattggacttttttttctgtttgtttgtttgtttttgagacagagtctctctctgtcaccaggctggagtgcagtggcacaatctcggctcactgcaacctctgcctcccaggttcaagcgattctcctgcctcagcttccctagtagctgggactacaggtgcccatcaccacgcccagctaatttttgtatttttagtgaagacgggggtttcaccatgttggccaggatggtctcgatctcttggccttgtgatccacccgcctcgacctcccaaagtgttgggattacaggcgtgagccactgcgcccggctgagaattggacactttcaactggggccctgagaggctggtggcagcacacccagggtcattcagtggggaaggtttccggagtagggacgaagatggagatggggttggcttgggatcaggagtgaggatgggaatgcagatggaatcagaggggaaatggagataagatttggaatggaggccaggtgcggtggctcacgtctggaatcccagcactttgggaggtcaaggtgggaggatcacttgaggccaggagttcagaccagcttgggcaacatggcaagaccccatctctacagaaaaaattttaaaatagctgggcatgatggcgcatgcctgtagtcccatctgctcaggaggcagaggtgcgaggattgcttgagcccaggaatttgaggctgcagtgagctatgcctgcaccactgcactccagcctgggagacagtggaaaatcccaacttaaaaaaaaaaaaaaagaatggaaagaaaggaggaaaaaaaaagaagagagagagaaacagagagaaagaaaaagaaaggagataaagaggaagggagggagggagtgaagaatgaaggaaggaaagaaggaaggaaggaaggagggaaggagggaaggaaagggggagcaaaggaaggaggaaaggaggaatggagggaggaagggagggagaggaaggaagggaaagaaagaagacagaaagaaaagaaaaagaaggccgggcatggtggctcactcctgtaatccctttgggaggccaagcactttgggaggccaagacaggcgaatcatttcaggtcaggagttcgagaccagcctggccaacatggtgaaatcccgtctctactaaatatataaaaattagctgggcatggtggcatgcacctgtagtcccagatactcgggaggctgaggcaggaaaattgcctgaacctgggagttggaggttacagtgagcggagatcacaccactgcactccagcctgggtgacagagcaagactccatctcgaaagaaagaaagagagagagtgagaaagagaaagaaaaagagaaggaaggagagagaaggaaggaaggaaagagaaagagaaaggaagggcagaagcaggaatgggggagatgagagtgggacagggtggggtcatttgggaagagatacacaggtgcatatgtgggggatcccaattgtcagcctggcctccctgcgtcccgccacccctatgccccccgcagatctgggacgtggtggagaaggcagacatcggctgcaccccgggcagtgggaaggattacgccggtgtcttctccgacgcagggctgaccttcacgagcagcagtggccagcagaccgcccagagggcaggtgaggtcgccaccaggggccggtgcagggacagacagcacctccacctcccagatgctgggagcagagctctggaaaccgggggcctgggttcaagccccgcctccaccaccacctagtaaatccctcccctctgagcctcagtttgctcttccatcaaatgggagcaggaacacccccacctcacacgatcgtgaggggtgaaccgaggacacctagtaggtgcctcatccatcttcttctcggtccgcctgccctgcagaacttcagtgcccgcagccagccgcccgccgacgccgttccgtgcagctcacggagaagcgaatggacaaaggtgggagcctttcctacccactcctgcccccgagccccaccccaggagaccccagcccggccgtgcaggagccagagagggaggaggggaggccctggcggcggggaagtcctccctggggtccgtcccgcgtccctcctgctgccggcccccggctgagggtgtggcctgggggaacacgtgctcccgcagtcggcaagtaccccaaggagctgcgcaagtgctgcgaggacggcatgcgggagaaccccatgaggttctcgtgccagcgccggacccgtttcatctccctgggcgaggcgtgcaagaaggtcttcctggactgctgcaactacatcacagagctgcggcggcagcacgcgcgggccagccacctgggcctggccaggagtaggtcccacggggtggggacagggggagggggccgtctgatgggggaggagactcctgtctgaggagggaggatgccctgtctggtgggggtggggctggaggaggccgctgtctgaggggggaggaggcccctgtctgagggggcaggaggtccctgtctcaggggggaggaggcccctgtctgaggagggaggaaacctccgtctgaggagggaggaggtccctgtctgaggagggaggaggccttgaggggggaggaggtccccgtctgaggagggaggaggcctctgtctgaggagagaggaggtacctgtctgaggggggaggaggcctctgtctgaggggggaggatgcccctgtctgagggggtaggaggaggcctctgtctcggggggaggagtcccctgtctgaggagggaggaggcctctgtctgaggggggaggatgccgctgtctgagagggtaggaggaggcctctgtctgttgggagaggaggcccctgtctgagggtgatgccgatgaggtgatgccctgccagcgtgaggtagagaagacccaggtctgaagaggggaggatcaagtcagagaagcgtagatgcccatctgagatggaggaggctcccgtccgaggggaggggacactcctgtctggaagggacagaggccttcagatgaggagccaggaggcccaggcctgagggaggagaagggcctagtctgatggggagaagggcccttgcctgaaggcagagcagtttcctgcctgggaaggtcatcccagccccacccatcagtctgaattggacatcaccagtgcccaggacattggaggtctgagggaaaagtctagaaagatgatggggctggtcacacactaattaccaatgggaaagctaaggtgagttccaagtttggcttcaccagagaaaactaatttgtgtggcattccagaaagacctgccaaactcgatgagtgaacaggcagcccttcttcattcatgcatgcattcagtttttgaatcaggtgagactttagatctcacgtgaaataagtcttaagtgaaacaaagagaaatttatcttataataagagaaaattggccgggcatggtggctcacaccggcaatcgcagcactttgggaggccgaggtggatggatcacttgaggtcaggagttcaagactagtctggccaacatggtgaaaccccgtctctactaaaaatgcaaaaatagcctggcgagctggcaggcgcctgtaatcccagctactcaggaggctgaggtgggagaatcgcttgaacctggtaggtttaggttgcagtgagctgagattgtgccactgcactccagcctgggcaacagagcaagactccgtctcaaaaacaaaacaaaacaaaacaaaaaaagaaaggaaaaagaaaattggccgggcacggtggctcacacctgtaatgcccacactttgcgaggccgagaagggtggattgcttgagtccagaaatttgagaccagcctgggcaacatggcagaaccccatatctacaaaaataaaataaaataattagccgggtgtgggggtgcacacctgtagtcccagctactcaggaggctgaggtgggaggatcgtttgaacccaggagatggaggcgtcaatgagccaaaatcacaccaccgcactccagcctgggcaacagagcaagaccctgtctcaaaaaagaaaaaaaaaaaaagagagagaaaagaaaagaaaatgaaaagaaaaaattcaagcaaatttagaatgatctccttcacaaagaggcgatagtgtgagggtcactgggaaaattagacaaaaagtctggtctactgaaatatggtttacatccacatggatggtgggctgtacttttctccagaattgtgtaattcctttggcccattgggggtcagaaaaagaatggctaaatgttactatcccaagacacttggattgattattccagagtgtgagtaaattcaggtatctcttttaggaattccatctactttgggctgggcttagtggctcacacctgtgatcccagcactttgggaggctgaggcagcgggatcgcttgagctctggagtttgagagcagtctgggcagcgtagtgagactttgtacggacgaaaacttttttttttttttttgagatggaatcttgctctgtcacccaggctgaagtacagtggcacaacctcggctcaccgcaacctccacctcatgggttcaagcgattctcctgcctcagcctcctgagtagctgagattattattatttgtttttttgagatggagtctcgctctgtcacacaggctgcagtacagtggtgcaatcttggctcactacaacctccgcctcccgtgttcaagtgattctcctgcctcagcctcccaagtagctgggattacaggcacctgccaccacacccagctaatttttgtatttttagtagaaaagaggtttcaccgtgttggccaggctggtgtcgaactcccaaccttcggggatctgcccgcctccgcctcccaaagtattgggattacaggcatgagccactgtgcctggctgaaaaatattaaaatatatatattttttaagggattccagctactttgttgttatggagatccagaacccaattaaagcctgtctatcatgtttgaggaaagtgcagtttgagtcaaagcctagtccagtccaatttcatttacttgctggtagtgtcaagctgtttttgtttatttatatatttatttagaggcaggatcttgctctttcgcccaggctggagtgcagtggtgcgatcacagctcactgcagcgtcaacctcttgggctcaaggagtccttctgtctcatcctcagccttctgagtagctaggactacaggtgcatgccagcatgcccagctaatttttaaattattatttgtagagagagggtctcagtgtgttgcccaggctggtctcaaactcctgggctcaagccatcctcccaccttggcctctcagagcgctgggatgatagcaccacatccagcctatcgagattttttttgtgtttttttctttgttttttgtttgtttgtttgtttgtttgagagggagtctcgctctgtcgccaggctggagtgcagttgcgcagtctcggctcactgtaacctccgcctcctggattcaagagattctcatccctcagcctcccgagtagctgggattacaggcgcatgccatcacacccagctaatttttgtattaggtggtttttaaaggccaccgcttcttcagtgttctgcaccaggtctgggaatgttctcagctcacctagtcatgttcagaatggacaaatccctcagaggaagcagacacggtttctcgggacggtgatcctttagagccacatgcacatgcttgctttcttttattattatctttttttgagatggagtctcactccgtcaccgaggctggagtgcagtggcataatcttggctcactacaacctctgcctcccgggttcaagcgattctcctgcctcagcctcccgagtatctgggactacaggtgcccgctgccaagcctggctaattttcatatttttagtagaggcgcggttttgccacattggccaggctgtctcgaactcctgacctcaagtgatccacccgcctcggcctcccaaagtgctggaattacagatgtgagccactgtgcctggccaaatgctttcgtttctttaaaaatcaaagggaaaggaatgactataatccagtctgcattgtatatgtccttataccagtacatttgtgggatataatttttagttctttttatggagaagaagttcccaaggcagatgtgtctggggctcgtgaaaattcatcctgaagtcctccatgtccgggatgtatttcactgctaggaatccctcctgggcagaggtaggatctaaaggtgtgaccgctgaggaagtaggtcggctctctttttgtttgttttttgtttttgttttcagatggagtctgtctctgtcgcctgggctggagtgtagtcgtgtgatctcagctcactgcaacctccacctcctgggttcaagtgattctgctgcctcagcctccacagtagctgggatcacaggcacgcgccaccacacccagctaatttttgtgtttttagtagagatggggtttcaccatgttgtccaggctggtctcaaagtcctgacctcaagcgatccacccacctcagcctcccaaagtgctgggattacaggggtgagccaccgtgcccagccttaatttttgtatttttagtagagatgggtttcaccatgttagctaggctggtctccaactcctggcctcaagtgatccacctgccttggcctccctaagtgctgggatttcaggcatgagccatggcaactggcctgctctgttctaaatgcagatctaaaccccctgcaggtaacctggatgaggacatcattgcagaagagaacatcgtttcccgaagtgagttcccagagagctggctgtggaacgttgaggacttgaaagagccaccgaaaaatgggtaaggccggggtacccccggtacaacccaccccagagtcagaccgtttaatttgcatgcacctgctatctctggtcttctctggaatcacagtgcaaccccacagcccaacctagaaaaatcaggaattgggtgacctacatggaggcacccccagacccttccagcctgtcccttggggtccctctgcaccagttcttcccctctaccaccctgctagatgacatctcctaataccccaacctcttctccatccagaatctctacgaagctcatgaatatatttttgaaagactccatcaccacgtgggagattctggctgtgagcatgtcggacaagaaaggtgagagaggatgctggctggtccccgggaggcagggaccccagggtgtctgagtgtcatctcattttatccaaactcaatcaaccctatgtttcttggcactttattctctgccctggttaccacagaggtgttgttaccaggaactgtgggaatccttagttcctgtctaacttggaagaaagaattcagccaagagtcacatagcaagggttaagtagcagagtttattgaaggaagaaacagctctgggctggtccccctggaaaaatagtagtagcaatgcttatttaaagagacagggccagcctcgatggctcacacctataatcccagcactttgggaggctgaggcaggggaatcacttcaggtcaggagttcaagaccagcctggtcaacgtggtgaaaccccgtctctactgaaagtacaaaacaattagccaggcagggggtggcgggcgcctataatcccagctactcgggaggctgaggcaggagatttggttgaacccgggaggtggaggttgcggtgagctgagattgtgccactgcactccagcctgggcaacaagagcaaaactccttctctaaataaataaaaagtgaccgtatgctctgaaagacgacacagacatggctgctcaacagaacgagccagcagcagatactgctggtagactctttttatgagactcttacatgatttttcgtgaaggggcgtgagtgggtgtcacttgtaagcatgttttgggaggtctctttgggcgagcaggctctgtggctgtaggtactagcatgcacgtggcatgtctcattagcatcgaaaatctccacccagaggtgtgttttttactatgataatgagcaaaacacaactctagggtgttttcggagcagtgcacatgctcatcatcggggaaaatccctagcaaagttatttccagctaggacctgataagtccccttcagggccagaggaccccaaccacaaggccatgtgtagctaaagtagccatcgtccttttcgctgactgccagtgagcagcgctgtcagtaggcagcctgtctgggacttcttttcccagaaagctcccctgcctgctcatttccgcctatctgcctactctaacagtgtcaaaagctagacagggtgggggtacagtctctaaaattgatgcttttctttctttcttttgtttttgagaaggagtctcactcggtcatccagccataatttatatggtttattataatttataataaatttaattataatatttatttatatatttattaattgtaatgtttataattataatatataattatatattacataatatatttcatatctacatatcacatattacatatgcaatatattatataccacatattacatatataacataccacatattacatatataatatatcatatattatatattacatatataatatatcatatattatatattacatatataatatatcatatattatatattacatatataatatatcatatattatatattacatatataatatatcatatattacatatattatatattacatatataatatatcatattacatatattatatattacatatataatatatcatattacatatattatatattacatatataacatatatattacatatatcatattacatatatcatatattacatatataatatatcatattacatatatatcatatattacatatataatatatcatattacatatatatcatatattacatattacatgtaatatgttatattacatataatatatattgcatatcacatatataatatgttatatgttgcatattacatatataatatattatatattgtatattacatatataatatatatgtaatatatacatattacacatgtaatatattatgtaaacatataatatgtattataatttataagaaatttaattataatataatttaatgaattataataaaccataattcattataatttaatacattataataaaccataatttattataatttaattttgttgtaatgtataattataatttactactaatatgtcatttgttattgttgacatgttaacatatataatgtatattttattagatatataatataaatgatgtatcatttattattgattacatatctataattataccatatcataacttattacaaaacattctatttaatttaaatatacccaaaatagtatcatttcaacattttgtaaaaagttgcaaaaccacaacccactaataatgtgactataaccttttaatatttgataataatctactagtatatcaaaattactgatgatatattttacttctgtttgcactaagtcttcaaaatccagcatgtgttttacaattcagtgcatctcatttaggatactagattttctttcttttttttttttgatacaggagcttgctctgtcacctaggatggagtgcagtggtgtaaacaggatgctaagttttctttttttagtagagacagggtgtcaccatgttggccaggctggtctcaaactcctggcctcaagcaatctgccttcctcagcctcccagagtgctggaattacaggcgtgagccaccgcgcccagcgcaggatgctaggttttcactggaaatactttgatctgtattttaggtttcataaaatttacagttgaaaaggtagattctcaggccgggtgcaaaggctcaagcctgtaatcccattactttcagaggctgaggccggcaaatcatttgaggtcggagtttgagaccagcctgggcaacatggcaaagccccgtctctacaaaaaaaaaaaagaaaagaaaagaaaagagaaagaaaaggtagatcctcatactcaagtagttgcaaaaatacttaaacgttttccactcaatcatcatttttaaaaaattaagatttaattcacttactatatgtcacccttttaaaatgtacaactcaggtcgggcacggtggctcacacctgtaatcccagcactttgggaggcccaggcaggcagatcacctgaggtcaggaggtggagaacagcctggccaacatggtgaaaccctgtctctactaaaaatacaaaaaattagcaggacatgcgggtgggtgcctgtaatcccagctactcaggaggctgaggcaggagaattgcttgaacccaggatatagaggttgtagtgagccaagatcacgccactgcactccagcctgggtgacagagcgagaccccatctcaaaaaataaataaataaaaaataataaaatatataattcagtggtgtttcatatatttaaaatgagcatcagttgtttgttttgtttcattgggtttggttttacagacaggatctcactctgttgcccaggctggagcacagtggtgcgatcatagctcactgcagccttgaactcctgggctcaagcaatcctcctgcctcagcctcccaaagtgctgtgattacaggcatgagccaccgcacctagctagatcatcaggtttaaagtttaagtctgaattaaattaaatacatttaaatacaagtacatcaaataaaagtacaaatccagtttctcactcaggcaaaccccatttcaagtgctcagcgctcccccacagcttggggctaccatatcagacaagcagatatattttggagatttctcttcctccctacacgtagatctctgagtcaaactacaaacagaatgtaaatcattaaatagtggtaactccggccaggcgcagtggctcacgcctgtaatctcagcacttgggaggctgaggcgggtggatcgtgaggtcaagagatcgagaccatcctggccaacatggtgaaaccccatctctactaaatatacaaaaattagctggacatggtggtgcgtgcctgcagtcccagctactcgagaggctgaggcaggagaattgcttgaacccaggaggcggaggttgcgttgagccgagatggcgccactgcactccagcctggcgacagagtcttgctctgtctcaaataattaataataataataataataataataataataataataaataatggtaactcccagccaccaccatcatcatctgtcatttgtcgccattgacagcgtttagttcacaggcttcagcaaagacaggctgagttagggagagctcctgcggagtggactaagagctgagacccaggagcctggccttgtccactccccgaccttgacactccgtgttctgtctctgcccgagcagggatctgtgtggcagaccccttcgaggtcacagtaatgcaggacttcttcatcgacctgcggctaccctactctgttgttcgaaacgagcaggtggaaatccgagccgttctctacaattaccggcagaaccaagagctcaaggtgggtcccggggtggcagaggcttcttggaggctgccagggggtaggtagcctgttgcacacacacttgcccggatcctttctctccctggcaggtgagggtggaactactccacaatccagccttctgcagcctggccaccaccaagaggcgtcaccagcagaccgtaaccatcccccccaagtcctcgttgtccgttccatatgtcatcgtgccgctaaagaccggcctgcaggaagtggaagtcaaggctgctgtctaccatcatttcatcagtgacggtgtcaggaagtccctgaaggtcgtggtgagtgcttggggcacccacaaacccttgtccttcagagagggctcctggtcttcgtactattgactcaggttggagatccaggctctgagacactaagaatcatagtgtccagcttaggaaatttggaagtcccagaatttcagaagcagagccaggattggggtaaagtgagtgagatgaccccaggcttagaattttaggtggtgccaaaaacctcgtcgaccatcaccaatcaataatttttttatactcgatttgaaattttttatttatttatttatttgtttgtttatttttttgagacagagtctcactctgttccccaggctggagtgcagtggcgcgatctcagctcactgcaatatccgcctcccgggttcacgccatcctcctgcctcagcctcccgagtagctgggactacaggcgccagccaccacgcccggctaatttttttgtatttttagtagagacagggtttcactgtgttagccaggatggtctcgatcttctgacctcgtgatccacccacctcggcctcccaaagtgctaggatcacaggcacgagccaccgcgcccggcaatgctagggtgatcctaaggacagtgccctgctgaccatctgtgtgtctgtctgttcttttattcatccaacgactccccccacctctaacactgcgtagccggaaggaatcagaatgaacaaaactgtggctgttcgcaccctggatccagaacgcctgggccgtggtgagtcggctgcagggggaggggctgaggggctggcagggtaaggggggtaaatgacctgggtttagtgaggtaggatagggcgggagggagctagagccatcggtatctctcactcaccctgcagaaggagtgcagaaagaggacatcccacctgcagacctcagtgaccaagtcccggacaccgagtctgagaccagaattctcctgcaaggtgagacacccttgaccccgaccccatgggtcccaggagggcatggatggagccaaattccatctcattctggaggtgtttaacccgcacctttctcttccccttcagctagaacagcccatctgtgatctgttttccctcttttacatttttttttttttttttttttgagacagagtctggctctgtcacccaggctggagtgcagtggcgcgacctcagctcgctgcaagctccgcctcccgggttcacgccattctcctgcctcagcctcccgagtagctgggactacagccacccgccaccacgcccggctaatttttttgtatttttagtagagacagggtttcaccgtgttagccaggatggtctcgatctcctgacctcgtgatccacccgcctcagcctcccaaagtgctgggattacaggcatgagccattatgcccggcctaaaaatttttttaaccatacagatattatttgctatgatcggttttatagaagcctccagatagcatttagttcagcaaagagctttcgctgatacatcagtttattttaatttttctagaccttctgtgcttcttagatgggaaaccagcttaaatgagactcaatagcctgtaatcccagcactttgggaggccgaggcaggcagaccacctgaggtaggagtttgagaccagcctggccaacatggtgaaaccctgtctctactaaaaatacaaaagttagctgggcgtggtggcacatgcctgtaatcccagccactcgggaggctgaagcaggataatcgattgaacgtgggaggcgtaggttgcagtaagccgagatcaggccactgcactccagcctgggcggcagagcaagactttgtctcaaacaaaaacaaacaaacaaacaaacaaaaagacaagcaacatagtacaagagcagaaattctggaggtcatttcttgccccaggagggaagactggagaaagaaagggacttgcaacctgtaagctataaggctttggggcaagagccttggttttttcacctttggtaggggtagaataatagtatctacctccaagggttggtgtgatgattttttttttttttttgaggcggagtctcactctgtcgccaggctagagtgcagtggcgtgatctcggctcactgcaaccccagcctcccgggttcaagtgattcttgtgcctcagcctcccaagtagctgggactacaggcgcccgccaccatgcccactaatttttgtatttttagtagagacggtgtttcaccatattggtcaggctggtcttgaactcctgacctcaggtgatccacccaccccagcctcccaaagtgctgagattacaggcttcagccacggcgcccagcctcgttgactattaagtgagacactctatggtattctcttagaacagtctggaaagtaacattaagcgtgatataagtattcctgaatattgttactggaattattttactgctggtgaaatgagacccaaggaccagggtgcccctgtgaagcacctcccactcctaacagtgcagacccccgaacagccactcagccatgcagcctcccctccccgcagtcacatcctccccagtcctcgcctgtccctaaccccttggccctggctggttgggaggctggaacccttttcacgccaccccaaggtgggtcacccacctggcttgagcaacgtcctcttcccacctgctgcagggaccccagtggcccagatgacagaggatgccgtcgacgcggaacggctgaagcacctcattgtgaccccctcgggctgcggggaacagaacatgatcggcatgacgcccacggtcatcgctgtgcattacctggatgaaacggagcagtgggagaagttcggcctagagaagcggcagggggccttggagctcatcaagaagggtgggctccctgcccctcttggagaccccagggacccctttccgagcgcatccctcccctaagatcccacctcatctcaagaccacgccctcccctgaggctccaccttctctcctagccactcccctcatttgaggccccacctcttctcaaggctacgccctctgaggccctgactcctcccaggccaggcttttcatgagaccccgcctctcctcaaggccatgcccatcccctgagggccccccacctcttctcaaggccacgccctctgaggccctgactcctcccaggccaggctcttcatgagaccccgcctctcctcaaggccatgcccatcccctgagggccccccacctcttctcaaggccacgccctctgaggccctgactcctcccaggccaggctcttcatgagaccccgcctctcctcaaggccatgcccatcccctgagggcctcccacctcttctcaaggccacgccctctgaggccctgactcctcccaggccagaatctcgagaccctgcctcttttcaaggccacgcccatcccctgggtccccacatcttctcaaggccacacccttctgtgaggcgccacctcctgtcccagccactctcatctgaggccccacgtcctctccaggccatgcctcttccctgagactccaccccctctctgagagccctcccctccctgaaagccccccaccctcaatatccttctcctctctgaatcccttgtcctcttgagaacttttccacctcctcgttctgatcccccaccctctttgagtccttccctttttaaggtcccctcctcccagaacccctccgccaccctgagcccctgtcccctctctgcaccccgcccctgccctttctggcgtgccccctctgctcagccccggctcttttgggggttcctctctcttctctgcagggtacacccagcagctggccttcagacaacccagctctgcctttgcggccttcgtgaaacgggcacccagcacctggtgagtcccaacagccagctcaggccatgcatactccccaccctcaacccccagcagggcccggaccctggccaggggtggtcccttaggccagccttgcccaaacagccctggacctgcagagtccaggcaagcgctggctgagtggccggcggtcattaagcatccttaagcacggaccgcatacaacagctgggtcctggggcctgggaaggcaaaccaggcaaactgggccaggccctggtccctcccccacgctcattggctggttgacatggcagtctctggatctcagagccgattggctcatgctctgtgcccactccaggctgaccgcctacgtggtcaaggtcttctctctggctgtcaacctcatcgccatcgactcccaagtcctctgcggggctgttaaatggctgatcctggagaagcagaagcccgacggggtcttccaggaggatgcgcccgtgatacaccaagaaatgattgtaagaggctgggatttagggcaaaatggaagagaggggctcctgagtctcgcaggatgaacacgagagagagccccacctccatgtgcccactgcccaattccctttgcaaagattgggctggggggtgggggcaggcagatatatgagccagaggcgtcactccagcattgcaaaaaccagagacctgcgaagcccagcgcaaaatgaagagacacggcccctcgctcagaaattattaagaatttcattaaaccaagtgcaggggtcctgcctgggaatccctttctcacattcaatccatcaacacctgcattctcccatgatgttataagaatcacctccttctctccatccttatggccagcccctggtccaagcaacactctccccgcccctccttatttggagaccttgtagaaaccacctcctggtcatcatcctggtggcctcccacttttgttggctctcagacactcaccacatagcagttggggtgattttttcaaatccagctggatcagttcttagaaagtcccgtggctccccctgtggcacttaaacacaaaactccttcgagcactggttctcgaagtgtgatcctcagaccagcggcagcaacagcacccatgacttactaaaaatgtgcattctgtggctgggctcgacggcccatgcctgtaatcccagcgctttgggaggccgaggcaggaggatggcttgagcccaggaggtcgaggctgcagtgagccatgatcatgacactgcactccaggctgataacagagtgagaccctgtctcaaaaacaaaacatattctgagaccggaccccagactcactgaatcagaaattctaggggcaggacccaggaatctgaggggtgtgagtgtgtgtgtgtgtgtgtgtgtgtgtgtgtgtgtgtgtgtgtgtgtttgagatggagttttgctcttgtcacccaggctggagtgcaatggcccgatcttggctcactgcaacctccacctcccaggttcaagcaattctcctacctcaacctcctcagtagctgggattacaggtgcccgctccaccatgcccagctgatttttgtatttttagtagagacggggtttcaccatgttggccaggctggtcttgaactcctgacctcaggtgatccgcccaccttggcctcccaaagtgctgggattacaggcatgagccaccgcgcccggcctaggaatctgagtttttaaaagtgcccgcattcctccaggtgatgctaatgtgtgcttgagatggagaatcactgcctcagtctcacctttcaggcttccagacttccagcctttcttttctttccaggctccatccattgataggagccttgctctattgttctacagggcctttgcacatgctgtttctgccacctagtatgctaatccctgccgtctgtgagagttgactccctcagggacactttttctgacctccccaactgggtcacactcccacagttcattatcgctgcgatgtcctctttcccttgcacagaactcatccacttataagtatatatctcttggctgggcgcagtggctcatgcctgtaatcccagcactgtgggaggccgaggcaggtggatcacctgaggtcaggagttcgggaccagcctgaccaacaggggaaaccccatctctactaaatacaaaaaaattagcttggtgtggtggtgcatgcttgtaatcccagctacttcggaggctgaggcaggagaattgcttgaatccaggaggcggaggttgcagggagtcgagattgcgccattgcactccagcctgggcaacaagagcaaaactgtcccaaaaaaaaaaaaaaaaagtgtatatctcttgaggagctggatggaccatgtccatcttccctactagacaaaagctctgtgagggctagagcctgtgtctggttttacaatggatcagaccgttgtacccattgtacattgcacattgtacattgacatttgcagaaggaacaaattgttgcatgaattaatactaagaagtttgaccttcctagggtagcggggtaacacctagaagagactcagccctgcccagaccccctgattctgaatctgcaaggggggatgactgccatgtgtggacacaccggtgaccccatccttgctttctgctctctatctcagggtggattacggaacaacaacgagaaagacatggccctcacggcctttgttctcatctcgctgcaggaggctaaagatatttgcgaggagcaggtcaacgtaagtgccctccatcttcccaccctaccctaccttacccgatgcagagcacagccaccttggagagtgagaggttgccttcagggaatttgcagctctcccagtgcaataacagacatcactgcagtcatgttaatagctaacatcttttgagcacttaactcatctaatacagacccgccctctaatagtttcacatgttaagtctcataatccttttagcagcctgaaaggtaagtcactcttattatccccagtttgcagatgagaaaactgaggcacaaagagatcaaaggtggggattctttctgtctgccttacaattttcagagggttttcagcccatttccaaaagtgctttctacatcagtgctacatgatcagtacagttgcgtacttgctacttccttaaagaaaacttgggatacagagctaagactatttccttagtccagaggatctttcaggtgattttcaaagggatccgtgactccaaacaggaaacggtgaacactgttggctcatcactgtctctttttcctctggttttgattctgaagcagggaagcttggaaagatgggccgctgagagtctggaatgcctttgtctgctttattgtggttgtttgtttgtttgtttattttttgtgatggagtctcactctgtcgcccaggctgcaatgcagtggcatgatctcagctcactgcaccctttgcctcccaggttcaagggactttactgtttcagcctccagagcatctgggattacaggcacccgccaccatacccggctaatttttgtctttttagtagacatgaggtttcaccatattggccaggctggtctcgaactcctgacctcaggtgatctgcctggcgtggcctcccaaagtgctgggattacaggcatgagccactgcacccagcctaattgttgtatttttagtagagatggggtttcaccatgttggccaggctggtttcgaactcctgacctcaagtgatccacccaccttagcctcccaaagtgctcggattacaggcgtgagtcactgcacctagctgatcgtggggttttgagtgggttgtttaacgtttagctttccaagtgggaagcccaggattccaccctcagctagtggcttctccccccttaggaaaagagatggaggggaggggccagtgaagagaaaaacaaacacagggctgttgcctctaacacccaagagggaccaaggcagagagagagagagagagagagagagagggagggagggagggagggagggagggagggaggtaggtagagagagagagagagagagagaggagaggtggggtcagacaaatctgacttcaaatcctgactcatgggcacttccacccttgagcctcactcaggatgtgcatctgtaaattggggataataaataacgatctctgtatttttaggcctctgagttgtcccagatataacacacatgtgacccagattatacaaaaattgatggggaatttatgtgcaggcaccaaggcatcaaatagagatgaaggtggcctcagggactctgccaggatgctttgctcctctctcccgtgatcttcattccgttcttggccaataattcagttcaggcagaatatggctgccttccttagagaaaatatcagatcaaggttagggccgccatattcccaggaaaggactctgattggctcagcctgggtcagatgactatatctggaccaatcagctaaggacaggaagtaggtctcagggggcagacatggctgtttccactgtggccacgtgaatggaagggagaagaagttcttacaaaaggagtggatgtcagagaggcaaatgggcaggaataaaagagatttgtttctgctacaacatagcaacattgtagcagagtatagcacaggctgtgaaaccagactcctggggtcaagagtgtgctgtaatcccaactactcaagatgctgaggcaggagaatcacttgaaccagggaggtggaggttgcagtgagccgagattgcgccactgcactccagcctgggcaacacagcaagactccttttcaaaaaaaaaaaaagtgtgctataactagcttgctggagcccagtgttaaatttccaggaatttttcaagctggtcattaaatacaattattattaaaaactaaatattaggccaggcacagtgagcctgtaatcccggcactttgggaagccaaggccggcagatcacctgaggtcaggagttcaaaaccaccctggccaacatggcaaaaccccgtctctactaaaaatacaaaaattagccgggcatggtggaggggggcgcctgtaatcccagctacgcaggaggctaaggcacaagaatcgcttgaacccgggaggcggaggttgcagtgagccgagattgcgccatgcactccagcctgggccagagcgagactccgtctcaaaaaaaaggccaggcgcggtggctcacgcctgtaatcccagcactttgggaggccgaggtgggcggatcacgaggtcaggagatcgagaccacggtgaaaccccgtctctactaaaaatacaaaaaattagccgggcgcggtggcggacgcctgtagtcccagctactgggaaggctgaggcaggagaatcacttgaacccgagaggcggagcttgcagtgagccgagatcgcgccactgcactccagcgtgggcgacagagcaagactccgtctcaaaaaaaaaaaaaagcaacaacaaaaaacccaaccaaccaaccaaacaaacaaagttataaaagttacagttaaataaattatattaaacacaaaggttagaaacactcaaactcatcgcttcctaaacgccttactcccataatctatactcttggggttacttatgtctgttggatctgtatagtgaaaatactatataatactgtggtactgcaaagctcttcccaactctacattcaacgacaccatattggtaggttgaaatcagtgatggaagtatttacatcatggaaatgagaaaacagtacaaatcatgtcttcccccatccccagaaggctgtgtttggatcctaactctgccacttatttcctaggtggtctttgcaaaattactgcatctctcagggctcagtatgctcatcaggttttatgagattaaatgtgtgggtatctgaatgacacaaagtaagtgtgagctatgatgatgaagaagataaagatgatgatgacgatgatgatgatgactggatgaggtgttcacagtggtatactgaatctggcgcatactagtttatgagtaacaatttggagaatgtctccccaggactttgttcagtgatgtcgcattgacaccgtgaaattggcccctggtgggagtatttacaccacagaaattgtaaatcattataaaccaaggatccctcaaccctcccactggagagctggctgttaaacttttaccagcacaccacggggtacgtggatttctccagatacataatagatatgcagcaacaaggcagctcatggtggctaaaatatctgggaaattctcaaaaatggacaaatctaagacaggtgtgtcccaaggacagaaatccctgatgctcaggaagtgctgctcgaatgatccttactaacgtgacagcaatgcccacatgaccggagaatctgatcctctttctcatagagcctgccaggcagcatcactaaagcaggagacttccttgaagccaactacatgaacctacagagatcctacactgtggccattgctggctatgctctggcccagatgggcaggctgaaggggcctcttcttaacaaatttctgaccacagccaaaggtgagggttggcctggaggggtgaagggagatgcatggctgaagttcagggcgggagatactgagctgggatgcatggcttttagctgagctgggacagatgaccctaagccaagctgagatggatagtcctaaggtatcaagctgggatgcataaccctgagctgagctgggatgcacggctctaagttttcgcaggtcctcattgtaaaccacacgagaaagtttgttgcgtcatttattcaacaaatgcgtattaagcattcatttcaaagggagaagtgagagttgatgaaacaagagaggtaaggcaggagccaagtaattgagagcctcgaatgtcagccaggacacccaaacaccaggaagtctagcatgcatctctttctgagctttctctgagccatccccaggctggacagagcagtgagcactggggatggggtatcttctttgcagataagaaccgctgggaggaccctggtaagcagctctacaacgtggaggccacatcctatgccctcttggccctactgcagctaaaagactttgactttgtgcctcccgtcgtgcgttggctcaatgaacagagatactacggtggtggctatggctctacccaggcaagtgggcccacagcccctaggcacatgcatccctgtctcctgcggcttcccactggcctcctagagaagacactgaggcccagcgaggcagttcttcattcccacgagccagtgtgattgcagtggagttgagaatcagtttttattacttgcaaacccatctataggttctagaatacaatctgggtactccaagctgtgtgttgagccttcttcttgccccaggtgtctagatcatgttctcagggcccaggttcaggtctaagcctctctctccacctggtgggctctagaccaggttcccagttctatctcacaatcttaccctgtcttgctggtgggttctagaccatgttcccagttctaccaggctcccaatgtcacattgcctcactggcgggctctatagtatgttcccagttaccctggggcattacgcaaaccctcttctaggccatggtttcagtaacttcaggcttcagcaacttcaggctccagttggcctcctttctttctggtggtctgtcactcacgttctcagtgttacagtgtcactcttgggttgtagattatatgctcagtatcctctggctacggtttcattctgttcttcatgagtgggttctagacatattctcagtgtctccaagccctggtctaagactctctcctcttgatgggtctagactgcatcctcagggtcgctagacattcagtcttacatttggactttctgatggattctagacatgttctcagcatctccaagtcctggtgtaagtttctgtctctcggagagttctgaacatgtcctcagagtccagtgacctccagttatcacccctgcactctctagtaggttctaggccacattttgatgtcccagctctgatttgaacctctttatcccccactggattctagccactttcccaggctcccagatcaccatctttctctcttgtgggttctaggccaccttcatggtgttccaagccttggctcaataccaaaaggacgcccctgaccaccaggaactgaaccttgatgtgtccctccaactgcccagccgcagctccaagatcacccaccgtatccactgggaatctgccagcctcctgcgatcagaagaggtacagtcacccagccaagccctcctcactctggctgtctccccctacactagccagggtttactgggaagcaagagggagggccaggtgaccatcacaggcagcagaaggcttaattcccaacatgctctcttctctcttttcactctgcagaccaaggaaaatgagggtttcacagtcacagctgaaggaaaaggccaaggcaccttgtcggtaaggaacagaaacccacacctgcctggcccatgcccctctgccccagagggaccatctcctcttgtccccagcagtcctagtcctgtgggctgacattgtgtctcctctcccatcttaccaggtggtgacaatgtaccatgctaaggccaaagatcaactcacctgtaataaattcgacctcaaggtcaccataaaaccagcaccggaaacaggtaaaaggaatcaaggccttatctgtcaccttcctcctacccctcttctaatgtcttccccgctcctgaatcaacacacaggtataccctctcccatctttctctcttctgtgtttctagaaaagaggcctcaggatgccaagaacactatgatccttgagatctgtaccaggtaagaagctaggtcaccggggttcatcttggccatccctctatctctagcaagaattcttgcaaataatatccatgatattcagtactttccaagtacactgtgtatctgatactgttctaagtatccaccatgaggtagacaacacagacagtccttgctttgcatgttaatgtgagaccacagcaatgaccacgtaagctgagactgtcaaagcatcttagtaatcaatggaggaaagtacacaatcattccatgacctttaaagttttctttttttctttttagagagatagggtcttgctctgtcagccaggctggagtgcagtggcacaatcatagctcactgtaacctcaaactccctggctcaagcgatcctcctgcctcagccactcaagtagctgggactacaggcgtgtgccatgacacctggctgatttttattttttattctttctagaggcagggcctcactgtgttgcccaggctggtctcgaactcctagccttgagcattcctctgccttgggctgccaaagttttgggatcacaagcatgagccactatgcccagcctaaatgtttctattacaacatttaaaattatcatactgccagttataaagatacagggaaatggccgggtgtggcggctcgcgcctgtaatcccagcactttgggaggctgaggcgggcagatcacgaggtcaggagatcgagaccatcctggctaacacggtgaaacaccgtctctactaaaaatacaaaaaaattagccgggcatggtggcgggtgcctgtagtcccagctacttgggaggctgaggcagaagaatggcgtgaacccaggaggcggagcttgcagtgagctgagatcacgccactgcactccagcctgggcgaaagagcaagactctgtctcaaaaaaaaaaaaaaaaaaaaaatagaataaaacaaaataaagatacagggaaatgaaattcatagtaagatgagtatttgactacaccgtaatttaaaacattagaacattgagatgcaaggtgtatttgttgttttttttttcctttgtatgacacttacggagagtactttagttcaaaaaaatgcttgccttcttctctttgtataatttacaacatggagtaaacatcttttctatgccttagtaccttgtcttgctcctttctaagtttggatcagcttccaatattttatcctttgagctttccatgacacaaaattcctccaagagttcctttaaagtgactttgtattctataatgtcccttcctctgggacatcttcatcctttttgtccccatgaccttccttatttatgctaatacatttgccttccctgagttcctctacactacctatctctcaaatggcagcagggtcaacatcaccatagtctgctattctttgataactccatttatgctgtctttgaagttcacttctggcattatcacttttcatttctttgctgcatttttatctttgttggccagttccctcttttcgtgatacattgttgtaaaatctcatgggagttagccacctggagacagggaggcaacagaactacacactttgctgtctgtgcataaattgaagagcagaagctcagtgaccaatcactgatggactttgaaaggagtgacagtaattggccctcaattatgatgcttatcttttatttatgtcgtgatttctagactgaagagttagcaacaaagtttataccatatgcaactactcgtgatcaatataccaaggtactgaaaaagaaccatgtcactgggctactagtgttatttaactgaatcatgcagagtgagggctgcctgtattcttgccttgttttctagaactgaagcatggagggtcaaataatgcatccaatgttatttagagctggaatttgaatccatgcagttgggtgcagagtctgagctcttaatcaccttgaccattacattaccttgctttttatttcctttggggaaatgtttcctaaaaaatgtaacgcccctctgtgctgctatgtgggaatcagaagtctcagtgcctgatcagacctccttgtccaggaacagacccttggggctgacccctccttgggacccaatgcccttctttctgcactatccaggtaccggggagaccaggatgccactatgtctatattggacatatccatgatgactggctttgctccagacacagatgacctgaagcaggtatgaagggctcaggagctgggataagtggaaaggagcctgggttctggaagaggctgcagggagagaggggtccaggagggatttttcacaggctccacctttccccagctggccaatggtgttgacagatacatctccaagtatgagctggacaaagccttctccgataggaacaccctcatcatctacctggacaaggtaaggctgcatcatcctcccctgggaggcttccaggggcaccctgacctctatctggctggtctttcttttcctttcagcttttgtctctgggtcagactaaccctgggccagaggagacagggtctgtgctgctgagttgtaggggaaggagcttgtaaaataagggggtcaacccagcatcttctataaacatctcatcttctgaccatttgcctcctccaacttgttatcagagtcttaaacaaccattgaaaaaaagcccttttggtttttttggttttttttttaagtgctttgtagagagcaaggtcttgcctcgttccctaacccaatcctgggctttgtttctttctttgatctatttctctcttctgttgttttctttctttcaggagacagggtcttgctctgtcacccagactggagtacagtgtcttgatactagctcactgcaaagtcaaattcctgggctcaagggatcctcctgcctcagccacctgaggagctggaactgcaggcctgcgacactgcacccagctaatttttttttcataaatattatgcttttgtacccagcttttttttttttttttttttaactgcagccttgacctcccaggcttacatgatcctcccacctctgcttcctgagtagctgtgattacaggtgcatgccaccatgcccagtgaattaaaaaaaaaaaaagtttgtagatatggggtcccactgtactgcctaggctggtcttaaactcctgagctcaagtgattctcccacctcagcctcctaaagtgctgagattacaggcataagcccctggtgcctggccccagctgaatttttgttcttgtttcttcataaatattctgtgtaagtacccagctgattgttttattttttgtagagatgggggtcttgatatgttgctcaagttggtctcaaactactggcctcaagcgatcttcctgcctcagcctcccaaagggctgggattccaagcatgagccaccacacctgccacctcttctgttattttctctccatctggcattctctgactctttcatctctaccatgatttgggctttctcctctcccttctcttatttcttcccattctcctatccccatatcctccctgctaactcctgatacccacagggcccctcaatcccattttagtcagcttaagtaacaatagctactaaaacaaaacccctaagaatatggggtcttaacacaacagacttgtatttctcactcatgtaaagtccagttggcatggggggtaaggaagggtccctctgctccatgtagtctctcagggatccaagcaccttccatcctgtggctctgcaatccttaggatcttctgtagttctctgcaggattcattcattctagatggaaataagattgtgcatgggttgtttttatgggcatagatagcaatctgttcagccacctggccacacctaattgaaagaggagctgagaaaggtagtctcactgtgagtctaggaagaaaagtaaatggatttgctgaattgctcattcatctttgccacttcctccttgatccttcagtttctccaccactgcctcagctcccaagacaatgctggactccctcccacatcaccccactgaccaagctcctccttccccctcaggtctcacactctgaggatgactgtctagctttcaaagttcaccaatactttaatgtagagcttatccagcctggagcagtcaaggtctacgcctattacaacctgggtgagcagccaacctagggcctggggtctgatggttccaggggcctgagagtcccaggtatatatgaattgtggggatctgagaatgaaggtctaaggagtccagggatttgagcattcgtagtatgaaggtcccacgggtctgagggtcccaaggatctatgagttgaggttctgaggttctgaggggatctgagaatgatggtctaagcaggccagggatttcaggattagtaatctgaaggtcccagggtctgagagtcccaaggatctatgagttggttctagggatctgagacttgggggtctgatgggttcaggggtctcagggtcttaggaatatgtgagttgcagggggttctgaaaataagggtctaaggattctagatatatgagggttggaggcctgcgtgtcccaggaatctatgaatttggggtctgagggtcccaggcttctgtgagttgagagtctaagagactcaagggtctgagaatcccaaagatcagaaagtagagggggtcttggggtctgagggatctgaggggttgaagacctagcatctccaggtctgaagactgagaactggggatctgggcctcccaggcatggtctttggagggaggcccttatcctctcatcttcacatcacatctgcccgcagaggaaagctgtacccggttctaccatccggaaaaggaggatggaaagctgaacaagctctgccgtgatgaactgtgccgctgtgctgagggtgagttccctggagccgggaacaggtgggtctgagcaagccacacttacccaggtcatctatcccatggtcagggacccccagacccatacccaggggataccaaggggggtaggctcccagggctggccacacccatgggcagtaggccccagataaggagtgggacttagaccctgtctccaccccaccctgcagagaattgcttcatacaaaagtcggatgacaaggtcaccctggaagaacggctggacaaggcctgtgagccaggagtggactatggtgagtgggtgatgggtgggggtcacgcatgtttagctgtgtgtgtccaattgtgtggtgggtggtaggtgtggttgtcatggtgtggcttcaggctgtgggtgtgggtgactgtggtgtgtgtgagagcatgtattgtgaggggccatgattgtgtggggaaccatgactgtgagtggcctaggtatgctcatgtgagaaaaggtagatgtggttgtatgcatcattgcgtgggtggctgtgaggttgtagttgtgtgtggctgtggttgtgtgaggctgtgtggttgtagatggcagtgagtgtgaggtcctgaagttacgtatatgactgtagttttccgtggctatggttgtgtgcatggccatgaggctacagtattttgtgcatatgagtcactctcattgcatagtatgaatagtatgttactagacattgtgggtggctgtgacctctgtgcatgcctatgagcacgactgtgtgtggatggtgacatgggaccctctatggttgtgtgtgtaatgaggggtgggccatagtgtgactggctgtgattctgcaactttctgcttgggagagagagccacatgcccgggtgcacttgcaaaccagggtgcccctcatggtcaacctagcccaccacccaaactgtctgcctctcccccacagtgtacaagacccgactggtcaaggttcagctgtccaatgactttgacgagtacatcatggccattgagcagaccatcaagtcaggtcaggctcagcacgctgcctcccgtggctcttccctggcttcctccccacgactcagcttcttccctctcccctccactccaggctcggatgaggtgcaggttggacagcagcgcacgttcatcagccccatcaagtgcagagaagccctgaagctggaggagaagaaacactacctcatgtggggtctctcctccgatttctggggagagaagcccaagtgagtgctttccctgcgcgtgcgcgcgaccgcccgactgccccgcccatgccacgcccacaccattgtcacgcccctgcgccacgcccacaccacgccccttcctgacctgccattcttccctccagcctcagctacatcatcgggaaggacacttgggtggagcactggcccgaggaggacgaatgccaagacgaagagaaccagaaacaatgccaggacctcggcgccttcaccgagagcatggttgtctttgggtgccccaactgaccacacccccattcccccactccagataaagcttcagttatatctcacgtgtctggagttctttgccaagagggagaggctgaaatccccagccgcctcacctgcagctcagctccatcctacttgaaacctcacctgttcccaccgcattttctcctggcgttcgcctgctagtgtgctgacttctttagccaaggagcatggacctgcctcacctgcacgtggcatgcacctgcgcctcacctccatttcacctgcacactcaccggcagctcacagccccttcacctcttcacttaccggcatcctcacctgttaatcttaccaatttttttttattttattattattactattttaagttccggggtacatgtgcaggatgtgcaggtttgttacataggtcaagtgtgccatggtggtttcctgcacctatcaacccatcacctaggttttttgtttgtgtgttttgaggcagagtcttgttctgtcgcccaggctggagtgcagtggcacaatctcggctcactgcaacctccacctcccgggttcaagtgattctcctgccttagcctcctgagtaggtgggattacaggcgcccgccaccttgcctgggtaatttttgtatttttggtagagacggggtttcaccatgttggccaggctggtcttgaactcctgatctcaagcgatccgcccgccttggcctcccaaagtgctgggattacaggcgtgagccatcacacccagccccctattacctagttattacgtccaggatgcattaggtcttttccctaatgttctccctgctcccaatgttaccaatattttcatctgaatctttacctgctcactcctctgcaccctcagctgaatccatgtatgggtttttgttgttgttgttttgtttttgtgggtttttctgtttttttttttttttttttttttttgagatggagtttcactcttgtcgcccaggctggagtgcaatggcgcgatctcggctcactgtgacccctcctcctgggttcaagcgattctcctgcctcagcctcccgagtagctgtggttacaggcacacggccaccacacctggctaatttttgtatttttattagagacggggtttcaccatgtcggccagaccggtctcgaactcctgacctcaggtgatctgcccgcctcggcctcccaaagtgctgggattgcaggcgtgagcctccgtgccccgccagggttttttgtttttgttttttagcatcctcacctggccccaacacctacatctctatcttaagcttacctgtatctttaccttaacagcattgttacctatattctcacctttttccacctacatcctctccggtgagtgtattttctctgcatcttcatctgggtcctcacctgcatctttacctgcatgcttttctaggtattttcttgggttcttgcccacattctcacctacattctcacctgcagatttacctatcttcttactgtaactgcccaatgggttcaccttgcccgctgcctagacagaaccgatttatcagacgggggatgcagtggagaaagagtaattcgtgcagaacaagctgtgcaggagaccagagttttattattattcaaatcagtctcctcgagcatttggggatcagcggttttaaagatagtttggtgggccagacgcagtggctcatgcctgtaatcccaacactttgggaggccgaggcaggtggatcacctgaggtcagcagttcgagaccagcctggccaacatgatgaaaccccgtctctactaaaaatacaaaaattagccaggcgtggtgatgcacacctgtagtcccagctacttgagaggctgaggcaggagaatcgcttgaacccgggaggtggaggttgcagtgagccgagattgcgccactgcactccagcctgggtgacagagcgagacttcatctcaaaataataataataataatagtttggcaggtagaggtttgggaagtgaggagtgttgattggtgaggttgaagt

SEQ ID NO: 8 - protein, 1663 aa, Homo sapiens
MGPTSGPSLLLLLLTHLPLALGSPMYSIITPNILRLESEETMVLEAHDAQGDVPVTVTVHDFPGKKLVLSSEKTVLTPATNHMGNVTFTIPANREFKSEKGRNKFVTVQATFGTQVVEKVVLVSLQSGYLFIQTDKTIYTPGSTVLYRIFTVNHKLLPVGRTVMVNIENPEGIPVKQDSLSSQNQLGVLPLSWDIPELVNMGQWKIRAYYENSPQQVFSTEFEVKEYVLPSFEVIVEPTEKFYYIYNEKGLEVTITARFLYGKKVEGTAFVIFGIQDGEQRISLPESLKRIPIEDGSGEVVLSRKVLLDGVQNPRAEDLVGKSLYVSATVILHSGSDMVQAERSGIPIVTSPYQIHFTKTPKYFKPGMPFDLMVFVTNPDGSPAYRVPVAVQGEDTVQSLTQGDGVAKLSINTHPSQKPLSITVRTKKQELSEAEQATRTMQALPYSTVGNSNNYLHLSVLRTELRPGETLNVNFLLRMDRAHEAKIRYYTYLIMNKGRLLKAGRQVREPGQDLVVLPLSITTDFIPSFRLVAYYTLIGASGQREVVADSVWVDVKDSCVGSLVVKSGQSEDRQPVPGQQMTLKIEGDHGARVVLVAVDKGVFVLNKKNKLTQSKIWDVVEKADIGCTPGSGKDYAGVFSDAGLTFTSSSGQQTAQRAELQCPQPAARRRRSVQLTEKRMDKVGKYPKELRKCCEDGMRENPMRFSCQRRTRFISLGEACKKVFLDCCNYITELRRQHARASHLGLARSNLDEDIIAEENIVSRSEFPESWLWNVEDLKEPPKNGISTKLMNIFLKDSITTWEILAVSMSDKKGICVADPFEVTVMQDFFIDLRLPYSVVRNEQVEIRAVLYNYRQNQELKVRVELLHNPAFCSLATTKRRHQQTVTIPPKSSLSVPYVIVPLKTGLQEVEVKAAVYHHFISDGVRKSLKVVPEGIRMNKTVAVRTLDPERLGREGVQKEDIPPADLSDQVPDTESETRILLQGTPVAQMTEDAVDAERLKHLIVTPSGCGEQNMIGMTPTVIAVHYLDETEQWEKFGLEKRQGALELIKKGYTQQLAFRQPSSAFAAFVKRAPSTWLTAYVVKVFSLAVNLIAIDSQVLCGAVKWLILEKQKPDGVFQEDAPVIHQEMIGGLRNNNEKDMALTAFVLISLQEAKDICEEQVNSLPGSITKAGDFLEANYMNLQRSYTVAIAGYALAQMGRLKGPLLNKFLTTAKDKNRWEDPGKQLYNVEATSYALLALLQLKDFDFVPPVVRWLNEQRYYGGGYGSTQATFMVFQALAQYQKDAPDHQELNLDVSLQLPSRSSKITHRIHWESASLLRSEETKENEGFTVTAEGKGQGTLSVVTMYHAKAKDQLTCNKFDLKVTIKPAPETEKRPQDAKNTMILEICTRYRGDQDATMSILDISMMTGFAPDTDDLKQLANGVDRYISKYELDKAFSDRNTLIIYLDKVSHSEDDCLAFKVHQYFNVELIQPGAVKVYAYYNLEESCTRFYHPEKEDGKLNKLCRDELCRCAEENCFIQKSDDKVTLEERLDKACEPGVDYVYKTRLVKVQLSNDFDEYIMAIEQTIKSGSDEVQVGQQRTFISPIKCREALKLEEKKHYLMWGLSSDFWGEKPNLSYIIGKDTWVEHWPEEDECQDEENQKQCQDLGAFTESMVVFGCPN

SEQ ID NO: 9 - DNA, 471 bp, Homo sapiens
  source 1..471: mol_type = genomic DNA
tgctgagctatgagccaaaccagggatttacagggaggaaaagctttcggactgctgaaggcccagcaggaagagaggctggatgagatcaacaagcaattcctagacgatcccaaatatagcagtgatgaggatctgccctccaaactggaaggcttcaaagagaaatacatggagtttgaccttaatggaaatggcgatattgatatcatgtccctgaaacgaatgctggagaaacttggagtccccaagactcacctagagctaaagaaattaattggagaggtgtccagtggctccggggagacgttcagctaccctgactttctcaggatgatgctgggcaagagatctgccatcctaaaaatgatcctgatgtatgaggaaaaagcgagagaaaaggaaaagccaacaggccccccagccaagaaagctatctctgagttgccctgatttgaagggaaaagggat

SEQ ID NO: 10 - protein, 147 aa, Homo sapiens
MSQTRDLQGGKAFGLLKAQQEERLDEINKQFLDDPKYSSDEDLPSKLEGFKEKYMEFDLNGNGDIDIMSLKRMLEKLGVPKTHLELKKLIGEVSSGSGETFSYPDFLRMMLGKRSAILKMILMYEEKAREKEKPTGPPAKKAISELP

SEQ ID NO: 11 - DNA, 14020 bp, Homo sapiens
  source 1..14020: mol_type = genomic DNA
gtagaatacagcaacgacagacattttgggagagaagcattttatcatagcttttagaagagaagtatttttcagcatcataagcacacaattccaaggacagataccttcaagggattgcttttgacagttatgacaaagtcttaaagaagaataaaaggacaaaggaaatcctccagcaacaaagctgccacttatagatgagaaagtgaatgggaataaggaagaaactcagaaaagggaagagagatcactaaaaaccctgatttggaaagtcccagtactaccctgagaggagaaagaaacaaattcacacagcacgtcaccgccagaagaagaaaggagggaagacaagggaacagagaggatctcaatcctaaaaggacaatgtggaaacatttaggggacagaggtgaatctgccaggccaatgtagtttagaatgtcactcaatcactgagaatgagaaaggagtctgccgatgggcaccatgtggacaggagatgaggcaataaacatatgtaacaattaaaagtgagaaataaagatctggttttggttcagttaacccatatttgagtcgtagttccagcatttactgtttgtgcggtcttaaataatttattgtctcaacctcagtttctgcataggtcaaatggttcaatattatctactttaaaggttattatgaagagttaataagataatgagggaaaaaaaggtacctggcacttagtaggtgctcaataaaggacggctttttttttttaagtattgcttctaaatttgtatgtaagaaaaaatgatataatacaatgatataagacaggggaccgcaggacaagtccagccacacccattcatttatgtattatctgactgcttttctgaaatgatgcaaggttgagtagttgtacctgaaacatttattatctggccctttacagaaaatgtttccagaccctggataagtggtaccagagccccctctgtttgtggtcccctctcttatacccactaggtgtgagaaaagacatagagtaggagagccctgccatccatcttacccacccaggggctttttctgatggatccaaaggaaggacaaggtcttattggtctcccagaactgacataacaactccgacatcagggaaaagccattggagactacatagctcgccagccccagccacctgctcatatatctaagccctccttgttctagaccagggaggagaatggaatgtcccttggactctgcatgtccccaatctgagaacctggatccaagagggagaagaagcccattggagatgatgccataaaggaagtggaagcgatatgataaaaatcatagtgcccattcccaaataatcccagaagcagaagggaaaggagagaaatatccacaaagacaggtgtgggtacacacaacatttttcatactttaagatcccagagggactcatggaaatgatacaagaaaatgactcataagaacaaatattaggaagccagtgccaagaatgagatgggaaattggggaaaatgttgggggcagattgcttagttctgttctaagcaagagggtgaacaaggaaggaacagctcactacaaagaacagacatcactgcatgtacacacaataatataagaactaacccatgattattttgcttgtcttcttgttcaaaatgattgaagaccaatgagatgagatcaaccttgataactggctgcgaagcccatgattagacacaagatggtatcagggcacttgctgctttgaataaatgtcagtctcctgtcttggaagaatgacctgacagggtaaagaggaacttgcagctgagaaaggctttagtgactcaagagctgaataattccccaaaagctggagcatcctggcatttccagctccccatctctgcttgttccacttccttggggctacatcaccatctacatcatcatcactcttccactccctcccttagtgccaactatgtttatagcgagatattttctgctcattggggatcggaaggaagtgctgtggcctgagcggtctccttgggaagacaggatctgatacatacgttgcacaacctatttgacataagaggtttcacttcctgagatggatgggatggtagcagatttgggtccaggttacagggccaggatgagacatggcagaactgtggagactgttacgtcagggggcattgccccatggctccaaaatttccctcgagcgaaagcatcaggggctcatgcaacctggatactagtgctgcttcaaccacactgtgctattggatgagtcacttccaccctcctagccttgatttcttcgtctgctgttcacattcaaatagctattcatgtcttcatctctgtggtcccaccatatcccaccagacaatcattagggctcctcttagctggcagattctgaggtcctggatgctacaattggaagatggagaagtagaagctcaaggtttctgacctgtatcccaagtcccagaagcagaatggactaactcagagctgatgctcgggtcccttgcatatctcccttcctgtcactggctttgatcctccttcgttcagcttgtaatcacatcaacagaccaaagacatctctgtgttctgtcaggagagttcacagagccaccaaccctccagaccctgctggttgccgcataaagactctgaggaagggtttgaggctgctgtgatcatgcaatgaatgcatgattgtaccactgcactccagcctgggggataaaggtagatcctgtctaggagagagagagagagaaagagaaagagagagagaagggagggagagacaaagaaaaagagagagagggagggagaaagaaagagagaaagaaaagagaaaagaaagaaaaagaaagaaagagagagagggagggagggagagagaaagaaagaaagaaagagaaagagagaaagagagaaagagaaagaaaggaagaaagaaagaaagaaagaaagaaagaaagaaagaaagaaagaaaagaaaagaaagaaagagagagagaaagaaaaagaaagaggaaggaaggaaggaaggaagaaagacaggctctgaggaaggtggcagttcctacaacgggagaaccagtggttaatttgcaaagtggatcctgtggaggcaaaacagaggagtcccctaggccacccagacagggcttttagctatctgcaggaccagacaccaaatttcaggagggctcagtgttaggaatggattatggcttatcaaattcacaggaaactaacatgttgaacagcttttagatttcctgtggaaaatataacttactaaagatggagttcttgtgactgactcctgatatcaagatactgggagccaaattaaaaatcagaaggctgcttggagagcaagtccatgaaatgctctttttcccacagtagaacctatttccctcgtgtctcaaatacttgcacagaggctcactcccttggataatgcagagcgagcacgatacctggcacatactaatttgaataaaaatgctgtcaaattcccattcacccattcaagcagcaaactctaccacctgaatgtacatgccaggcactgtgctagacttggctcaaaaagatttcagtttcctggaggaaccaggaggagcaaggtttcaactcagtgctataagaagtgttacaggctggacacggtggctcacgcctgtaatcccaacactttgggaggccgaggcgggcagatcacaaggtcaggagatcgagaccatcctggctaacatggtgaaaccctgtctctactaaaaatacaaaaaattagccgggcgtggcggcaggtgcctgtagtcccagctgctggggaggctgaggcaggagaatggtgtgaacccgggaggcggaacttgcagggggccgagatcgtgccactgcactccagcctgggcgacagagtgagactctgtctcaaaaaaaaaaaaaaagtgttatgatgcagacctgtcaaagaggcaaaggagggtgttcctacactccaggcactgttcataacctggactctcattcattctacaaatggagggctcccctgggcagtaccctggagcaggcactttgctggtgtctcggttaaagagaaactgataactcttggttggtattaccaagagatagagtctcagatggatattcttacagaaacaatattccacttttcagagttcaccaaaaaatcattttaggcagagctcatctggcattgatctggttcatccatgagattggctagggtaacagcacctggtcttgcagggttgtgtgagcttatctccagggttgccccaactccgtcaggagcctgaaccctgcataccgtatgttctctgccccagccaagaaaggtcaattttctcctcagaggctcctgcaattgacagagagctcctgaggcagagaacagcacccaaggtagagacccacaccctcaatacagacagggagggctattggcccttcattgtacccatttatccatctgtaagtgggaagattcctaaacttaagtacaaagaagtgaatgaagaaaagtatgtgcatgtataaatctgtgtgtcttccactttgtcccacatatactaaatttaaacattcttctaacgtgggaaaatccagtattttaatgtggacatcaactgcacaacgattgtcaggaaaacaatgcatatttgcatggtgatacatttgcaaaatgtgtcatagtttgctactccttgcccttccatgaaccagagaattatctcagtttattagtcccctcccctaagaagcttccaccaatactcttttcccctttcctttaacttgattgtgaaatcaggtattcaacagagaaatttctcagcctcctacttctgcttttgaaagccataaaaacagcgagggagaaactggcagataccaaacctcttcgaggcacaaggcacaacaggctgctctgggattctcttcagccaatcttcattgctcaagtatgactttaatcttccttacaactaggtgctaagggagtctctctgtctctctgcctctttgtgtgtatgcatattctctctctctctctctttctttctctgtctctccctctccttccctctctgcctccctctctcagctttttgcaaaaatgccaggtgtaatataatgcttatgactcgggaaatattctgggaatggatactgcttatctaacagctgacaccctaaaggttagtgtcaaagcctctgctccagctctcctagccaatacattgctagttggggtttggtttagcaaatgcttttctctagacccaaaggacttctctttcacacattcattcatttactcagagatcatttctttgcatgactgccatgcactggatgctgagagaaatcacacatgaacgtagccgtcatggggaagtcactcattttctcctttttacacaggtgtctgaagcagccatggcagaagtacctgagctcgccagtgaaatgatggcttattacaggtcagtggagacgctgagaccagtaacatgagcaggtctcctctttcaagagtagagtgttatctgtgcttggagaccagatttttcccctaaattgcctctttcagtggcaaacagggtgccaagtaaatctgatttaaagactactttcccattacaagtccctccagccttgggacctggaggctatccagatgtgttgttgcaagggcttcctgcagaggcaaatggggagaaaagactccaagcccacaatacaaggaatccctttgcaaagtgtggcttggagggagagggagagctcagattttagctgactctgctgggctagaggttaggcctcaagatccaacagggagcacccagggtgcccacctgccaggcctagaatctgccttctggactgttctgcgcatatcactgtgaaacttgccaggtgtttcaggcagctttgagaggcaggctgtttgcagtttcttatgaacagtcaagtcttgtacacagggaaggaaaaataaacctgtttagaagacataattgagacatgtccctgtttttattacagtggcaatgaggatgacttgttctttgaagctgatggccctaaacagatgaaggtaagactatgggtttaactcccaacccaaggaagggctctaacacagggaaagctcaaagaagggagttctgggccactttgatgccatggtattttgttttagaaagactttaacctcttccagtgagacacaggctgcaccacttgctgacctggccacttggtcatcatatcaccacagtcactcactaacgttggtggtggtggccacacttggtggtgacaggggaggagtagtgataatgtttcccatttcatagtaggaagacaaccaagtcttcaacataaatttgattatccttttaagagatggattcagcctatgccaatcacttgagttaaactctgaaaccaagagatgatcttgagaactaacatatgtctaccccttttgagtagaatagttttttgctacctggggtgaagcttataacaacaagacatagatgatataaacaaaaagatgaattgagacttgaaagaaaaccattcacttgctgtttgaccttgacaagtcattttacccgctttggacctcatctgaaaaataaagggctgagctggatgatctctgagattccagcatcctgcaacctccagttctgaaatattttcagttgtagctaagggcatttgggcagcaaatggtcatttttcagactcatccttacaaagagccatgttatattcctgctgtcccttctgttttatatgatgctcagtagccttcctaggtggcccagccatcagcctagctaggtcagttgtgcaggttgggaggcagccacttttctctggctttattttattccagtttgtgatagcctcccctagcctcataatccagtcctcaatcttgttaaaaacatatttctttagaagttttaagactggcataacttgttggctgcagctgtgggaggagcccattggcttgtctgcctggcctttgcccccattgcctcttccagcagcttggctctgctccaggcaggaaattctctcctgctcaactttcttttgtgcacttacaggtctctttaactgtctttcaagcctttgaaccattatcatgccttaaggcaacctcagtgaagccttaatacggagcttctctgaataagaggaaagtggtaacatttcacaaaaagtactctcacaggatttgcagaatgcctatgagacagtgttatgaaaaaggaaaaaaaagaacagtgtagaaaaattgaatacttgctgagtgagcataggtgaatggaaaatgttatggtcatctgcatgaaaaagcaaatcatagtgtgacagcattagggatacaaaaagatatagagaaggtatacatgtatggtgtaggtggggcatgtacaaaaaagatgaacaaagtagaaatgggatttattctaaaagaatagcctgtaaggtgtcagaaagcccacattctagtcttgagtctgcctctaacctgctgtgtgcccttgagtacacacttaacctccttgagcttcagagagggataatctttttattttattttattttattttgttttgttttgttttgttttgttttatgagacagagtctcactctgttgcccaggctggagtgcagtggtacaatcttggcttactgcatcctccacctcctgagttcaagcgattctccttcctcagtctcctgaatagctaggattacaggtgcaccccaccacacccagctaatttttgtatttttagtagagaaggggtttcgccatgttggccaggctggttttgaagtcctgacctaaatgattcatccacctcggcttcccaaagtgctgggattacaggcatgagccaccacgcctggcccagagagggatgatctttagaagctcgggattctttcaagccctttcctcctctctgagctttctactctctgatgtcaaagcatggttcctggcaggaccacctcaccaggctccctccctcgctctctccgcagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaaggtagttagccaagagcaggcagtagatctccacttgtgtcctcttggaagtcatcaagccccagccaactcaattcccccagagccaaagccctttaaaggtagaaggcccagcggggagacaaaacaaagaaggctggaaaccaaagcaatcatctctttagtggaaactattcttaaagaagatcttgatggctactgacatttgcaactccctcactctttctcaggggcctttcacttacattgtcaccagaggttcgtaacctccctgtgggctagtgttatgaccatcaccattttacctaagtagctctgttgctcggccacagtgagcagtaatagacctgaagctggaacccatgtctaatagtgtcaggtccagtgttcttagccaccccactcccagcttcatccctactggtgttgtcatcagactttgaccgtatatgctcaggtgtcctccaagaaatcaaattttgccgcctcgcctcacgaggcctgcccttctgattttatacctaaacaacatgtgctccacatttcagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaaggtaaatggaaacatcctggtttccctgcctggcctcctggcagcttgctaattctccatgttttaaacaaagtagaaagttaatttaaggcaaatgatcaacacaagtgaaaaaaaatattaaaaaggaatatacaaactttggtcctagaaatggcacatttgattgcactggccagtgcatttgttaacaggagtgtgaccctgagaaattagacggctcaagcactcccaggaccatgtccacccaagtctcttgggcatagtgcaatgtcaattcttccacaatatggggtcatttgatggacatggcctaactgcctgtgggttctctcttcctgttgttgaggctgaaacaagagtgctggagcgataatgtgtccatccccctccccagtcttccccccttgccccaacatccgtcccacccaatgccaggtggttccttgtagggaaattttaccgcccagcaggaacttatatctctccgctgtaacgggcaaaagtttcaagtgcggtgaacccatcattagctgtggtgatctgcctggcatcgtgccacagtagccaaagcctctgcacaggagtgtgggcaactaaggctgctgactttgaaggacagcctcactcagggggaagctatttgctctcagccaggccaagaaaatcctgtttctttggaatcgggtagtaagagtgatcccagggcctccaattgacactgctgtgactgaggaagatcaaaatgagtgtctctctttggagccactttcccagctcagcctctcctctcccagtttcttcccatgggctactctctgttcctgaaacagttctggtgcctgatttctggcagaagtacagcttcacctctttcctttccttccacattgatcaagttgttccgctcctgtggatgggcacattgccagccagtgacacaatggcttccttccttccttccttcagcatttaaaatgtagaccctctttcattctccgttcctactgctatgaggctctgagaaaccctcaggcctttgaggggaaaccctaaatcaacaaaatgaccctgctattgtctgtgagaagtcaagttatcctgtgtcttaggccaaggaacctcactgtgggttcccacagaggctaccaaattacatgtatcctactcatggggcctaggggttggggtgaccctgcactgctgtgtccctaaccacaagacccccttctttcttcagtggtgttctccatgtcctttgtacaaggagaagaaagtaatgacaaaatacctgtggccttgggcctcaaggaaaagaatctgtacctgtcctgcgtgttgaaagatgataagcccactctacagctggaggtaagtgaatgctatggaatgaagcccttctcagcctcctgctaccacttattcccagacaaccaccttctccccgcccccatccctaggaaaagctgggaacaggtctatttgacaattttgcattaatgtaaataaatttaacataatttttaactgcgtgcaaccttcaatcctgctgcagaaaattaaatcattttgccgatgttattatgtcctaccatagttacaaccccaacagattatatattgttagggctgctctcatttgatagacaccttgggaaatagatgacttaaagggtcccattatcatgtccactccactcccaaaattaccaccactatcacctccagctttctcagcaaaagcttcatttccaagttgatgtcattctaggaccataaggaaaaatacaataaaaagcccctggaaactaggtacttcaagaagctctagcttaattttcacccccccaaaaaaaaaaaattctcacctacattatgctcctcagcatttggcactaagttttagaaaagaagaagggctcttttaataatcacacggaaagttgggggcccagttacaactcaggagtctggctcctgatcatgtgacctgctcgtcagtttcctttctggccaacccaaagaacatctttcccatagcatctttgtcccttgccccacaaaaattcttctttctctttcgctgcagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaagaaaaaaaaagggtctttcctgatcattgacttgtcttggatttgacactgaacagtaaagacaaacagggctgtgagagttcttgggggactaaagcccactcctcattgctgagtgctgcaaagtacctagaaatatccttggccaccgaagactatcctcctcacccatcccctttatttctgttgttcaacagaaggatattcagtgcacatttggaacaggatcagctgaagcactgcagggagtcaggactggtagtaacagctaccagtgatttatctatcaatgcaccaaacatctgttgagcaagcgctatgtactaggagctgggagtacagagatgagaacagtcacaagtccctcctcagataggagaggcagctagttataagcagaaacaaggtaacatgacaagtagagtaagataaagaacaagaggagtagccaggaaggagggaggagaacgacataagaatcaagcctaaagggataaacagaagatttccacacatgggctgggcatggtggcttacgcctgtaatcccagcactttgggtggcaggggcagaaagatcgcttgagcccaggagttcaagaccagcctgggcaacatagtgagactcccatctctacaaaaaataaataaataaataaaacaatcagccaggcatgctggcatgcacctgtagtcctagctacttgggaagctgacactggaggattgcttgagcccagaagttcaagactgcagtgagctgtgatcgcaccattgcactccagcctgggtgacagagtgagaccctgtctctaaaaaatgttcccagatagaaaagaaaagaaaaagccctcaggtagaggaaccagtgtgaacaagagcatgggtgtataagaatgaatgatgcaaaaagtacactttcagaattgcgggaatcaccagaagcaaagtcaaagcgcaaaacaagccagaggaaaagtagttgtaactcacaccccagataaagagataacttgtttcacatgtaaagaactgcctcaaaacatttgaaaaaaaagaacaaaaatccagcaaaacaagaggcaagggatcttaacagtctgttcacagaaaagaaaatactatttgatcttgggcagagtaaaatgatgcttaacattgtaataagaaagtcaaattaaaagcactttgagatactagtattttcccatcagattgacaaaaatcaaaagtttaacaacagaccttgttggtcaaactgtcgggaaatccccactgttaaatattacgcgtgggactataaattgatatgacccttatagaacaaaatttgctaactatgaaaatcacaagtgcacttcccctttgatccagcaatttcactcctggagatttatcccacagatggacataacccatgtgaaatggaaaatgatcaaaattattcattgcacatcatttgtaataggaaaaattggaagtaacccaagtgtctatcaacaagagactgcctaaatgaagtaaaggacatagaatactaggcagctatagaaaagaatgagaaagcactctggtattgtttggttctgtgtcccagcccaaatctcatgtcaaattgtaatccccgatgttggaggtggggcctggtgtggggtgattggatcatgggggtggagttcttatgaatggtttagcactatccctttggtgctgttctcgtgacagagttcccacaagatctggttgtttaaaagtatgtggcatcctttctctctctctctctctctctcagtcctgctcctgccatataagacatccactcccgctttgtcttctgcatgagtaaaagcttcctaaggcctccccagaagcagatgctgccatgcttcctgtggaacagcctgcgaagctgtgagccaattaaacctcttttctttataaattatgcagtctcaggtatttccttatagcaatgcaaggactgactaatacatgctgtctg

SEQ ID NO: 12 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 13 - DNA, 11856 bp, Homo sapiens
  source 1..11856: mol_type = genomic DNA
ttgcctatgattcttcaacttgcttacctggatctgaattcttccataacattttaaatcaatccaggtgtacccatcttcagaaataatggaccccaatgtctcagaattacagagtcagagtcctcaacatgtccaaagcatttcgtggaaattattccaccatgaagactagaatggcctctcttcatggattcagcttgctaaggaggcttagttcagagggactaagacactctccttttagcaaataactaaccaaaagggcaaggaagggaggtaaaagacgtcataaccaggccagtagaatcccctggtctggccattacatgtgatgatttcaaatgggttcttagtaccttaattttgagggaaaagagtagaggaaaccagccaatgctttgcatgcttagtttctgctgatgacactgaactcagagaactgagatctttggagaaaccaaagatggctcaaatgactcgtgataataaatgaacgtattactgctcgctgacaaattgaagtttcctaattataaagcagaaccagtggccaccaccatagaaagcctaccccacagatggagaagaggcaaagcactgaaactcagtccgagggacctccccagccatgggggcagggccaaatggggcttcttcaggaccagcaaagccatttttctcatcagcaaactagcttcagagaagtttgcaatcagggcactctcttccaagcctagagacccagggaaaggggtacgggggtgtcccaaggcaaagagaatctacactttttgcccccggagaggctacttccctcccaagatgcctgggattttccacttcagcagggggaaggtaagtcacatagcaaaataatgagggcacagaacagatgacctccctatagagttttgaatgagaaacacagcagggcagatgtgccccttctctagtctaggaggagctaggtccagcccctgaacatcctccccctcagaaaagctgaggccagactaagaattcaccagaccaaggagctacaacaggacatcagagctgaggctgcaaagccaggactgagaccagaccaggcaggaaactgtcaagagctttggtcaccaggcctggctgccctccaacatcagctggctctttctaaattgacacaccacatgtccctaaaattctctcttcaagtaataccaccatcaaagcaggacatttcccagagccttagagcctggtgtctgctcagtgggactcaaccccagaagaagctgttaaatcacccactgtttcagtttacaaacttcttacgacttggcaacaagtgaaactacattctggcagcaactgcaagttccctagtacccaggacttcccgttttttcttgctgtactccctcctgttaaatcacagactcatccatctccaacccccagaatatagagaaagagcacaacactacatcttaactcctgagacgtggagaacacttctcctcctgagagcttaagtaccaaatggaagctacttttcccccttggtctcaaatgtattactagattctgaactggactccaccatcacgtaagaaagcagtcatgggcagtaattctgggagatccagataggacatgccagccccacactggtggcataggaagccaagttgctgcttcctccctgtgcactcccatttgtctggcctctcttgatctcagctggcgctcacttcacatcagctatgatgcaatccagcaactaaagtattagttaataaatgctgacagcacagccttttctggtcacgtattcatactaaaatacgggggagagttggggggagagggggatatatgggaaatctctgtaccttcctctccattttgctatgacctaaagctgccctttaaaaaatacaaggggctgggcacagtggttcacgcctgtaaacccagcactttgggaggccgaggcgcgtggatcacctgaggtcaggagttcaagacccgcctggccaacatggcaaaaccccgtttctactaaaaatacaaaaagtagctgggcgtggtcgcatgcatctgtagtcccagctactcaggaggctgaggcaagagaattgcttgaacctgggaggcggcggttgaagtgagccaagatcatgccattgccctccagcctgggcaacagagcaagactccttctcaagagaaaaaacaaaacaaaacaagaaaaaacaaagaatgagctctccacgcgaaaaatccattgagatgcaaaggaaggaagctatcattgtggaattgcacatgtcagttacattaacgtttttggagcaaggtagagctcatctctcccacaagcaaattccagcccaaagcattgatactaataaagtgccatgctgcgatgtgcagggggcagacagtgtctccaagctccctacacacatgccttcccacagtttgccctttcttgaccccagaagcatcaggccccttcaccctcgagggccactatcaggagtttgaattaatggcaatcaccatgcacagggaaggctgtggaattctgacataaaaacacttagtggagggcttggaaaaagtctagtaggagcaagacgcaagctggactaattatctaaaacaagagacctggtttggggatcttaatgttctcaaaaaagaaaattattattatttttcattttgcactttgtgccataaaacattttcaacaaaacatagaatctcatttcttttgagggaaaatgattgggagaccagctcattgctggcacagaggcctggttcattcataattccttcataggcaagacaccaggtgaaccgatatagccgagctggaagagctctccaaggcagagactctgagccaaggaatgttcaaagagctagcatgtattgtgggattactatgcgccaggaattttttacactgcatcacgttccatcttcacaacagccctagaaaggaagaactattattacccccgttttataggtgaataaacaagggcacaggtccttgatgtaacagccaggatcaaacagctgggaagacgagaaaacctttcccaggctaggataacagaggatttggttgaaaatacaggcaattaggtgctacctctgggaaaaggggccaggagaggaaggagacacttttccctgcatgccctgatgtcctatttgaacattttatcatgaacacgaacttcctatttaaaaaacactttttattgaaaagataaatctgtgtgttgtattgtgtcactcagttcaagtacttgaaatttattgaattgtattttctaaaaaatagatagttgagtaaaagcaagctcacattacatagacggatcacagtgcacggctgcggagctgggagcagtggcttcgtttcatgcaggaaagagaacttggttcaggagtgtctacgttgcttaagacaggagagcactaaaaatgaaaccatccagccatcctcccccattttcattttcacaccaaagaatcccaccgcggcagaggaccaccgtctctgtttagacaatcggtgaagaatggatgacctcactttccccaacaggcgggtcctgaaatgttatgcacgaaacaaaacttgagtaaatgcccaacagaggtcactgttttatcgatcttgaagagatctcttcttagcaaagcaaagaaaccgattgtgaaggtaacaccatgtttggtaaataagtgttttggtgttgtgcaagggtctggtttcagcctgaagccatctcagagctgtctgggtctctggagactggagggacaacctagtctagagcccatttgcatgagaccaaggatcctcctgcaagagacaccatcctgagggaagagggcttctgaaccagcttgacccaataagaaattcttgggtgccgacgcggaagcagattcagagcctagagccgtgcctgcgtccgtagtttccttctagcttcttttgatttcaaatcaagacttacagggagagggagcgataaacacaaactctgcaagatgccacaaggtcctcctttgacatccccaacaaagaggtgagtagtattctccccctttctgccctgaaccaagtgggcttcagtaatttcagggctccaggagacctggggcccatgcaggtgccccagtgaaacagtggtgaagagactcagtggcaatggggagagcactggcagcacaaggcaaacctctggcacagagagcaaagtcctcactgggaggattcccaaggggtcacttgggagagggcagggcagcagccaacctcctctaagtgggctgaagcaggtgaagaaagtggcagaagccacgcggtggcaaaaaggagtcacacactccacctggagacgccttgaagtaactgcacgaaatttgaggatggccaggcagttctacaacagccgctcacagggagagccagaacacagaagaactcagatgactggtagtattaccttcttcataatcccaggcttggggggctgcgatggagtcagaggaaactcagttcagaacatctttggtttttacaaatacaaattaactggaacgctaaattctagcctgttaatctggtcactgaaaaaaaattttttttttttcaaaaaacatagctttagcttattttttttctctttgtaaaacttcgtgcatgacttcagctttactctttgtcaagacatgccaaagtgctgagtcactaataaaagaaaaaaagaaagtaaaggaagagtggttctgcttcttagcgctagcctcaatgacgacctaagctgcacttttccccctagttgtgtcttgccatgctaaaggacgtcacattgcacaatcttaataaggtttccaatcagccccacccgctctggccccaccctcaccctccaacaaagatttatcaaatgtgggattttcccatgagtctcaatattagagtctcaacccccaataaatataggactggagatgtctgaggctcattctgccctcgagcccaccgggaacgaaagagaagctctatctcccctccaggagcccagctatgaactccttctccacaagtaagtgcaggaaatccttagccctggaactgccagcggcggtcgagccctgtgtgagggaggggtgtgtggcccagggagggctggcgggcggccagcagcagaggcaggctcccagctgtgctgtcagctcacccctgcgctcgctcccctccggcacaggcgccttcggtccagttgccttctccctggggctgctcctggtgttgcctgctgccttccctgccccagtacccccaggagaagattccaaagatgtagccgccccacacagacagccactcacctcttcagaacgaattgacaaacaaattcggtacatcctcgacggcatctcagccctgagaaaggaggtgggtaggcttggcgatggggttgaagggcccggtgcgcatgcgttccccttgcccctgcgtgtggccgggggctgcctgcattaggaggtctttgctgggttctagagcactgtagatttgaggccaacggggccgactagactgacttctgtatttatcctttgctggtgtcaggaagttcctttcctttctggaaaatgcagaatgggtctgaaatccatgcccacctttggcatgagctgagggttattgcttctcagggcttccttttccctttccaaaaaattaggtctgtgaagctcctttttgtcccccgggctttggaaggactagaaaagtgccacctgaaaggcatgttcagcttctcagagcagttgcagtactttttggttatgtaaactcaatggctaggattcctcaaagccattccagctaagattcatacctcagagcccaccaaagtggcaaatcataaataggttaaagcatctccccactttcaatgcaaggtattttggtcctgtttggtagaaagaaaagaacacaggaggggagattgggagcccacactcgaattctggttctgccaaaccagccttgtgatcttgggtaaattccctaccacctctggactccatcagtaaaattgggcgtggactaggtgatctcatagatccttcctgctggaacattctatggcttgaattatattctcctaattattgtcaaaattgctgttattaagtatctactgtgtgccaggcactttaaataaatattgtgtctaatcttcaaaacaaatttgcaaggaaggtttttggagataaggaaactgagactcaggattaagtaacacacctaaagtcacaggtgagcttggaactgaacccaagtgtgcccccactccactggaatttgcttgccaggatgccaatgagttgtagcttcatttttcttagagactttcctggctgtggttgaacaatgaaaaggccctctagtggtgtttgttttagggacacttaggtgataacaattctggtattctttcccagacatgtaacaagagtaacatgtgtgaaagcagcaaagaggcactggcagaaaacaacctgaaccttccaaagatggctgaaaaagatggatgcttccaatctggattcaatgaggtaccaacttgtcgcactcacttttcactattccttaggcaaaacttctccctcttgcatgcagtgcctgtatacatatagatccaggcagcaacaaaaagtgggtaaatgtaaagaatgttatgtaaatttcatgaggaggccaacttcaagcttttttaaaggcagtttattcttggacaggtatggccagagatggtgccactgtggtgagattttaacaactgtcaaatgtttaaaactcccacaggtttaattagttcatcctgggaaaggtactctcagggccttttccctctctggctgcccctggcagggtccaggtctgccctccctccctgcccagctcattctccacagtgagataacctgcactgtcttctgattattttataaaaggaggttccagcccagcattaacaagggcaagagtgcaggaagaacatcaagggggacaatcagagaaggatccccattgccacattctagcatctgttgggctttggataaaactaattacatggggcctctgattgtccagttatttaaaatggtgctgtccaatgtcccaaaacatgctgcctaagaggtacttgaagttctctagaggagcagagggaaaagatgtcgaactgtggcaattttaacttttcaaattgattctatctcctggcgataaccaattttcccaccatctttcctcttaggagacttgcctggtgaaaatcatcactggtcttttggagtttgaggtatacctagagtacctccagaacagatttgagagtagtgaggaacaagccagagctgtgcagatgagtacaaaagtcctgatccagttcctgcagaaaaaggtgggtgtgtcctcattccctcaacttggtgtgggggaagacaggctcaaagacagtgtcctggacaactcagggatgcaatgccacttccaaaagagaaggctacacgtaaacaaaagagtctgagaaatagtttctgattgttattgttaaatctttttttgtttgtttggttggttggctctcttctgcaaaggacatcaataactgtattttaaactatatattaactgaggtggattttaacatcaatttttaatagtgcaagagatttaaaaccaaaggcgggggggcgggcagaaaaaagtgcatccaactccagccagtgatccacagaaacaaagaccaaggagcacaaaatgattttaagattttagtcattgccaagtgacattcttctcactgtggttgtttcaattctttttcctaccttttaccagagagttagttcagagaaatggtcagagactcaagggtggaaagaggtaccaaaggctttggccaccagtagctggctattcagacagcagggagtagacttgctggctagcatgtggaggagccaaagctcaataagaaggggcctagaatgaaacccttggtgctgatcctgcctctgccatttctacttaagccagggtttctcatatgttaacatgcatgggaattccctgggcatcttcttgtggtgtggagtctgacttagcaagcctcgggtgggtttgagggtcaaatttctaccaggcttatatccctggtgatgctgcagaattccaggaccacacttggaggtttaaggccttccacaagttacttatcccatatggtgggtctatggaaaggtgtttcccagtcctctttacaccaccggatcagtggtctttcaacagatcctaaagggatggtgagagggaaactggagaaaagtatcagatttagaggccactgaagaacccatattaaaatgcctttaagtatgggctcttcattcatatactaaatatgaactatgtgccaggcattatttcatatgacagaatacaaacaaataagatagtgatgctggtcaggcttggtggctcatgcctgtattccctaaactttgggagcctaaggtgagaactccttgaactcctaaggccaggagttcaagaccagcctggataacatagcaagaccccatctctacaaaaaaccaaaaccaaacaaacaaaaatgatagtggtgcttccctcaggatgcttgtggtctaatgggagacagaacagcaaagggatgattagaagttggttgctgtgagccaggcacagtgctgatataatcccagcgctatgggaggctgaggtgggtggatcatttgaggccaggagtttaagaccagcctggtcaacatggtaaaaccccatctctacttaaaaatacaaaaaagttagccaggcatggtggcatacacctgtaacccagctactcaggaggctgaggcacatgaatcacttgaacccaggaggcagaggttgctgtgcaccactgcactccagcctgggtgacagaacgagaccttgactcaaaaaaaaaaaaaagaagtttgttgctatggaagggtcctactcagagcaggcaccccagttaatctcattcaccccacatttcacatttgaacatcatcccatagcccagagcatccctccactgcaaaggatttattcaacatttaaacaatcctttttactttcattttccttcaggcaaagaatctagatgcaataaccacccctgacccaaccacaaatgccagcctgctgacgaagctgcaggcacagaaccagtggctgcaggacatgacaactcatctcattctgcgcagctttaaggagttcctgcagtccagcctgagggctcttcggcaaatgtagcatgggcacctcagattgttgttgttaatgggcattccttcttctggtcagaaacctgtccactgggcacagaacttatgttgttctctatggagaactaaaagtatgagcgttaggacactattttaattatttttaatttattaatatttaaatatgtgaagctgagttaatttatgtaagtcatatttatatttttaagaagtaccacttgaaacattttatgtattagttttgaaataataatggaaagtggctatgcagtttgaatatcctttgtttcagagccagatcatttcttggaaagtgtaggcttacctcaaataaatggctaacttatacatatttttaaagaaatatttatattgtatttatataatgtataaatggtttttataccaataaatggcattttaaaaaattcagcaactttgagtgtgtcacgtgaagcttaatataaacaagtttcttgtcactgccaccaccacgaccaaaaaaagctaatcaatcactatatataatacatatatatactatatataataaatatatatactatatataatacatatatacactatatataatacatatatactatatatacacatatatactatatatacacatatatattatgaatgtatatatatagtatatatagtatatatactatgtatgtatatatatagtatatatagtatatatactatgtatgtgtatatatagtatatatagtatatatagtatatatactatgtatgtatatatatagtatatatagtatatatactgtgtatgtatatatatagtatatatatactatatatgcatacatagtatatatgcatatatactatatatactatatatttatatatactatatactatatatactatatactgtatatatactatatatgtatgtatacgatatatatatatactatatatgtacacacacacatatatatatacatataagcacctactacatgccaggcatcattaaatgtgttgcatccatcacgtcatttaaccccagcacttgcacactcctttctggttgtggaagactaagtaatttatctaagtcacccagctggaaggtcaggcagggacccagatttgaaatccaagtctacctacctacaggtcccctactcttaacctgtaggtcccactgcctacccaggaactgagggatgatgtagaaaatcccaaaacatgttaatatagggaatacctataaacatgcaatcaaagtctttgggactatacaaccactgtataaagcataacaatgtacaagcttccaaacaataactagaagttctgcctccctcttctgggttcctaaagcactgcacctatctacctgtcaaagcatctaccacattgtaccacaccttaaaatcaatggtttttttcttctcagccagcatgtggatgcctcaataaagcagactcctttcatgacctaaaactaatttcaggggggaaaaaaagacgagctgggcgcagtggctcacgcctataatcccagcactttgggaggctgaggcgggaggatcacttgaggtcagaagaccagcctggccaacatggcaaaaccccgtctctactaaaaatacaaaaattagctgggcgtggtggcgcacctataatcccagctactcaggaagctgagacatgataatcgcttgagcctgggaggtagagcctggggctgcactccatcctgggcaacagagggagattctgtctcaaaaaataataataataatataaataaataaataatttttttaaaaaaagactctttcctatattaatctttgcatcctgtgcccatggccccaaacctgaccaatgaaggccccagtaaatattttttgaacaaaagaaaatatagatgatcaaagataagaaagataagaagacacctcaattcttgtaggcatatagtggtaggggaaatcactaattctgtgtgtttatgtatcttcatcttcaggaaaaatagcaagaattagaaagaaacactcaaaggacttccagagaaggcaatgaatagccagtgcatgtacacctacctccatttcctttgccactggcaataatcttcatctgccctctcccctccccgacctaaagtggtagtgagagaggcaatctggacaacttaacatggcctcgtttatccatctcaacacgacactgggaatttggtgcagatttcccctttctaacccctgcaatataaaagttctgtctccttataaagcttttccccattctacccgctacagacttcttcctccaggggaggagttcttctgggtaagggaaagtccacagaggtcaaaaaccgagaggagctgagccagaaaagagccacatactctgatttctagaaaccacgca

SEQ ID NO: 14 - protein, 212 aa, Homo sapiens
MNSFSTSAFGPVAFSLGLLLVLPAAFPAPVPPGEDSKDVAAPHRQPLTSSERIDKQIRYILDGISALRKETCNKSNMCESSKEALAENNLNLPKMAEKDGCFQSGFNEETCLVKIITGLLEFEVYLEYLQNRFESSEEQARAVQMSTKVLIQFLQKKAKNLDAITTPDPTTNASLLTKLQAQNQWLQDMTTHLILRSFKEFLQSSLRALRQM

SEQ ID NO: 15 - DNA, 50 bp, synthetic construct
  misc_feature 1..50: note = Synthetic oligonucleotide primer
  misc_feature 42: note = n is a, c, g, t or u
  source 1..50: mol_type = other DNA
tcgtcggcagcgtcagatgtgtataagagacagcctacgggnggcwgcag

SEQ ID NO: 16 - DNA, 55 bp, synthetic construct
  misc_feature 1..55: note = Synthetic oligonucleotide primer
  source 1..55: mol_type = other DNA
gtctcgtgggctcggagatgtgtataagagacaggactachvgggtatctaatcc

SEQ ID NO: 17 - DNA, 20 bp, synthetic construct
  misc_feature 1..20: note = Synthetic oligonucleotide primer
  source 1..20: mol_type = other DNA
tgggctggtgctgtgggtag

SEQ ID NO: 18 - DNA, 21 bp, synthetic construct
  misc_feature 1..21: note = Synthetic oligonucleotide primer
  source 1..21: mol_type = other DNA
gatggcgaaggcggttctcac